SEQUENCE LISTING (131 sequences)

SEQ ID NO: 1 - protein, 465 aa, Homo sapiens
MNVTSLFSFTSPAVKRLLGWKQGDEEEKWAEKAVDALVKKLKKKKGAMEELEKALSCPGQPSNCVTIPRSLDGRLQVSHRKGLPHVIYCRVWRWPDLQSHHELKPLECCEFPFGSKQKEVCINPYHYKRVESPVLPPVLVPRHSEYNPQHSLLAQFRNLGQNEPHMPLNATFPDSFQQPNSHPFPHSPNSSYPNSPGSSSSTYPHSPTSSDPGSPFQMPADTPPPAYLPPEDPMTQDGSQPMDTNMMAPPLPSEINRGDVQAVAYEEPKHWCSIVYYELNNRVGEAFHASSTSVLVDGFTDPSNNKNRFCLGLLSNVNRNSTIENTRRHIGKGVHLYYVGGEVYAECLSDSSIFVQSRNCNYHHGFHPTTVCKIPSGCSLKIFNNQEFAQLLAQSVNHGFETVYELTKMCTIRMSFVKGWGAEYHRQDVTSTPCWIEIHLHGPLQWLDKVLTQMGSPHNPISSVS

SEQ ID NO: 2 - DNA, 20 bp, Artificial Sequence
  Description of Artificial Sequence: Synthetic primer
cactggtggggtgaattctt

SEQ ID NO: 3 - DNA, 19 bp, Artificial Sequence
  Description of Artificial Sequence: Synthetic primer
agcactgggcagcactgta

SEQ ID NO: 4 - DNA, 20 bp, Artificial Sequence
  Description of Artificial Sequence: Synthetic primer
tggtcctgagtgtccagttg

SEQ ID NO: 5 - DNA, 20 bp, Artificial Sequence
  Description of Artificial Sequence: Synthetic primer
agaagaggcagtgctgagga

SEQ ID NO: 6 - DNA, 21 bp, Artificial Sequence
  Description of Artificial Sequence: Synthetic primer
ttgccaaattcttgaaacagc

SEQ ID NO: 7 - DNA, 20 bp, Artificial Sequence
  Description of Artificial Sequence: Synthetic primer
gtcccctagaagccaccttt

SEQ ID NO: 8 - DNA, 20 bp, Artificial Sequence
  Description of Artificial Sequence: Synthetic primer
gcacaatcatctggctcaag

SEQ ID NO: 9 - DNA, 20 bp, Artificial Sequence
  Description of Artificial Sequence: Synthetic primer
gagttcagcagagcctccag

SEQ ID NO: 10 - DNA, 20 bp, Artificial Sequence
  Description of Artificial Sequence: Synthetic primer
aggtctggttccctgtgatg

SEQ ID NO: 11 - DNA, 20 bp, Artificial Sequence
  Description of Artificial Sequence: Synthetic primer
ctttctgtccagcaggcaac

SEQ ID NO: 12 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 13 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 14 - DNA, 164 bp, Homo sapiens
tgtcatcctagactctttcctctccttcctctccacttctgatcagtcacctgcccattctttttccctaacatagaattcatcccattgactgtcctttaccatcactgccttaattcaggctttcgtctcttgtgaataaaatcatagtggtttcccctaaa

SEQ ID NO: 15 - DNA, 58 bp, Homo sapiens
ctctttcctctccttcctctccacttctgatcagtcacctgcccattctttttcccta

SEQ ID NO: 16 - DNA, 8 bp, Homo sapiens
gtctagac

SEQ ID NO: 17 - DNA, 10 bp, Homo sapiens
vhgtctgbvb

SEQ ID NO: 18 - DNA, 9 bp, Homo sapiens
aswtcaaag

SEQ ID NO: 19 - DNA, 10 bp, Homo sapiens
  modified_base (4)..(7): a, c, t, g, unknown or other
  misc_feature (4)..(7): This region may encompass 3-4 nucleotides
ttcnnnngaa

SEQ ID NO: 20 - DNA, 17 bp, Homo sapiens
aggtcatggagaggtca

SEQ ID NO: 21 - DNA, 14 bp, Homo sapiens
tttcctagaaagca

SEQ ID NO: 22 - DNA, 14 bp, Homo sapiens
aaagatcaaaggaa

SEQ ID NO: 23 - DNA, 9 bp, Homo sapiens
gccacgtgt

SEQ ID NO: 24 - DNA, 8 bp, Homo sapiens
tgacgtca

SEQ ID NO: 25 - DNA, 10 bp, Homo sapiens
tatgacgtaa

SEQ ID NO: 26 - DNA, 14 bp, Homo sapiens
  modified_base (6)..(6): a, c, t, g, unknown or other
  modified_base (9)..(9): a, c, t, g, unknown or other
ccgcgnggnggcag

SEQ ID NO: 27 - DNA, 13 bp, Homo sapiens
cgaaaccgaaact

SEQ ID NO: 28 - DNA, 10 bp, Homo sapiens
gggaatttcc

SEQ ID NO: 29 - DNA, 9 bp, Homo sapiens
  modified_base (4)..(6): a, c, t, g, unknown or other
gccnnnggc

SEQ ID NO: 30 - DNA, 13 bp, Homo sapiens
aggggattcccct

SEQ ID NO: 31 - DNA, 19 bp, Homo sapiens
gagggcagccaagcgtgac

SEQ ID NO: 32 - DNA, 13 bp, Homo sapiens
  modified_base (7)..(7): a, c, t, g, unknown or other
aggtcanaggtca

SEQ ID NO: 33 - DNA, 15 bp, Homo sapiens
gggtcattgggttca

SEQ ID NO: 34 - DNA, 15 bp, Homo sapiens
aaggtcaaaaggtca

SEQ ID NO: 35 - DNA, 16 bp, Homo sapiens
aaaggtcaaaaggtca

SEQ ID NO: 36 - DNA, 11 bp, Homo sapiens
gggcgggaagg

SEQ ID NO: 37 - DNA, 11 bp, Homo sapiens
taggtgtgaag

SEQ ID NO: 38 - DNA, 11 bp, Homo sapiens
aaggtgtgaag

SEQ ID NO: 39 - DNA, 7 bp, Homo sapiens
atgccaa

SEQ ID NO: 40 - DNA, 5 bp, Homo sapiens
ccaat

SEQ ID NO: 41 - DNA, 6 bp, Homo sapiens
atccac

SEQ ID NO: 42 - DNA, 9 bp, Homo sapiens
aswtcaaag

SEQ ID NO: 43 - DNA, 14 bp, Homo sapiens
cccccgcccccgcc

SEQ ID NO: 44 - skipped

SEQ ID NO: 45 - DNA, 6 bp, Homo sapiens
gaggaa

SEQ ID NO: 46 - DNA, 11 bp, Homo sapiens
rcaggaagtgg

SEQ ID NO: 47 - DNA, 10 bp, Homo sapiens
gcgggggcgg

SEQ ID NO: 48 - DNA, 10 bp, Homo sapiens
agcggaagtg

SEQ ID NO: 49 - DNA, 9 bp, Homo sapiens
dkhgcgtgh

SEQ ID NO: 50 - DNA, 8 bp, Homo sapiens
cmggaagt

SEQ ID NO: 51 - DNA, 11 bp, Homo sapiens
gccccgccccc

SEQ ID NO: 52 - DNA, 15 bp, Homo sapiens
dddscctgrggshdd

SEQ ID NO: 53 - DNA, 10 bp, Homo sapiens
accggaagtg

SEQ ID NO: 54 - DNA, 13 bp, Homo sapiens
aaaaagcggaagt

SEQ ID NO: 55 - DNA, 14 bp, Homo sapiens
aaaaagaggaagta

SEQ ID NO: 56 - DNA, 11 bp, Homo sapiens
ggccacaccca

SEQ ID NO: 57 - DNA, 11 bp, Homo sapiens
catgactcatc

SEQ ID NO: 58 - DNA, 14 bp, Homo sapiens
vddscctgrggshv

SEQ ID NO: 59 - DNA, 9 bp, Homo sapiens
  modified_base (4)..(6): a, c, t, g, unknown or other
gccnnnggc

SEQ ID NO: 60 - DNA, 14 bp, Homo sapiens
ggggcccaaggggg

SEQ ID NO: 61 - DNA, 11 bp, Homo sapiens
gccacgccccc

SEQ ID NO: 62 - DNA, 16 bp, Homo sapiens
  modified_base (11)..(11): a, c, t, g, unknown or other
dddddhwttcnwrgrw

SEQ ID NO: 63 - DNA, 17 bp, Homo sapiens
taagccacgcccccttt

SEQ ID NO: 64 - DNA, 15 bp, Homo sapiens
dddycccwrgggavh

SEQ ID NO: 65 - DNA, 19 bp, Homo sapiens
bbdccrshagrkggcrsbv

SEQ ID NO: 66 - DNA, 10 bp, Homo sapiens
  modified_base (1)..(1): a, c, t, g, unknown or other
ngcgccaaah

SEQ ID NO: 67 - DNA, 11 bp, Homo sapiens
dgvgkgggcgk

SEQ ID NO: 68 - DNA, 9 bp, Homo sapiens
tyaaggtca

SEQ ID NO: 69 - DNA, 13 bp, Homo sapiens
dtgtttacwywdb

SEQ ID NO: 70 - DNA, 15 bp, Homo sapiens
  modified_base (2)..(2): a, c, t, g, unknown or other
bnhtgtttacwtavs

SEQ ID NO: 71 - DNA, 10 bp, Homo sapiens
trtttatytd

SEQ ID NO: 72 - DNA, 13 bp, Homo sapiens
dtgtttatkkttd

SEQ ID NO: 73 - DNA, 13 bp, Homo sapiens
drybtgtttwyhd

SEQ ID NO: 74 - DNA, 19 bp, Homo sapiens
  modified_base (4)..(9): a, c, t, g, unknown or other
vbknnnnnndvwgataasv

SEQ ID NO: 75 - DNA, 11 bp, Homo sapiens
dvagatarvrd

SEQ ID NO: 76 - DNA, 9 bp, Homo sapiens
dvwgatarv

SEQ ID NO: 77 - DNA, 14 bp, Homo sapiens
ddvagataagrddd

SEQ ID NO: 78 - DNA, 11 bp, Homo sapiens
  modified_base (1)..(1): a, c, t, g, unknown or other
ntgggtggtyb

SEQ ID NO: 79 - DNA, 9 bp, Homo sapiens
dkgktgccm

SEQ ID NO: 80 - DNA, 16 bp, Homo sapiens
  modified_base (4)..(5): a, c, t, g, unknown or other
dhsnnvdcggacgtwv

SEQ ID NO: 81 - DNA, 14 bp, Homo sapiens
vsrwbvwkskvgrh

SEQ ID NO: 82 - DNA, 20 bp, Homo sapiens
  modified_base (6)..(6): a, c, t, g, unknown or other
vvrrvngaaasygaaasyvv

SEQ ID NO: 83 - DNA, 10 bp, Homo sapiens
raaabyraaw

SEQ ID NO: 84 - DNA, 9 bp, Homo sapiens
cagggggtg

SEQ ID NO: 85 - DNA, 21 bp, Homo sapiens
  modified_base (5)..(5): a, c, t, g, unknown or other
ddrdnwgctgastcagcaddd

SEQ ID NO: 86 - DNA, 22 bp, Homo sapiens
gggmggrgsvrsrssvssssss

SEQ ID NO: 87 - DNA, 11 bp, Homo sapiens
  modified_base (1)..(1): a, c, t, g, unknown or other
nsggccggmkv

SEQ ID NO: 88 - DNA, 7 bp, Homo sapiens
sccggag

SEQ ID NO: 89 - DNA, 17 bp, Homo sapiens
  modified_base (8)..(8): a, c, t, g, unknown or other
bywttswnwtgywrwdd

SEQ ID NO: 90 - DNA, 9 bp, Homo sapiens
btcaaggab

SEQ ID NO: 91 - DNA, 13 bp, Homo sapiens
wwtaagtawwhdh

SEQ ID NO: 92 - DNA, 14 bp, Homo sapiens
  modified_base (13)..(13): a, c, t, g, unknown or other
wwaavtaggtcand

SEQ ID NO: 93 - DNA, 13 bp, Homo sapiens
rrsbsaraggkmr

SEQ ID NO: 94 - DNA, 13 bp, Homo sapiens
ragktcaagktca

SEQ ID NO: 95 - DNA, 19 bp, Homo sapiens
ddrcwdgyhkgrrcwygyh

SEQ ID NO: 96 - DNA, 16 bp, Homo sapiens
bywttvwhatgcadwh

SEQ ID NO: 97 - DNA, 14 bp, Homo sapiens
drmagwgaaagtdh

SEQ ID NO: 98 - DNA, 11 bp, Homo sapiens
btgtggtkdbb

SEQ ID NO: 99 - DNA, 11 bp, Homo sapiens
  modified_base (1)..(1): a, c, t, g, unknown or other
nbbtgtggtyw

SEQ ID NO: 100 - DNA, 7 bp, Homo sapiens
  modified_base (1)..(1): a, c, t, g, unknown or other
ncaggtg

SEQ ID NO: 101 - DNA, 16 bp, Homo sapiens
yywttstbmtkswdwh

SEQ ID NO: 102 - DNA, 22 bp, Homo sapiens
  modified_base (18)..(18): a, c, t, g, unknown or other
svvvvrrrggcggrrsbnvvsv

SEQ ID NO: 103 - DNA, 11 bp, Homo sapiens
vrtsrsswgwb

SEQ ID NO: 104 - DNA, 13 bp, Homo sapiens
  modified_base (12)..(12): a, c, t, g, unknown or other
vvvwggvswgrnb

SEQ ID NO: 105 - DNA, 15 bp, Homo sapiens
  modified_base (9)..(9): a, c, t, g, unknown or other
dhrstttcnbttyyh

SEQ ID NO: 106 - DNA, 19 bp, Homo sapiens
  modified_base (5)..(10): a, c, t, g, unknown or other
bykbnnnnnnbwgataavv

SEQ ID NO: 107 - DNA, 11 bp, Homo sapiens
vycagctgyvg

SEQ ID NO: 108 - DNA, 10 bp, Homo sapiens
rrwcaygtgv

SEQ ID NO: 109 - DNA, 17 bp, Homo sapiens
vrvsyvmbvksaggtca

SEQ ID NO: 110 - DNA, 12 bp, Homo sapiens
gacgtgtmhhwd

SEQ ID NO: 111 - DNA, 9 bp, Homo sapiens
kgggwgskv

SEQ ID NO: 112 - DNA, 22 bp, Homo sapiens
kmddkgmakkmtgggwrdksbh

SEQ ID NO: 113 - DNA, 20 bp, Artificial Sequence
  Description of Artificial Sequence: Synthetic primer
agctgcactgtgacaagctg

SEQ ID NO: 114 - DNA, 20 bp, Artificial Sequence
  Description of Artificial Sequence: Synthetic primer
ctgtcattcgttcgtcctca

SEQ ID NO: 115 - DNA, 19 bp, Artificial Sequence
  Description of Artificial Sequence: Synthetic primer
ctgcaggacttcaccaagg

SEQ ID NO: 116 - DNA, 20 bp, Artificial Sequence
  Description of Artificial Sequence: Synthetic primer
ttggcctggatacagaagga

SEQ ID NO: 117 - DNA, 20 bp, Artificial Sequence
  Description of Artificial Sequence: Synthetic primer
tccttctgaaccacctccac

SEQ ID NO: 118 - DNA, 20 bp, Artificial Sequence
  Description of Artificial Sequence: Synthetic primer
ggggctacagagggaagaga

SEQ ID NO: 119 - DNA, 19 bp, Artificial Sequence
  Description of Artificial Sequence: Synthetic primer
acgacgagctcgacttcaa

SEQ ID NO: 120 - DNA, 20 bp, Artificial Sequence
  Description of Artificial Sequence: Synthetic primer
gcaaagtgcgtgaggagttt

SEQ ID NO: 121 - DNA, 19 bp, Artificial Sequence
  Description of Artificial Sequence: Synthetic primer
ctgctcaaggagttcccgt

SEQ ID NO: 122 - DNA, 20 bp, Artificial Sequence
  Description of Artificial Sequence: Synthetic primer
gtctgccaccatcttcgact

SEQ ID NO: 123 - DNA, 276 bp, Homo sapiens
ggggtgtaggtgtaatttgaatgcagcatgttccagaaagatcagtgtggaaagtggacctagtagcagaattgctagataacaactaaggatgggaagcgggggaagcacatgggaggattccccagcttctgctaaaattcaaccagggtccattctctgtgtggattaacttttggggaatacaataacagctgaaaaagcaatggaaattttctaatttttcttgggttattcaaatcatgattatttggaaaactgtgtgtggttttttta

SEQ ID NO: 124 - DNA, 12 bp, Homo sapiens
agtctctgtgtc

SEQ ID NO: 125 - DNA, 12 bp, Homo sapiens
agtctcagtgtc

SEQ ID NO: 126 - DNA, 29 bp, Homo sapiens
cgataagtgtctggctctgcttatcagcc

SEQ ID NO: 127 - DNA, 29 bp, Homo sapiens
cgataagtgcctggctctgcttatcagcc

SEQ ID NO: 128 - DNA, 11 bp, Homo sapiens
aagtgtctggg

SEQ ID NO: 129 - DNA, 11 bp, Homo sapiens
aagtgcctggg

SEQ ID NO: 130 - DNA, 12 bp, Homo sapiens
agtctctgtgtc

SEQ ID NO: 131 - DNA, 12 bp, Homo sapiens
agtctcagtgtc

(19) United States
(12) Patent Application Publication
(10) Pub. No.: US 2019/0002886 A1
(43) Pub. Date: Jan. 3, 2019

(54) SIGNALING CENTERS OF ERYTHROID DIFFERENTIATION

(71) Applicant: THE CHILDREN'S MEDICAL CENTER CORPORATION, Boston, MA (US)

(72) Inventors: Leonard ZON, Wellesley, MA (US); Avik CHOUDHURI, Belmont, MA (US); Eirini TROMPOUKI, Freiburg (DE)

(73) Assignee: THE CHILDREN'S MEDICAL CENTER CORPORATION, Boston, MA (US)

(21) Appl. No.: 16/016,007

(22) Filed: Jun. 22, 2018

Related U.S. Application Data
(60) Provisional application No. 62/523,499, filed on Jun. 22, 2017.

(51) Int. Cl. C12N 15/113; C12N 5/074; A61P 37/00; A61P 35/02; C12N 5/0789

(52) U.S. Cl. CPC C12N 15/1138; C12N 5/0696; C12N 2310/20; A61P 35/02; C12N 5/0647; A61P 37/00

(57) ABSTRACT

Described herein are methods, compounds, pharmaceutical compositions, and kits for modulating erythropoiesis by altering occupancy at genomic signaling-centers.

### CROSS-REFERENCE TO RELATED APPLICATION

This Application claims benefit under 35 U.S.C. § 119(e) of the U.S. Provisional Application No. 62/523,499 filed Jun. 22, 2017, the contents of which are incorporated herein by reference in their entirety.

### GOVERNMENT SUPPORT

This invention was made with Government support under Grant No.: R01HL04880-24 awarded by the National Institutes of Health. The Government has certain rights in the invention.

### FIELD OF THE INVENTION

Embodiments of the invention relate generally to compounds, methods, compositions, and kits for modulating erythropoiesis by altering occupancy at genomic signaling-centers that have binding sites for lineage-specific regulators and signal-responsive transcription factors.

### BACKGROUND

Hematopoietic progenitors respond to developmental and environmental cues to differentiate through characteristic intermediate cell identities, which are largely controlled by transcription. During physiological processes, like hematopoietic differentiation, there is a rapid turnover of distinct cell stages with differing transcription programs and gene expression. As in most differentiation processes, erythropoiesis is accompanied by differential genomic binding of signal-responsive and lineage-restricted transcription factors that regulate these expression differences. Transcription factors preferentially accumulate to proximal and distal DNA regulatory elements, namely enhancers (Heinz et al., 2015). At least one million enhancers have been identified in the human genome yet complete understanding of how enhancers evolve and change in protein complement during a continuous process, such as differentiation remains elusive (Bulger and Groudine, 2011; Consortium, 2012).

It is widely accepted that lineage or “master” regulators exert control over the transcriptional programs that govern cell fate decision and ultimately cell differentiation. The lineage regulators GATA2 and GATA1 control the expression programs of cells at different stages of erythropoiesis; GATA2 maintains the identity of hematopoietic stem and progenitor cells, while GATA1 is indispensable for establishing the erythroid program. During erythroid differentiation, GATA2 is down-regulated while GATA1 is up-regulated and is known to replace GATA2 on a number of regulatory elements, comprising a “GATA switch” (Bresnick et al., 2010; Cantor and Orkin, 2002). Many genome-wide studies of erythroid differentiation show that these transcription factors occupy thousands of genomic regions thought to act in transcription regulation (Cheng et al., 2009; Dore et al., 2012; Fujiwara et al., 2009). Correlation of the GATA-occupied sites with gene expression shows that only a small proportion of GATA-bound genes is highly expressed during differentiation (Bresnick et al., 2010; Cantor and Orkin, 2002). This strongly indicates that other transcriptional regulators contribute to the control of stage-specific gene expression.

Signaling pathways converge on signal-induced transcription factors, which also control gene expression by binding transcriptional regulatory elements. The same signaling pathways play important roles in regulating expression of multiple cell types and can exert tissue-specific functions using the same sets of signal-induced transcription factors albeit at different transcriptional regulatory elements. Members of the TGFβ, BMP and Wnt pathways are critical for multiple tissues and co-localize with lineage. specific factors in different cell types (Mullen et al., 2011; Trompouki et al., 2011). BMP signaling is important during developmental erythropoiesis in Xenopus and zebrafish but can also boost adult hematopoietic regeneration and differentiation of hematopoietic progenitors into erythroid and myeloid lineages (Detmer and Walker, 2002; Fuchs et al., 2002; Lenox et al., 2005; Schmerer and Evans, 2003; Zhang and Evans, 1996).

While a lot of progress has been made, there remains a need in the art to identify factors that contribute to the control of stage-specific gene expression, not only to gain insights into the regulators of erythropoiesis, but also to identify targets for therapeutics to increase or decrease red blood cell production.

### SUMMARY OF THE INVENTION

Embodiments of the invention are based on the discovery stage-specific genomic signaling-centers that drive erythropoiesis of CD34⁺ cells. These stage specific signaling-centers have been determined to have DNA binding sites for both lineage-specific regulators (e.g. GATA) and for signal-responsive transcription factors, as well as in some instances tissue specific factors. Gene expression at these signaling centers can be modulated using agents that alter occupancy of the signaling centers, e.g. modulate binding of signal-responsive transcription factors, or modulate binding of other regulatory factors to the signaling-center.

Accordingly, described herein are methods for modulating erythropoiesis using agents that alter binding of these factors to the stage-specific signaling centers. In one aspect of the invention, a method for modulating erythropoiesis is provided. The method comprises contacting a CD34⁺ cell with an agent that alters occupancy at a signaling center in the genome of the cell, wherein the signaling center comprises 1) a DNA binding site for a lineage-specific regulator, and 2) a DNA binding site for a signal-responsive transcription factor, wherein increasing gene expression at the signaling center promotes erythropoiesis. In certain embodiments, the signaling center further comprises a tissue-specific transcription factor DNA binding motif. Non-limiting examples include a binding motif for PU.1; FL1; KROX; ETV6; CETS1PS4; FL11; SP1C; ETS; ETS1; SP11; SP1B, KLF1, NFE4, EKLF, SP2, KROX, KLF16, AP2, PLAG1, SP3, FKLF, SP4 (See for example, Boeva et al: analysis of genomic sequence motifs for deciphering transcription factor binding and transcriptional regulation in eukaryotic cells, (2016) Frontier Genetics 7:24), which is incorporated herein by reference in its entirety.

In one embodiment, the agent that alters occupancy at the signaling center is an agent that induces binding of the signal-responsive transcription factor to the signaling center.

In one embodiment, the agent that alters occupancy at the signaling center is an agent that inhibits binding of the signal-responsive transcription factor to the signaling center.

In one embodiment, the signal-responsive transcription factor is selected from the group consisting of SMAD1, SMAD5, SMAD8, β-catenin, LEF/TCF, STAT5, RARA, BCL11A, TCF7L2, CREB3L, CREB, CREM, CTCF, IRF7, RELB, AP2B, NFKB2, PAX, PPARG, RXRA, RARG, RARB, E2F6m TBX20, TBX1, NFIA, NFIB, ZN350, TCF4, EGR1, and THRB

In one embodiment, the agent that alters occupancy at the signaling center in the genome is an agonist of a signaling pathway selected from the group consisting of: nuclear hormone receptor, cAMP pathway, MAPK pathway, JAK-STAT pathway, NFKB pathway, Wnt pathway, TGF-β pathway, LIF pathway, BDNF pathway, PGE2 pathway, and NOTCH pathway.

In one embodiment, the agent that alters occupancy at the signaling center is a small molecule, a nucleic acid RNA, a nucleic acid DNA, a protein, a peptide, or an antibody.

In one embodiment, the lineage-specific regulator is the transcription factor GATA1 or GATA2.

In one embodiment, the signaling center comprises the signal-responsive binding site for transcription factor SMAD1 and the lineage-specific regulator binding site for the transcription factor GATA1, and wherein the agent that alters occupancy at the signaling center increases expression of one or more genes selected from Table 4 (D5 SE genes), or from Lengthy Table S6, See Lengthy Table S6 for Signaling-center location and signaling-centers that co-bind SMAD1 and GATA1 at days 0, 3, and 4. Note that in Lengthy Table 6, the data is separated by commas and depicts the following order of data regarding the SE signaling-centers for Day 0, Hour 6, Day 3, Day 4, or Day 5 of differentiation: Chromosome (e.g. chr 2), Start (e.g. 87752755), End (87958941), ID (e.g. 42_MACS_Peak_28785_locistitched), Gene_ID (e.g. NR_024204), Gene_Name (e.g. NCRNA00152), D0_GATA1 bound (e.g. 5), D0_GATA2 bound (e.g. 12), D0_SMAD1 bound (e.g. 3), D0_GATA1+SMAD1 (e.g. 2), D0_GATA2+SMAD1 (e.g. 2), H6_GATA1 bound (e.g. 0), H6_GATA2 bound (e.g. 17), H6_SMAD1 bound (e.g. 13), H6_GATA1+SMAD1 (e.g. 0), H6_GATA2+SMAD1 (e.g. 5), D3_GATA1 bound (e.g. 11), D3_GATA2 bound (e.g. 13), D3_SMAD1 bound (e.g. 11), D3_GATA1+SMAD1 (e.g. 10), D3_GATA2+SMAD1 (e.g. 10), D4_GATA1 bound (e.g. 7), D4_GATA2 bound (e.g. 18), D4_SMAD1 bound (e.g. 17), D4_GATA1+SMAD1 (e.g. 7), D4_GATA2+SMAD1 (e.g. 11), D5_GATA1 bound (e.g. 11), D5_GATA2 bound (e.g. 1), D5_SMAD1 bound (e.g. 9), D5_GATA1+SMAD1 (e.g. 8), D5_GATA2+SMAD1 (e.g. 1); e.g. are for Day 5, but data for all days is separated by comas in the same order.

In one embodiment, the signaling center comprises the signal-responsive binding site for transcription factor SMAD1 and the lineage-specific regulator binding site for the transcription factor GATA1, and wherein the agent that alters occupancy at the signaling center increases expression of one or more genes selected from Lengthy Table 51, Table 5, or Table 6 (Note: genes can be cross referenced with lengthy Table 51 to find the signaling center location).

In one embodiment, the signaling center comprises the signal-responsive transcription factor binding site for SMAD1 and the lineage-specific regulator binding site for the transcription factor GATA2, and the agent that alters occupancy at the signaling center increases expression of one or more genes selected from Table 3 (H6 SE genes), or from Lengthy Table S6. See Lengthy Table 6 for signaling-center location, and signaling centers that co-bind SMAD1 and GATA2 at days 0, 3, and 4.

In one embodiment, the signaling center comprises the signal-responsive transcription factor binding site for SMAD1 and the lineage-specific regulator binding site for the transcription factor GATA2, and the agent that alters occupancy at the signaling center increases expression of one or more genes selected from Lengthy Table 51, Table 5, or Table 6 (Note: genes can be cross referenced with lengthy Table 51 to find the signaling center location).

In one embodiment, the signaling center comprises the signal-responsive transcription factor binding site and a GATA1 or GATA2 binding site, and the agent that alters occupancy at the signaling center increases expression of one or more genes selected from Lengthy Table 51, Lengthy Table 51, Table 5, or Table 6 (Note: genes can be cross referenced with lengthy Table 51 to find the signaling center location).

In one embodiment, the signaling center comprises the signal-responsive transcription factor binding site for SMAD1, and the agent that alters occupancy at the signaling center increases expression of one or more genes selected from Lengthy Table 51.

In certain embodiments, the agent that alters occupancy at the signaling center is an agent that activates the transcription factor SMAD1. In one embodiment, the agent is an agonist of a BMP receptor kinase or a checkpoint kinase 1 (CHK1) inhibitor.

In one embodiment the agent that activates SMAD1 is selected from the group consisting of: PD407824, MK-8776, LY-2606368 and LY-2603618, BMP4, BMP2, BMP7, isoliquirtigenin, apigenin, 4′-hydroxychalcone, and diosmetin.

In one embodiment, the signaling center comprises the signal-responsive binding site for transcription factor SMAD1 and the lineage-specific regulator binding site for the transcription factor GATA1 or GATA2, and wherein co-binding of either SMAD1/GATA1 or SMAD/GATA2 at the signaling center alters expression of long non-coding RNAs (IncRNAS), See e.g. the IncaRNAs listed in Lengthy Table S5.

In one embodiment, the CD34⁺ cell is ex vivo and derived from a source selected from the group consisting of: bone marrow, peripheral blood, cord blood and derived from induced pluripotent stem cells.

In one embodiment, the CD34+ cell is in vivo and an effective amount of an agent that alters occupancy of the signaling center is administered to a subject, i.e. the contacting step is performed in vivo.

In one embodiment, the CD34⁺ cell is ex vivo. In one embodiment, the cells treated with the agent are transplanted back to the subject. In one embodiment, the cell is contacted additional agents known to modulate eyrthropoeisis, e.g. EPO, or other agents.

Another aspect of the invention provides methods for treating diseases associated with aberrant erythropoiesis. The methods comprise correcting the DNA of a CD34⁺ cell that is present at the site of a signaling center, wherein the signaling center associated with normal erythropoiesis comprises 1) a DNA binding site for a lineage-specific regulator, and 2) a DNA binding site for a signal-responsive transcription factor.

In one embodiment, the correction of the DNA restores the binding of the signal-responsive transcription factor to the signaling center. Restoring binding of the signal-responsive transcription factor at a signaling center can be accomplished by either creating the normal binding site for the signal-responsive transcription factor, or by destroying an aberrant binding site not normally present that disrupts binding of the signal-responsive transcription factor.

In one embodiment, the lineage-specific regulator is transcription factor GATA1 or GATA2.

In one embodiment, the signal-responsive transcription factor is selected from the group consisting of SMAD1, SMAD5, SMAD8, β-catenin, LEF/TCF, STAT5, RARA, BCL11A, TCF7L2, CREB3L, CREB, CREM, CTCF, IRF7, RELB, AP2B, NFKB2, PAX, PPARG, RXRA, RARG, RARB, E2F6m TBX20, TBX1, NFIA, NFIB, ZN350, TCF4, EGR1, and THRB

In one embodiment, the signaling center further comprises a tissue-specific transcription factor DNA binding motif, non-limiting example include motifs in progenitor cells: e.g. PU.1, FL1, KROX, ETV6, CETS1PS4, FL11, SP1C, ETS, ETS1, SP11, SP1B; or binding motif of erythroid cells, e.g. KLF1, NFE4, EKLF, SP2, KROX, KLF16, AP2, PLAG1, SP3, FKLF, SP4, See e.g FIG. 27.

In one embodiment, the DNA is corrected using a gene editing tool.

In one embodiment the gene editing tool is CRISPER technology or TALEN Technology, tools that are well known to those of skill in the art, See e.g. WO 2013/163628. US 2016/0208243, and US 2016/0201089.

In one embodiment, the disease associated with aberrant erythropoiesis is selected from the group consisting of: leukemia, lymphoma, inherited anemia, inborn errors of metabolism, aplastic anemia, beta-thalassemia, Blackfan-Diamond syndrome, globoid cell leukodystrophy, sickle cell anemia, severe combined immunodeficiency, X-linked lymphoproliferative syndrome, Wiskott-Aldrich syndrome, Hunter's syndrome, Hurler's syndrome Lesch Nyhan syndrome, osteopetrosis, chemotherapy rescue of the immune system, and an autoimmune disease.

In one embodiment, the signal-responsive binding site is the binding site for the transcription factor SMAD1, and wherein restoring binding of SMAD1 to the signaling center increases expression of one or more genes selected from Tables 3-4, or from Lengthy Table 6. See Lengthy Table S6 for Signaling-center location and Signaling-centers that co-bind SMAD1 and GATA at days 0, 3, and 4.

In one embodiment, the CD34⁺ cell is in vivo. In one embodiment, the CD34⁺ cell is ex vivo and the CD34⁺ cell is transplanted into the subject after correction of the DNA at the site of the signaling-center.

In certain embodiments of these aspects, the CD34⁺ cell is present in a population of CD34⁺ cells. In one embodiment, the population of CD34⁺ cells comprises hematopoietic stem cells e.g. that are CD34⁽⁺⁾⁽⁻⁾, CD38⁽⁺⁾⁽⁻⁾, CD45RA⁻, CD49f⁺ and CD90⁺. In one embodiment, the population of CD34⁺ cells comprises hematopoietic progenitor cells, e.g. that are that are CD34⁺, CD45RA⁺, CD38⁺. In one embodiment, the population of CD34⁺ cells comprise erytrhoid lineage committed cells, e.g. that are that are CD34⁺, CD38⁺ and CD45RA⁻.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIGS. 1A-1B show schematics, images and graphs which indicate BMP signaling affects erythroid differentiation of human CD34⁺ cells. (FIG. 1A) Schematic of human CD34+ cells from mobilized peripheral blood as they differentiate towards erythrocytes. Summary of experiments performed at Day 0 (D0), Hour 6 (H6), Day 3 (D3), Day 4 (D4) and Day 5 (D5) are also shown. (FIG. 1B) FACS analysis for CD71 and CD235a on BMP4- and dorsomorphin-treated CD34⁺ cells. CD34⁺ cells were treated with rhBMP4 or dorsomorphin at D3 of differentiation and analysis was done at D5 of differentiation. FACS analysis and fold changes of the number of CD71 and CD235a double positive cells are as indicated (*=p-value<0.05).

FIGS. 2A-2D are graphs indicating GATA2 and GATA1 lose and gain bound regions, respectively, but SMAD1 binding is more versatile during differentiation. (FIG. 2A) Region heatmap depicting signal of ChIP-Seq reads for GATA2 (red), GATA1 (blue) and SMAD1 (green) at D0, H6, D3, D4 and D5 of differentiation. (FIG. 2B) Binary plots showing temporal dynamics of SMAD1, GATA2 and GATA1 binding during the time-course. Rows are regions representing the union of peaks identified separately at D0 through D5. Rows are colored if that region is considered enriched for a factor at that time-point. Rows are ranked by how frequently that region is considered a peak across the whole time-course. (FIG. 2C) Gene tracks representing binding of GATA2, GATA1 and SMAD1 at D0, H6, D3 and D5 of differentiation at an exemplary “progenitor gene” (FLT3) and an “erythroid gene” (ALAS2). (FIG. 2D) Paired-time-point heatmaps comparing SMAD1-enriched regions between D0 and H6, H6 and D3, D3 and D4, D4 and D5 (Top Panel). Gene tracks depicting how SMAD1 binding changes between consecutive time points (Bottom Panel). See also FIG. 8.

FIGS. 3A-3E indicate that genes co-bound by GATA1/2 and SMAD1 show higher expression. (FIG. 3A) Heat map depicting correlation of gene expression profiles of all the protein-coding RNAs from D0 through D8 of erythroid differentiation. Progenitor and erythroid clusters separate around D3. (FIG. 3B) Pathway analysis comparing genes that undergo “GATA-switch” and subsequently experience increase or decrease in expression from H6 to D5. (FIG. 3C) Boxplots showing distribution of Reads Per Kilobase per Million (RPKM) expression values for genes bound either by GATA factors and SMAD1 together or only by GATA factors during subsequent stages erythroid differentiation. Results of KS significance test are also presented. (FIG. 3D) qPCR analysis of genes bound by GATA1 and SMAD1 (HBB, ALAS2, SLC4A1, DYRK3 and UROS) or by only GATA1 (SH2D6, NFATC3, KCNK5, ZFP36L1 and LMNA) after continuous dorsomorphin treatment for two days starting from D3. (FIG. 3E) Representative gene tracks that show ChIP seq binding for GATA1 and SMAD1, and RNAseq expression for a gene co-bound by GATA1 and SMAD1 (ALAS2) versus a gene bound by GATA1 alone (NFATC3) at D5 of differentiation.

FIGS. 4A-4D indicate that novel lncRNAs are expressed during human erythroid differentiation. (FIG. 4A) Heat maps depicting the annotated, novel and union of both lncRNAs during human erythroid differentiation. A progenitor and an erythroid lncRNA cluster are observed around D3 of differentiation. (FIG. 4B) Graph of supervised hierarchical clustering of novel lncRNAs according to their expression throughout the erythroid differentiation time-course. (FIG. 4C) Top Panel: pie charts showing percentages of lncRNA.genes bound and non-bound by GATA2 at H6. Only GATA2-bound and GATA2+SMAD1 co-bound lncRNA.genes are also shown. Bottom Panel: pie charts showing percentages of lncRNA.genes bound and non-bound by GATA1 at D5. Only GATA1-bound and GATA1+SMAD1 co-bound lncRNA.genes are also shown. (FIG. 4D) Representative gene tracks (showing GATA2/1 and SMAD1 binding and RNAseq expression) of two novel lncRNAs that are targets of “GATA-switch”. One is upregulated and the other is downregulated from H6 to D5. Gradual changes in RPKM-values in each example are indicated at H6, D3 and D5. See also FIG. 9.

FIGS. 5A-5D Indicate co-binding of GATA1/2 and SMAD1 at stage-specific super. enhancers (FIG. 5A) Percentage of SEs bound by GATA2 (Top Left Panel) or GATA1 (Top Right Panel) out of total number of SEs present at various stages of erythroid differentiation. Percentages of GATAThound SEs that are co-bound by SMAD1 (Bottom Left Panel) and percentages of GATA1 bound SEs that are co-bound by SMAD1 (Bottom Right Panel) at each stage of differentiation. (FIG. 5B) Heatmaps showing occupancy of SMAD1 at H6 specific SEs (GATA2-bound), SEs shared between H6 and D5 (GATA2 or GATA1 bound) and D5-specific SEs (GATA1 bound). (FIG. 5C) Representative gene tracks of H6 specific SEs co-bound by GATA2 and SMAD1 at H6 (GATA2, CEBPA), shared H6 and D5 SEs co-bound by GATA2 and SMAD1 at H6 and co-bound by GATA1 and SMAD1 at D5 (TAL1, LYL1), and D5 specific SEs cobound by GATA1 and SMAD1 at D5 (BRD4, BCL11A). (FIG. 5D) Boxplots representing the correlation of GATA/SMAD1 co-bound versus GATA-only bound SEs with the corresponding gene expression at H6 and D5 of human erythroid differentiation. Y-axis in Left Panel represents Log 2[(H6 RPKM/D5 RPKM)] where as Y-axis in Right Panel represents Log 2[(D5 RPKM/H6 RPKM)]. See also FIG. 10.

FIGS. 6A-6D. GATA1/2 and SMAD1 co-bound regions but not GATA-only regions are located in open chromatin. (FIG. 6A) Representative ATAC-seq tracks for two progenitor-specific genes (CD38,FLI1) and two erythroid-specific genes (HBE1, GYPA) over the course of differentiation (D0, H6, D1, D2, D3, D4 and D5). (FIG. 6B) Representative gene tracks showing GATA2, GATA1 and ATAC-seq peaks at D3, D4 and D5 of differentiation. GATA1 binding at D3 is followed by an ATAC-seq peak at D4. (FIG. 6C) Correlation plots comparing median peak intensities for ChIP-seq and ATAC-seq at regions that are co-bound by GATA2/1 and SMAD1 versus the GATA2/1 alone. Time-points compared are as indicated. (FIG. 6D) Representative gene tracks for a progenitor-specific gene (FLT3) and an erythroid-specific gene (ALAS2) showing binding of GATA2, GATA1 and SMAD1 at regions that are enriched with ATAC-seq peaks during the course of differentiation (H6, D4 and D5). See also FIG. 11.

FIGS. 7A-7B Indicate regions co-bound by GATA2/1 and SMAD1 are hotspots for cell-type-specific transcription factors. (FIG. 7A) Bar charts depicting the enrichment of specific transcription factor motifs at regions co-bound by GATA+SMAD1 (left) versus by GATA only (right) at H6 (Left Panel) and D5 (Right Panel). Length of the bar indicates the fraction of peaks containing a given motif, and the number associated with the bar represents the corresponding −log 10(p-value) obtained from the hyper-geometric test to assess the significance of motif enrichment. (FIG. 7B) Relative enrichment of PU.1 and KLF1 binding at GATA2/1+SMAD1 versus GATA2/1 sites at respective time-points, as indicated.

FIGS. 8A-8F are ChIP-Seq graphs of binding data (FIGS. 8A-8b, FIGS. 8d-8F) indicating co-bound GATA/SMAD regions during erythropoiesis. FIG. 8C, is a chart of representative genes undergoing the GATA switch. FIG. 8E are maps of the ingenuity analysis showing predicted upstream regulators of the co-bound genes during erythopoeisis Day 0, hour 6, day 3, day 4, day 5.

FIGS. 9A-9B Indicate that lncRNA gene expression depends on GATA/SMAD1 binding. Related to FIG. 4. (FIG. 9A) Box plots correlating the expression of non-GATA2-bound, only GATA2-bound and GATA2+SMAD1 co-bound lncRNAs at H6 of differentiation. (FIG. 9B) Box plots correlating expression of non-GATA1-bound, only GATA1-bound and GATA1-SMAD1 co-bound lncRNAs at D5 of differentiation. Results of Welch's t-test for significance are also presented in both cases.

FIGS. 10A-10D Indicate that GATA2/1 and SMAD1 co-localize at tissue-specific SEs. Related to FIG. 5. (FIG. 10A) Left Panel: A comparative classification to identify the top 150 most H6-specific, D5-specific and shared SEs based on H3K27ac signal in the union of enhancers separately defined in H6 and D5. The plot compares Log 2(fold change) of H3K27ac signal for individual SEs at H6 and D5. H6 and D5-specific SEs are shown in blue and red, respectively. SEs shared between H6 and D5, which have the most equivalent H6 and D5 signal, are indicated in violet. Right Panel: Heat map depicting “GATA-switch” at SEs shared between H6 and D5. (FIG. 10B) Boxplots showing expression-correlation of all the H6-specific SEs in comparison with the D5-specific SEs (Left Panel) and vice-versa (Right Panel). Y-axis in Left Panel represents Log 2[(H6-RPKM/D5-RPKM)] where as Y-axis in Right Panel represents Log 2[(D5-RPKM/H6-RPKM)]. (FIG. 10C) Ingenuity analysis heatmaps that reveal predicted upstream regulators, diseases and bio-functions and canonical pathways for all SEs at H6 and D5. (FIG. 10D) Ingenuity analysis heatmaps that reveal predicted upstream regulators, diseases and bio-functions and canonical pathways for all SEs co-bound by GATA2/1 and SMAD1 at H6 and D5.

FIG. 11 indicates ATAC-seq peaks reveal tissue specificity. Related to FIG. 6. GREAT analysis showing progenitor-specific and erythroid-specific signatures of ATAC-seq peak-enriched regions at H6 and D5, respectively.

FIG. 12 is a schematic of the involvement of SMA1 in erythroid differentiation. SMAD1 co-localizes with GATA1 as differentiation progresses into ProE cells.

FIG. 13 Indicates that BMP-signaling factor SMAD1 defines critical “signaling centers in various hematopoietic cells. Left side: graph of overlap of CH1Pseq of SMAD1. TCFL2 on GATA2/1 and C/EBPa-sites at representative genes K562 and U937 cells, respective (Trompuki and Brown wt al., Cell 2011). Right Side: Graph of overlap of pCREB-, SMAD1-, TCF7L2- and Gata2-CH1Pseq on ATACseq peaks at representative genes in progenitor CD34 cells.

FIG. 14 Indicates that over-expression of BMP help regenerate hematopoietic system after irradiation. The graphs show the recovery of hematopoietic precursors in post-irradiated Zebrafish and concomitant analysis of gene expression of key hematopoietic genes after BMP and WNT Stimulation. BMP and WNT signaling promote recovery of post-irradiation hematopoietic system indicating active participation of Signaling Centers to activate critical gene-networks required for hematopoietic regeneration.

FIG. 15 Indicates that BMP-signaling promotes differentiation in human CD34⁺ cells. Facs analysis and graphs show that BMP signaling induce erythroid differentiation whereas inhibition of BMP signaling inhibits erythroid commitment in human CD34 cells. This observation indicates a role of signaling pathways in defining cell-fate during human erythropoiesis.

FIG. 16 is a schematic depicting a working Hypothesis: i.e. SMAD1, in close proximity to lineage restricted master regulators, defines Signaling Centers that change at every step of human erythropoiesis, in turn, determines stage-specific gene expression.

FIG. 17 are RNAseq graphs of gene expression dynamics during human erythropoiesis indicating that global clustering of RNAseq as well as expression of representative erythroid-specific genes specifies day 3 of differentiation as erythroid commitment time-point for human CD32 progenitors.

FIG. 18 are ATACseq graphs that indicate that co-binding of GATA factors and SMAD1 marks the formation of stage specific “Signaling-Centers.” Global clustering of ATACseq peaks supports day 3 as erythroid commitment time-point. ATACseq peaks identifies open chromatin regions that remarkably overlaps with GATA and SMAD1 co-bound regions.

FIG. 19 are super enhancer peak graphs that indicate Signaling-Centers mark stage-specific super enhancers. SMAD1 occupied Signaling Centers mark Super Enhancers (SE) that define distinct stages of erythroid differentiation.

FIG. 20 Are graphs that depict differential enrichment of tissue-specific factor motifs at SMAD1+GATA and GATA-only sites. SMAD1+GATA-co-occupied signaling hotspots are enriched with cell-type specific transcription factors

FIG. 21 Top panel is a graph and sequence (SEQ ID NO: 14) showing the Pu1, GATA2, and SMAD1 motif. Lower panel is a graph indicating that disrupting PU1 and GATA motif in the GATA2+SMAD1-cobound enhancer region using Crisper severely decreases gene expression of the nearby gene (LHFPL2). GATA (SEQ ID NO: 15), PU1 (SEQ ID NO: 15), SMAD1 (SEQ ID NO: 15), GATA-PU1 (SEQ ID NO: 15); the X on the sequence of SEQ ID NO:15 represents disruption of binding. This observation indicates that lineage-restricted master regulators play critical role in the formation of stage-specific Signaling Centers.

FIGS. 22A-22C is a schematic pie chart (FIG. 22A) showing signaling centers mark the SNPs associated with red blood cells trait, and gene-track graphs (FIGS. 22B and 22C) showing SNPs within GATA1+SMAD1 co-bound peaks. Analysis of human single nucleotide polymorphisms (SNPs) revealed that SMAD1-binding at erythroid stage remarkably overlaps with red-blood-cell-trait-associated variations. Out of 108 genes reported to be associated with RBC-related SNPs, 72 genes (67%) have at least one variation within close proximity of SMAD1 binding site. Representative RBC-associated SNPs on CCND3 and HBS1L gene that are located right on GATA1+SMAD1 co-bound peaks are shown in the right panel. More than 80% of the RBC-trait-related SNPs are located within active/open chromatin regions during human erythropoiesis that are significantly enriched with SMAD1 binding.

FIG. 23 is a table showing RBC trait related SNPS often creates or disrups signaling factor motifs. Representative examples of signaling transcription factor motifs that are either created or destroyed due to RBC-associated SNPs are shown.

FIG. 24 is a schematic of the working model: SMAD1, along with GATA-transcription factors occupies genomic regions where various signaling pathways converge to define stage-specific Signaling Centers. Such signaling hotspots are functionally important and are perturbed directly by RBC-trait-associated SNPs that are identified in genome-wide association studies.

FIG. 25 is a schematic showing the practical implication of the study presented herein. The study shows a direct involvement of Signaling Centers to counteract hazardous environment and indicates a mechanism of how individuals with distinct genetic makeup can differentially respond to various environmental stress.

FIG. 26 is a schematic of the showing the link between master transcription factors and cell-extrinsic signaling pathways.

FIGS. 27A-27B are graphs indicating that SMAD1 and GATA co-bound signaling centers contain stage specific transcription factor motifs. FIG. 27A, GATA2 peaks at H6. FIG. 27B, GATA1 peaks at D5.

FIGS. 28A-28B is a chart (FIG. 28A) and schematic of (FIG. 28B) of FHS indicates loss of SMAD1 binding correlates with decreased gene expression in a cis-acting manner.

FIGS. 29A-29B are graphs of PU.1 and SMAD1 binding that indicate PU1 directs SMAD1 binding at the signaling-centers.

FIGS. 30A-30B show a graph of PU.1 mRNA (FIG. 30A) and gel of PU.1 mRNA (FIG. 30A).

FIG. 31 shows a subset of enhancers that are transcriptional signaling centers Enhancers are defined by taking intersection of ATACseq and H3K27ac ChIPseq, and overlapped the signaling centers (i.e. GATA+SMAD1 co-bound regions) with them. It was observed that only a subset of enhancers are signaling centers.

FIGS. 32A and 32B show signaling STF motifs preferentially targeted by RBC-SNPs. (FIG. 32A) Frequency of H3K27ac peak-associated (Top Panel) and ATAC-seq peak-associated (Bottom Panel) RBC-SNPs at motifs related to STF (signaling transcription factor), blood MTF (known master transcription factors relevant for blood development), blood MTF or STF and Other TF (Transcription factors that may not be directly related to blood). “No motif” indicates examples where SNPs are located on DNA sequences that do not reveal any known transcription factor motif (n, %) shows total number and percent frequency of SNPs in each class, respectively. (FIG. 32B) Representative family of STFs and the associated DNA binding motifs that are targeted by the SNPs. Examples of genes nearest to the enhancers harboring the SNPs are also shown.

FIG. 33 shows STF motif abundance does not govern appearance of more SNPs within STF motifs relative to MTFs. Bar graph showing the occurrence of SNPs within STF motifs relative to the abundance of STF motifs in H3K27ac-positive enhancers. SMAD, TCF, CREB, NR (RXR, ROR, RAR) and FOX motifs are used as STFs and GATA, SPI1, RUNX, and MYB motifs are used as MTFs in this analysis. Within enhancers, 57 SNPs target only STF motifs where as 8 SNPs target only MTF motifs, resulting in a 7.13-fold (=57/8) more occurrence of SNPs within STF motifs relative to MTFs. Within enhancers, STF motifs occur in 16 Mbp of DNA sequence and MTF motifs occur in 26 Mbp of DNA sequence. The ratio (16 Mbp/26 Mbp=0.62) represents the occurrence of STF motifs within enhancers relative to MTF motifs. The grey bar shows the ratio of these two ratios (7.13/0.62=11.5) that represents the observed frequency of occurrence of SNPs in STF motifs relative to their abundance. The white bar is set at 1 that represents the expected value of this ratio if SNP occurrence at STF motifs and their abundance compared to MTFs are exactly proportional to each other. A 2×2 chi-squared test is performed to show that the observed ratio is significantly different than the expected ratio (p=1.6e-16).

FIGS. 34A-34C show RBC-SNPs within regulatory DNA elements show high enrichment for SMAD1-signaling centers. (FIG. 34A) Frequency of appearance of H3K27ac peak associated RBC-SNPs at SMAD1+GATA co-bound, only SMAD1-bound and only GATA-bound genomic regions. (n, %) shows total number and percent frequency of SNPs in each class, respectively. (FIG. 34B) Frequency of appearance of ATAC-seq peak associated RBC-SNPs at SMAD1+GATA co-bound, only SMAD1-bound and only GATA-bound genomic regions. (n, %) shows total number and percent frequency of SNPs in each class, respectively. (C) Red lines on the gene tracks showing the position of six representative SNPs (rs1051130, rs737092, rs2979489, rs7606173, rs13220662 and rs12718598) and their nearest genes (CCND3, RBM38, RBPMS, BCL11A, HBS1L and IKZF1, respectively). The binding of GATA2/1 and SMAD1, and the peaks of H3K27ac and ATAC-seq are also shown with respect to the SNP co-ordinates. The potential binding sites of signaling factors that these SNPs could target (e.g. SMAD, NRSA, TCF7L, CREB, RXR/FOX) are as indicated.

FIGS. 35A-35E show SNP associated with mean corpuscular volume alters SMAD1 motif in signaling center. (FIG. 35A) Alleles of SNP rs9467664 are shown with their frequency of appearance and also their impact on probable transcription factor binding are as indicated. (FIG. 35B) Schematic representation of MCV. (FIG. 35C) HIST1H4A gene track showing the position of SNP rs9467664 (red line) with respect to GATA/SMAD1 binding, H3K27ac- and ATAC-seq peaks. (FIG. 35D) Oligonucleotide sequences with T- and A-allele, associated with the SNP rs9467664, are compared with the known SMAD1 motif, as indicated. T-allele represents the strongest conserved nucleotide in the SMAD1 motif that is lost in A-allele. (FIG. 35E) RNA-seq expression values (RPKM) are shown for the gene HIST1H4A at different stages of CD34+ erythroid differentiation, as indicated.

FIGS. 36A and 36B show SNP associated with mean corpuscular volume alters SMAD1 binding in signaling center. (FIG. 36A) Representative gel-shift assay with A- and T-allele of rs9467664. Competitor oligonucleotides have been used in each case to show binding specificity, as indicated and G1ER extracts were used as negative control for the binding assays. S1-FB=SMAD1 overexpressing clone. (FIG. 36B) HIST1H4A QTL analysis for the SNP rs9467664 using genotype and gene expression data from Framingham Heart Study (FHS). Boxplots represents the distribution of HIST1H4A transcript expression in individuals with AA, AT and TT genotype, as indicated.

FIG. 37A-37C show SNP associated with mean corpuscular volume alters SMAD1 binding in erythroid-specific signaling center. (FIG. 37A) Schematic representation of MCV. (FIG. 37B) RNA-seq expression values (RPKM) are shown for the gene RBM38 at different stages of CD34+ erythroid differentiation, as indicated. (FIG. 37C) RBM38 gene track showing the position of SNP rs737092 (red line) with respect to GATA/SMAD1 binding, H3K27ac- and ATAC-seq peaks. The SNPs falls in a typical erythroid signaling center that is co-bound by GATA1, SMAD1, erythroid factor KLF1 and only open in an erythroid stage. SNP rs737092 targets a SMAD motif that falls in between GATA sites.

FIGS. 38A-38C show SNP associated with mean corpuscular volume alters signal responsiveness of erythroid-specific signaling center. (FIG. 38A) Alleles of SNP rs737092 are shown with their frequency of appearance and also their impact on probable transcription factor binding are as indicated. (FIG. 38B) Oligonucleotide sequences with T- and C-allele, associated with the SNP rs737092, are compared with the known SMAD motif, as indicated. T-allele represents the strongest conserved nucleotide in the SMAD motif that is lost in C-allele. (FIG. 38C) T and C alleles show altered responsiveness in the presence of BMP that correlates with loss of SMAD1 binding with C allele.

FIG. 39 shows PU1 occupancy at indicated sites at D0 (left pie chart), and KLF1 occupancy at indicated site at D5 (right pie chart).

FIG. 40 shows a western blot of Flag-SMAD1 protein expression in the indicated conditions, e.g., with the addition of doxycycline (DOX).

FIG. 41 shows a representative model for how stress induced growth factors activate STFs, leading to altered RBC-traits.

FIGS. 42A and 42B show enhances in indicated samples. FIG. 42A shows a plot comparing Log 2(fold change) of D5 or D3 H3K27ac signal for individual enhancers at H6 and D3, as indicated in each chart Enhancers at later and earlier stages are show. Shared enhancers are shown in the overlap. FIG. 42B shows H3K27ac Peak in progenitor and erythrocyte genes, as indicated.

FIG. 43 shows the enrichment of the indicated genes relative to the input in the indicated conditions.

### REFERENCE TO LENGTHY TABLE

The specification includes lengthy Tables: Lengthy Table S1; Lengthy Table S5; and Lengthy Table S6.

Lengthy Table S1 has been submitted by EFS web in electronic format as follows: File Name: Lengthy Table S1.txt; Date created: Jun. 21, 2017; File Size 1,144,715 Bytes and is incorporated by reference in its entirety.

Lengthy Table S5 has been submitted by EFS web in electronic format as follows: File Name: Lengthy Table S5.txt; Date created: Jun. 21, 2017; File Size 57,523 Bytes and is incorporated by reference in its entirety.

Lengthy Table S6 has been submitted by EFS web in electronic format as follows: File Name: Lengthy Table S6.txt; Date created: Jun. 21, 2017; File Size 304,777 Bytes and is incorporated by reference in its entirety.

Note lengthy Table S1 and Table S6 include columns separated by commas to delineate columns. Please refer to the end of the specification for access instructions.

### DETAILED DESCRIPTION

All references cited herein are incorporated by reference in their entirety.

Embodiments of the invention relate generally to methods for modulating erythropoiesis comprising contacting a population of CD34⁺ cells with an agent that alters occupancy at stage-specific signaling centers.

As used herein, a “signaling center” refers to a region of genomic DNA that comprises at least a DNA binding site for a lineage specific regulator, and a DNA binding site for a signal-responsive transcription factor. Activation of the signaling centers at various stages of differentiation increase gene expression of associated genes and drive erythropoiesis, i.e. eyrthroid differentiation.

As used herein, “signal-responsive transcription factor/s” refers to transcription factors that are activated by extracellular stimulation of a signaling pathway, i.e. receptor mediated signaling. “Signal-responsive transcription factor/s” include, but are not limited to transcription factors activated by: receptor kinases, nuclear hormone receptors, the cAMP pathway, MAPK pathway, JAK-STAT pathway, NFKB pathway, Wnt pathway, TGF-β pathway, LIF pathway, BDNF pathway, PGE2 pathway, and NOTCH pathway. “Signal-responsive transcription factors” are not limited to functioning in a specific lineage of development. Activated signal-responsive transcription factors bind to genomic DNA and modulate gene expression. As used herein, “a signal-responsive transcription factor” does not include GATA1 or GATA2.

As used herein, to “alter occupancy” refers to inhibiting or promoting binding of a factor at the signaling-center, e.g. a signal-responsive transcription factor, or a tissue specific transcription factor etc. In one embodiment, an agent that alters occupancy at the signaling center increases the associated gene expression by 5%, 10%, 15%, 20%, 25%, 30%, 33%, 35%, 40%, 45%, 50%, 52% 55%, 60%, 65%, 67%, 69%, 70%, 74%, 75%, 76%, 77%, 80%, 85%, 90%, 95% or more than 95%. In one embodiment, gene expression may be increased by 1, 2, 3, 4, 5, 6, 7, 8, 9, 10, 11, 12, 13, 14, 15, 16, 17, 18, 19, 20 21, 22, 23, 24, 15, 26, 27, 28, 29, 30, 31, 32, 33, 34, 35, 36, 37, 38, 39, 40, 41, 42, 43, 44, 45, 46, 47, 48, 49, 50, 51, 52, 53, 54, 1-5, 1-10, 1-20, 1-30, 1-40, 1-50, 2-5, 2-10, 2-20, 2-30, 2-40, 2-50, 3-5, 3-10, 3-20, 3-30, 3-40, 3-50, 4-6, 4-10, 4-20, 4-30, 4-40, 4-50, 5-7, 5-10, 5-20, 5-30, 5-40, 5-50, 6-8, 6-10, 6-20, 6-30, 6-40, 6-50, 7-10, 7-20, 7-30, 7-40, 7-50, 8-10, 8-20, 8-30, 8-40, 8-50, 9-10, 9-20, 9-30, 9-40, 9-50, 10-20, 10-30, 10-40, 10-50, 20-30, 20-40, 20-50, 30-40, 30-50 or 40-50 times the wild type level, or such level as is presented by a subject having a disease or disorder associated with the aberrant expression of that gene.

Genomic “Hotspots” that Function to Regulate Stage-Specific Gene Expression

Data presented herein show a genome-wide analysis that has identified signaling centers and their characteristic occupancies, which are important for erythropoiesis. Studies presented herein demonstrate that signal responsive factors together with lineage regulators mark genomic “hotspots” that function to regulate stage specific gene expression.

Accordingly, embodiments of the invention relate to the use of agents that alter occupancy at these signaling centers, e.g. binding of signal-responsive transcription factors or other factors to the signaling center.

In one embodiment, the agent that alters occupancy at the signaling center in the genome is an agonist or antagonist of a signaling pathway that is selected from the group consisting of: nuclear hormone receptor, cAMP pathway, MAPK pathway, JAK-STAT pathway, NFKB pathway, Wnt pathway, TGF-β pathway, LIF pathway, BDNF pathway, PGE2 pathway, and NOTCH pathway.

Wnt Signaling Pathway

The Wnt signaling pathways are a group of three well-characterized and highly conserved signal transduction pathways: the canonical Wnt pathway, the noncanonical planar cell polarity pathway, and the noncanonical Wnt/calcium pathway. All three pathways are activated by binding a Wnt-protein Ligand to a Frizzled family receptor, which passes the biological signal to the Dishevell protein inside the cell. Wnt signaling is reviewed in Clever, H. Cell, 149, 2012. Non-limiting agonists of the Wnt signaling pathway include e.g., PP2A, ARFGAP1, β-Catenin, Wnt3a, WAY-316606, lithium, IQ1, BIO(6-bromoindirubin-3′-oxime), and 2-amino-4-[3,4-(methylenedioxy)benzyl-amino]-6-(3-methoxyphenyl)pyrimidine. Non-limiting antagonists of the Wnt signaling pathway include e.g., C59, IWP, XAV939, Niclosamide, IWR, and hexachlorophene.

Nuclear Hormone Receptor (NHR) Signaling Pathway

Nuclear hormone receptor proteins form a class of ligand activated proteins that, when bound to specific sequences of DNA serve as on-off switches for transcription within the cell nucleus. This class includes receptors for thyroid and steroid hormones, retinoids, and vitamin D. Nuclear hormone receptor signaling controls the development and differentiation of skin, bone and behavioral centers in the brain, as well as the continual regulation of reproductive tissues. Nuclear hormone receptor signaling is reviewed in Aranda, A. and Pascual, A. Physiological Reviews, 81(3), 2001. Non-limiting agonists of the nuclear hormone receptor signaling pathway include e.g., thiazolidinediones, estadiol, dexamethasone, and testosterone. Non-limiting antagonists of the nuclear hormone receptor signaling pathway include e.g., mifepristone.

cAMP Signaling Pathway

cAMP signaling, also known as adenylyl cyclase pathway mediate cellular processes in humans, such as increase in heart rate, cortisol secretion, and breakdown of glycogen and fat. cAMP is for the maintenance of memory in the brain, relaxation in the heart, and water absorbed in the kidney. In humans, cAMP activates protein kinase A (PKA, cAMP-dependent protein kinase), one of the first few kinases discovered. It has four sub-units two catalytic and two regulatory. cAMP binds to the regulatory sub-units, breaking the sub-units from the catalytic sub-units. The Catalytic sub-units make their way in to the nucleus to influence transcription. The cAMP signaling pathway is reviewed in Yan, K., et al. Molecular Medicine Reports, 13(5), 2016. Non-limiting agonists of cAMP signaling pathway include e.g., bucladesine, Salmeterol, Theophylline, Desmopressin, Rimonabant, Haloperidol, and Metoclopramide. Non-limiting antagonists of cAMP signaling pathway include e.g., 9-Cyclopentyladenine monomethanesulfonate, 2′,5′-Dideoxyadenosine, 2′,5′-Dideoxyadenosine 3′-triphosphate tetrasodium salt, KH7, LRE1, NKY80, and MDL-12,330A.

MAPK Signaling Pathway

Mitogen-activated protein kinases (MAPKs) are a highly conserved family of serine/threonine protein kinases involved in a variety of fundamental cellular processes such as proliferation, differentiation, motility, stress response, apoptosis, and survival. A broad range of extracellular stimuli including mitogens, cytokines, growth factors, and environmental stressors stimulate the activation of one or more MAPKK kinases (MAPKKKs) via receptor-dependent and -independent mechanisms. MAPKKKs then phosphorylate and activate a downstream MAPK kinase (MAPKK), which in turn phosphorylates and activates MAPKs. Activation of MAPKs leads to the phosphorylation and activation of specific MAPK-activated protein kinases (MAPKAPKs), such as members of the RSK, MSK, or MNK family, and MK2/3/5. These MAPKAPKs function to amplify the signal and mediate the broad range of biological processes regulated by the different MAPKs. While most MAPKKK, MAPKK, and MAPKs display a strong preference for one set of substrates, there is significant cross-talk in a stimulus and cell-type dependent manner. MAPK signaling is reviewed in Zhang, W. and Liu, H. T. Cell Research, 12, 2002. Non-limiting agonists of MAPK signaling pathway include e.g., β-Arrestin, D1 dopamine receptor, SKF38393, and isoprenaline hydrochloride. Non-limiting antagonists of MAPK signaling pathway include e.g., Selumetinib (AZD6244), PD032590, Trametinib (GSK1120212), Trametinib (GSK1120212), and U0126-EtOH.

JAK-STAT Signaling Pathway

The JAK-STAT signalling cascade consists of three main components: a cell surface receptor, a Janus kinase (JAK) and two Signal Transducer and Activator of Transcription (STAT) proteins. Disrupted or dysregulated JAK-STAT functionality can result in immune deficiency syndromes and cancers. Binding of various ligands, such as interferon, interleukin, and growth factors to cell surface receptors, activate associated JAKs, increasing their kinase activity. Activated JAKs phosphorylate tyrosine residues on the receptor, creating binding sites for proteins possessing SH2 domains. SH2 domain containing STATs are recruited to the receptor where they are also tyrosine-phosphorylated by JAKs. These activated STATs form hetero- or homodimers and translocate to the cell nucleus where they induce transcription of target genes. STATs may also be tyrosine-phosphorylated directly by receptor tyrosine kinases, such as the epidermal growth factor receptor, as well as by non-receptor (cytoplasmic) tyrosine kinases such as c-src. JAK-STAT signaling is reviewed in Shuai, K. and Liu, B. Nature Immunology Reviews, 3, 2003. Non-limiting agonists of the JAK-STAT signaling pathway include e.g., Serotonin (5-hydroxytryptamine, 5-HT), and type I TNF receptor. Non-limiting antagonists of the JAK-STAT signaling pathway include e.g., jakinibs, Tofacitinib, Baricitinib, Ruxolitinib, and AZD1480.

NFkB Signaling Pathway

NF-κB (nuclear factor kappa-light-chain-enhancer of activated B cells) is a protein complex that controls transcription of DNA, cytokine production and cell survival. NF-κB is found in almost all animal cell types and is involved in cellular responses to stimuli such as stress, cytokines, free radicals, heavy metals, ultraviolet irradiation, oxidized LDL, and bacterial or viral antigens. NF-κB plays a key role in regulating the immune response to infection, with κ light chains being critical components of immunoglobulins. Incorrect regulation of NF-κB has been linked to cancer, inflammatory and autoimmune diseases, septic shock, viral infection, and improper immune development. NF-κB has also been implicated in processes of synaptic plasticity and memory. NF-κB signaling is reviewed in Gilmore, T. D. Oncogene 25, 2006. Non-limiting agonists of the NFkB signaling pathway include e.g., Betulinic acid, (R)-2-Hydroxyglutaric acid disodium salt, and Prostratin. Non-limiting antagonists of the NFkB signaling pathway include e.g., JSH-23, Rolipram, GYY 4137, p-XSC, wortmannin, and CV3988.

TGFβ Signaling Pathway

The transforming growth factor beta (TGFβ) signaling pathway is involved in many cellular processes in both the adult organism and developing embryo including cell growth, cell differentiation, apoptosis, and cellular homeostasis. TGFβ superfamily ligands bind to a type II receptor, which recruits and phosphorylates a type I receptor. The type I receptor then phosphorylates receptor-regulated SMADs (R-SMADs) which can now bind the coSMAD SMAD4. R-SMAD/coSMAD complexes accumulate in the nucleus where they act as transcription factors and participate in the regulation of target gene expression. Huang, F. and Chen, Y. G. Cell and Bioscience, 2(9), 2012. Non-limiting agonists of the TGFb signaling pathway include e.g., 7-[4-(4-cyanophenyl)phenoxy]-heptanohydroxamic acid (A-161906). Non-limiting antagonists of the TGFb signaling pathway include e.g., SB431542, LDN-193189, Galunisertib (LY2157299), and LY2109761.

LIF Signaling Pathway

Leukemia inhibitory factor, or LIF, is an interleukin 6 class cytokine that affects cell growth by inhibiting differentiation. When LIF levels drop, the cells differentiate. LIF derives its name from its ability to induce the terminal differentiation of myeloid leukemic cells, thus preventing their continued growth. LIF binds to the specific LIF receptor (LIFR-α) which forms a heterodimer with a specific subunit common to all members of that family of receptors, the GP130 signal transducing subunit. This leads to activation of the JAK-STAT and MAPK signaling cascades. Aspects of LIF signaling are reviewed in Onishi, K, and Zandstra, P. W. Development, 142(13), 2015, and Ohtsuka, S. et al. JAK-STAT, 4, 2015. Non-limiting antagonists of the LIF signaling pathway include e.g., hLIF-05.

BDNF Signaling Pathway

Brain-derived neurotrophic factor is a protein that, in humans, is encoded by the BDNF gene. BDNF is a neurotrophin essential for growth, differentiation, plasticity, and survival of neurons. BDNF is also required for processes such as energy metabolism, behavior, mental health, learning, memory, stress, pain and apoptosis. BDNF is a member of the neurotrophin family of growth factors, which are related to the canonical Nerve Growth Factor. BDNF acts on certain neurons of the central nervous system and the peripheral nervous system. BDNF itself is important for long-term memory. Moreover, neurotrophins are proteins that help to stimulate and control neurogenesis, or the process of generating new neurons, BDNF being one of the most active. BDNF signaling is reviewed in Baydyuk, M., and Xu, B. Front. Cell. Neurosci, 8(254), 2014. Non-limiting antagonists of the BDNF signaling pathway include e.g., AZ623, AZD6918, and cyclotraxin-B.

PGE2 Signaling Pathway

Prostaglandin E₂ (PGE2), an essential homeostatic factor, is also a key mediator of immunopathology in chronic infections and cancer. PGE2 promotes the balance between its cyclooxygenase 2-regulated synthesis and the pattern of expression of PGE2 receptors. PGE2 enhances its own production but suppresses acute inflammatory mediators, resulting in its predominance at late/chronic stages of immunity. PGE2 supports activation of dendritic cells but suppresses their ability to attract naive, memory, and effector T cells. PGE2 selectively suppresses effector functions of macrophages and neutrophils and the Th1-, CTL-, and NK cell-mediated type 1 immunity, but it promotes Th2, Th17, and regulatory T cell responses. PGE2 modulates chemokine production, inhibiting the attraction of proinflammatory cells while enhancing local accumulation of regulatory T cells cells and myeloid-derived suppressor cells. PGE₂ signaling is reviewed in Kalisnki, P. The Journal of Immunology, 188, 2012. Non-limiting agonists of the PGE₂ signaling pathway include e.g., 7,8-dihydroxyflavone. Non-limiting antagonists of the PGE₂ signaling pathway include e.g., SC-560, IMS2186, and sulforaphane.

Notch Signaling Pathway

The Notch signaling pathway is a highly conserved cell signaling system present in most multicellular organisms. Mammals possess four different notch receptors, referred to as NOTCH1, NOTCH2, NOTCH3, and NOTCH4. The notch receptor is a single-pass transmembrane receptor protein. It is a hetero-oligomer composed of a large extracellular portion, which associates in a calcium-dependent, non-covalent interaction with a smaller piece of the notch protein composed of a short extracellular region, a single transmembrane-pass, and a small intracellular region. The receptor is normally triggered via direct cell-to-cell contact, in which the transmembrane proteins of the cells in direct contact form the ligands that bind the notch receptor. Ligand binding to the receptor activates a cleavage cascade, resulting in the release of the intracellular region and its translocation into the nucleus, where is activates its transcriptional targets. Notch signaling is reviewed in Kopan, R. Cold Spring Harbor Perspectives in Biology, 2012. Non-limiting agonists of the Notch signaling pathway include e.g., MRK-0003, PPAR, and valproic acid. Non-limiting antagonists of the Notch signaling pathway include e.g., IMR-1, RAPT (N-[N-(3,5-difluorophenacetyl)-1-alanyl]-S-phenylglycine t-butyl ester), and LY3039478.

SAPK/JNK Signaling Pathway

Stress-activated protein kinases (SAPK)/Jun amino-terminal kinases (JNK) are members of the MAPK family and are activated by a variety of environmental stresses, inflam-matory cytokines, growth factors, and GPCR agonists. Stress signals are delivered to this cascade by small GTPases of the Rho family (Rac, Rho, cdc42). As with the other MAPKs, the membrane proximal kinase is a MAPKKK, typically MEKK1-4, or a member of the mixed lineage kinases (MLK) that phosphorylates and activates MKK4 (SEK) or MKK7, the SAPK/JNK kinases. Alternatively, MKK4/7 can be activated by a member of the germinal center kinase (GCK) family in a GTPase-independent manner. SAPK/JNK translocates to the nucleus where it can regulate the activity of multiple transcription factors. SAPK/JNK signaling is reviewed in Bogoyevitch Mass., e. al. (2010) c-Jun N-terminal kinase (JNK) signaling: recent advances and challenges. Non-limiting agonists of the SAPK/JNK signaling pathway include e.g., germinal centre kinase, IL-16, PKC6, and TRAF2. Non-limiting antagonists of the SAPK/JNK signaling pathway include e.g., SP600125.

ESC Pluripotency and Differentiation Signaling Pathway

Two distinguishing characteristics of embryonic stem cells (ESCs) are pluripotency and the ability to self-renew. These traits, which allow ESCs to grow into any cell type in the adult body and divide continuously in the undifferentiated state, are regulated by a number of cell signaling pathways. In human ESCs (hESCs), the predominant signaling pathways involved in pluripotency and self-renewal are TGF-β, which signals through Smad2/3/4, and FGFR, which activates the MAPK and Akt pathways. The Wnt pathway also promotes pluripotency, although this may occur through a non-canonical mechanism involving a balance between the transcriptional activator, TCF1, and the repressor, TCF3. Signaling through these pathways supports the pluripotent state, which relies predominantly upon three key transcription factors: Oct-4, Sox2, and Nanog. These transcription factors activate gene expression of ESC-specific genes, regulate their own expression, suppress genes involved in differentiation, and also serve as hESCs markers. Other markers used to identify hESCs are the cell surface glycolipid SSEA3/4, and glycoproteins TRA-1-60 and TRA-1-81. In vitro, hESCs can be coaxed into derivatives of the three primary germ layers, endoderm, mesoderm, or ectoderm, as well as primordial germ cell-like cells. One of the primary signaling pathways responsible for this process is the BMP pathway, which uses Smad1/5/9 to promote differentiation by both inhibiting expression of Nanog, as well as activating the expression of differentiation-specific genes. Notch also plays a role in differentiation through the notch intracellular domain (NICD). As differentiation continues, cells from each primary germ layer further differentiate along lineage-specific pathways. ESC signaling is reviewed in Bilic J, et al. (2012) Stem Cells. Non-limiting antagonists of the ESC signaling pathway include e.g., ERK activators.

B Cell Receptor Signaling Pathway

The B cell antigen receptor (BCR) is composed of membrane immunoglobulin (mIg) molecules and associated Igα/Igβ (CD79a/CD79b) heterodimers (α/β). The mIg subunits bind antigen, resulting in receptor aggregation, while the α/β subunits transduce signals to the cell interior. BCR aggregation rapidly activates the Src family kinases Lyn, Blk, and Fyn as well as the Syk and Btk tyrosine kinases. This initiates the formation of a ‘signalosome’ composed of the BCR, the aforementioned tyrosine kinases, adaptor proteins such as CD19 and BLNK, and signaling enzymes such as PLCγ2, PI3K, and Vay. Signals emanating from the signalosome activate multiple signaling cascades that involve kinases, GTPases, and transcription factors. This results in changes in cell metabolism, gene expression, and cytoskeletal organization. The complexity of BCR signaling permits many distinct outcomes, including survival, tolerance (anergy) or apoptosis, proliferation, and differentiation into antibody-producing cells or memory B cells. The outcome of the response is determined by the maturation state of the cell, the nature of the antigen, the magnitude and duration of BCR signaling, and signals from other receptors such as CD40, the IL-21 receptor, and BAFF-R. Many other transmembrane proteins, some of which are receptors, modulate specific elements of BCR signaling. A few of these, including CD45, CD19, CD22, PIR-B, and FcγRIIB1 (CD32), are indicated here in yellow. The magnitude and duration of BCR signaling are limited by negative feedback loops including those involving the Lyn/CD22/SHP-1 pathway, the Cbp/Csk pathway, SHIP, Cbl, Dok-1, Dok-3, FcγRIIB1, PIR-B, and internalization of the BCR. In vivo, B cells are often activated by antigen-presenting cells that capture antigens and display them on their cell surface. Activation of B cells by such membrane-associated antigens requires BCR-induced cytoskeletal reorganization. Please refer to the diagrams for the PI3K/Akt signaling pathway, the NF-κB signaling pathway, and the regulation of actin dynamics for more details about these pathways. BCR signaling is reviewed in Dal Porto J M, et al. (2004) Mol. Immunol. Non-limiting antagonists of the BCR signaling pathway include e.g., fostamatinib, GS-1101 (formally CAL-101), Ibrutinib (PCI-32765), aAVL-292, and Sorafenib.

ErbB/HER Signaling Pathway

The ErbB receptor tyrosine kinase family consists of four cell surface receptors: ErbB1/EGFR/HER1, ErbB2/HER2, ErbB3/HER3, and ErbB4/HER4. ErbB receptors are typical cell membrane receptor tyrosine kinases that are activated following ligand binding and receptor dimerization. Ligands can either display receptor specificity (i.e. EGF, TGF-α, AR, and Epigen bind EGFR) or bind to one or more related receptors; neuregulins 1-4 bind ErbB3 and ErbB4 while HB-EGF, epiregulin, and β-cellulin activate EGFR and ErbB4. ErbB2 lacks a known ligand, but recent structural studies suggest its structure resembles a ligand-activated state and favors dimerization. The ErbB receptors signal through Akt, MAPK, and many other pathways to regulate cell proliferation, migration, differentiation, apoptosis, and cell motility. ErbB family members and some of their ligands are often over-expressed, amplified, or mutated in many forms of cancer, making them important therapeutic targets. For example, researchers have found EGFR to be amplified and/or mutated in gliomas and NSCLC while ErbB2 amplifications are seen in breast, ovarian, bladder, NSCLC, as well as several other tumor types. In addition, NRG or TPA stimulation promotes ErbB4 cleavage by γ-secretase, releasing an 80 kDa intracellular domain that translocates to the nucleus to induce differentiation or apoptosis. Upon activation and cleavage, ErbB4 can also form a complex with TAB2 and N-CoR to repress gene expression. Signaling through ErbB networks is modulated through dense positive and negative feedback and feed forward loops, including transcription-independent early loops and late loops mediated by newly synthesized proteins and miRNAs. ErbB/HER Signaling is reviewed in Arteaga C L and Engelman J A (2014) Cancer Cell. Non-limiting antagonists of the ErbB/signaling pathway include e.g., Gefitinib, Bosutinib, Cetuximab, Vandetanib, Neratinib, Selumetinib, Decomitinib, and Pimasertib.

The signaling centers described herein comprise both a DNA binding site for a lineage-specific regulator and a DNA binding site for a signal-responsive transcription factor. Some signaling centers also comprise a tissue-specific transcription factor. Increasing expression at these signaling centers promotes erythropoiesis. Provided herein are methods for modulating erythropoiesis comprising contacting a population of cells comprising CD34⁺ cells (e.g. stem or progenitor cells or erythroid lineage committed cells) with an agent that alters occupancy (binding) at these signaling centers.

In one embodiment, the agent that alters occupancy at the signaling center is an agent that induces binding of the signal-responsive transcription factor.

In one embodiment, the agent that alters occupancy at the signaling center is an agent that inhibits binding of the signal-responsive transcription factor.

Also provided are methods for treating disease associated with aberrant erythropoiesis comprising correcting tile DNA at the signaling-center to restore normal occupancy at the signaling center, e.g. normal binding status of the signal-responsive transcription factor, or tissue-specific transcription factor, etc.

In one embodiment, signal-responsive transcription factor is selected from the group consisting of SMAD1, SMAD5, SMAD8, β-catenin, LEF/TCF, STAT5, RARA, BCL11A, TCF7L2, CREB3L, CREB, CREM, CTCF, IRF7, RUB, AP2B, NFKB2, PAX, PPARG, RXRA, RARE, RARB, E2F6m TBX20, TBX1, NFIA, ZN350, TCF4, EGR1 and THRB. Example signaling pathways, transcription factors, and binding motifs are found in Table 1.

In one embodiment, the signal-responsive transcription factor is a transcription factor selected from Table 1.

TABLE 1
Binding Motifs of known signaling pathway transcription factors

| e.g. | | | |
|---|---|---|---|
| Signaling | Transcription | | |
| Pathway | factor | e.g. DNA binding motif | SEQ ID NO. |
| TGFβ/BMP | SMAD1 | GTCTAGAC | SEQ ID NO.: 16 |
| TGFβ/BMP | SMAD5 | GTCTAGAC | SEQ ID NO.: 16 |
| TGFβ/BMP | SMAD8 | GTCTAGAC | SEQ ID NO.: 16 |
| TGFβ/BMP | SMAD3 | VHGTCTGBVB | SEQ ID NO.: 17 |
| Wnt | LEF/TCF | A(C/G)(A/T)TCAAAG | SEQ ID NO.: 18 |
| JAK | STAT5 | TTC(N₃₋₄)GAA | SEQ ID NO.: 19 |
| NHR | RARA | AGGTCATGGAGAGGTCA | SEQ ID NO.: 20 |
| NFKB | BCL11A | TTTCCTAGAAAGCA | SEQ ID NO.: 21 |
| Wnt | TCF7L2 | AAAGATCAAAGGAA | SEQ ID NO.: 22 |
| cAMP | CREB3L | GCCACGTGT | SEQ ID NO.: 23 |
| cAMP | CREB | TGACGTCA | SEQ ID NO.: 24 |
| cAMP | CREM | TATGACGTAA | SEQ ID NO.: 25 |
| NFKB | CTCF | CCGCGNGGNGGCAG | SEQ ID NO.: 26 |
| cAMP | IRF7 | CGAAACCGAAACT | SEQ ID NO.: 27 |
| NFKB | RELB | GGGAATTTCC | SEQ ID NO.: 28 |
| | AP2B | GCCNNNGGC | SEQ ID NO.: 29 |
| NFKB | NFKB2 | AGGGGATTCCCCT | SEQ ID NO.: 30 |
| Wnt | PAX | GAGGGCAGCCAAGCGTGAC | SEQ ID NO.: 31 |
| NHR | PPARG | AGGTCANAGGTCA | SEQ ID NO.: 32 |
| NHR | RXRA | GGGTCATTGGGTTCA | SEQ ID NO.: 33 |
| NHR | RARG | AAGGTCAAAAGGTCA | SEQ ID NO.: 34 |
| NHR | RARB | AAAGGTCAAAAGGTCA | SEQ ID NO.: 35 |
| ErbB/HER | E2F6m | GGGCGGGAAGG | SEQ ID NO.: 36 |
| | TBX20 | TAGGTGTGAAG | SEQ ID NO.: 37 |
| | TBX1 | AAGGTGTGAAG | SEQ ID NO.: 38 |
| | NFIA | ATGCCAA | SEQ ID NO.: 39 |
| | NFIB | CCAAT | SEQ ID NO.: 40 |
| | ZN350 | ATCCAC | SEQ ID NO.: 41 |
| Wnt | TCF4 | A(C/G)(A/T)TCAAAG | SEQ ID NO.: 42 |
| BCR | EGR1 | CCCCCGCCCCCGCC | SEQ ID NO.: 43 |
| | AHR | DKHGCGTGH | SEQ ID NO.: 49 |
| | AP2A | DDDSCCTGRGGSHDD | SEQ ID NO.: 52 |
| | AP2C | VDDSCCTGRGGSHV | SEQ ID NO.: 58 |
| BCR | BCL6 | DDDDDHWTTCNWRGRW | SEQ ID NO.: 62 |
| | COE1 | DDDYCCCWRGGGAVH | SEQ ID NO.: 64 |
| | CTCFL | BBDCCRSHAGRKGGCRSBV | SEQ ID NO.: 65 |
| ErbB/HER | E2F5 | NGCGCCAAAH | SEQ ID NO.: 66 |
| BCR | EGR2 | DGVGKGGGCGK | SEQ ID NO.: 67 |
| | ERR3 | TYAAGGTCA | SEQ ID NO.: 68 |
| WNT | FOXA1 | DTGTTTACWYWDB | SEQ ID NO.: 69 |
| WNT | FOXC1 | BNHTGTTTACWTAVS | SEQ ID NO.: 70 |
| WNT | FOXJ2 | TRTTTATYTD | SEQ ID NO.: 71 |
| WNT | FOXJ3 | DTGTTTATKKTTD | SEQ ID NO.: 72 |
| WNT | FOXO3 | DRYBTGTTTWYHD | SEQ ID NO.: 73 |
| | GATA1 | VBKNNNNNNDVWGATAASV | SEQ ID NO.: 74 |
| | GATA3 | DVAGATARVRD | SEQ ID NO.: 75 |
| | GATA4 | DVWGATARV | SEQ ID NO.: 76 |
| | GATA6 | DDVAGATAAGRDDD | SEQ ID NO.: 77 |
| | GLIS3 | NTGGGTGGTYB | SEQ ID NO.: 78 |
| | Hic1 | DKGKTGCCM | SEQ ID NO.: 79 |
| | HINFP1 | DHSNNVDCGGACGTWV | SEQ ID NO.: 80 |
| SAPK/JNK | HSF2 | VSRWBVWKSKVGRH | SEQ ID NO.: 81 |
| TGFβ | IRF1 | VVRRVNGAAASYGAAASYVV | SEQ ID NO.: 82 |
| TGFβ | IRF7 | RAAABYRAAW | SEQ ID NO.: 83 |
| | KLF8 | CAGGGGGTG | SEQ ID NO.: 84 |
| | MAFG | DDRDNWGCTGASTCAGCADDD | SEQ ID NO.: 85 |
| | MAZ | GGGMGGRGSVRSRSSVSSSSSS | SEQ ID NO.: 86 |
| | MBD2 | NSGGCCGGMKV | SEQ ID NO.: 87 |
| | MECP2 | SCCGGAG | SEQ ID NO.: 88 |
| ESC | NANOG | BYWTTSWNWTGYWRWDD | SEQ ID NO.: 89 |
| | NKX28 | BTCAAGGAB | SEQ ID NO.: 90 |
| | NKX31 | WWTAAGTAWWHDH | SEQ ID NO.: 91 |
| | NR1D1 | WWAAVTAGGTCAND | SEQ ID NO.: 92 |
| | NR2C2 | RRSBSARAGGKMR | SEQ ID NO.: 93 |
| | NR6A1 | RAGKTCAAGKTCA | SEQ ID NO.: 94 |
| | P63 | DDRCWDGYHKGRRCWYGYH | SEQ ID NO.: 95 |
| | PO5F1 | BYWTTVWHATGCADWH | SEQ ID NO.: 96 |
| | PRDM1 | DRMAGWGAAAGTDH | SEQ ID NO.: 97 |
| TGFβ | RUNX2 | BTGTGGTKDBB | SEQ ID NO.: 98 |
| TGFβ | RUNX3 | NBBTGTGGTYW | SEQ ID NO.: 99 |
| | SNAI2 | NCAGGTG | SEQ ID NO.: 100 |
| ESC | SOX2 | YYWTTSTBMTKSWDWH | SEQ ID NO.: 101 |
| TGFβ | SP2 | SVVVVRRRGGCGGRRSBNVVSV | SEQ ID NO.: 102 |
| | SRBP1 | VRTSRSSWGWB | SEQ ID NO.: 103 |
| | SRBP2 | VVVWGGVSWGRNB | SEQ ID NO.: 104 |
| | STAT2 | DHRSTTTCNBTTYYH | SEQ ID NO.: 105 |
| | TAL1 | BYKBNNNNNNBWGATAAVV | SEQ ID NO.: 106 |
| | TFAP4 | VYCAGCTGYVG | SEQ ID NO.: 107 |
| | TFE3 | RRWCAYGTGV | SEQ ID NO.: 108 |
| | THB | VRVSYVMBVKSAGGTCA | SEQ ID NO.: 109 |
| | XBP1 | GACGTGTMHHWD | SEQ ID NO.: 110 |
| | ZIC1 | KGGGWGSKV | SEQ ID NO.: 111 |
| | ZN148 | KMDDKGMAKKMTGGGWRDKSBH | SEQ ID NO.: 112 |

Table 1 shows exemplary binding motifs of known signaling pathway transcription factors. A=adenine, C=cytosine, G=guanine, T=thymine, R=G or A (purine), Y=T or C (pyrimidine), K=G or T (keto), M=A or C (amino), S=G or C (strong bonds), W=A or T (weak bonds), B=G, T, or C (all but A), D=G, A, or T (all but C), H=A, C, or T (all but G), V=G, C, or A (all but T), N=A, G, C, or T (any).

In certain embodiments, the signaling center further comprises tissue specific transcription factor motif. Accordingly, in one embodiment, the agent that alters occupancy at the signaling center is an agent that induces or inhibits binding of the tissue specific transcription factor.

In one embodiment, correction of DNA at the signaling center restores binding of the tissue-specific transcription factor.

Example tissue specific transcription factors, and binding motifs of the signaling-centers are found in Table 2.

| Transcription | DNA | |
|---|---|---|
| factor | binding motif | SEQ ID NO. |
| PU.1 | GAGGAA | SEQ ID NO.: 45 |
| FL1 | (G/A)CAGGAAGTGG | SEQ ID NO.: 46 |
| KROX | GCGGGGGCGG | SEQ ID NO.: 47 |
| ETV6 | AGCGGAAGTG | SEQ ID NO.: 48 |
| FLI1 | C(C/A)GGAAGT | SEQ ID NO.: 50 |
| SP1C | GCCCCGCCCCC | SEQ ID NO.: 51 |
| ETS1 | ACCGGAAGTG | SEQ ID NO.: 53 |
| SP11 | AAAAAGCGGAAGT | SEQ ID NO.: 54 |
| SP1B | AAAAAGAGGAAGTA | SEQ ID NO.: 55 |
| KLF1 | GGCCACACCCA | SEQ ID NO.: 56 |
| NFE4 | CATGACTCATC | SEQ ID NO.: 57 |
| AP2 | GCCNNNGGC | SEQ ID NO.: 59 |
| PLAG1 | GGGGCCCAAGGGGG | SEQ ID NO.: 60 |
| SP3 | GCCACGCCCCC | SEQ ID NO.: 61 |
| SP4 | TAAGCCACGCCCCCTTT | SEQ ID NO.: 63 |

A Role for Co-Localization of SMAD1 with GATA1 in Erythropoiesis

Some embodiments of the invention are based on the discovery of a role for bone morphogenetic protein (BMP)-signal-responsive transcription factor SMAD1 in human erythropoiesis, in particular co-localization of SMAD1 with GATA1 or GATA2 temporally during different stages of erythrocyte development. How differential genomic binding of signal-responsive and lineage-restricted transcription factors can specify intermediate stages of erythropoiesis was investigated. Using a human erythroid differentiation system, the co-operation of the BMP responsive signaling transcription factor SMAD1 with the erythroid transcription factors GATA2 and GATA1 was extensively characterize in a detailed time-course. It was determined that BMP signaling promotes erythroid differentiation. In addition, SMAD1 is co recruited with GATA factors at stage specific genes that are required to have high expression in each stage. It was also determined that GATA-SMAD1 co-enriched regions were located within super-enhancers and span accessible chromatin. Co-bound regions harbor cell type and stage-specific transcription factor motifs, in contrast to GATA only regions.

Accordingly, also provided herein are methods for promoting erythropoiesis (erythroid differentiation) by treating cells in vivo, or ex vivo with an activator of SMAD1.

SMAD1

SMAD1 is a transcriptional modulator activated by BMP type 1 receptor kinase. In response to BMP (bone morphogenetic protein) ligands (e.g. BMP4, as well as other BMPs) SMAD1 is phosphorylated and activated by the BMP receptor kinase. The phosphorylated form of SMAD1 is the active form which is known to form a complex with SMAD4. SMAD1 is a target for SMAD-specific E3 ubiquitin ligases, such as SMURF1 and SMURF2, and undergoes ubiquitination and proteasome-mediated degradation. Alternatively spliced transcript variants encoding SMAD1 have been observed. (Andreas von Bubnoff and Ken W. y. Cho, Intracellular BMP signaling in Vertebrates: Pathway or network? Dev. Biol., 2001, 239: 1-14). Synonyms of SMAD1 include e.g. SMAD family member 1; BSP1; JV41; BSP-1; JV4-1; MADH1; MADR1; mothers against decapentaplegic homolog 1; MAD homolog 1; Mad-related protein 1; TGF-beta signaling protein 1; mothers against DPP homolog 1; SMAD, mothers against DPP homolog 1; MAD, mothers against decapentaplegic homolog 1; transforming growth factor-beta signaling protein 1; transforming growth factor-beta-signaling protein 1. Human SMAD1, Gene ID: 4086, is a 465 aa protein, see Genebank accession AAH01878.

SEQ ID NO: 1 is an amino acid sequence encoding SMAD1.

(SEQ ID NO: 01)
MNVTSLFSFT SPAVKRLLGW KQGDEEEKWA EKAVDALVKK
LKKKKGAMEE LEKALSCPGQ PSNCVTIPRS LDGRLQVSHR
KGLPHVIYCR VWRWPDLQSH HELKPLECCE FPFGSKQKEV
CINPYHYKRV ESPVLPPVLV PRHSEYNPQH SLLAQFRNLG
QNEPHMPLNA TFPDSFQQPN SHPFPHSPNS SYPNSPGSSS
STYPHSPTSS DPGSPFQMPA DTPPPAYLPP EDPMTQDGSQ
PMDTNMMAPP LPSEINRGDV QAVAYEEPKH WCSIVYYELN
NRVGEAFHAS STSVLVDGFT DPSNNKNRFC LGLLSNVNRN
STIENTRRHI GKGVHLYYVG GEVYAECLSD SSIFVQSRNC
NYHHGFHPTT VCKIPSGCSL KIFNNQEFAQ LLAQSVNHGF
ETVYELTKMC TIRMSFVKGW GAEYHRQDVT STPCWIEIHL
HGPLQWLDKV LTQMGSPHNP ISSVS

Tagged recombinant SMAD 1 protein, e.g. GST-tagged, is available from Creative Biomart Recombinat proteins, 45-1 Ramsey Road, Shirley, N.Y. 11967, USA, and can be used in assays to identify agents that activate SMAD1 (SMAD1 activators).

As used herein, the terms “an agent that activates the transcription factor SMAD 1”, or “Activator of SMAD1” or “SMAD1 activators” refer to agents that lead to phosphorylation of the SMAD1 transcription factor and translocation of SMAD1 to the nucleus, e.g. where it can bind to genomic DNA. Any activator of SMAD1 can be used in methods of the invention. The activator can be a small molecule, a nucleic acid RNA, a nucleic acid DNA, a protein, a peptide, or an antibody. Cell assays to identify activators of SMAD1 are known in the art, See for example Vrijens, et al. Identification of small molecule activators of BMP signaling PloS-ONE 8(3): e59045 (2013), incorporated herein by reference in its entirety. BMP signaling regulation of SMAD1 is reviewed in Andreas von Bubnoff and Ken W. Y. Cho: Review Intracellular BMP signaling regulation in vertebrates: pathway or network? Developments Biology 239:1-14, (2001), incorporated herein by reference in its entirety.

Many activators of SMAD1 are known in the art and include for example, BMP receptor kinase agonists, i.e. agents upregulate BMP receptor signaling, such as BMP protein (e.g. BMP 2, 4, and/or 7). These recombinant BMP proteins are commercially available, e.g. from humanzyme, Inc. (Chicago, Ill.).

Activators of SMAD1 also include agents that inhibit checkpoint kinase 1 (CHK1), e.g. small molecules PD407824, MK-8776, LY-2606368 and LY-2603618.

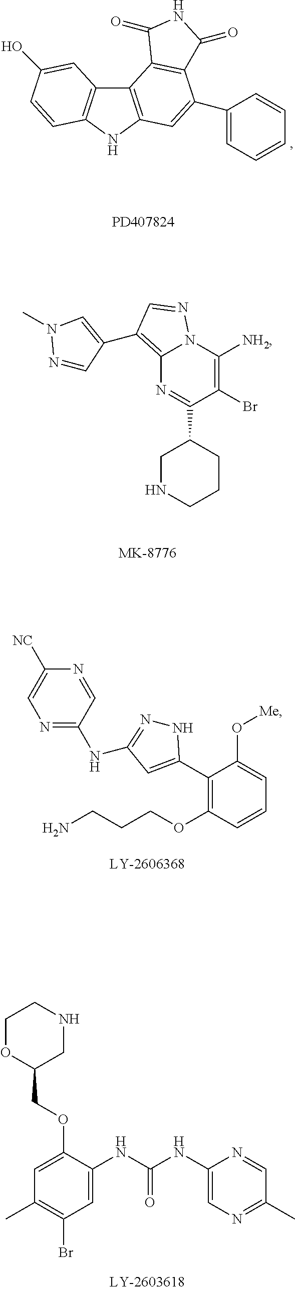 [SMILES: O=C1NC(=O)c2c1c(-c1ccccc1)cc1[nH]c3ccc(O)cc3c21 ; Cn1cc(-c2cnn3c(N)c(Br)c([C@@H]4CCCNC4)nc23)cn1 ; COc1cccc(OCCCN)c1-c1cc(Nc2cnc(C#N)cn2)n[nH]1 ; Cc1cnc(NC(=O)Nc2cc(Br)c(C)cc2OC[C@@H]2CNCCO2)cn1]

In certain embodiments, more than one activator of SMAD1 is used, e.g. in one embodiment, a combination of a BMP protein agonist and a CHK1 inhibitor are used.

In certain embodiments, the activator of SMAD1 is not a BMP protein. In certain embodiments, the activator of SMAD1 is not a BMP2 protein. In one embodiment, the activator of SMAD1 is not a BMP7 protein. In one embodiment, the activator of SMAD1 is not BMP4 protein.

In certain embodiments, the activator of SMAD1 is not a CHK1 inhibitor. In one embodiment, the activator of SMAD1 is not PD407824. In one embodiment the activator of SMAD1 is not MK-8776. In one embodiment the activator of SMAD1 is not LY-2606368. In one embodiment, the activator of SMAD1 is not LY-2603618.

Additional, small molecule activators of BMP signaling and SMAD1 are known in the art, and include for example those described in Vrijens, et al. Identification of small molecule activators of BMP signaling PloS-ONE 8(3): e59045 doi:10.1371/journal.pone.0059045 (2013), which are isoliquirtigenin; apigenin; 4′-hydroxychalcone; and diosmetin.

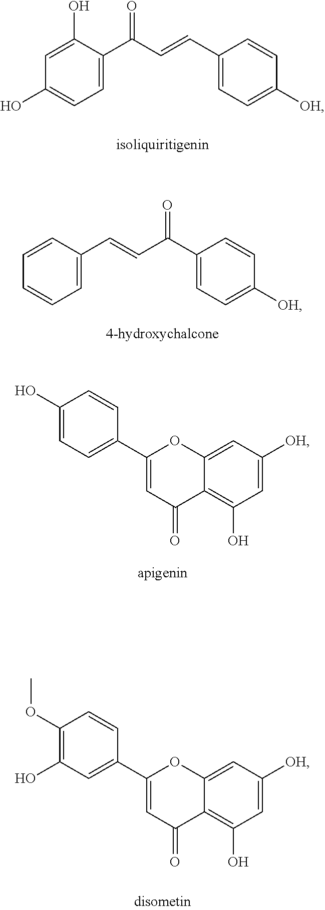 [SMILES: O=C(/C=C/c1ccc(O)cc1)c1ccc(O)cc1O ; O=C(/C=C/c1ccccc1)c1ccc(O)cc1 ; O=c1cc(-c2ccc(O)cc2)oc2cc(O)cc(O)c12 ; COc1ccc(-c2cc(=O)c3c(O)cc(O)cc3o2)cc1O]

Vrijens, et al. supra describes a high throughput screening assay that can be used to identify yet unknown small molecule activators of SMAD1, the cell screening method is incorporated by reference in its entirety.

In one embodiment the activator of SMAD1 is not isoliquirtigenin. In one embodiment the activator of SMAD1 is not apigenin. In one embodiment the activator of SMAD1 is not 4′-hydroxychalcone. In one embodiment the activator of SMAD1 is not diosmetin.

Promoting Erythroid Differentiation

In the methods described herein, an agent that alters occupancy at a signaling center is used to promote erythroid differentiation (erythropoiesis). In certain embodiments, the agent is administered as a therapeutic adjunct to other agents that promote differentiation. There are many established protocols for in vitro erythroid differentiation (erythropoiesis), that can be used in adjunct to the methods described herein. See for example those described in: Baek et al. In vitro clinical grade generation of red blood cells from human umbilical cord blood CD34⁺ cells, Transfusion 2008 48:2235-2245; Sankaran, V. G., Orkin, S. H., and Walkley, C. R., 2008, Rb intrinsically promotes erythropoiesis by coupling cell cycle exit with mitochondrial biogenesis Genes Dev 22, 463-475; Lapillonne, et al. Red blood cell generation from human induced pluripotent stem cells: perspectives for transfusion medicine, Haematologica 2010, 95: 1651-1659; Neildez-Nguyen T M, et al. Human erythroid cells produced ex vivo at large scale differentiate into red blood cells in vivo Nat. Biotechnology 2002(20): 467-72; Park et al. Poly-1-lysine increases the ex vivo expansion and erythroid differentiation of human hematopoietic stem cells, as well as erythroid enucleation efficacy Tissue Eng. Part A March 2104, Vol. 20, No. 5-6: 1072-1080; Giarranta M C, et al. Proof of principle for transfusion of in vitro generated red blood cells Blood 2011, 118: 5071-5079; Sankaran, V. G., Orkin, S. H., and Walkley, C. R. (2008b). Rb intrinsically promotes erythropoiesis by coupling cell cycle exit with mitochondrial biogenesis. Genes Dev 22, 463-475; and Trompouki, E et al. (2011). Lineage regulators direct BMP and Wnt pathways to cell-specific programs during differentiation and regeneration. Cell 147, 577-589.

In certain embodiments, the agent that alters occupancy is administered as a therapeutic adjunct to in vivo erythropoietin treatment, e.g. the use of erythropoietin (EPO) to induce erythropoiesis is exemplified by Royet et al., U.S. Pat. No. 5,482,924; Goldberg et al., U.S. Pat. No. 5,188,828; Vance et al., U.S. Pat. No. 5,541,158; and Baertschi et al., U.S. Pat. No. 4,987,121, all references hereby incorporated in their entirety. The erythropoietin dosage regimen may vary widely, but can be determined routinely by a physician using standard methods. Dosage levels of the order of between about 1 EPO unit/kg and about 5,000 EPO units/kg body weight are useful for all methods of use disclosed herein.

In one embodiment, cells are contacted with the agent ex vivo and differentiation continues to occur in vivo after transplantation of cells (See e.g. Neildez-Nguyen T M, et al. Human erythroid cells produced ex vivo at large scale differentiate into red blood cells in vivo Nat. Biotechnology 2002(20): 467-72.

In one embodiment, eyrthroid differentiation occurs in vitro prior to transplantation of the cells (See e.g. Park et al. Poly-1-lysine increases the ex vivo expansion and erythroid differentiation of human hematopoietic stem cells, as well as erythroid enucleation efficacy. Tissue Eng. Part A March 2104, Vol. 20, No. 5-6: 1072-1080; Giarranta M C, et al. Proof of principle for transfusion of in vitro generated red blood cells. Blood 2011, 118: 5071-5079.

In certain embodiments, the effect of the agent that alters occupancy on the promotion of differentiation of erythroid progenitors can be tested in vitro using the colony formation assay. For example, the assay consists of growing CD34⁺ cells, e.g. erythroid lineage committed cells, in a semi-solid medium (methylcellulose) for two weeks (Yu et al., U.S. Pat. No. 5,032,507). Conditioned medium consisting of phytohemagglutinin-treated lymphocytes (PHA-LCM) can be supplemented with erythropoietin to induce differentiation and preferably, between about 0.1 ng/ml and about 10 mg/ml of the agent. In one embodiment, differentiation is induced and agent administered as described in Example 1.

For promoting erythroid differentiation in vivo, the agent that alters occupancy can be administered by any suitable route, including orally, parentally, by inhalation spray, rectally, transdermally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Transdermal means including, but not limited to, transdermal patches may be utilized to deliver the agents to the treatment site.

A further object of the present invention is to provide pharmaceutical compositions comprising the agents as an ingredient for use in promoting red blood cell production. Dosage and administration of the pharmaceutical compositions will vary depending on the disease being treated, based on a variety of factors, including the type of injury, the age, weight, sex, medical condition of the individual, the severity of the condition, the route of administration, and the particular compound employed, as above. Thus, the dosage regimen may vary widely, but can be determined routinely by a physician using standard methods.

The dosage range for the agent that alters occupancy and gene expression of the associated gene depends upon the potency, and are in amounts large enough to produce the desired effect e.g., an increase in the efficiency and/or rate of erythroid differentiation. The dosage should not be so large as to cause adverse side effects.

Generally, the dosage will vary with the particular compound used, and with the age, condition, and sex of the patient. The dosage can be determined by one of skill in the art and can also be adjusted by a physician in the event of any complication. Dosage for in vivo use can be determined by in vitro assay in the presence of and absence of the agent. Typically, the dose will range from 0.001 mg/kg body weight to 5 g/kg body weight. In some embodiments, the dose will range from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dose range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

The methods and compositions provided herein are clinically useful as a therapeutic adjunct for increasing red blood cell production, e.g. in treating congenital or acquired aplastic or hypoplastic anemia and amelioration of anemia associated with cancer, AIDS, chemotherapy, radiotherapy, and for bone marrow transplantation. In one embodiment, the subject is selected as having been diagnosed with a disorder that results in a decreased red blood cell production, e.g. congenital or acquired aplastic or hypoplastic anemia, or anemia associated with cancer, AIDS, chemotherapy, radiotherapy, bone marrow transplantation. The methods described herein are also useful for increasing red blood cells in long distance runners and in patients undergoing elective surgery, or countering hypoxia at high altitude.

In a further aspect, the present invention provides kits for promoting erythropoiesis. The kits comprise an effective amount of the agent that alters occupancy at the signaling center, and instructions for using the amount effective of the agent that alters occupancy (e.g. an agent that activates SMAD1, or other agent) as a therapeutic adjunct, and e.g. a pharmaceutically acceptable carrier. In one embodiment, the kit further comprises a means for delivery of the active agent to a mammal. Such devices include, but are not limited to matrical or micellar solutions, polyethylene glycol polymers, carboxymethyl cellulose preparations, crystalloid preparations (e.g., saline, Ringer's lactate solution, phosphate-buffered saline, etc.), viscoelastics, polyethylene glycols, and polypropylene glycols. In one embodiment, the kits also comprise an amount of erythropoietin effective to induce erythropoiesis.

Populations of Cells Comprising CD34⁺ Cells

CD34⁺ cells can be obtained from blood products. A blood product includes a product obtained from the body or an organ of the body containing cells of hematopoietic origin. Such sources include unfractionated bone marrow, umbilical cord, peripheral blood, liver, thymus, lymph and spleen. All of the aforementioned crude or unfractionated blood products can be enriched for cells having hematopoietic stem cell characteristics in a number of ways. For example, the more mature, differentiated cells are selected against, via cell surface molecules they express. Optionally, the blood product is fractionated by selecting for CD34⁺ cells.

CD34⁺ cells include a subpopulation of cells capable of self-renewal and pluripotentcy. Such selection is accomplished using, for example, commercially available magnetic anti-CD34 beads (Dynal, Lake Success, N.Y.). Unfractionated blood products are optionally obtained directly from a donor or retrieved from cryopreservative storage.

Isolated populations of cells can be obtained by selecting for or against specific populations. For example, in certain embodiments, the population of CD34⁺ cells used in methods of the invention, can comprise 1) an isolated population of hematopoietic stem cells having the following markers: CD34⁽⁺⁾⁽⁻⁾, CD38⁽⁺⁾⁽⁻⁾, CD45RA⁻CD49f⁺CD90⁺; or an isolated population of hematopoietic progenitor cells that are that are CD34⁺CD45RA⁺CD38⁺; 3) or an isolated population of erythroid lineage committed cells that are CD34⁺CD38⁺CD45RA⁻.

In one embodiment, the population of CD34⁺ cells is derived from peripheral blood e.g., as described in Sankaran, V. G. et al. (See Sankaran, V. G. et al. (2008) Rb intrinsically promotes erythropoiesis by coupling cell cycle exit with mitochondrial biogenesis. Genes Dev 22, 463-475).

In one embodiment, the population of CD34⁺ cells is derived from induced pluripotent stem cells such as those described in Lapillonne et al. (See Lapillonne, et al. Red blood cell generation from human induced pluripotent stem cells: perspectives for transfusion medicine. Haematologica (2010) 95: 1651-1659).

In certain embodiments, the population of CD34⁺ cells are contacted with an agent that alters occupancy, or that corrects the DNA at the signaling-center ex vivo, and after the contacting step the cells are transplanted into a subject.

In one embodiment, the eyrthroid differentiation into red blood cells (i.e. into erythrocytes that are CD34⁻, CD59⁺ and glycophorin⁺/CD235a⁺ continues to occur in vivo after transplantation of the cells into the subject (e.g. See Neildez-Nguyen T M, et al. Human erythroid cells produced ex vivo at large scale differentiate into red blood cells in vivo. Nat. Biotechnology (2002) 20: 467-72).

In one embodiment, eyrthroid differentiation into red blood cells (i.e. into erythrocytes that are CD34⁻, CD59⁺ and glycophorin⁺/CD235a⁺ occurs in vitro prior to transplantation of the cells in to the subject (See e.g. Park et al. Poly-1-lysine increases the ex vivo expansion and erythroid differentiation of human hematopoietic stem cells, as well as erythroid enucleation efficacy. Tissue Eng. Part A (2104) 20, No. 5-6: 1072-1080; and Giarranta M C, et al. Proof of principle for transfusion of in vitro generated red blood cells. Blood 20011, 118: 5071-5079).

Sources for HSC expansion (CD34″ expansion) can include aorta-gonad-mesonephros (AGM) derived cells, embryonic stem cell (ESC) and induced pluripotent stem cells (iPSC). ESC are well-known in the art, and may be obtained from commercial or academic sources (Thomson et al., 282 Sci. 1145-47 (1998)). iPSC are a type of pluripotent stem cell artificially derived from a non-pluripotent cell, typically an adult somatic cell, by inducing a “forced” expression of certain genes (Baker, Nature Rep. Stem Cells (Dec. 6, 2007); Vogel & Holden, 23 Sci. 1224-25 (2007)). ESC, AGM, and iPSC may be derived from animal or human sources. The AGM stem cell is a cell that is born inside the aorta, and colonizes the fetal liver. Signaling pathways can increase AGM stem cells make it likely that these pathways will increase HSC in ESC.

Bone marrow can be obtained by puncturing bone with a needle and removing bone marrow cells with a syringe (herein called “bone marrow aspirate”). Hematopoietic progenitor CD34⁺ cells can be isolated from the bone marrow aspirate by using surface markers specific for hematopoietic progenitor cells, or alternatively whole bone marrow can be used. Hematopoietic progenitor cells can also be obtained from peripheral blood of a progenitor cell donor. Prior to harvest of the cells from peripheral blood, the donor can be treated with a cytokine, such as e.g., granulocyte-colony stimulating factor, to promote cell migration from the bone marrow to the blood compartment. Cells can be collected via an intravenous tube and filtered to isolate cells for treatment and subsequent transplantation. The white blood cell population obtained (i.e., a mixture of stem cells, progenitors and white blood cells of various degrees of maturity) can be treated and transplanted as a heterogeneous mixture or hematopoietic progenitor cells can further be isolated using cell surface markers known to those of skill in the art.

Hematopoietic progenitor cells and/or a heterogeneous hematopoietic progenitor cell population can also be isolated from human umbilical cord and/or placental blood. The CD34⁺ enriched human stem cell fraction can be separated by a number of reported methods, including affinity columns or beads, magnetic beads or flow cytometry using antibodies directed to surface antigens such as the CD34⁺. Further, physical separation methods such as counterflow elutriation may be used to enrich hematopoietic progenitors.

The CD34⁺ progenitors are heterogeneous, and may be divided into several subpopulations characterized by the presence or absence of coexpression of different lineage associated cell surface associated molecules. The most immature progenitor cells do not express any known lineage-associated markers, such as HLA-DR or CD38, but they may express CD90 (thy-1). Other surface antigens such as CD33, CD38, CD41, CD71, HLA-DR or c-kit can also be used to selectively isolate hematopoietic progenitors. The separated cells can be incubated in selected medium in a culture flask, sterile bag or in hollow fibers. Various hematopoietic growth factors may be utilized in order to selectively expand cells. Representative factors that have been utilized for ex vivo expansion of bone marrow include, c-kit ligand, IL-3, G-CSF, GM-CSF, IL-1, IL-6, IL-11, flt-3 ligand or combinations thereof. The proliferation of stem cells can be monitored by enumerating the number of stem cells and other cells, by standard techniques (e. g., hemacytometer, CFU, LTCIC) or by flow cytometry prior and subsequent to incubation.

Common methods used to physically separate specific cells from within a heterogenous population of cells within a hematopoietic cell preparation include but are not limited to flow-cytometry using a cytometer which may have varying degrees of complexity and or detection specifications, magnetic separation, using antibody or protein coated beads, affinity chromatography, or solid-support affinity separation where cells are retained on a substrate according to their expression or lack of expression of a specific protein or type of protein.

In general, cells useful for the invention can be maintained and expanded in culture medium that is available to and well-known in the art. Such media include, but are not limited to, Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®, and serum-free medium for culture and expansion of hematopoietic cells SFEM®. Many media are also available as low-glucose formulations, with or without sodium pyruvate. Cells can be cultured on feeder layers. Synthetic biodegradable matrices include synthetic polymers such as polyanhydrides, polyorthoesters, and polylactic acid; see also, for example, U.S. Pat. No. 4,298,002 and U.S. Pat. No. 5,308,701.

In one embodiment, expanded hematopoietic stem and/or progenitor cells are treated ex vivo prior to transplantation to an individual in need thereof by contacting the expanded population of hematopoetic cells with an agent that alters occupancy, and alternatively in adjunct with a protocol for differentiation. Contacting is performed in vitro by adding the agent directly to suitable cell culture medium for hematopoietic cells. The concentration of compound can be determined by those of skill in the art, for example by performing serial dilutions and testing efficacy in an erythroid differentiation cell culture model, or other suitable system. Example concentration ranges for the treatment of the CD34⁺, hematopoietic stem and/or progenitor cells include, but are not limited to, about 1 nanomolar to about 10 millimolar; about 1 mM to about 5 mM; about 1 nM to about 500 nM; about 500 nM to about 1,000 nM; about 1 nM to about 1,000 nM; about 1 uM to about 1,000 uM; 1 uM to about 500 uM; about 1 uM to about 100 uM; about 1 uM to about 10 uM. In one embodiment, the range is about 5 uM to about 500 uM.

Cells can be treated for various times. Suitable times can be determined by those of skill in the art. For example, cells can be treated for minutes, 15 minutes, 30 minutes etc, or treated for hours e.g., 1 hour, 2 hours, 3 hours, 4 hours, up to 24 hours or even days. In one embodiment the cells are treated for 2 days prior to transplant.

The population of CD34⁺ cells that has been treated to promote differentiation, or to undergo gene correction, can be transplanted into a subject to regenerate erythroid hematopoietic cells in an individual having a disease that affects erythropoiesis, a disease associated with erythropoiesis. Such diseases can include, but are not limited to, cancers (e.g., leukemia, lymphoma), blood disorders (e.g., inherited anemia, inborn errors of metabolism, aplastic anemia, beta-thalassemia, Blackfan-Diamond syndrome, globoid cell leukodystrophy, sickle cell anemia, severe combined immunodeficiency, X-linked lymphoproliferative syndrome, Wiskott-Aldrich syndrome, Hunter's syndrome, Hurler's syndrome Lesch Nyhan syndrome, osteopetrosis), chemotherapy rescue of the immune system, and other diseases (e.g., autoimmune diseases, diabetes, rheumatoid arthritis, system lupus erythromatosis). In certain embodiments, the subject is selected for having been diagnosed with a disease associated with erythropoiesis. Methods for diagnosis of such diseases are well known to those of skill in the art. Most advanced regimes are disclosed in publications by Slavin S. et al., e.g., J Clin Immunol 2002; 22:64, and J Hematother Stem Cell Res 2002; 11:265, Gur H. et al. Blood 2002; 99:4174, and Martelli M F et al, Semin Hematol 2002; 39:48, which are incorporated in their entirety by reference.

Exemplary methods of administering treated cells to a subject, particularly a human subject, include injection or transplantation of the cells into target sites in the subject. The cells can be inserted into a delivery device which facilitates introduction, by injection or transplantation, of the cells into the subject. Such delivery devices include tubes, e.g., catheters, for injecting cells and fluids into the body of a recipient subject. In a preferred embodiment, the tubes additionally have a needle, e.g., a syringe, through which the cells of the invention can be introduced into the subject at a desired location. The cells can be inserted into such a delivery device, e.g., a syringe, in different forms. For example, the cells can be suspended in a solution, or alternatively embedded in a support matrix when contained in such a delivery device.

Unless otherwise defined herein, scientific and technical terms used in connection with the present application shall have the meanings that are commonly understood by those of ordinary skill in the art to which this disclosure belongs. A subset of definitions are provided below to help describe embodiments of the invention.

As used herein, a “subject” refers to, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject is optionally a mammal such as a primate or a human, individual.

“Expansion” or “expanded” in the context of cells refers to an increase in the number of a characteristic cell type, or cell types, from an initial population of cells, which may or may not be identical. It is contemplated herein that a CD34⁺ hematopoietic stem cell or progenitor cell can be expanded in culture prior to contacting CD34⁺ cells with an agent that alters occupancy at a signaling center, or with gene correction technology, and prior to transplantation into an individual in need thereof. Expansion can occur before or after inducing erythroid differentiation and/or concurrently with treatment of an agent that alters occupancy and gene expression at the signaling center.

As used herein, the term “promoting eyrthroid differentiation” refers to an increase in the efficiency or rate of eyrthroid differentiation, i.e., the amount of differentiation into eyrthroblasts and subsequent erythrocytes. Promotion of differentiation can be assessed by measuring erythroid development or gene expression under in vitro conditions in the presence and absence of the agent that alters occupancy at the signaling center (e.g. SMAD1 activator as described in Example 1). The effects seen under in vitro conditions correlates to effects expected in vivo. Differentiation can be measured by monitoring an increase in cells that are CD71⁺ and CD235⁺ in the presence of the agent as compared to the absence of the agent, during the differentiation process.

In certain embodiments, the presence of the agent increases the numbers of cells expressing CD71⁺ and CD235⁺ in a population already undergoing differentiation, e.g. there is an increase by at least 10%, 20%, at least 30%, at least 40%, at least 50%, at least 60%, at least 70%, at least 80%, at least 90%, at least 95%, at least 99%, at least 1-fold, at least 1.5×, at least 1.5 fold, at least 2-fold, at least 5-fold, at least 10-fold, at least 100-fold, at least 500-fold, at least 1000-fold or higher than observed in the absence of the agent (See e.g. Example 1, BMP4).

Erythropoiesis can be measured by monitoring the levels of CFU-Es, or the levels of eyrthrocytes in vitro or in vivo in a subject's blood before and after transplant of cells treated with the agent that alters gene expression at the signaling center. In certain embodiments, the numbers of erythrocytes increases by at least 20%, at least 30%, at least 40%, at least 50%, at least 60%, at least 70%, at least 80%, at least 90%, at least 95%, at least 99%, at least 1-fold, at least 1.5 fold, at least 2-fold, at least 5-fold, at least 10-fold, at least 100-fold, at least 500-fold, at least 1000-fold or higher in individuals. Erythropoiesis can also be assessed using a bone marrow aspirate sample and monitoring colony forming unit cells (CFU-Cs) and CFU-Es, methods are well known to those of skill in the art.

The term “CFU-E” or “erythroid colony-forming unit” as used herein refers to a progenitor cell derived from an hematopoietic stem cell which, when induced by erythropoietin, becomes committed to proliferate and differentiate to generate a colony of about 15-60 mature erythrocytes (which can be recognized in 7 days in a human bone marrow culture).

As used herein “a population of CD34⁺ cells” encompasses a heterogeneous or homogeneous population of cells that can include, hematopoietic stem cells and/or hematopoietic progenitor cells, and/or erytrhoid lineage committed cells. Specific markers are well known to those of skill and include, but are not limited to: markers for hematopoietic stem cell, e.g. cells that are CD34⁽⁺⁾⁽⁻⁾CD38⁽⁺⁾⁽⁻⁾CD45RA⁻CD49f⁺ and CD90⁺; markers for hematopoietic progenitor cells, e.g. cells that are CD34⁺CD45RA⁺CD38⁺; markers for erytrhoid lineage committed cells, e.g. cells that are CD34⁺CD38⁺CD45RA⁻. In addition, differentiated hematopoietic cells, such as white blood cells, can be present in a population of hematopoietic CD34⁺ cells. It is also contemplated herein that the population of CD34⁺ cells are isolated and expanded ex vivo prior to transplantation. Populations can be isolated using cell sorting techniques and markers well known to those of skill in the art. In some embodiments, the population of CD34⁺ cells is in vivo when contacted with the agent or gene correction technology.

As used herein, the term “hematopoietic progenitor cells” encompasses pluripotent cells capable of differentiating into several cell types of the hematopoietic system, including, but not limited to, granulocytes, monocytes, erythrocytes, megakaryocytes, B-cells and T-cells. Hematopoietic progenitor cells are committed to the hematopoietic cell lineage and generally do not self-renew; hematopoietic progenitor cells can be identified, for example by cell surface markers such as Lin-KLS⁺Flk2⁻CD34⁺. The presence of hematopoietic progenitor cells can be determined functionally as colony forming unit cells (CFU-Cs) in complete methylcellulose assays, or phenotypically through the detection of cell surface markers using assays known to those of skill in the art.

As used herein, the term “hematopoietic stem cell (HSC)” refers to a cell with multi-lineage hematopoietic differentiation potential and sustained self-renewal activity. “Self renewal” refers to the ability of a cell to divide and generate at least one daughter cell with the identical (e.g., self-renewing) characteristics of the parent cell. Hematopoietic stem cells can be identified with the following stem cell marker profile: Lin⁻ KLS⁺Flk2⁻CD34⁻.

As used herein, the term “erytrhoid lineage committed cells”, or hEPs, refers to cells that committed to become erythrocytes versus megakaryocytes. For example, hEPs are a CD71^{int/+}CD105⁺ fraction of a human megakaryocyte/erythrocyte progenitor population (hMEP; Lineage⁻ CD34⁺CD38⁺IL-3Rα⁻ CD45RA⁻) population (See Mori et al. Prospective isolation of human erythroid lineage-committed progenitors, Proc. Natl. Acad. Sci. U.S.A. 2015, 112(31): 9638-9643). Erytrhoid lineage committed cells include proerythroblasts.

As used herein, “erythroid differentiation” or “erythropoiesis” refers to the process of making erythrocytes, e.g. differentiation from the earliest stages includes the following steps of development that occur within the bone marrow 1.) A Hemocytoblast, a multipotent hematopoietic stem cell (e.g. CD34⁺CD38⁺CD45RA⁻CD90⁺), becomes 2.) a common myeloid progenitor or a multipotent stem cell (e.g. CD34⁺CD38⁺CD45RA⁻CD61⁻CD71⁻CD123⁺), and then a megacaryocyte erythrocyte progenitor cells (CD34⁺CD38⁺CD45RA⁻CD61⁻CD71⁻CD123⁻) differentiate into proerythroblasts (CD34⁺CD38⁺CD71⁺). The proerythroblasts differentiate into basophilic erythroblasts (CD34⁻CD38⁺CD71⁺) which in turn differentiate into polychromatic erythroblast (CD34⁻CD38⁻CD71⁺), then into a red blood cell; markers for erythrocytes include for example CD34⁻, CD59⁺ and glycophorin⁺/CD235a⁺.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention.

Some embodiments of the technology described herein can be defined according to any of the following numbered paragraphs:
1) A method for modulating erythropoiesis comprising contacting a CD34⁺ cell with an agent that alters occupancy at a signaling center in the genome of the cell, wherein the signaling center comprises 1) a DNA binding site for a lineage-specific regulator, and 2) a DNA binding site for a signal-responsive transcription factor, wherein increasing gene expression at the signaling center promotes erythropoiesis.
2) The method of paragraph 1, wherein the signaling center further comprises a tissue-specific transcription factor DNA binding motif
3) The method of paragraph 1, wherein the agent that alters occupancy at the signaling center is an agent that induces binding of the signal-responsive transcription factor to the signaling center.
4) The method of paragraph 1, wherein the agent that alters occupancy at the signaling center is an agent that inhibits binding of the signal-responsive transcription factor to the signaling center.
5) The method of paragraph 1, wherein the signal-responsive transcription factor is selected from the group consisting of SMAD1, SMAD5, SMAD8, β-catenin, LEF/TCF, STAT5, RARA, BCL11A, TCF7L2, CREB3L, CREB, CREM, CTCF, IRF7, RELB, AP2B, NFKB2, PAX, PPARG, RXRA, RARG, RARB, E2F6m TBX20, TBX1, NFIA, NFIB, ZN350, TCF4, EGR1, and THRB.
6) The method of paragraph 1, wherein the agent that alters occupancy at the signaling center in the genome is an agonist of a signaling pathway selected from the group consisting of: nuclear hormone receptor, cAMP pathway, MAPK pathway, JAK-STAT pathway, NFKB pathway, Wnt pathway, TGFβ/BMP pathway, LIF pathway, BDNF pathway, PGE2 pathway, and NOTCH pathway.
7) The method of paragraph 1, wherein the agent that alters occupancy at the signaling center is selected from the group consisting of: a small molecule, a nucleic acid RNA, a nucleic acid DNA, a protein, a peptide, and an antibody.
8) The method of paragraph 1, wherein the lineage-specific regulator is the transcription factor GATA1 or GATA2.
9) The method of paragraph 1, wherein the signaling center comprises the signal-responsive binding site for transcription factor SMAD1 and the lineage-specific regulator binding site for the transcription factor GATA1, and wherein the agent that alters occupancy at the signaling center increases expression of one or more genes selected from Table 4 (D5 SE genes), or from Lengthy Table S6.
10) The method of paragraph 1, wherein the signaling center comprises the signal-responsive transcription factor binding site for SMAD1 and the lineage-specific regulator binding site for the transcription factor GATA2, and wherein the agent that alters occupancy at the signaling center increases expression of one or more genes selected from Table 3 (H6 SE genes), or from Lengthy Table S6.
11) The method of paragraph 9 or 10, wherein the agent that alters occupancy at the signaling center signaling center is an agent that activates the transcription factor SMAD1. (in specification: see end of paragraphs for definition)
12) The method of paragraph 11, wherein the agent is an agonist of a BMP receptor kinase.
13) The method of paragraph 11, wherein the agent that activates the transcription factor SMAD1 is a checkpoint kinase 1 (CHK1) inhibitor.
14) The method of paragraph 11, wherein the agent that activates SMAD1 is selected from the group consisting of: PD407824, MK-8776, LY-2606368 and LY-2603618, BMP4, BMP2, BMP7, isoliquirtigenin, apigenin, 4′-hydroxychalcone, and diosmetin.
15) The method of paragraph 1, wherein the signaling center comprises the signal-responsive binding site for transcription factor SMAD1 and the lineage-specific regulator binding site for the transcription factor GATA1 or GATA2, and wherein co-binding of either SMAD1/GATA1 or SMAD/GATA2 at the signaling center alters expression of long non-coding RNAs (IncRNAS), e.g. an IncaRNA from lengthy Table S5.
16) The method of paragraph 1, wherein the CD34⁺ cell is derived from a source selected from the group consisting of: bone marrow, peripheral blood, cord blood and derived from induced pluripotent stem cells.
17) The method of paragraph 1, wherein the CD34⁺ cell is a hematopoietic stem cell or a hematopoietic progenitor cell.
18) A method for treating a disease associated with aberrant erythropoiesis comprising correcting the DNA of a CD34⁺ cell that is present at the site of a signaling center, wherein the signaling center associated with normal erythropoiesis comprises 1) a DNA binding site for a lineage-specific regulator, and 2) a DNA binding site for a signal-responsive transcription factor.
19) The method of paragraph 18, wherein the correction of the DNA restores the binding of the signal-responsive transcription factor to the signaling center.
20) The method of paragraph 18, wherein the lineage-specific regulator is transcription factor GATA1 or GATA2.
21) The method of paragraph 18, wherein the signal-responsive transcription factor is selected from the group consisting of SMAD1, SMAD5, SMAD8, β-catenin, LEF/TCF, STAT5, RARA, BCL11A, TCF7L2, CREB3L, CREB, CREM, CTCF, IRF7, RELB, AP2B, NFKB2, PAX, PPARG, RXRA, RARG, RARB, E2F6m TBX20, TBX1, NFIA, NFIB, ZN350, TCF4, EGR1, and THRB.
22) The method of paragraph 18, wherein the signaling center further comprises a tissue-specific transcription factor DNA binding motif
23) The method of paragraph 18, wherein the DNA is corrected using a gene editing tool.
24) The method of paragraph 23, wherein the gene editing tool is CRISPER technology or TALEN Technology.
25) The method of paragraph 18, wherein the disease associated with aberrant erythropoiesis is selected from the group consisting of: leukemia, lymphoma, inherited anemia, inborn errors of metabolism, aplastic anemia, beta-thalassemia, Blackfan-Diamond syndrome, globoid cell leukodystrophy, sickle cell anemia, severe combined immunodeficiency, X-linked lymphoproliferative syndrome, Wiskott-Aldrich syndrome, Hunter's syndrome, Hurler's syndrome Lesch Nyhan syndrome, osteopetrosis, chemotherapy rescue of the immune system, and an autoimmune disease.
26) The method of paragraph 18, wherein the signal-responsive binding site is the binding site for the transcription factor SMAD1, and wherein restoring binding of SMAD1 to the signaling center increases expression of one or more genes selected from Table 4 (D5 SE genes), from Table 3 (H6 SE genes), or from Lengthy Table S6.
27) The method of paragraph 18, wherein the correction of the DNA restores binding of the native signal-responsive transcription factor to the signaling center restoring wild-type expression of one or more genes selected from Table 5 or Table 6.
28) The method of paragraph 18, wherein the CD34⁺ cell is a hematopoietic stem cell or a hematopoietic progenitor cell.
29) The method of paragraph 18, wherein the CD34⁺ cell is in vivo.
30) The method of paragraph 18, wherein the CD34⁺ cell is in vitro and derived from a source selected from the group consisting of: bone marrow, peripheral blood, cord blood and derived from induced pluripotent stem cells.
31) The method of paragraph 30, wherein the CD34⁺ cell is transplanted into the subject after correction of the DNA at the site of the signaling center.

### EXAMPLES

### Example 1

BMP Signaling Cooperates with GATA Factors to Govern Stage-Specific Gene Expression During Erythroid Differentiation.

Few studies have defined how multiple signaling programs influence stage-specific gene expression during intermediate stages of erythropoiesis. Instead, there has been a focus on gene expression driven by lineage-specific regulators at extreme stages. Previous and current suggest that, in various hematopoietic cell-lines, BMP-signal responsive transcription factor SMAD1 strikingly marks genomic regions which are co-occupied by critical effector transcription-factors of other signaling pathways. Such regions were defined as “Signaling Centers”. In this study presented herein, SMAD1 was utilized as a surrogate molecule to identify critical Signaling Centers formed at every step of human erythropoiesis. It was investigated how SMAD1, as part of Signaling Centers, localizes with hematopoietic lineage-restricted GATA transcription factors. Such interactions specify intermediate cell-types by defining stage-specific active enhancer elements and thereby orchestrate temporal gene expression patterns. By overlapping RNAseq and ChIPseq for SMAD1, GATA factors and H3K27Ac, as well as ATACseq to investigate open chromatin regions at specific stages, human erythroid differentiation has been extensively mapped in CD34⁺ cells. Surprisingly, SMAD1-binding gradually shifts from GATA2 to GATA1-occupied enhancer regions and marks the genes that are responsible for differentiation. Such regions correlate with open chromatin and super-enhancers at every stage, whereas GATA-only regions are associated with genes with low/basal level of expression during differentiation. In contrast to GATA-only sites, SMAD1-GATA co-bound enhancer regions harbor cis-acting motifs and display enriched binding of cell-type specific transcription factors (e.g. SPI1 and FLI1 in progenitor vs. KLF1 and NFE4 in differentiated cells).

CRISPR-CAS9 mediated perturbations of such transcription factor motifs along with GATA motif severely downregulate expression of the nearby gene indicating that lineage-restricted master regulators play critical role in the formation of stage-specific Signaling Centers. Analysis of human single nucleotide polymorphisms (SNPs) revealed that SMAD1-binding at erythroid stage remarkably overlaps with red-blood-cell-trait-associated variations. SNPs were associated with six erythrocyte traits: Hemoglobin concentration (Hb), Hematocrit (Hct), Mean corpuscular volume (MC), Mean corpuscular hemoglobin (MCH), Mean corpuscular hemoglobin concentration (MCHC), Red blood cell count (RBC). Out of 108 genes reported to be associated with RBC-related SNPs, 72 genes (67%) have at least one variation within close proximity of SMAD1 binding site. Moreover, many of these SNPs either destroy or create effector transcription factor motifs of various signaling pathways that include nuclear hormone receptor-, BMP-, WNT-, cAMP, MAPK-, JAK-STAT-, TGFB- and NFKB-signaling as well as others, See for example Tables 5 and 6 herein at the end of the specification (Note: genes can be cross referenced with lengthy Table 51 to find the signaling center location). This observation clearly shows that naturally occurring human variations can directly impact genomic regions where signaling factors converge. Taken together, the study presented herein indicates that SMAD1 binding, in close proximity to lineage-restricted master transcription factors, defines cell-fate by marking the functionally active Signaling Centers that are stage-specific. This provides an opportunity to investigate the formation and implications of such signaling hotspots and indicates a mechanism of how individuals with distinct genetic makeup can differentially respond to various environmental stress.

BMP Signaling Affects the Erythroid Differentiation Potential of CD34⁺ HSPCs

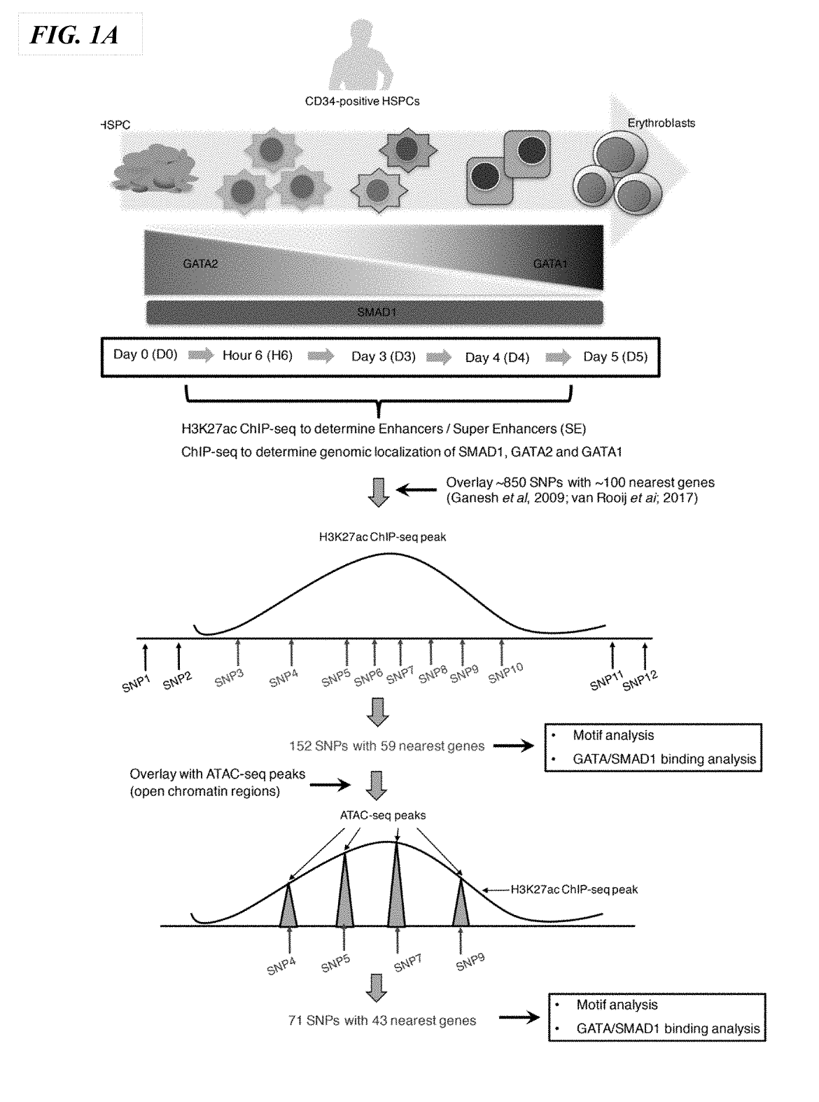

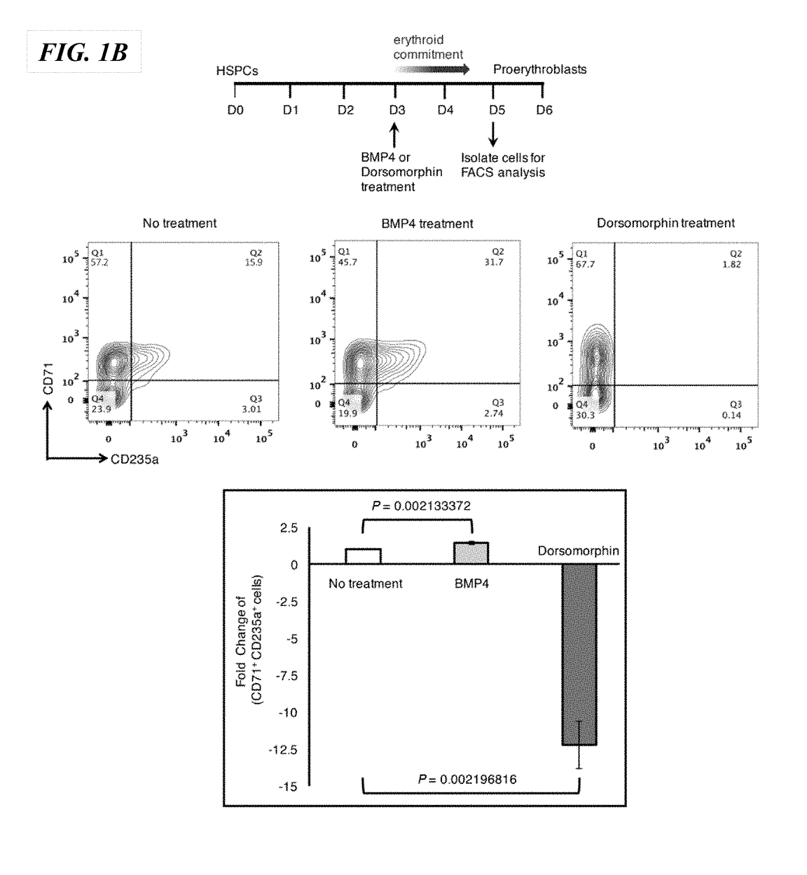

To determine the key time-points defining the stages of erythroid commitment, primary human stem and progenitor CD34⁺ cells (CD34⁺ HSPCs) from mobilized peripheral blood were used as a model of erythroid differentiation (Sankaran et al., 2008a) (FIG. 1A). Immunohistochemistry targeting GATA2, GATA1, and β-globin at 6 hours (H6), 3 days (D3), 4 days (D4), and 5 days (D5) of erythroid differentiation was used (FIG. 1B). High GATA2 and low or absent GATA1 expression is expected at progenitor stages, and this ratio should invert during differentiation, with GATA1 replacing GATA2 during a “GATA switch.” β-globin expression is a hallmark of cells that have committed to the erythroid lineage. Consistent with this model, it was observed that GATA2 is abundantly expressed during the initial stages of differentiation but its expression drops significantly by D4, whereas GATA1 protein is readily observed from D3 of differentiation onward (Bresnick et al.,2010; Dore et al., 2012). The GATA switch marks a cell's commitment to the erythroid fate and is accompanied by expression of β-globin (FIG. 1B). These observations indicate that progenitor cells commit to an erythrocyte fate around D3.

To establish the role of BMP signaling in erythroid differentiation, differentiating CD34⁺ cells were treated with human recombinant BMP4 (hrBMP4) to activate the pathway, or with dorsomorphin, a known inhibitor of BMP signaling. Knowing that erythroid lineage commitment occurs around D3, differentiating cells were treated for two days starting from D3, to test the effects of these signals on erythrocyte commitment. FACS analysis of the erythroid markers CD71 and CD235a at the end of D4 shows a mild but statistically significant 1.5-2-fold increase in erythroid cell counts upon BMP4 treatment. In contrast, treatment with dorsomorphin significantly reduces the erythroid differentiation potential, establishing the importance of BMP signaling in erythroid differentiation (FIG. 1C).

### SMAD1, GATA2, and GATA1 Co-Bind Genomic Regions in a Timepoint-Specific Manner

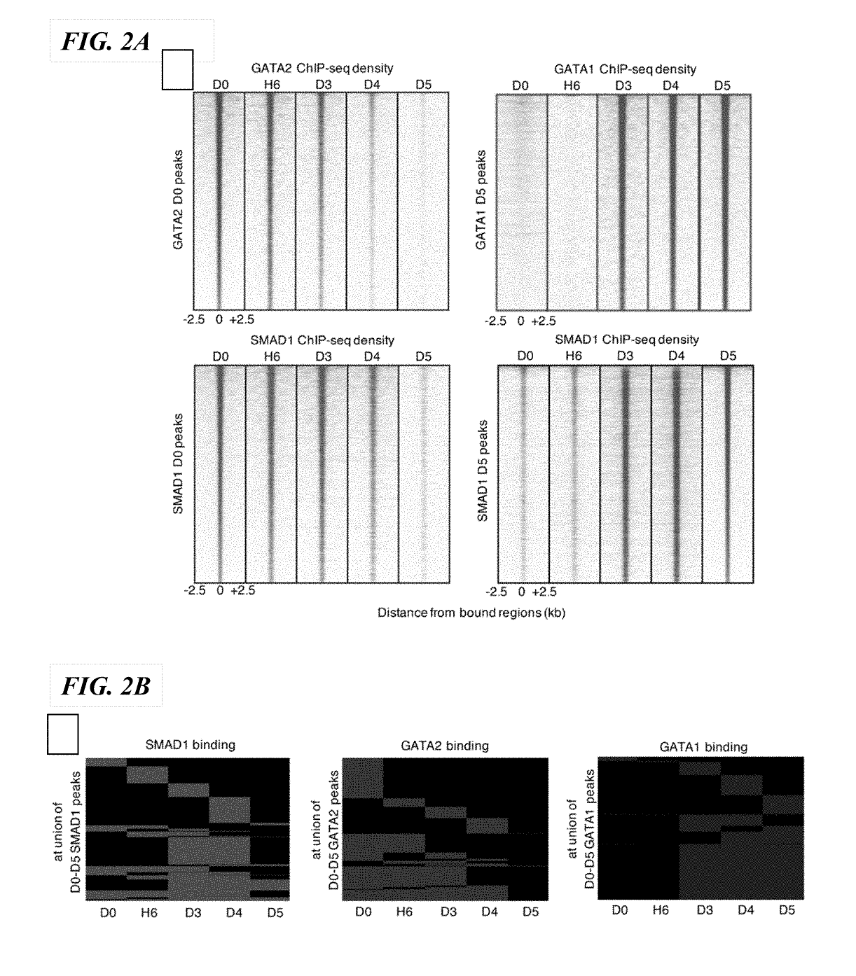

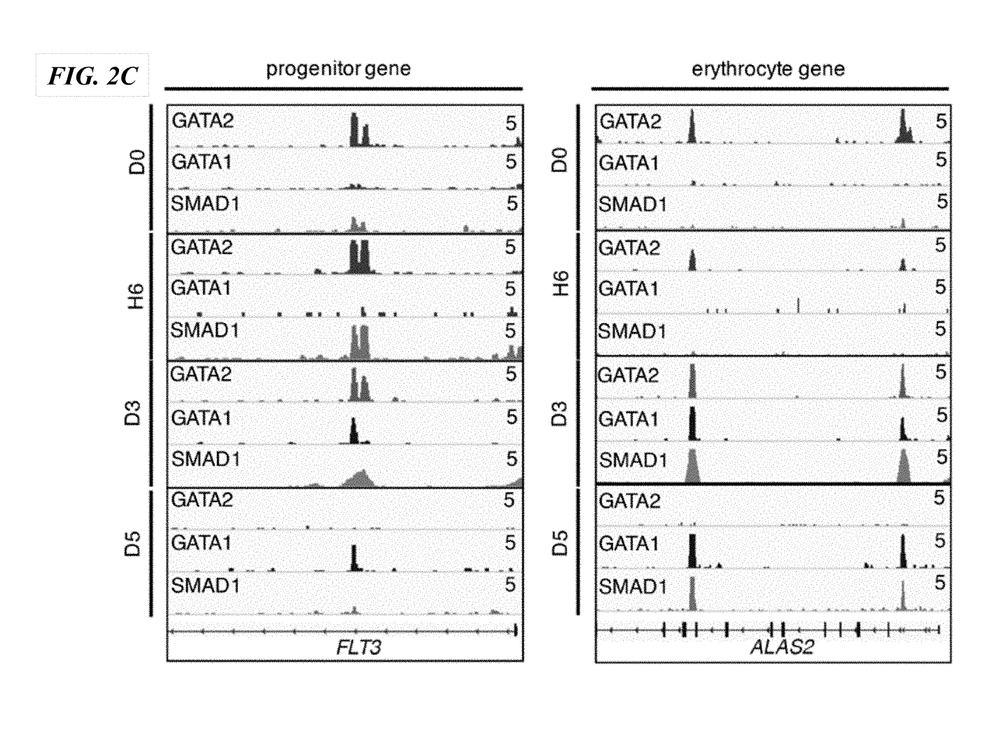

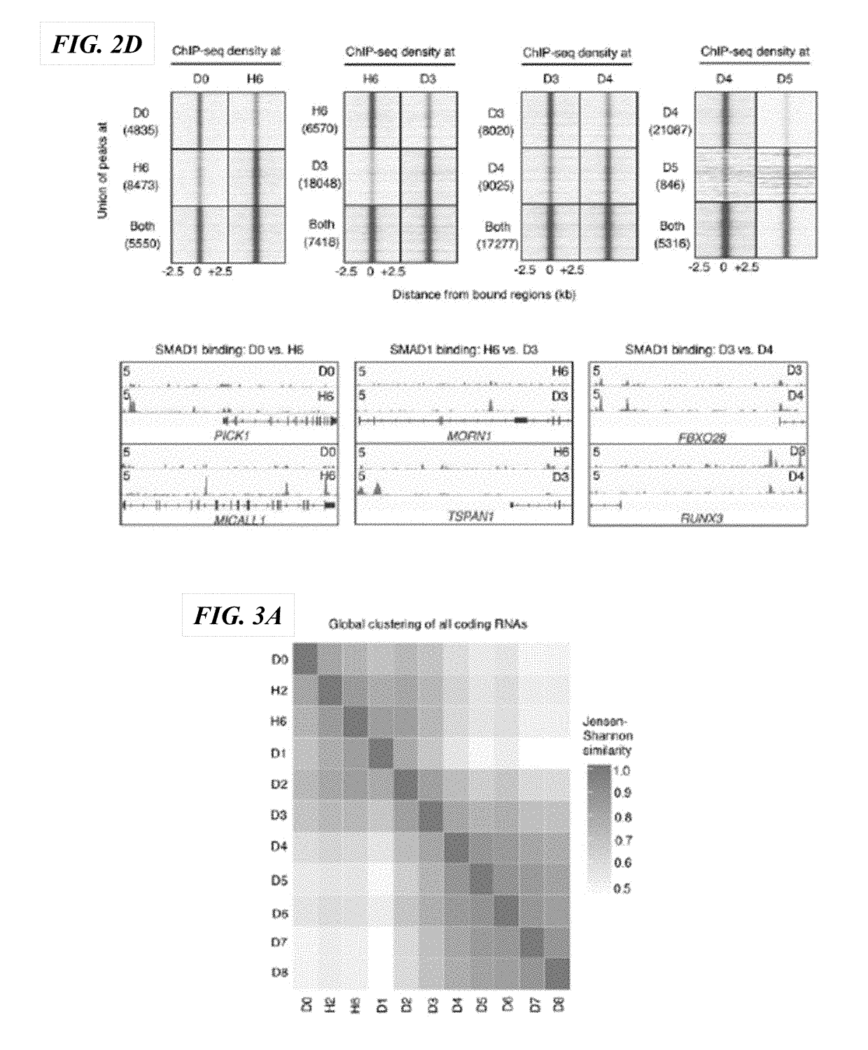

BMP signaling affects gene expression through several BMP-responsive transcription factors including SMAD1 (Singbrant et al., 2010), so the binding of SMAD1 to regulatory elements during erythropoiesis was interrogated. To investigate the localization of SMAD1 on chromatin and its relationship to GATA factors binding during subsequent stages of erythroid commitment, ChIP-seq experiments targeting SMAD1, GATA2, GATA1 and H3K27ac were performed on D0 (progenitor stage before the addition of differentiation media), H6, D3, D4 and D5 after pulse treatment of human CD34⁺ cells with hrBMP4 for two hours. In progenitor cells, GATA2 binds a large number of genes that are key for multiple distinct blood lineages (4017 genes, Table S1). Gradually, genome-wide GATA2 occupancy and expression decreases, and is nearly absent by D4 (FIGS. 2A, B and C). In contrast, GATA1 binding near erythroid genes is observed and maintained from D3 onwards in accordance with the immunofluorescence results (FIGS. 2A, 2B, 2C and FIG. 1B).

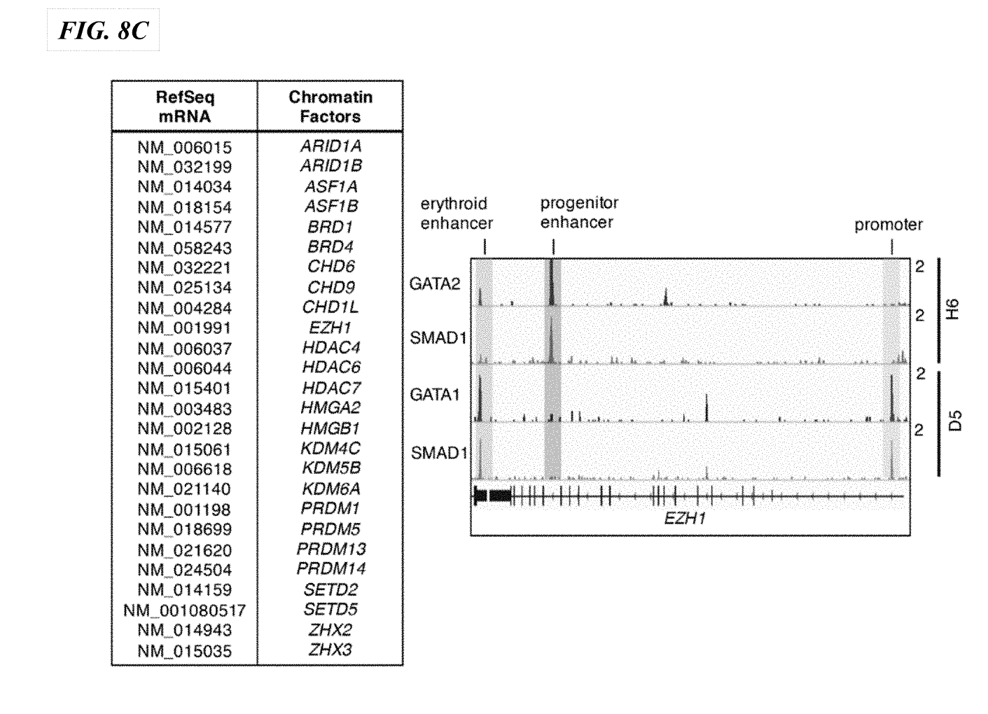

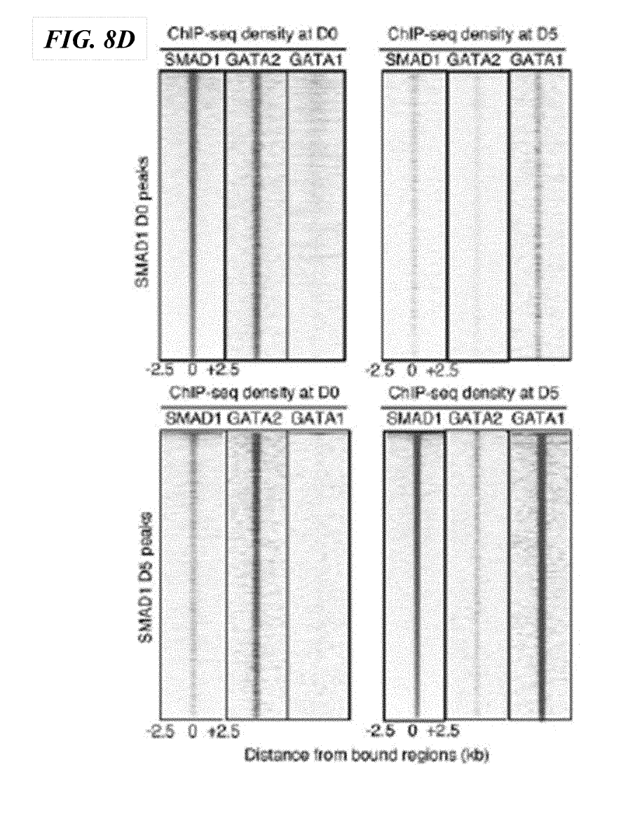

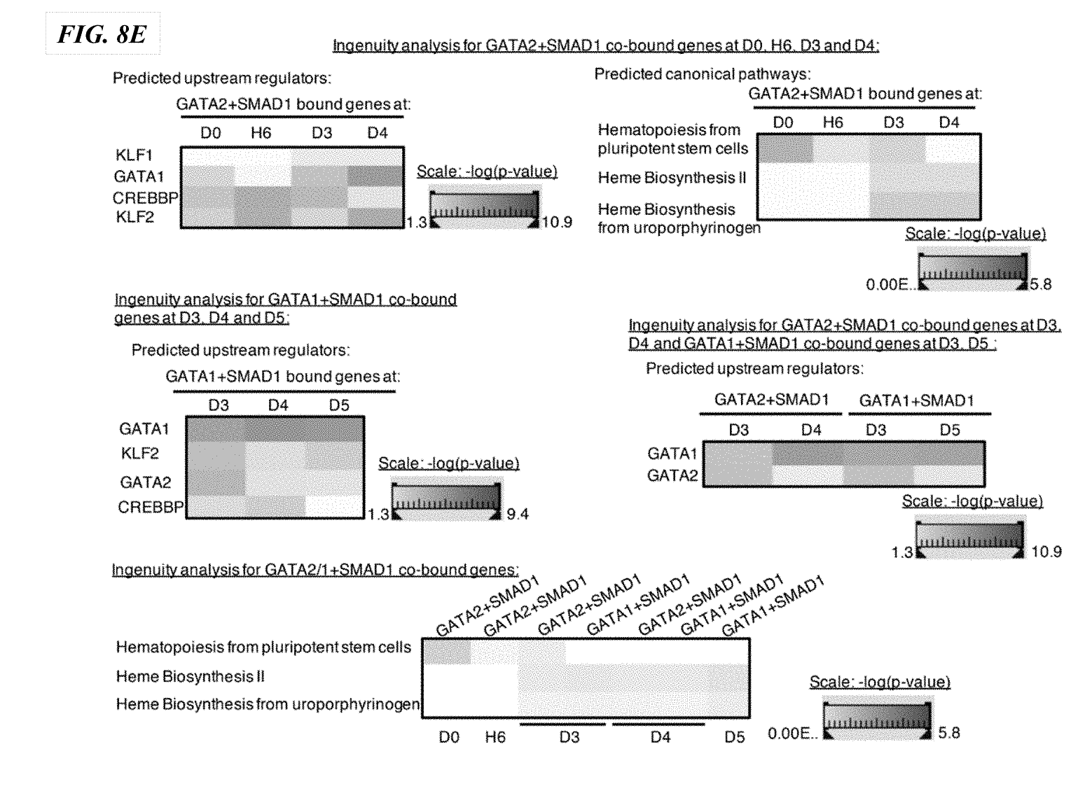

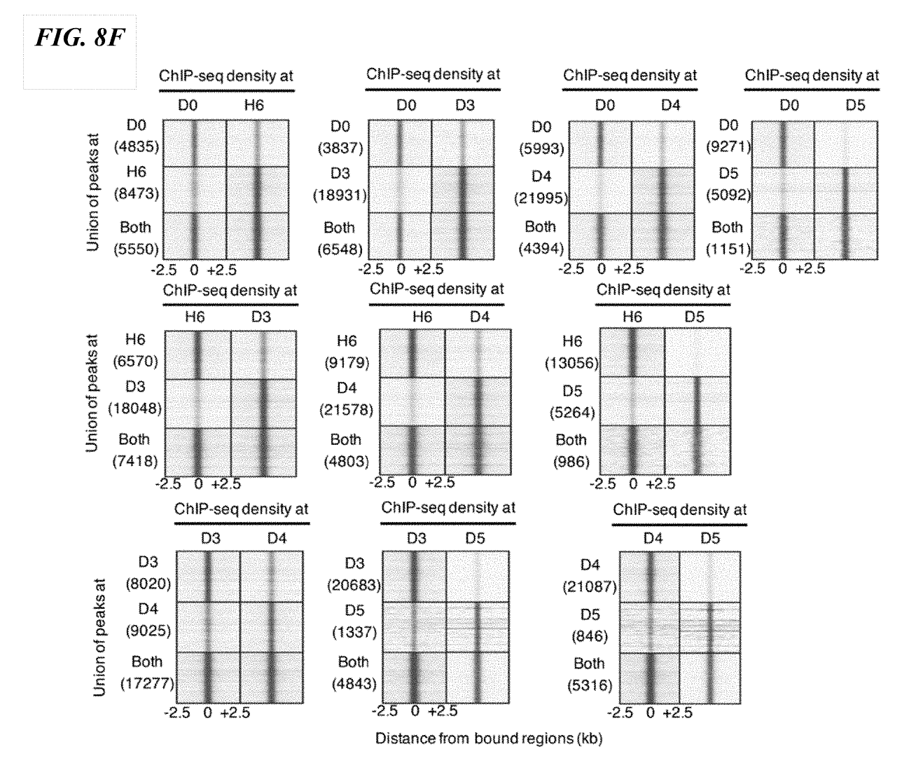

During differentiation, a prominent switch from GATA2 to GATA1 binding is observed in regions bound by GATA2 during early stages (H6, FIGS. 8A, 8B) consistent with replacement of GATA2 by GATA1 as the master regulator. As the cells commit to an erythroid fate, 1475 or 57% of genes bound by GATA2 at H6 are bound by GATA1 at D5, indicating the “GATA switch” is driven primarily by transcriptional regulation (data not shown). Key chromatin factor-encoding genes are associated with regions that undergo this switch (FIG. 8C Top Panel, and data not shown). For example, data presented herein confirms a previously observed “GATA switch” on EZH1 and further indicates that the switch need not occur at the same binding site (Xu et al., 2015) (FIG. 8C Bottom Panel). ASF1, which was recently found to play a role in congenital dyserythropoietic anemias, also shows replacement of GATA2 by GATA1 (Iolascon et al., 2013). The key transcriptional co-factors HMGA1 and BRD4, which have established roles in erythropoiesis, are also regulated by timepoint-specific GATA members (Isern et al., 2011; Stonestrom et al., 2015).

SMAD1 Binds DNA Near Key Cell-Type Specific Genes at all Time Points.

Incontrast to GATA2 that loses sites and GATA1 that gains sites, SMAD1 both gains and loses binding sites during erythropoiesis (FIGS. 2A, 2B). SMAD1 co-binds DNA with timepoint-specific GATA family members. In progenitor cells, SMAD1 co-binds with GATA2 on progenitor-specific genes; after the fate-switch, SMAD1 co-binds with GATA1 on erythroid genes as shown by Ingenuity Pathway Analysis (FIGS. 8D and 8E). At D0, 81% of genes bound by SMAD1 are also occupied by GATA2; at D5, 82% of genes bound by SMAD1 are also occupied by GATA1. Additionally, 81% and 84% of GATA1-bound genes are also bound by SMAD1 at D3 and D4, respectively (See Lengthy Table 51). Representative gene tracks at different stages of differentiation in FIG. 2C show SMAD1 and GATA factor co-localization and how their binding progressively changes from progenitor-specific to erythroid-specific genes. It is worth noting that co-occupancy by both GATA2 and GATA1 does occur at D3 on progenitor and erythrocyte genes, again emphasizing this as a transitional stage (FIG. 2C). SMAD1 binding varies not only between pre- and post-commitment cells but also between all time points. Comparison of SMAD1 binding between D0 and H6 shows that 46% of SMAD1 bound regions are unique to D0 and 54% are shared with H6 (FIG. 2D, Top Panel). Approximately, 22% of SMAD1 sites remain common between D0 to D3, and 18% between D3 and D5. At D4, SMAD1 binding overlaps with ˜15% of the D0 binding sites and 19% of D5 genomic sites (FIG. 8F). Examples of stage-specific gene-tracks that gained or lost SMAD1 binding between subsequent stages are shown in FIG. 2D, (Bottom Panel). Taken together, these observations depict variable genomic occupancy by SMAD1 during erythroid differentiation on stage-specific genes. These data indicate that SMAD1 co-operates with GATA factors and may regulate stage-specific gene expression.

Co-Binding of SMAD1 and GATA Factors Determines Stage-Specific Gene Expression

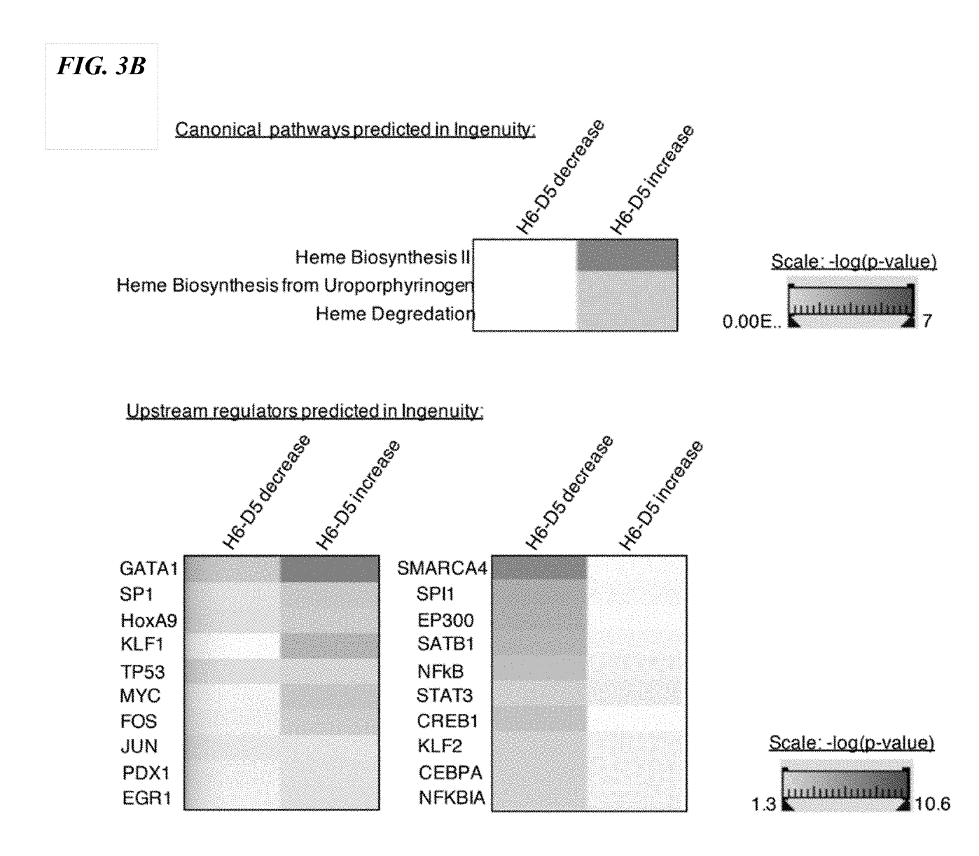

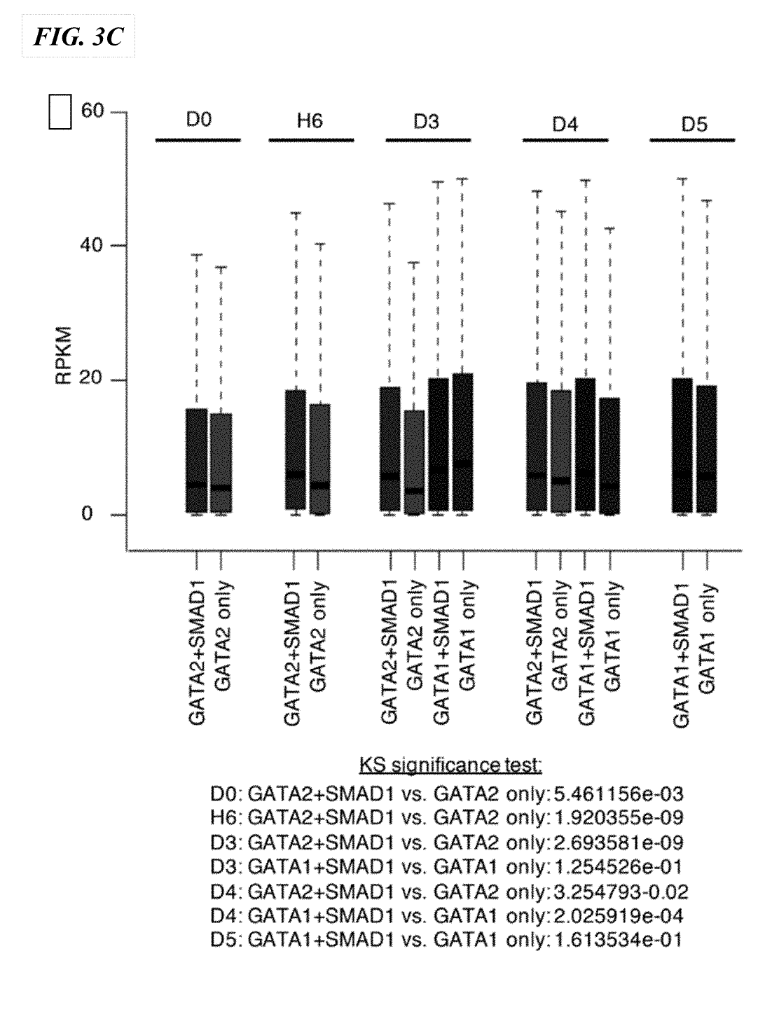

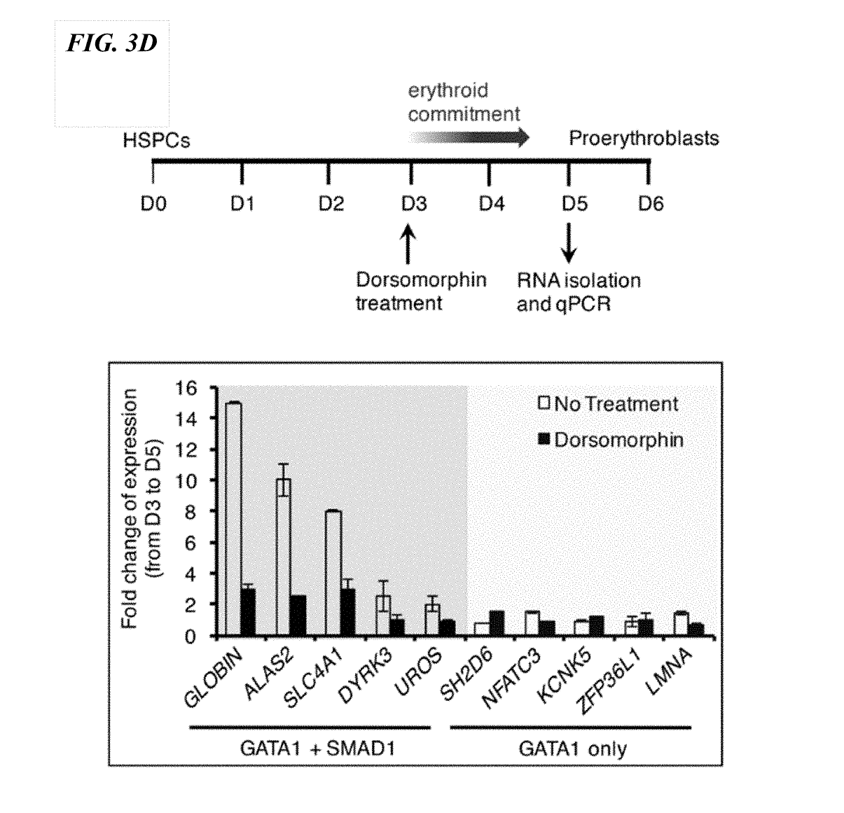

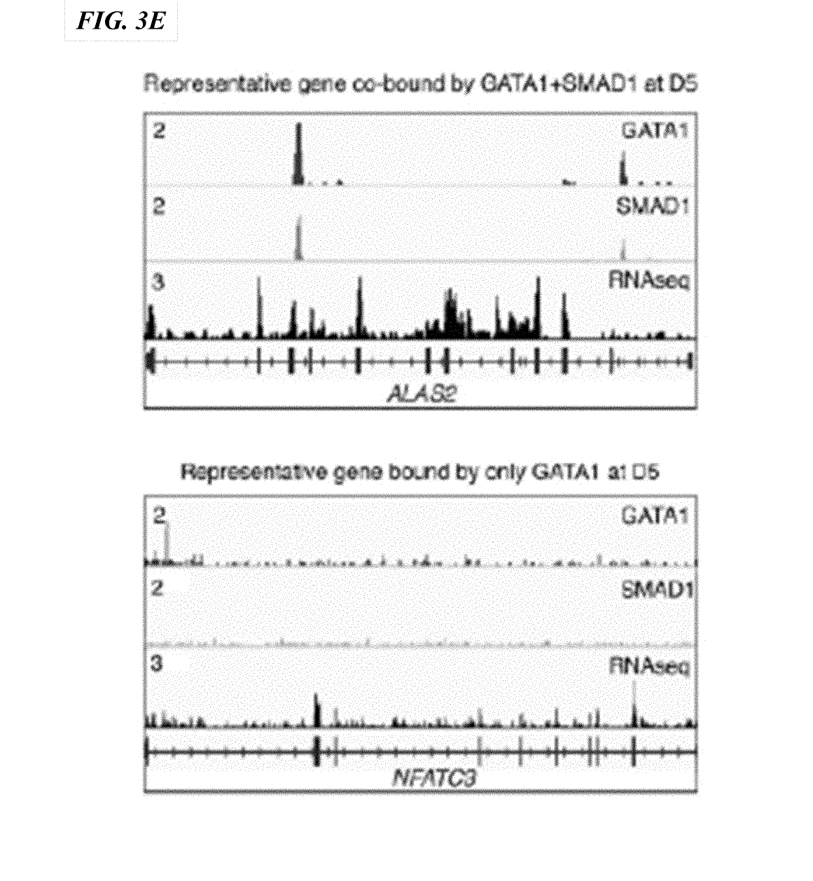

It was next asked whether SMAD1 and GATA factors regulate expression of key timepoint-specific genes during erythropoiesis. For this purpose, RNA-seq was performed, after a 2 hr pulse of hrBMP, on progenitor and differentiating cells at 2 and 6 hours of erythroid differentiation and daily from days 1 through 8. The genome-wide expression profiles cluster into two groups, before and after D3 in accordance with the timing of a “GATA switch” (FIG. 3A and data not shown). The 1475 genes associated with regions that undergo a switch of GATA binding also change expression more than 1.75-fold from H6 to D5, with 30% increasing, 37% decreasing, and 33% remaining stable. Pathway analysis showed that the upregulated genes are associated with erythroid-specific biological functions and their predicted upstream regulators are erythroid transcription factors (FIG. 3B). Comparison of ChIP-seq and RNA-seq shows that co-localized regions, either co-bound by SMAD1 and GATA2 at earlier time points or by SMAD1 and GATA1 at later time points, were associated with genes exhibiting higher expression compared to regions occupied only by GATA factors (FIG. 3C). Interestingly, GATA1/SMAD1 co-bound regions show lower expression compared to GATA1-only regions at D3. It is possible that during the transitional stage at D3, some erythroid-specific genomic regions are still repressed by GATA2 and will transition to activation after SMAD1/GATA1 fully occupy the respective regions. Taken together, these results confirm the dominant roles of GATA2 and GATA1 in erythropoietic gene expression and indicate that key stage specific genes are regulated by both SMAD1 and GATA factors.

To investigate whether BMP signaling affects gene expression through SMAD1 binding, CD34⁺ cells were treated with BMP-blocking dorsomorphin at D3, and RNA was isolated at the beginning of D5 of differentiation for qPCR analysis (FIG. 3D, Top Panel). Expression quantification via qPCR for five genes which are co-bound by SMAD1 and GATA1 at D5 (HBB, ALAS2, SLC4A1, DYRK3, UROS) and five genes which had high-confidence binding of only GATA1 (NFATC3, SH2D6, ZFP36L1, KCNK5, LMNA) showed that inhibition of BMP signaling by dorsomorphin selectively decreases expression of GATA1/SMAD1 co-bound genes, but not of those occupied by GATA1 alone (FIG. 3D, Bottom Panel; FIG. 3E). It is also worth noting that GATA1/SMAD1 co-bound genes exhibit higher expression than the GATA1 only genes. Thus BMP signaling actively regulates gene expression.

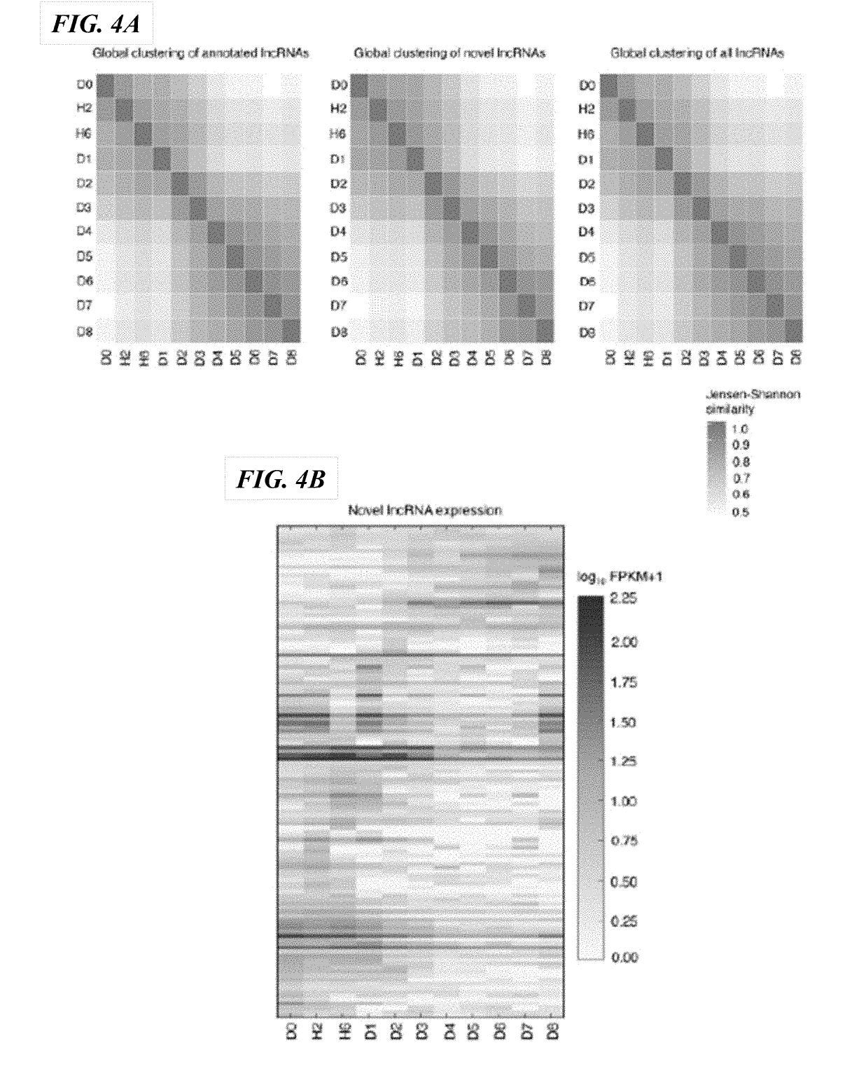

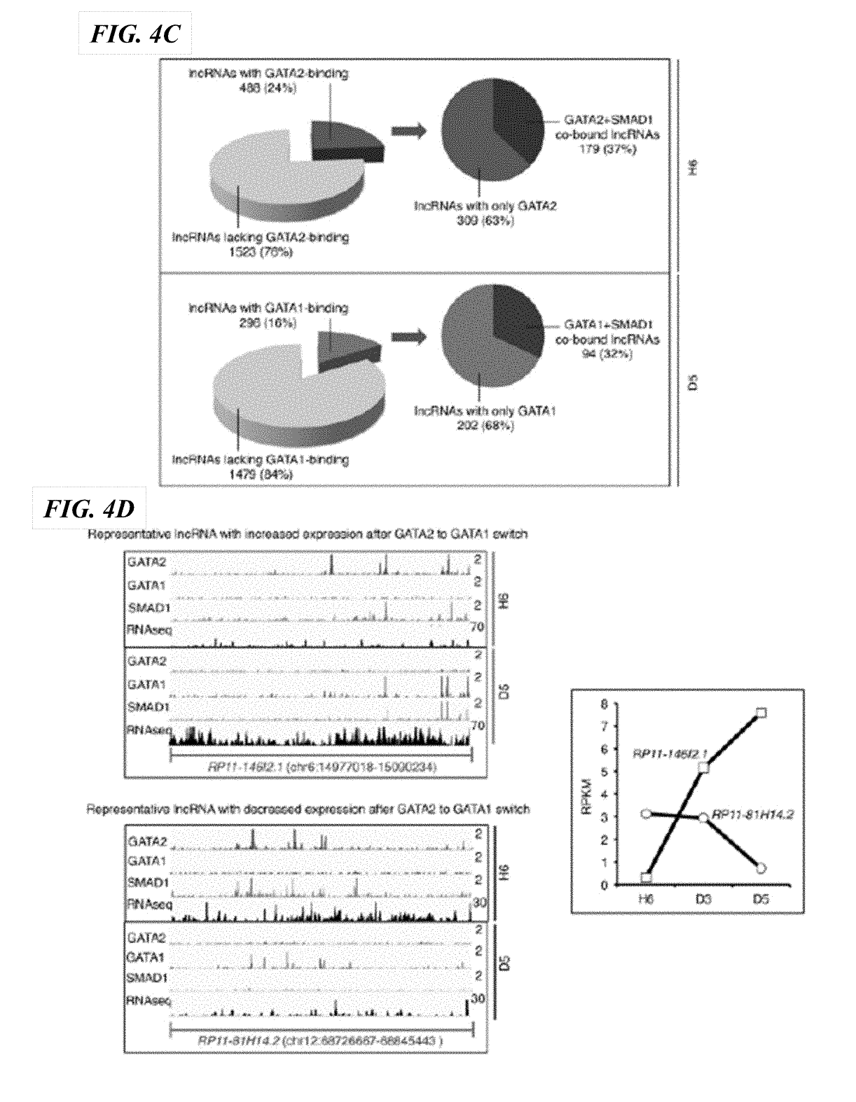

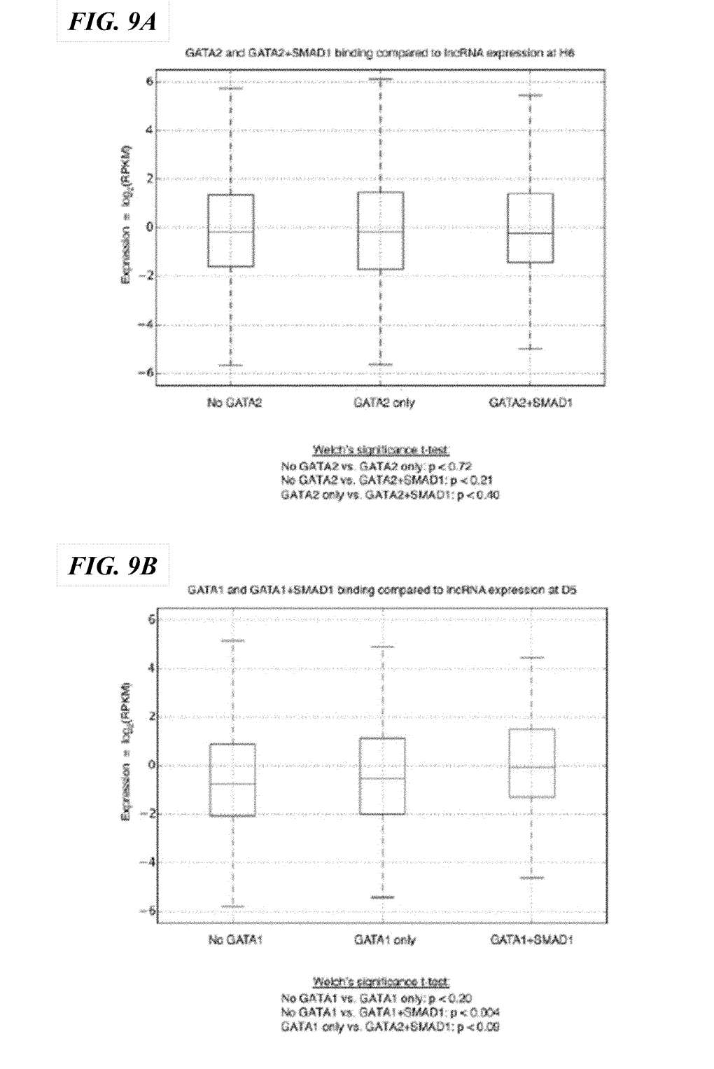

Besides protein coding genes, long non-coding RNAs (lncRNAs) have been proposed to play key roles in regulating mammalian hematopoiesis and erythropoiesis (Alvarez-Dominguez et al., 2014; Paralkar et al., 2014; Paralkar and Weiss, 2013; Satpathy and Chang, 2015). The RNA-seq data presented herein was analyzed for lncRNA expression associated with erythropoiesis (Hung and Chang, 2010; Rinn, 2014; Rinn and Chang, 2012). The expression of was quantified for known lncRNAs and further identified 142 putative novel lncRNAs from datasets presented herein (data not shown). Clustering all timepoints by genome-wide lncRNA expression reveals two predominant groups corresponding to the progenitor and erythroid states (FIG. 4A). Clustering the novel lncRNAs according to their expression levels across the timecourse revealed a broad range of expression dynamics (FIG. 4B), including a number of progenitor- and erythroid-specific lncRNAs. LncRNA genes are frequently bound by key transcription factors (Alvarez-Dominguez et al., 2014; Paralkar et al., 2014). To investigate the role of GATA/SMAD1 binding in lncRNA expression during erythropoiesis, the distributions of lncRNA expression at H6 and D5 were compared. From the 2,011 lncRNAs with non-zero expression at H6, 488 (24%) are bound by GATA2, of which 179 (37%) are also bound by SMAD1. At D5, 1,775 lncRNAs were identified with non-zero expression. Of these, 296 (16%) are bound by GATA1, and 94 of these 296 (32%) lncRNA genes are co-bound by SMAD1 (FIG. 4C and data not shown). While lncRNA-genes bound by GATA2 at H6—with or without SMAD1 co-binding do not exhibit higher expression than those without GATA2, lncRNA-genes bound by GATA1 at D5 do show higher expression than those that are not (p<0.01, Welchs t-test), and in particular, lncRNA-genes co-bound by GATA1/SMAD1 show even higher expression than those unbound by GATA1 (p<0.004; FIGS. 9A and 9B). Representative examples of lncRNAs expressed in both progenitor and erythroid cells showing upregulation or downregulation upon GATA switch are shown in FIG. 4D. Taken together, these observations indicate that GATA and SMAD1 are significant regulators of mRNA and lncRNA expression during erythroid differentiation. However, it cannot preclude the existence of other regulators with significant impact.

Super-Enhancers (SEs) are Occupied by GATA and SMAD1

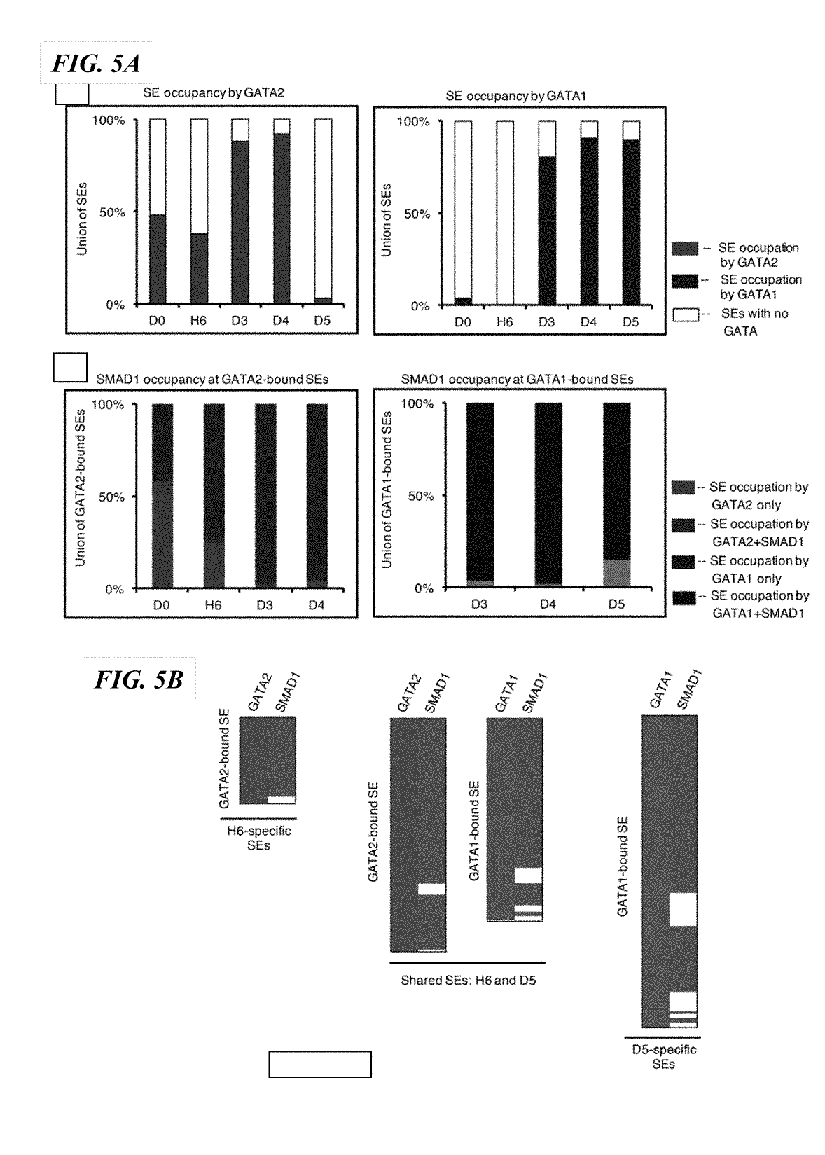

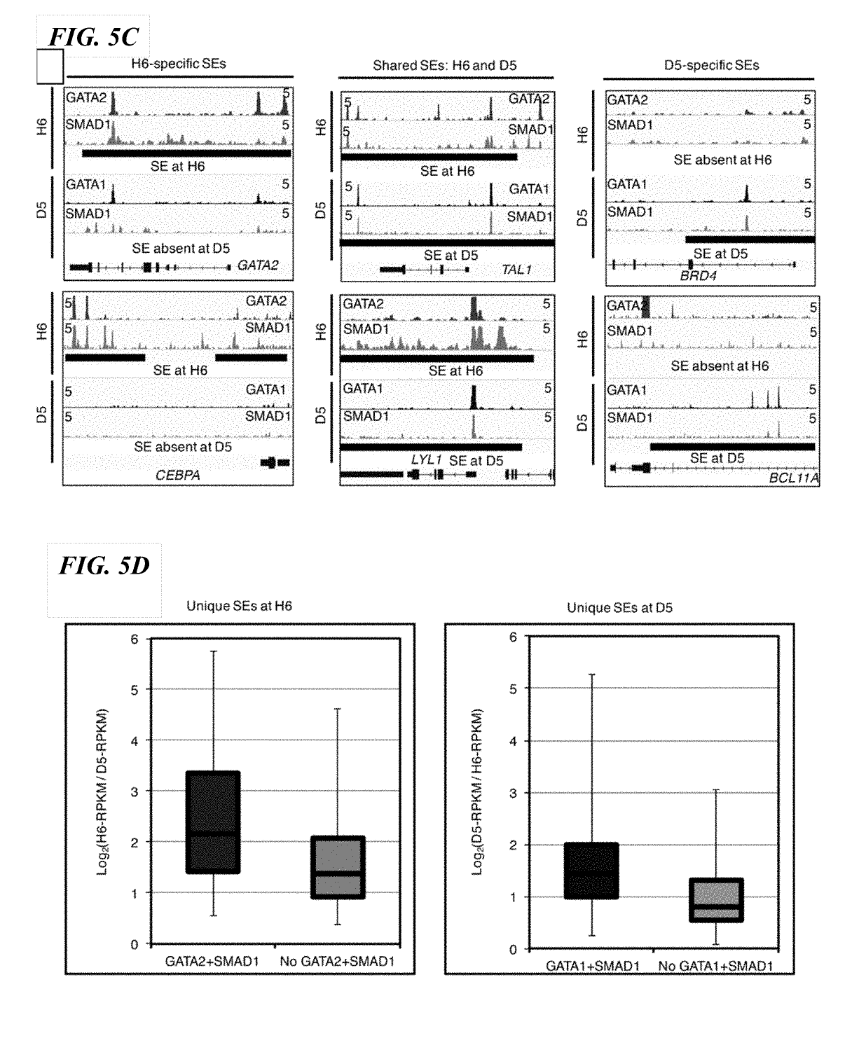

Genes that are important for controlling and defining cell identity are associated with super-enhancers, large genomic regions that act as platforms for gene regulation by both lineage and signaling transcription factors (Hnisz et al., 2013; Hnisz et al., 2015; Whyte et al., 2013). Utilizing ChIP-Seq targeting histones marked with H3K27-acetylation, which marks active enhancers, SEs associated with multiple time-points during CD34⁺ differentiation were identified, and SE-occupancy by GATA factors and SMAD1 was investigated (See Lengthy Table S6). Identified SEs were associated with genes that play key roles at different stages of blood development, including GATA2, FLI1, CEBPA at H6 and GATA1, BCL11A, GFI1B at D5. Consistent with the GATA switch, SEs detected in progenitors were bound primarily by GATA2, and SEs detected at D5 were bound primarily by GATA1 (FIG. 5A, Top Panels).

Approximately, 47% (329 out of 698) and 38% (170 out of 446) of SEs are bound by GATA2 at D0 and H6, respectively, compared to 89% (371 out of 415) of SEs that are bound by GATA1 at D5. Cells during transition at D3 and D4 showed SE-occupancy by both GATA factors, and the GATA2 to GATA1 switch at SEs was a prominent feature at D3 of differentiation and appears to be complete by D5 (FIG. 5A, Top Panels). Overlapping of GATA1/2 and SMAD1 ChIP-seq with predicted SEs at all the differentiation stages shows that substantial fractions of GATA2-bound and GATA1-bound SEs are also co-bound by SMAD1. Specifically, 42% (138 out of 329) of GATA2 bound SEs at D0, 70% (119 of 170) GATA2-bound SEs at H6, 97% (380 of 390) GATA2-bound SEs at D3, and 95.5% (423 out of 443) of GATA2-bound SEs at D4 are also bound by SMAD1. Correspondingly, 97% (338 out of 348), 99% (440 out of 445) and 83% (307 out of 371) of GATA1-bound SEs are also bound by SMAD1 at D3, D4 and D5, respectively (FIG. 5A, Bottom Panels).

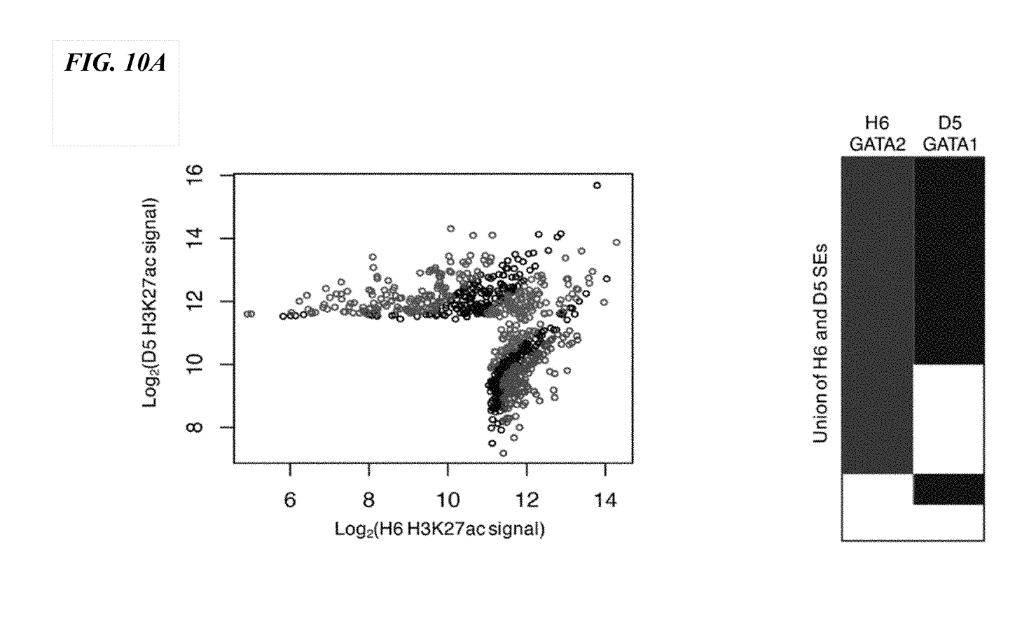

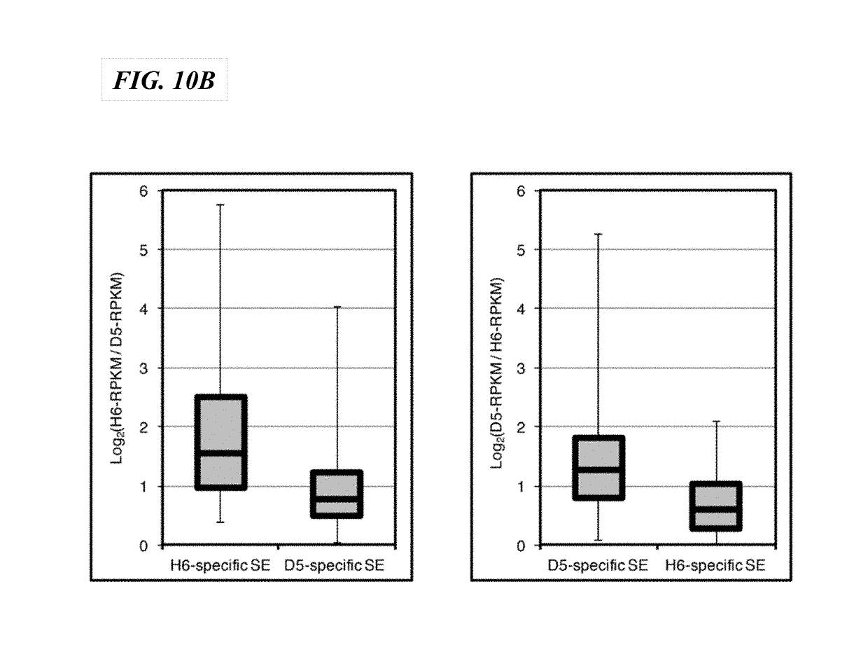

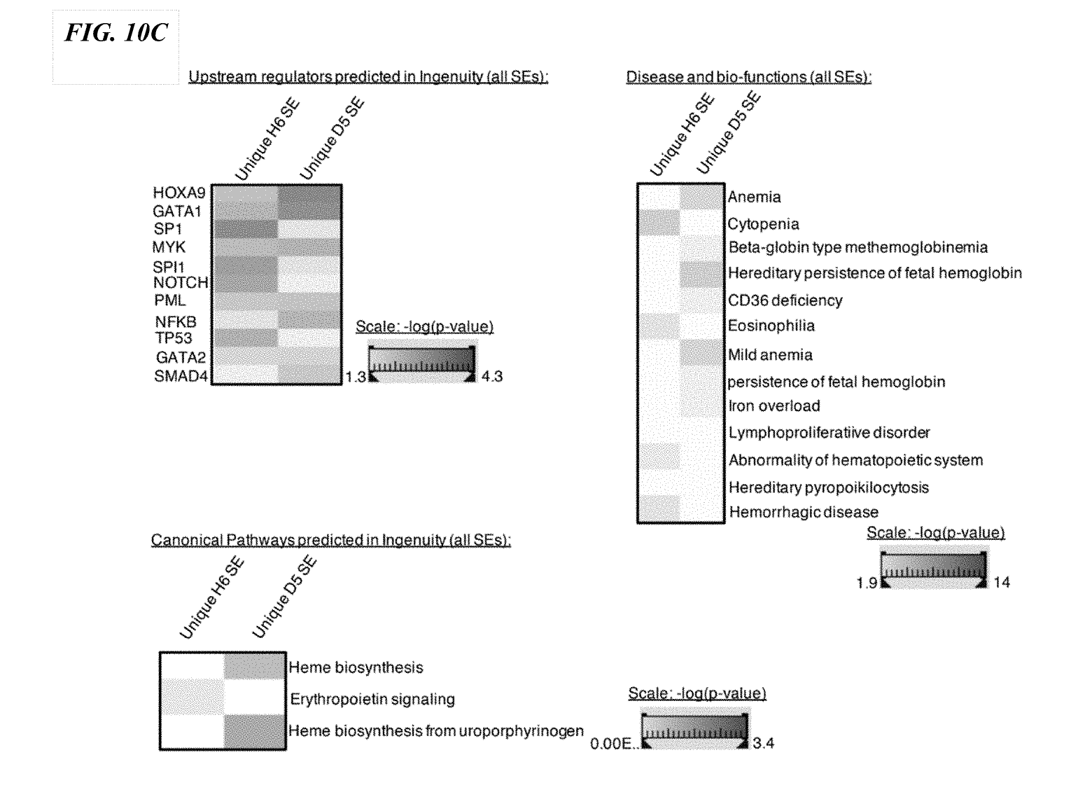

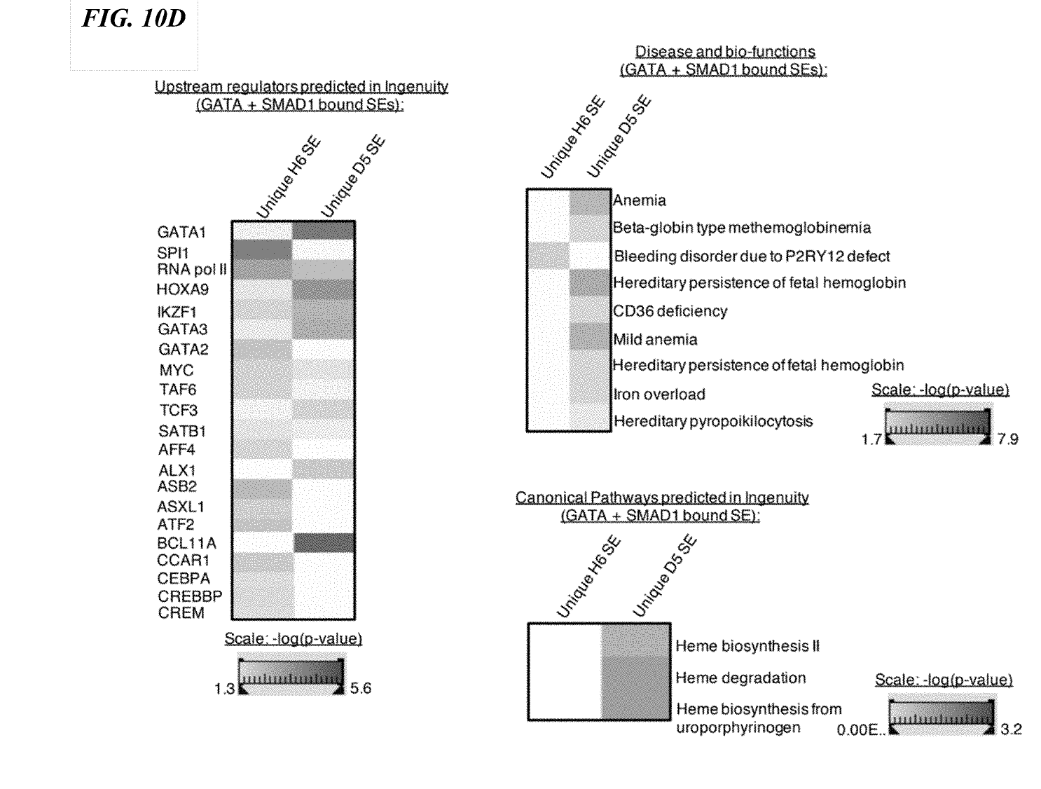

To show the interplay of SMAD1 and GATA at SEs during“GATA switch,” stage-specific SEs was determined and shared SEs at H6 and D5 based on their H3K27-acetylation signal at each timepoint (FIG. 10A, Left Panel). These lists comprise SEs that are lost (top 150 H6-specific SEs), acquired (top 150 D5-specific SEs) and are shared between the timepoints (top 150 common SEs) as the cells differentiated (See Lengthy Table S6; and Table 3, H6 SE at end of specification; and Table 4, D5 SE at end of specification).

The list validates a switch from GATA2 binding at H6 to GATA1 binding at D5 at about 50% of SEs (FIG. 10A, Right Panel). The majority of these GATA-bound SEs at both the stages were also bound by SMAD1 (FIG. 5B). Individual gene tracks for such GATA/SMAD1 co-bound stage-specific (e.g. GATA2, CEBPA at H6 and BCL11A, BRD4 on D5) and common SE-associated genes (e.g. TAL1 and LYL1 at both H6 and D5) are shown in FIG. 5C. These results indicate that SMAD1 plays a role in either establishing or maintaining the regulation of SE-associated genes along with the stage-specific GATA factors.

Previous work has shown the genes associated with super-enhancers tend to be more highly expressed, so it was next tested whether these timepoint-specific SEs correlated with gene expression (Whyte et al., 2013). Comparison of RPKM values of the genes associated with the top 150 SEs at each stage clearly showed higher expression of stage-specific SEs-associated genes compared to the SE-associated genes at the other stage (FIG. 10B). Additionally, SEs bound by both GATA and SMAD1 were associated with stage-specific high expression compared to the SEs defined at alternative stages (FIG. 5D). Ingenuity pathway analysis indeed showed that stage-specific SE-associated genes, as well as genes associated with GATA1/SMAD1-cobound SEs at D5, are related to erythroid-specific biological functions and are predicted to be regulated by erythrocyte-specific transcription factors (FIGS. 10C and 10D). Results presented herein indicate that SMAD1, in association with GATA factors, mark critical stage-pecific regulatory elements that guide the cells during differentiation.

SMAD1 Co-Localizes with GATA at Open Chromatin Regions

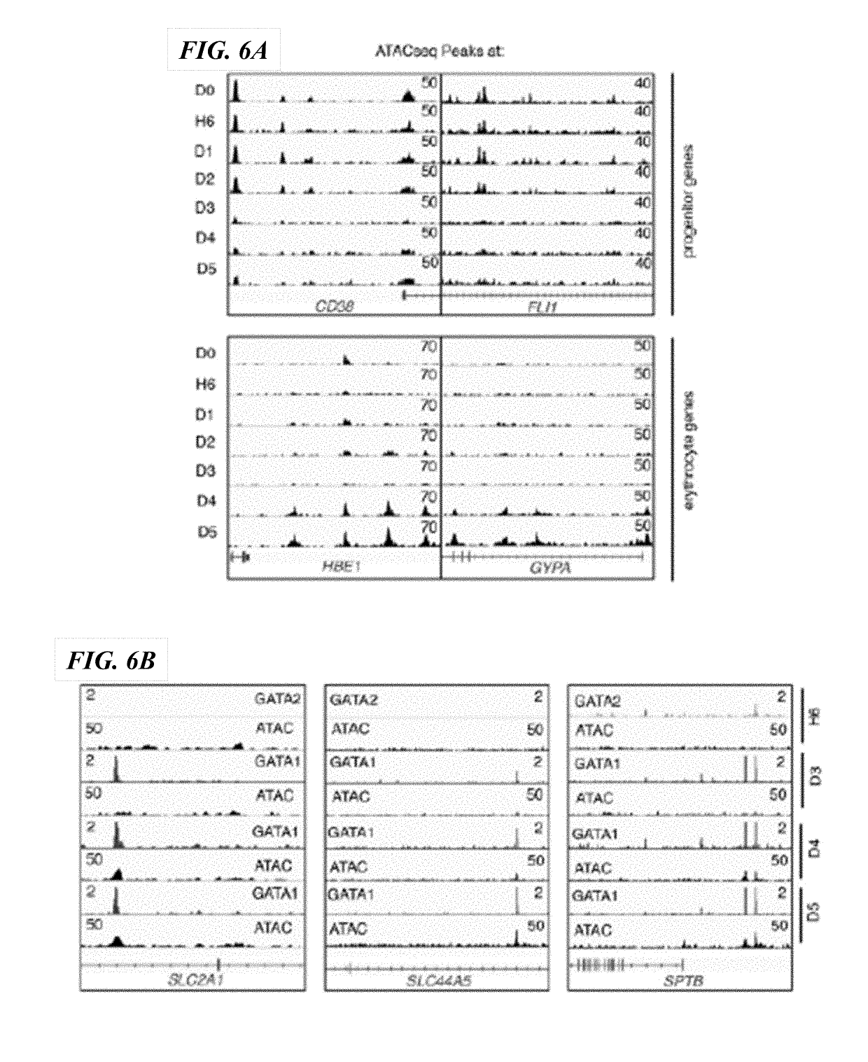

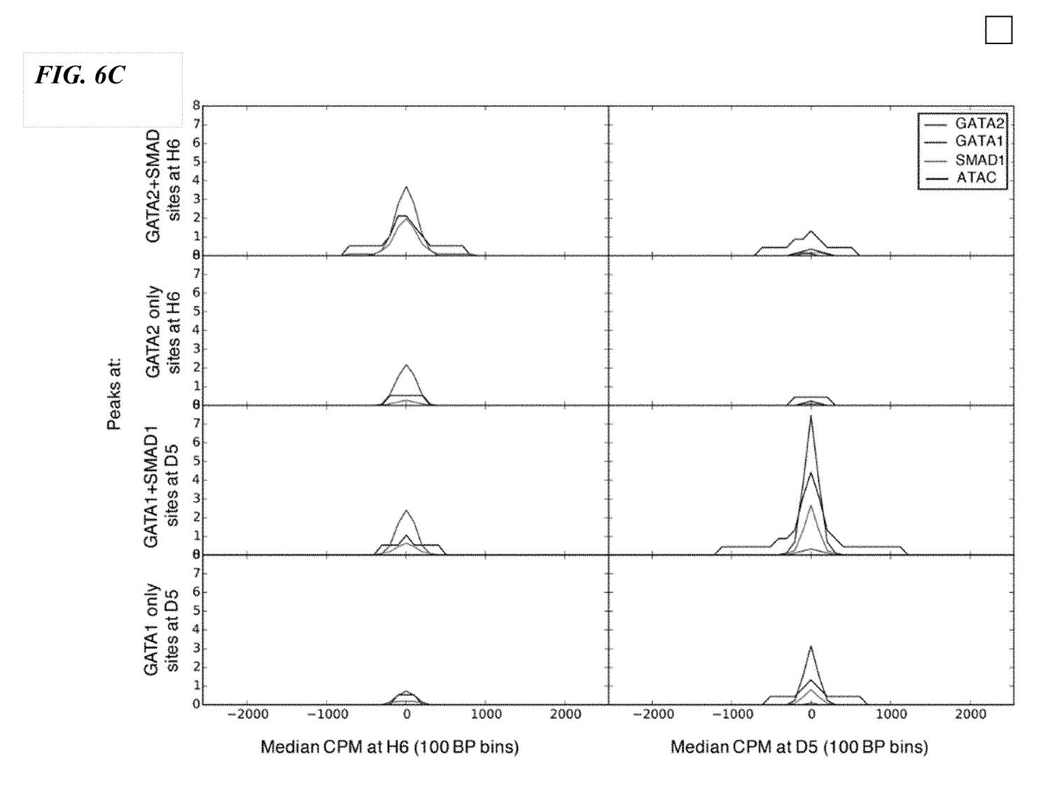

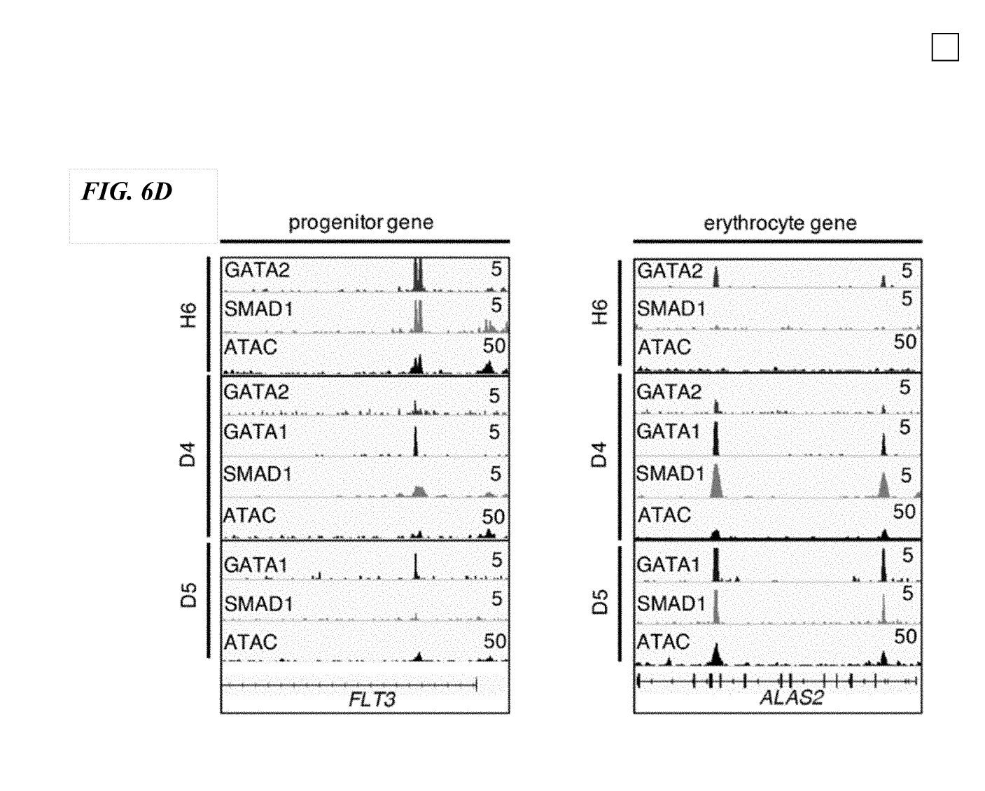

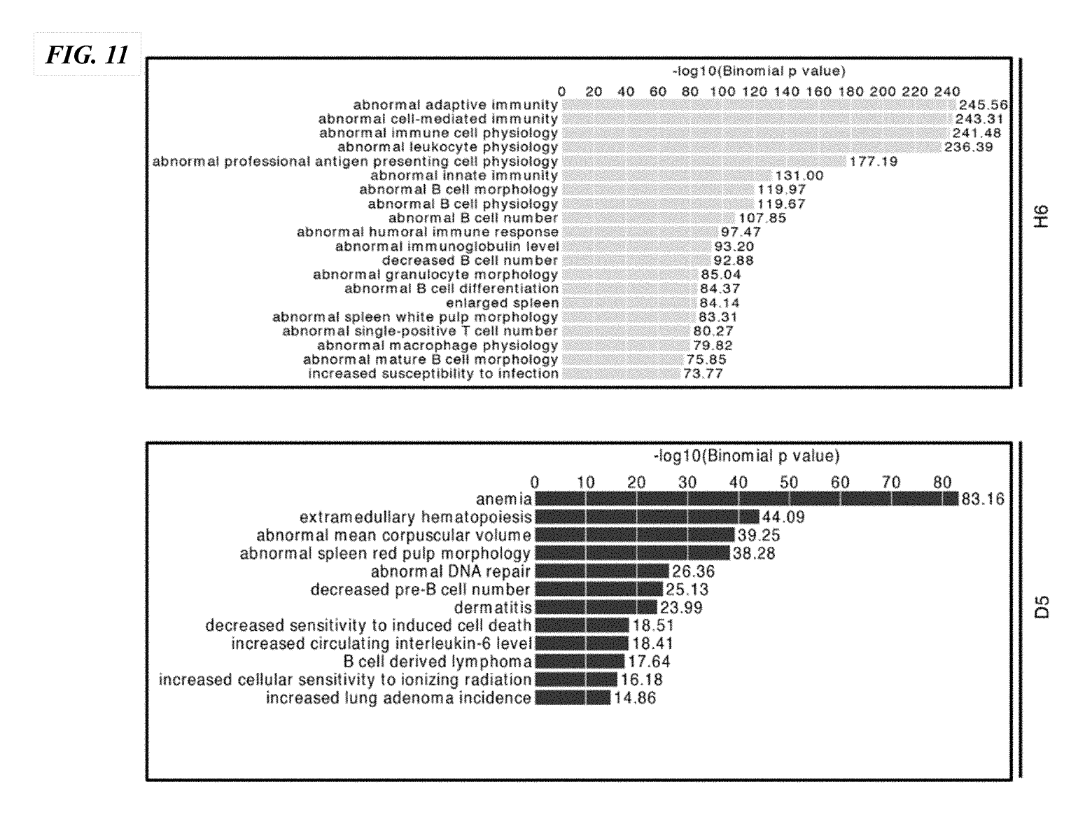

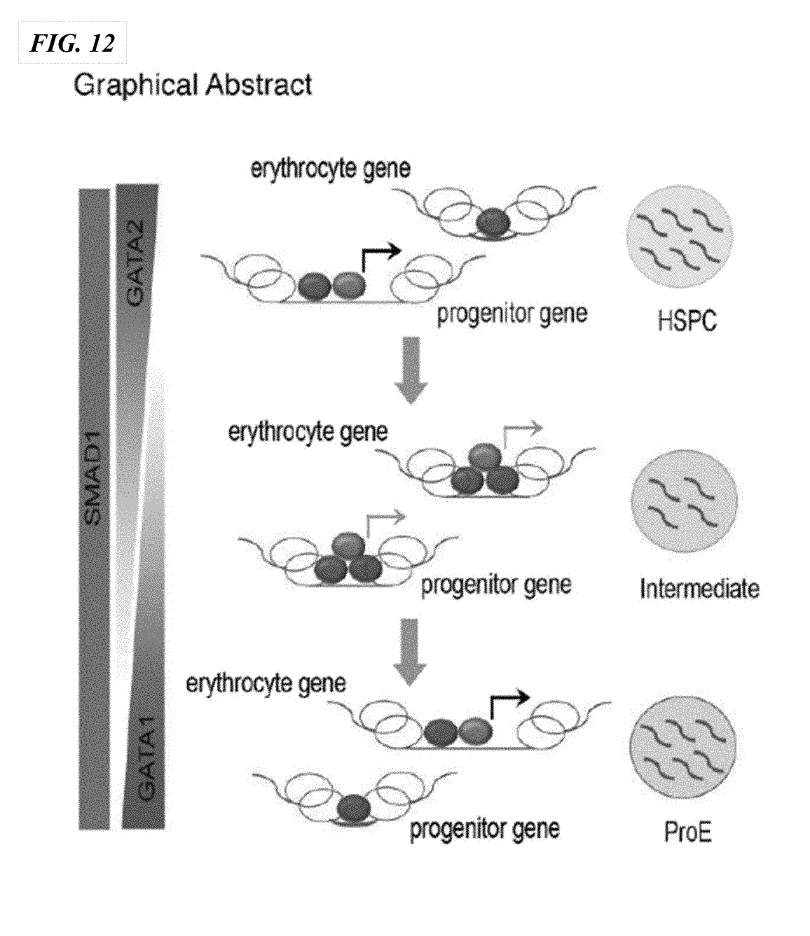

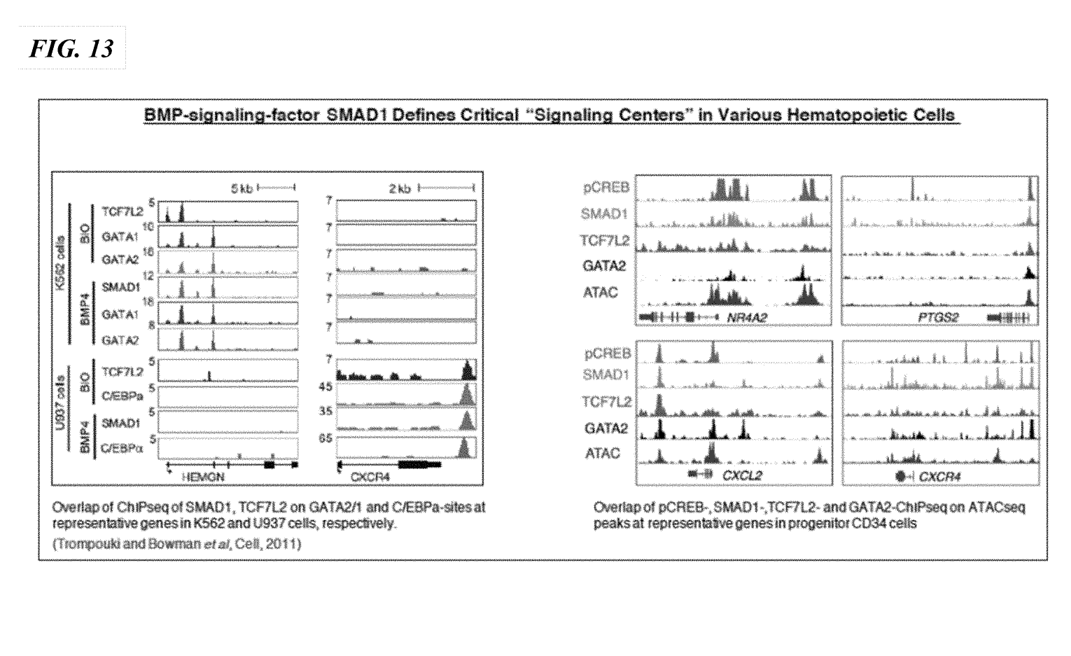

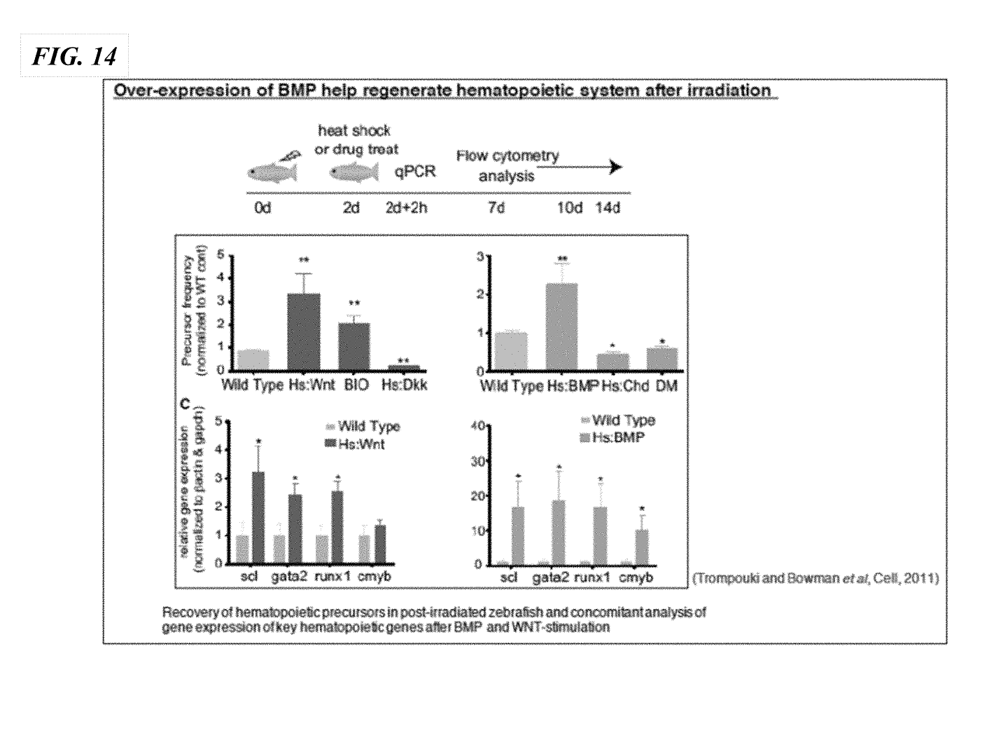

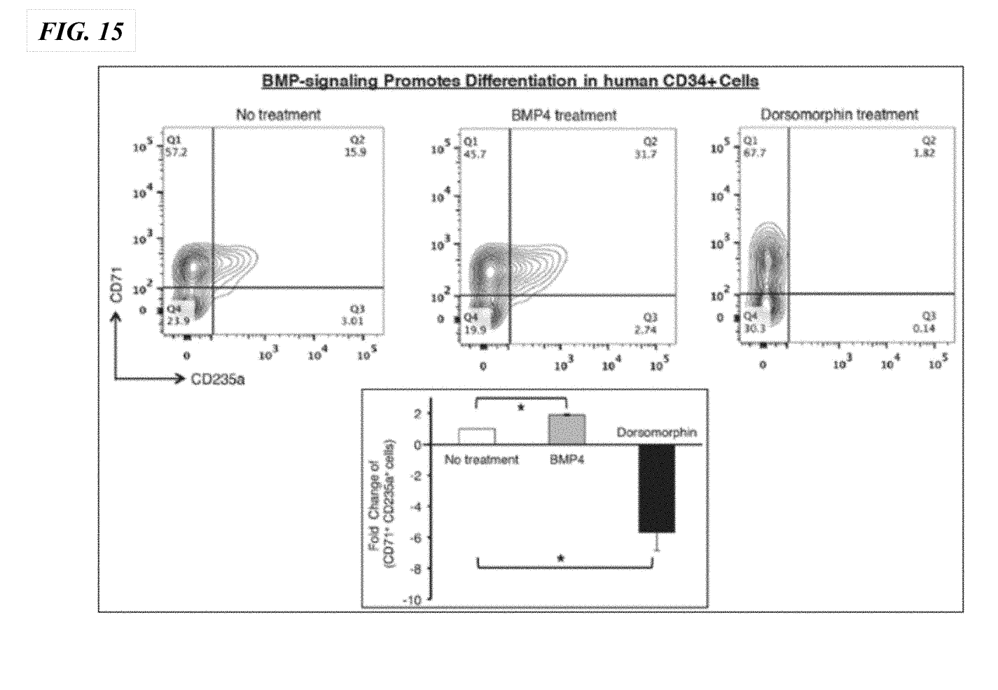

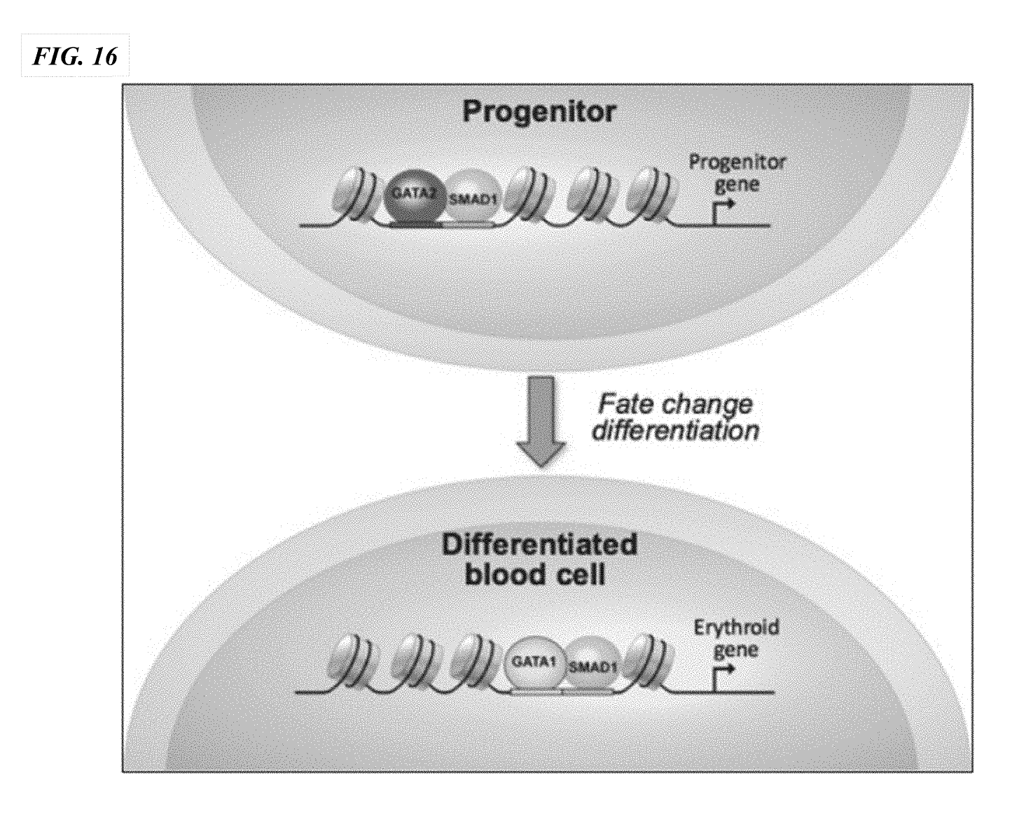

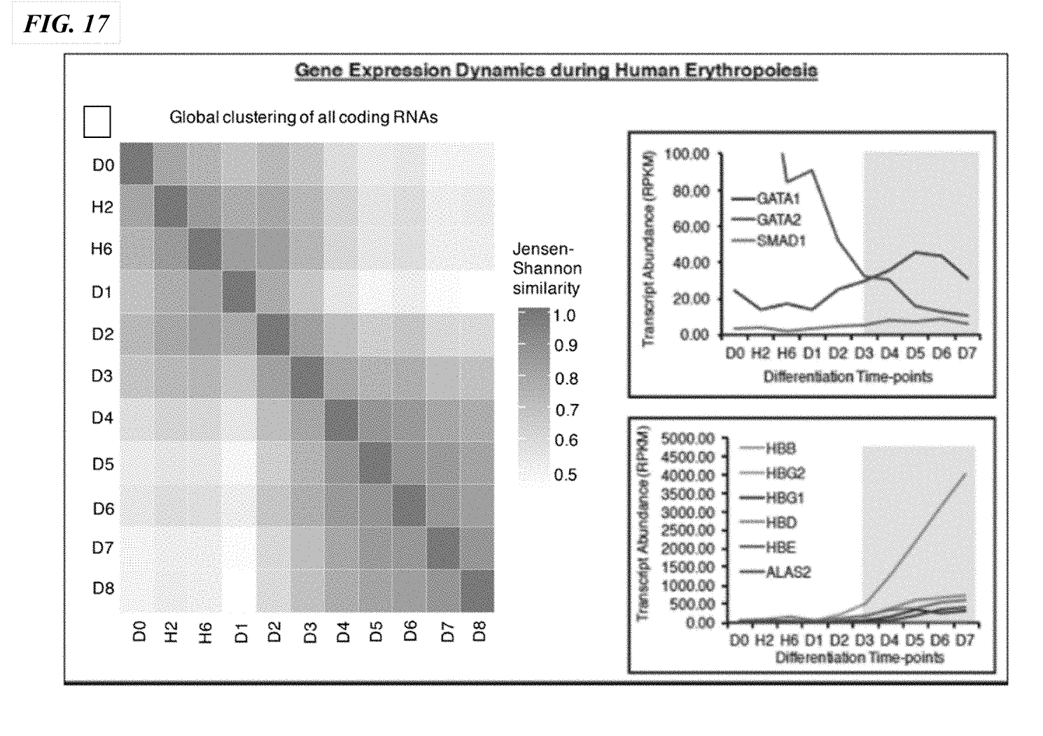

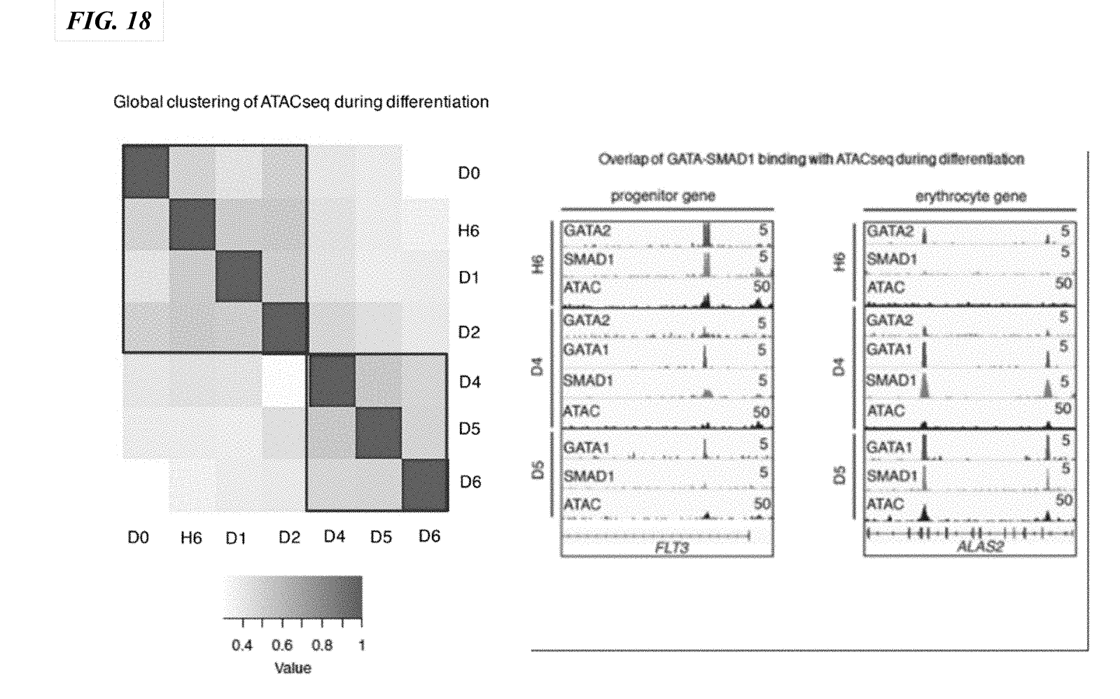

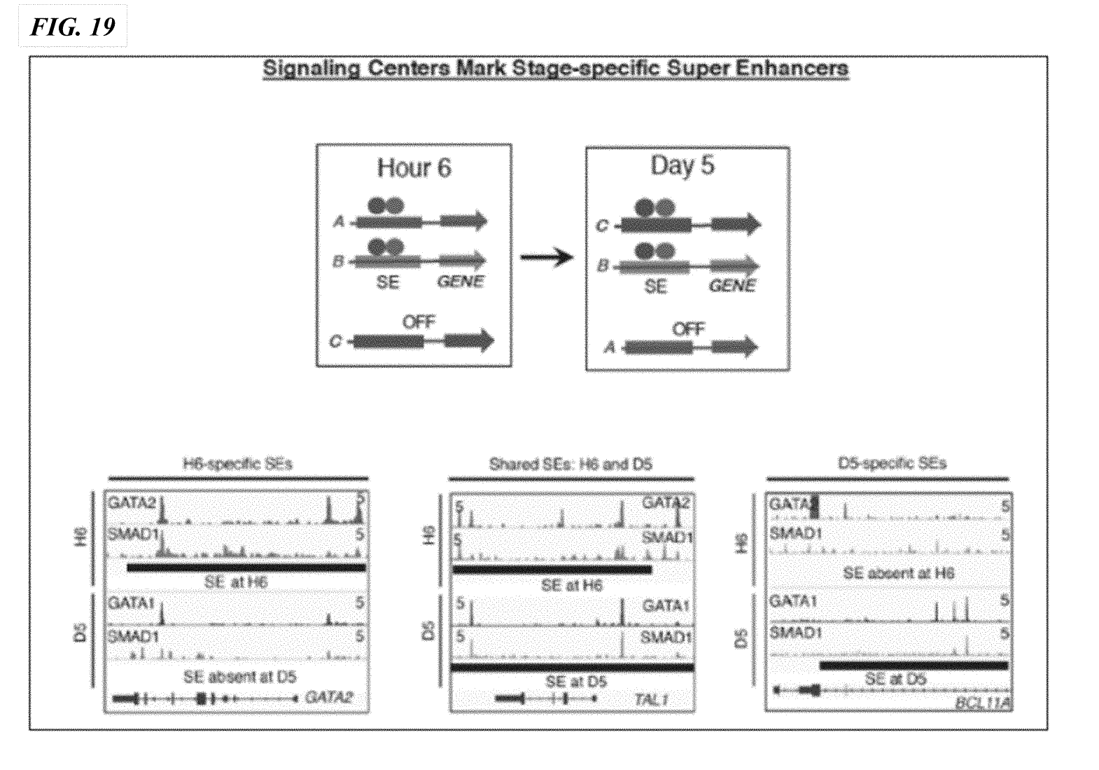

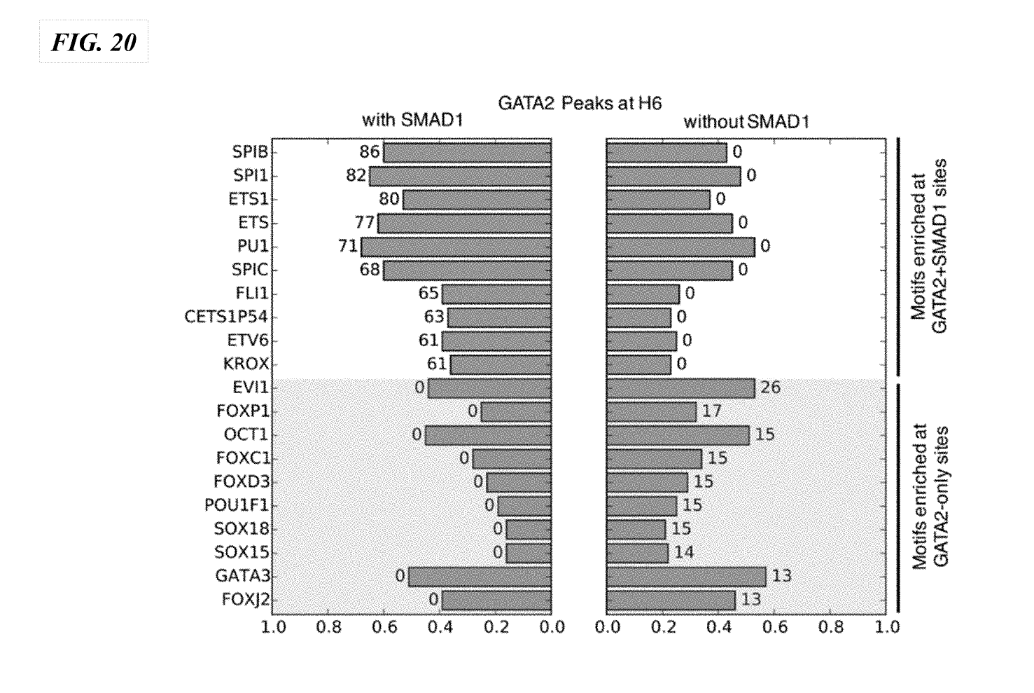

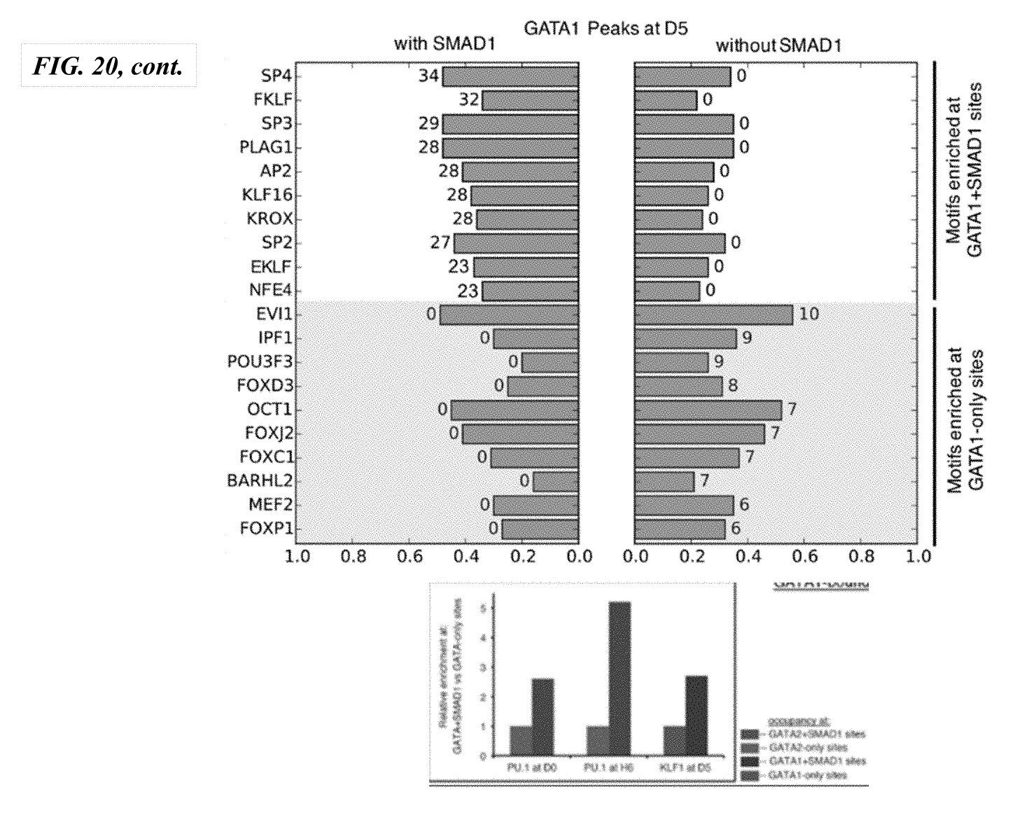

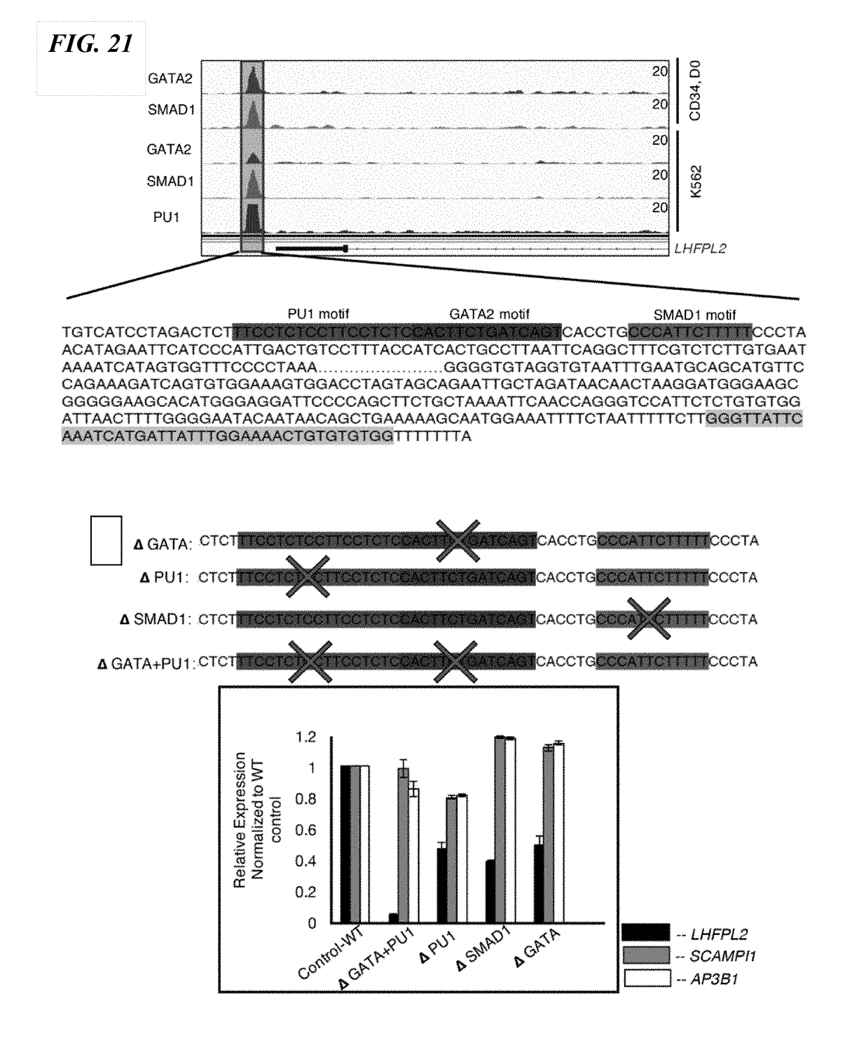

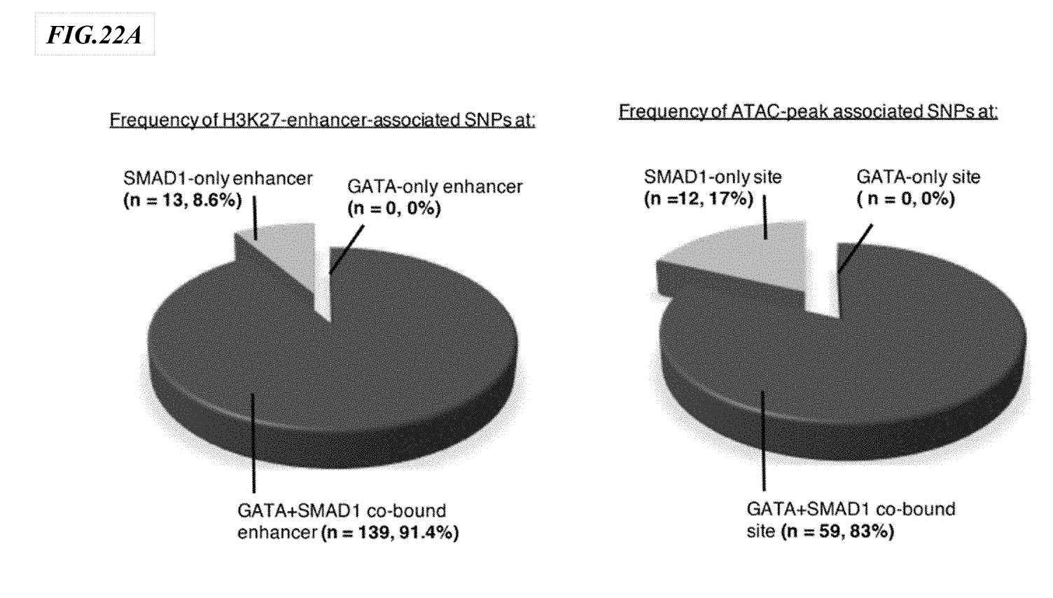

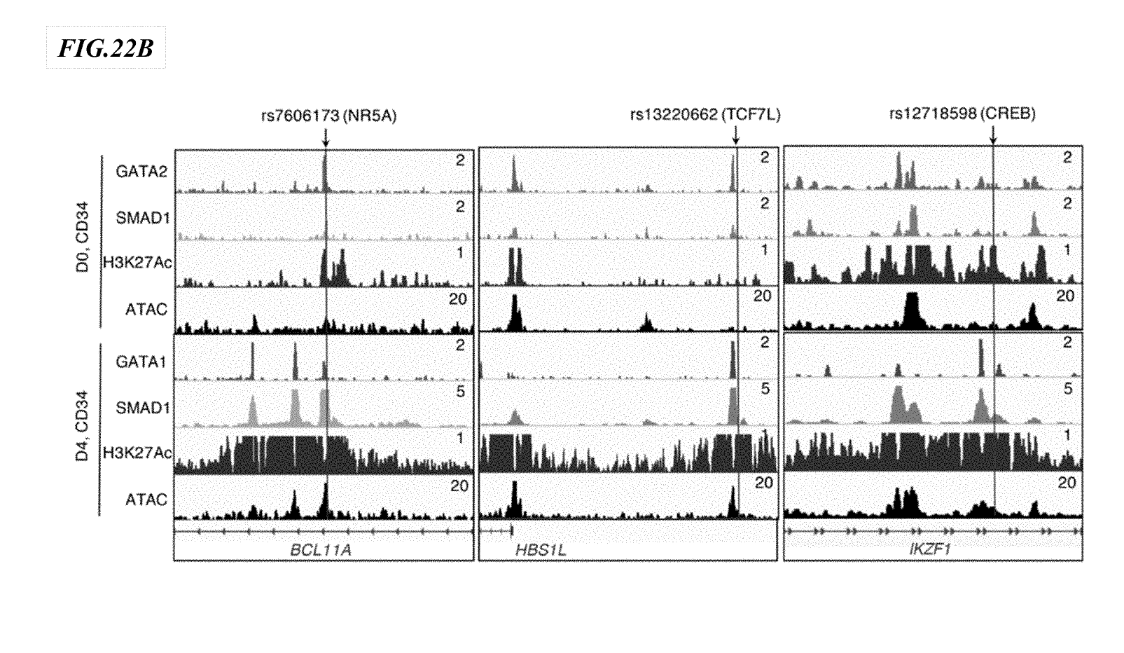

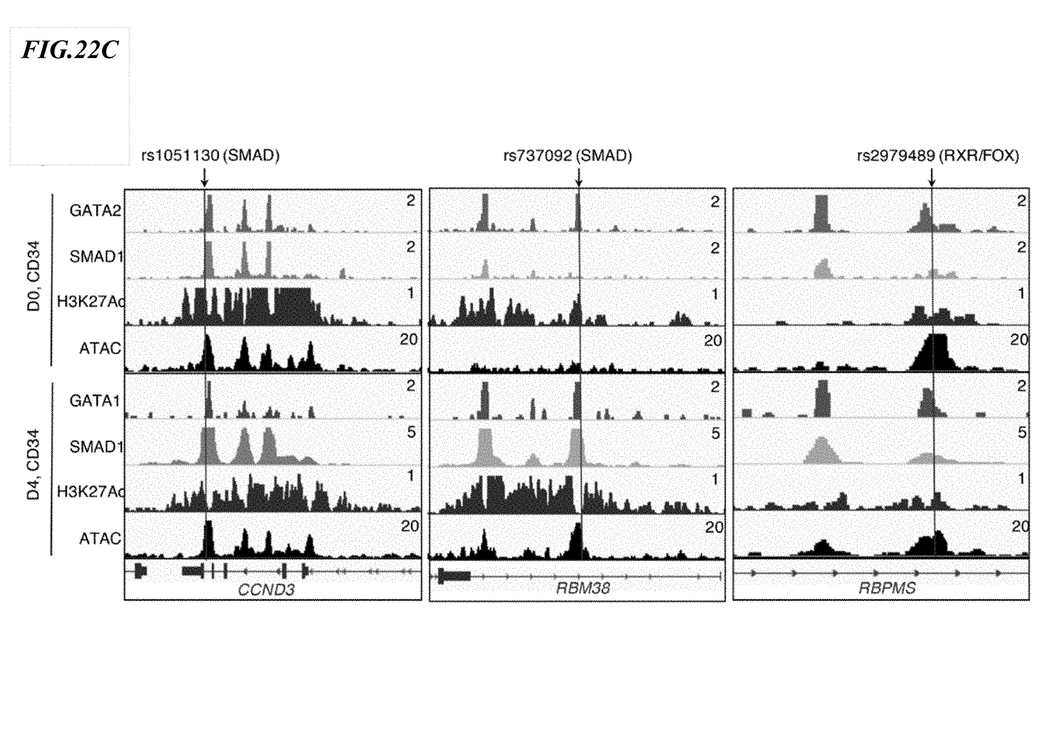

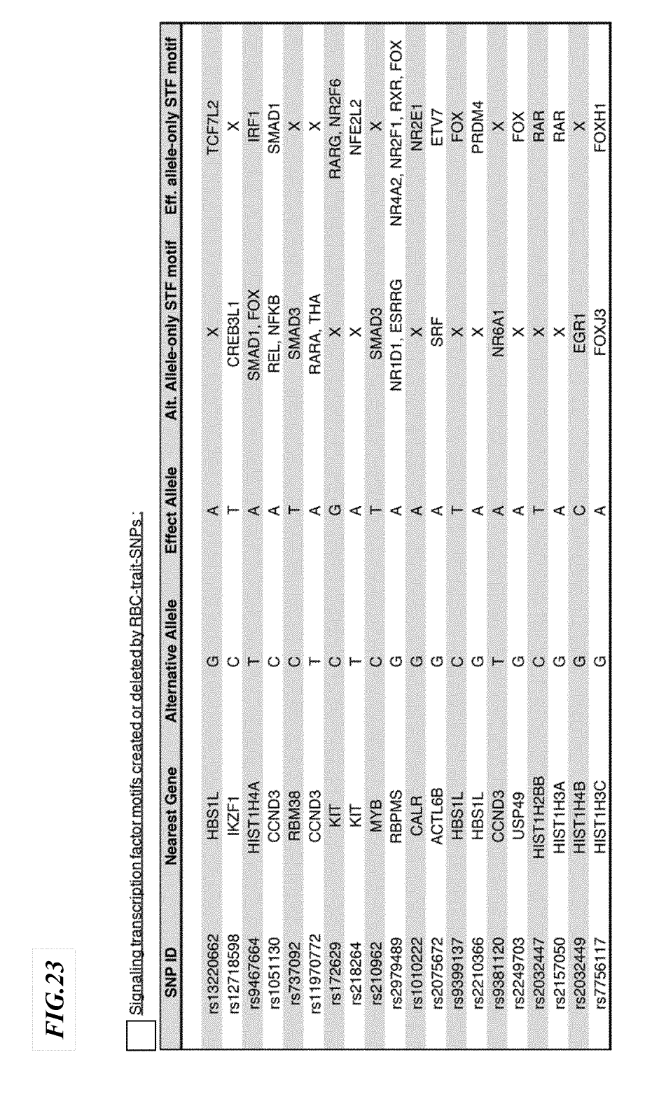

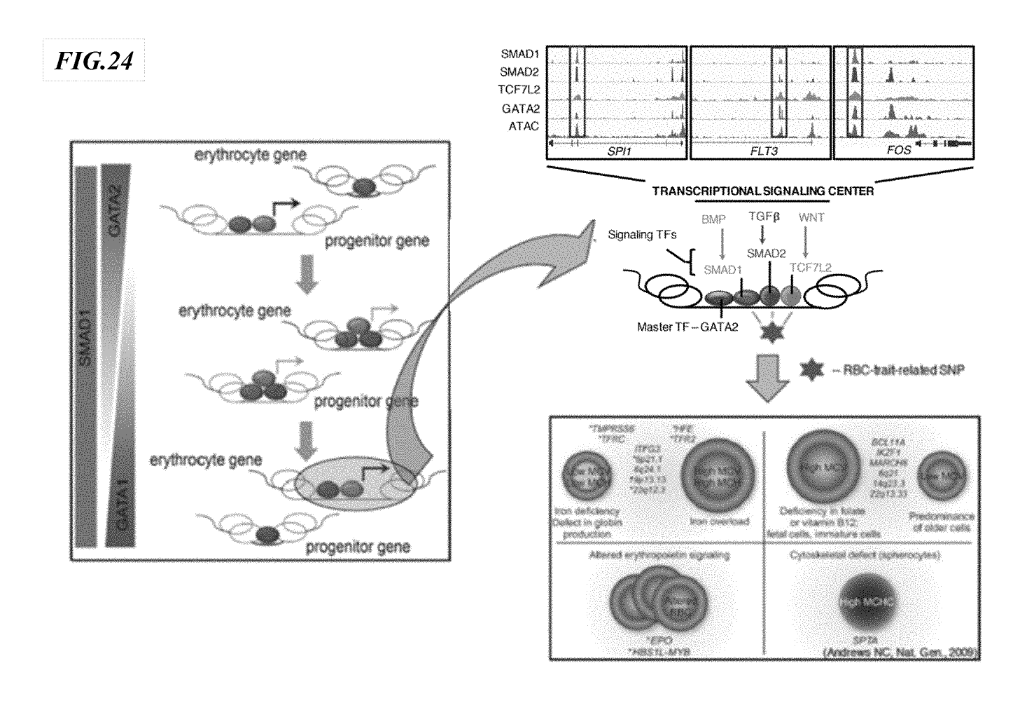

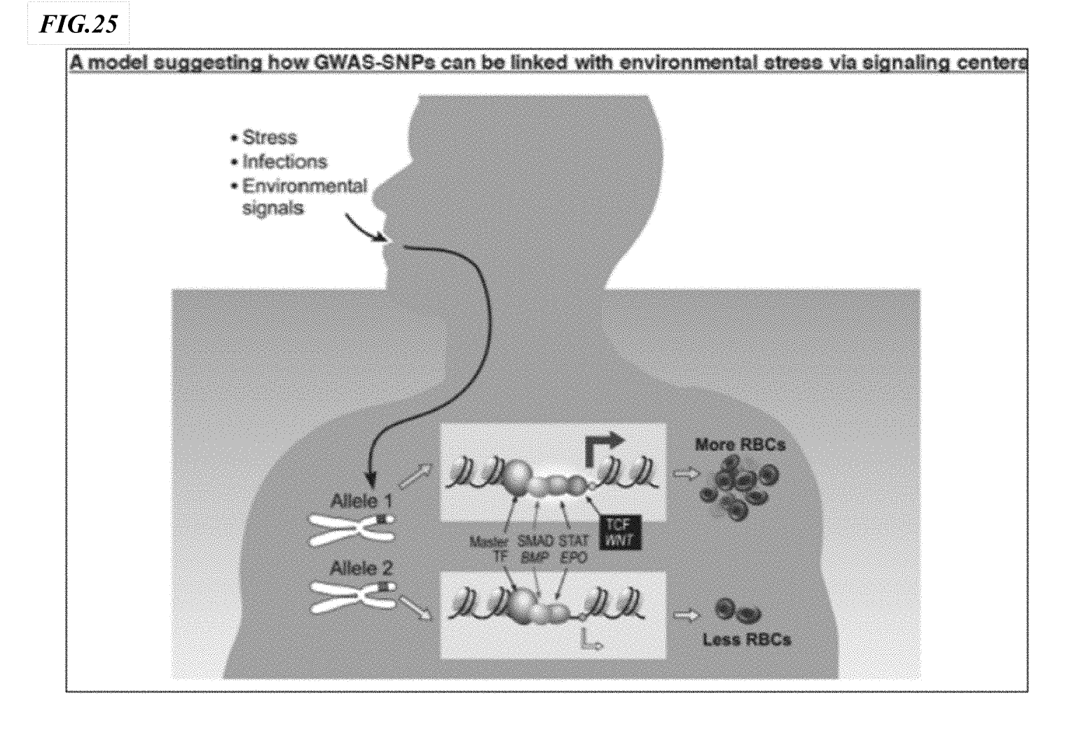

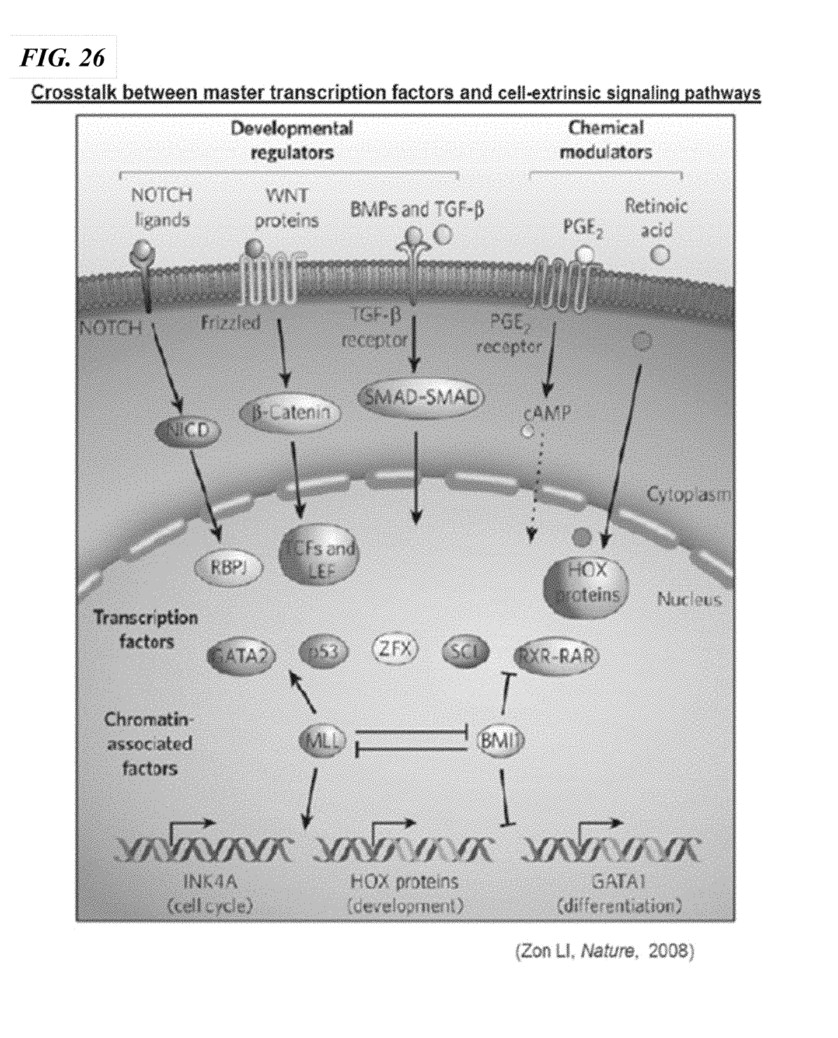

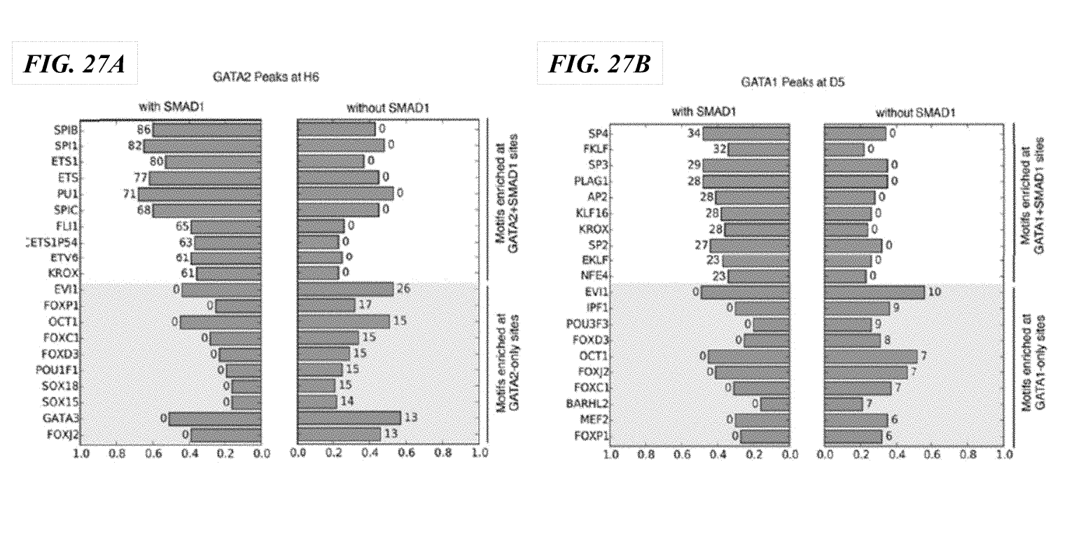

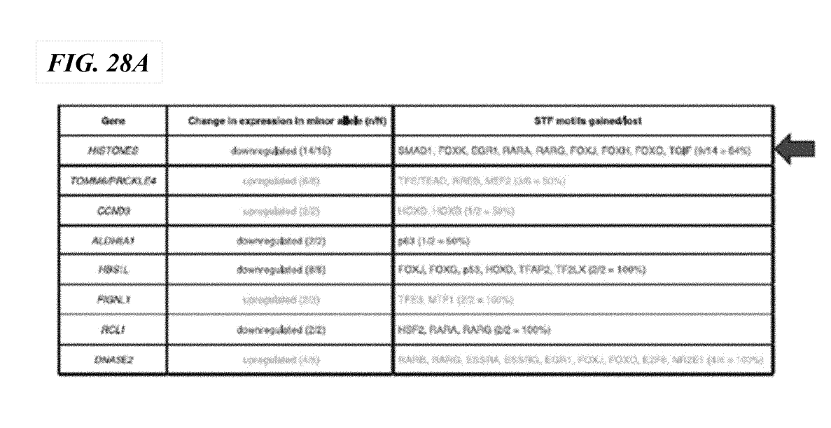

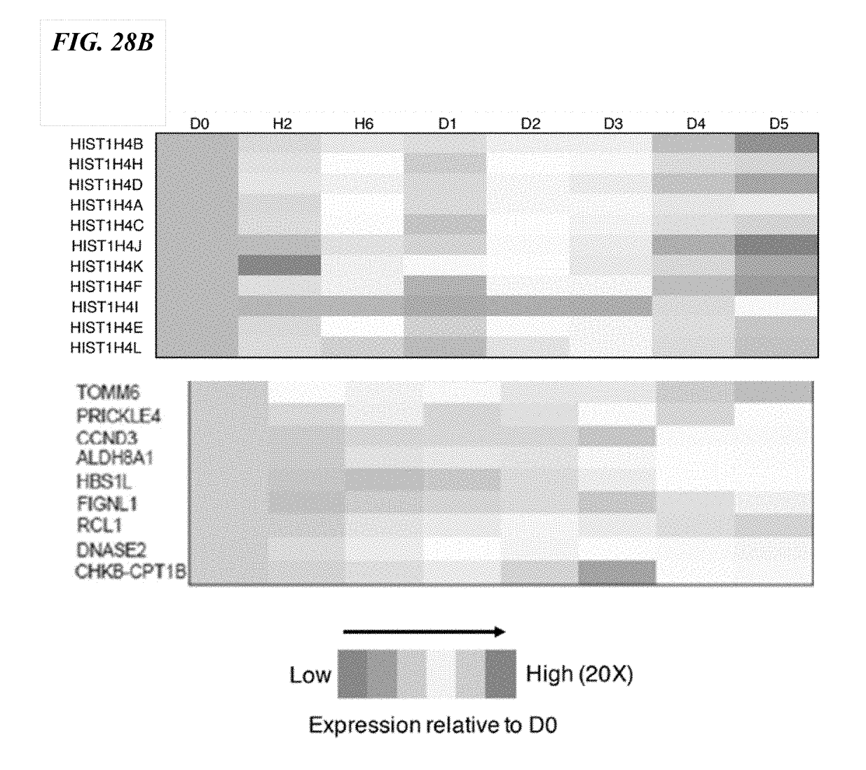

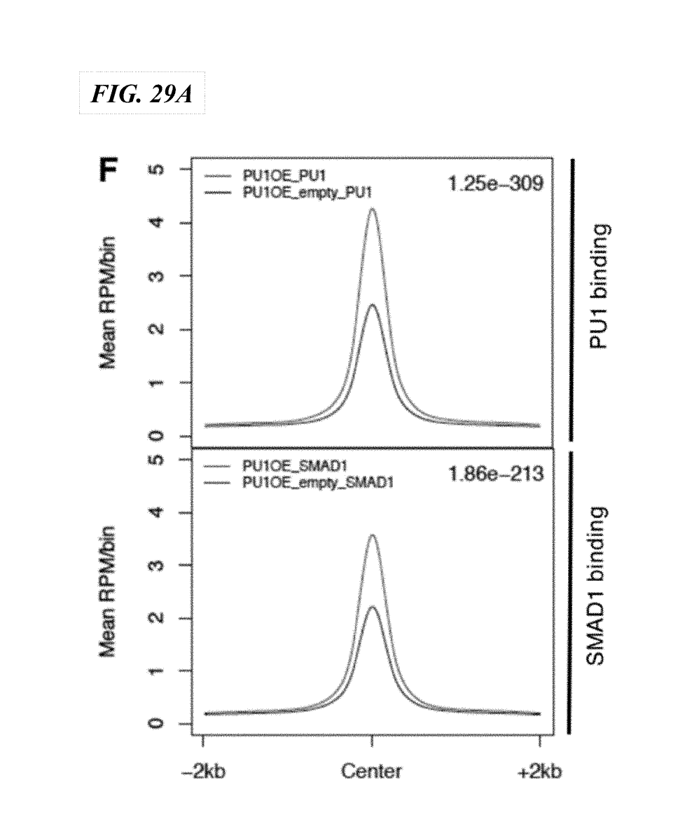

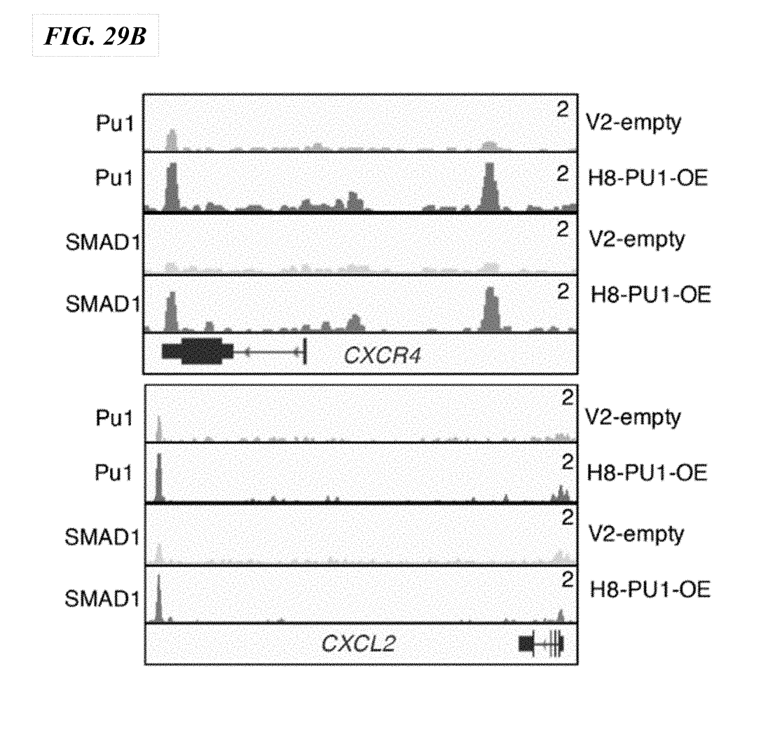

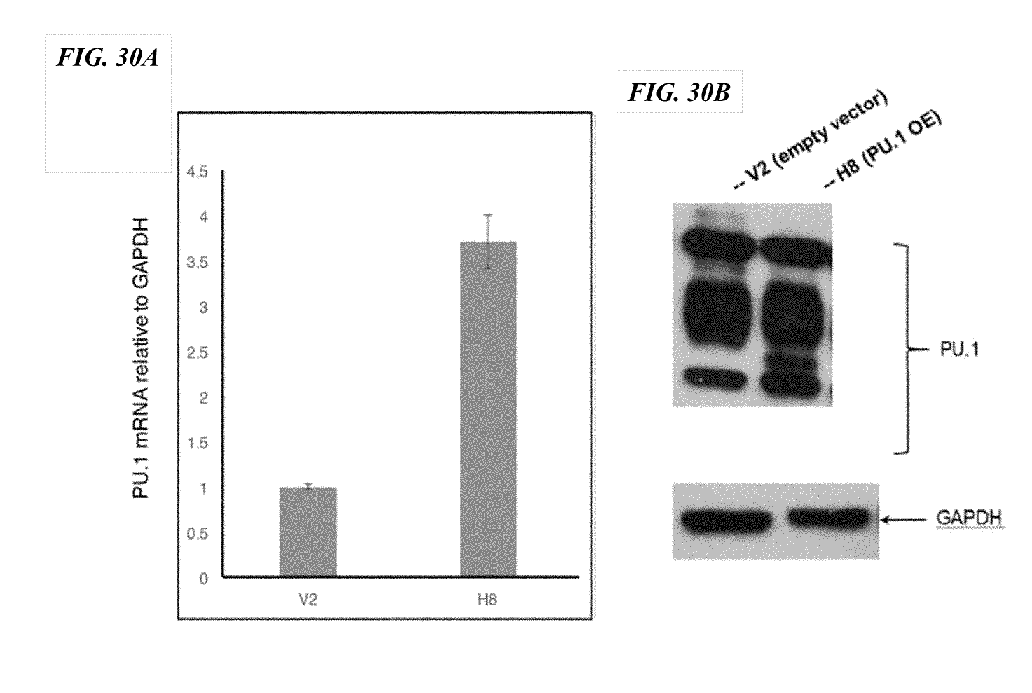

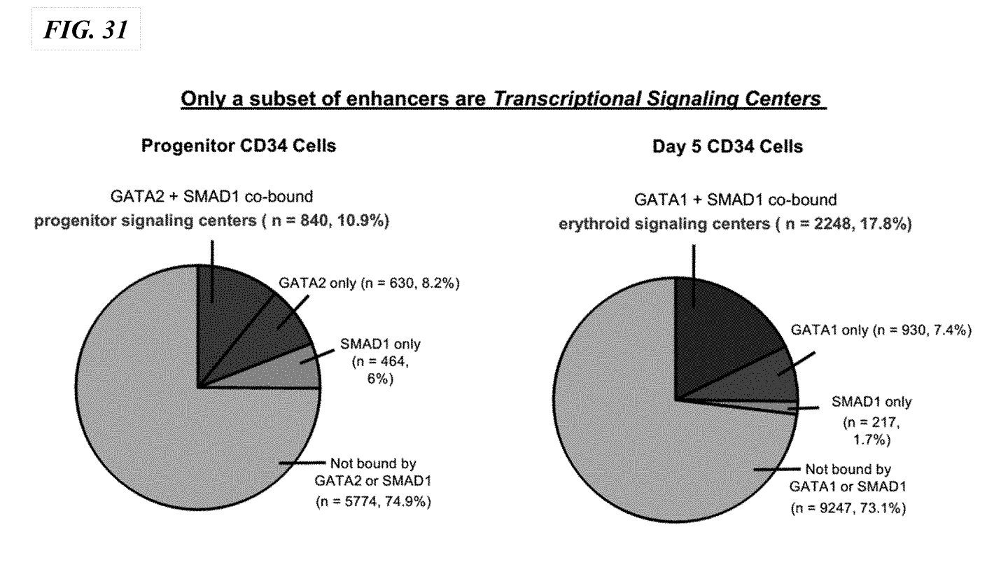

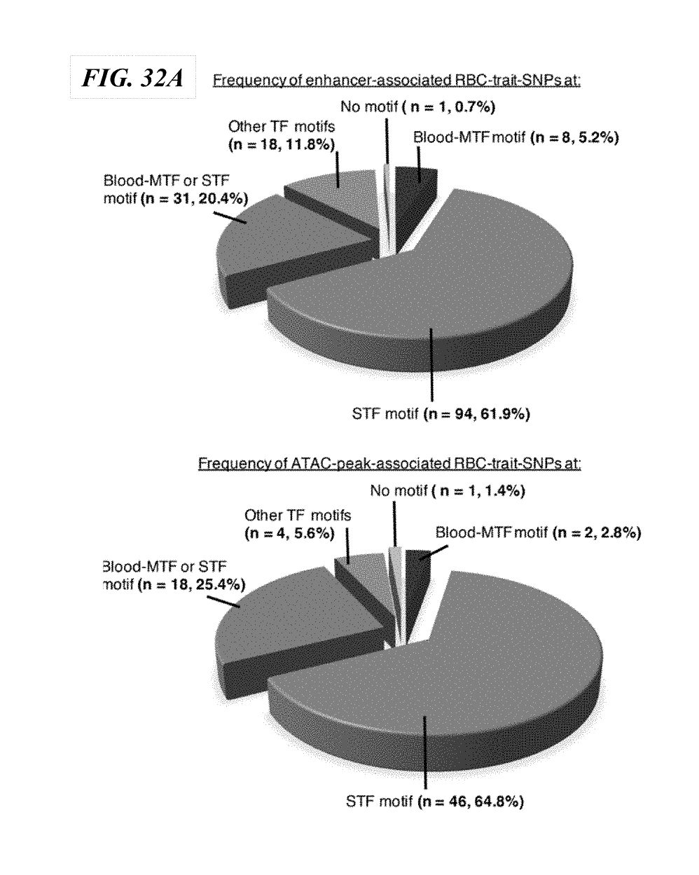

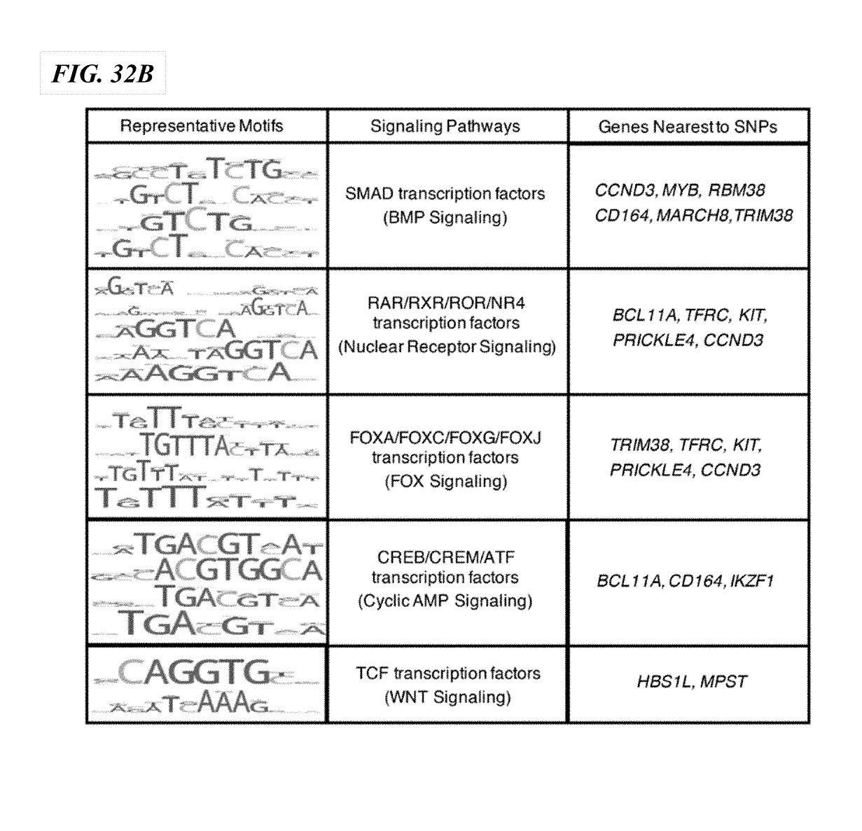

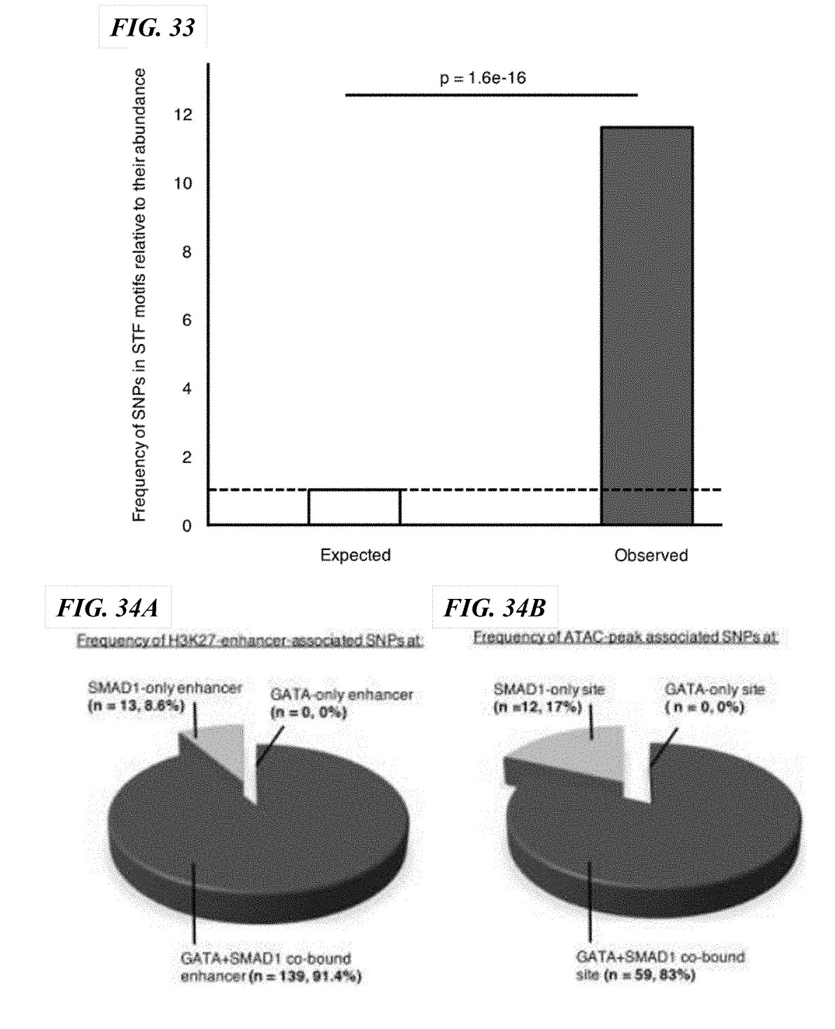

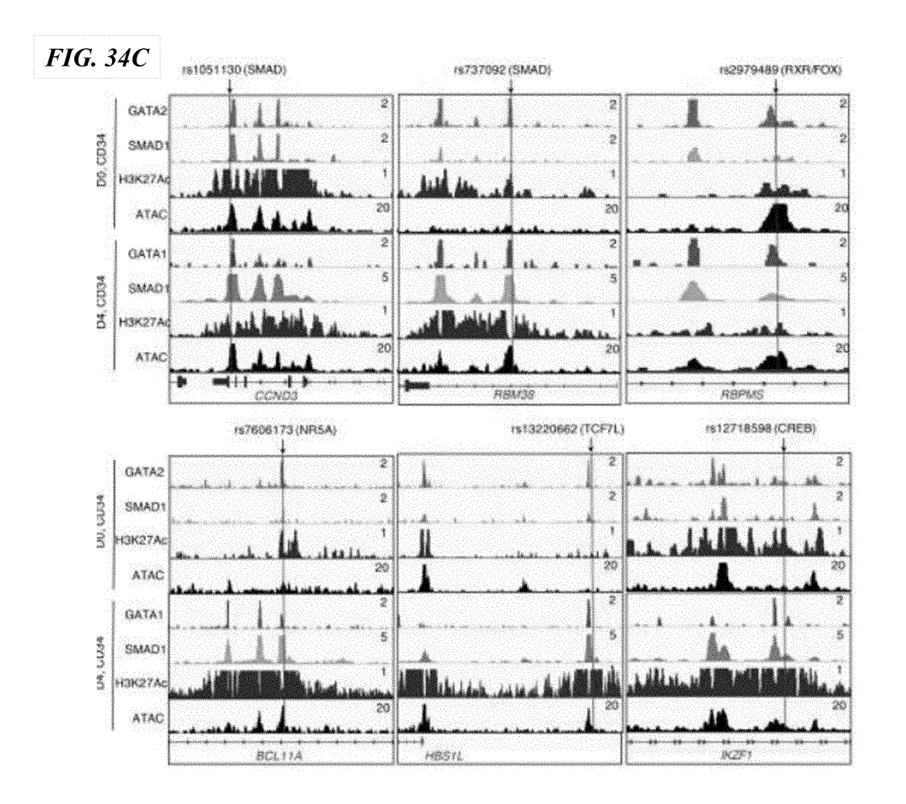

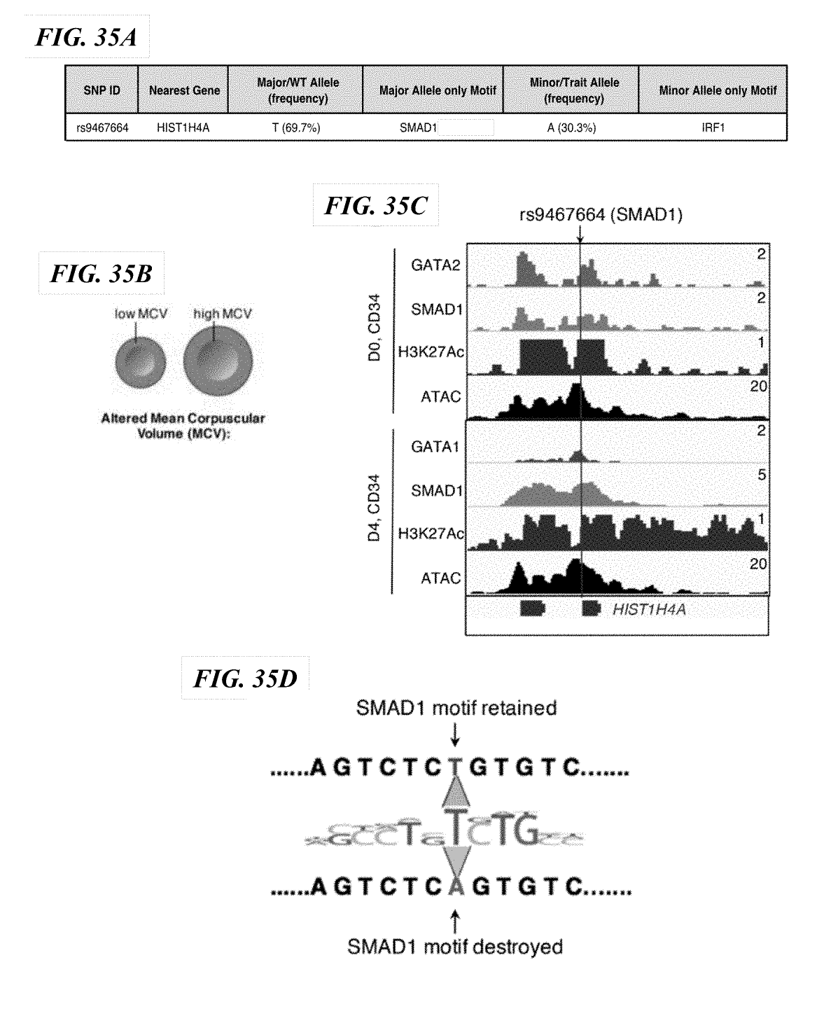

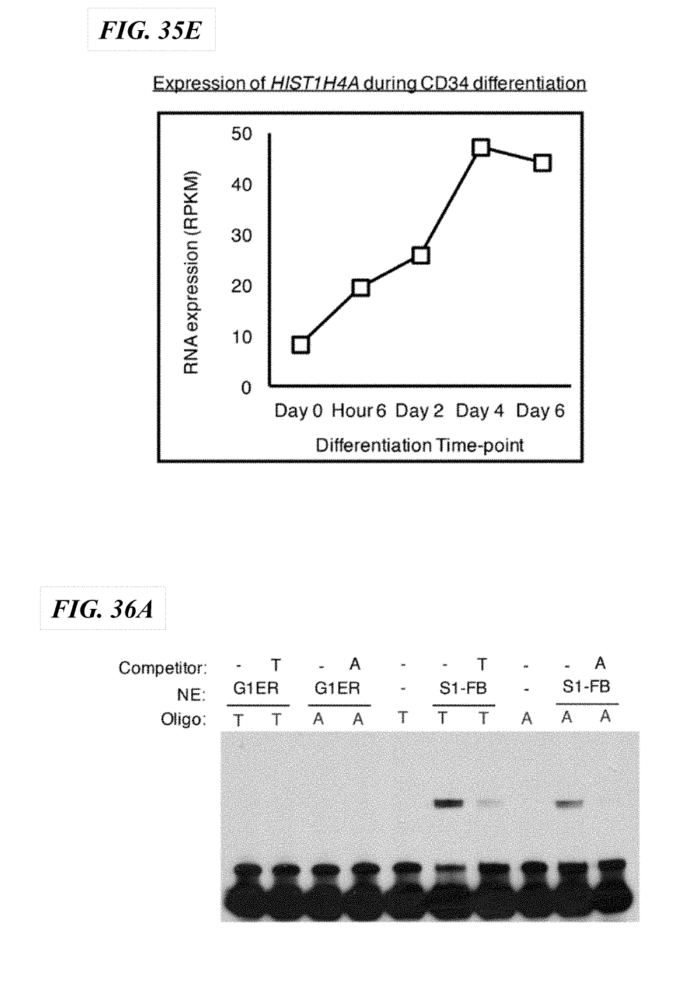

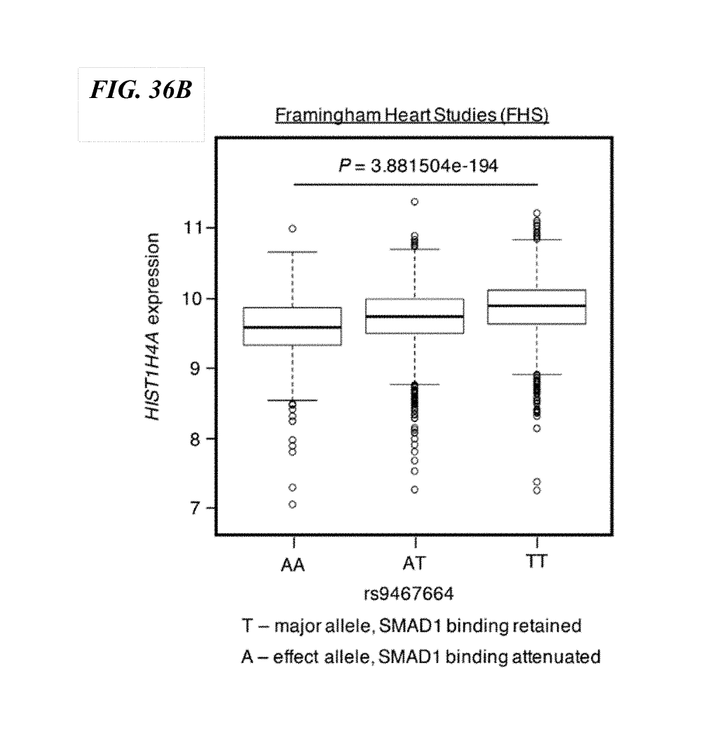

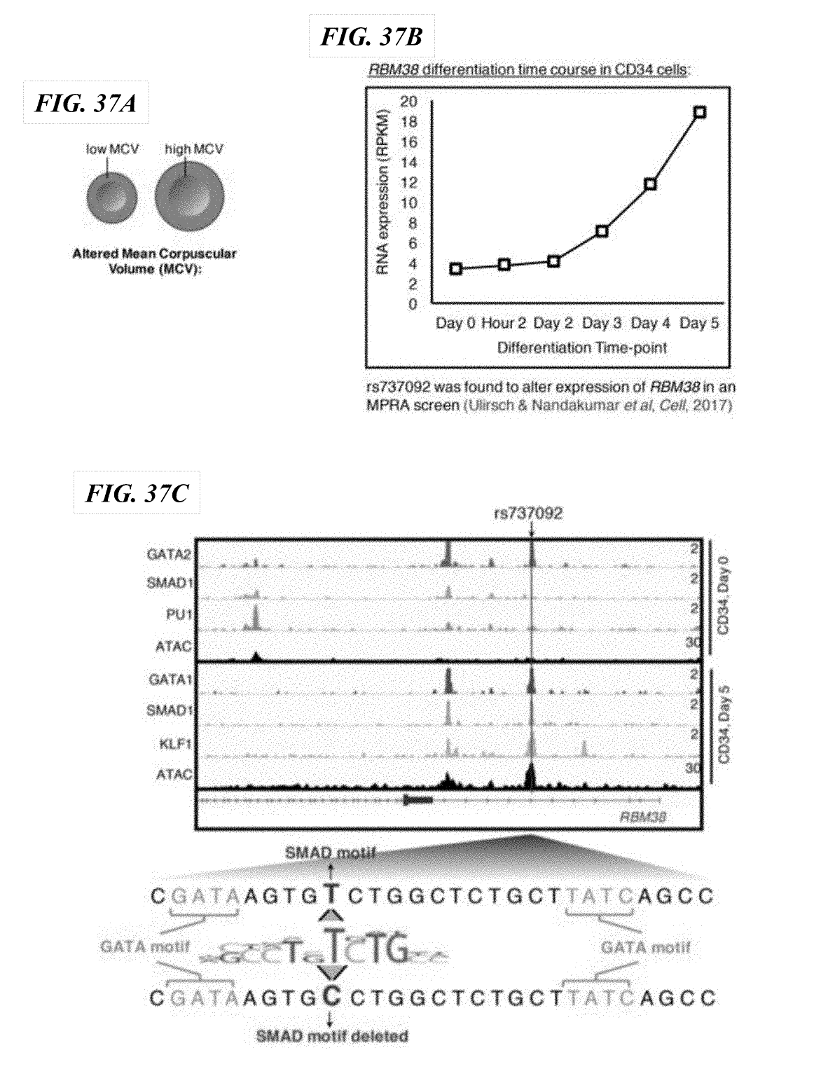

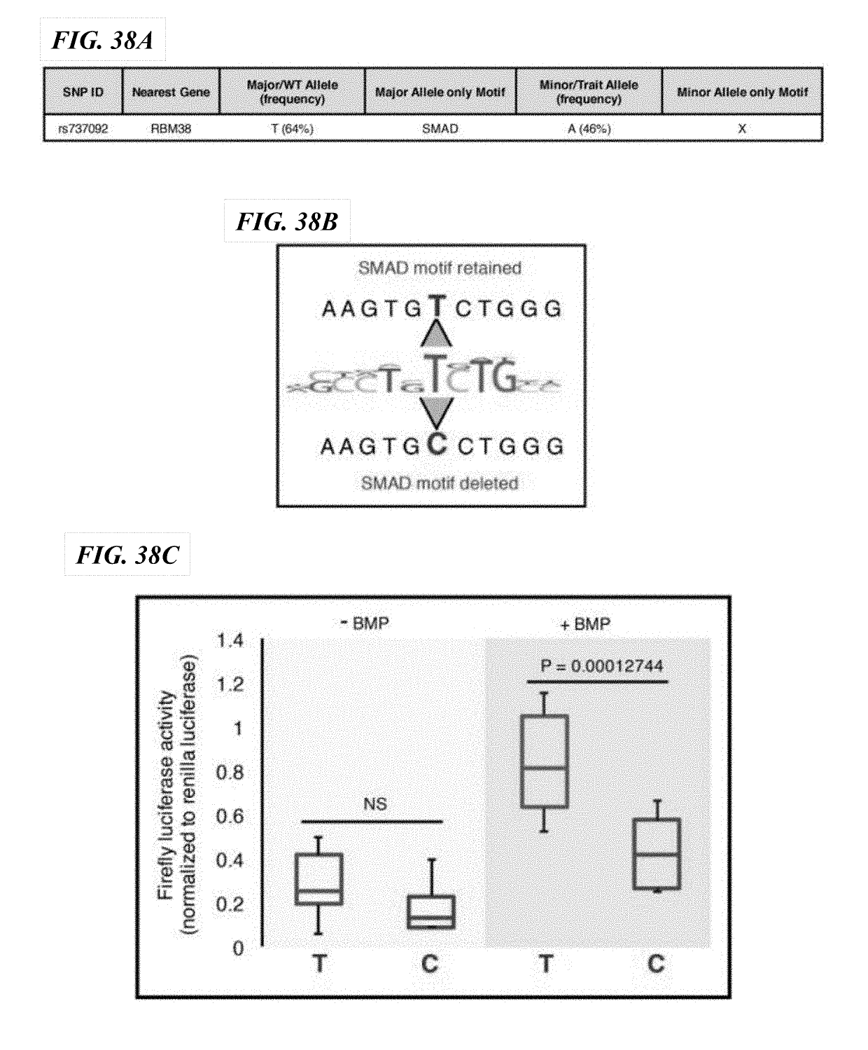

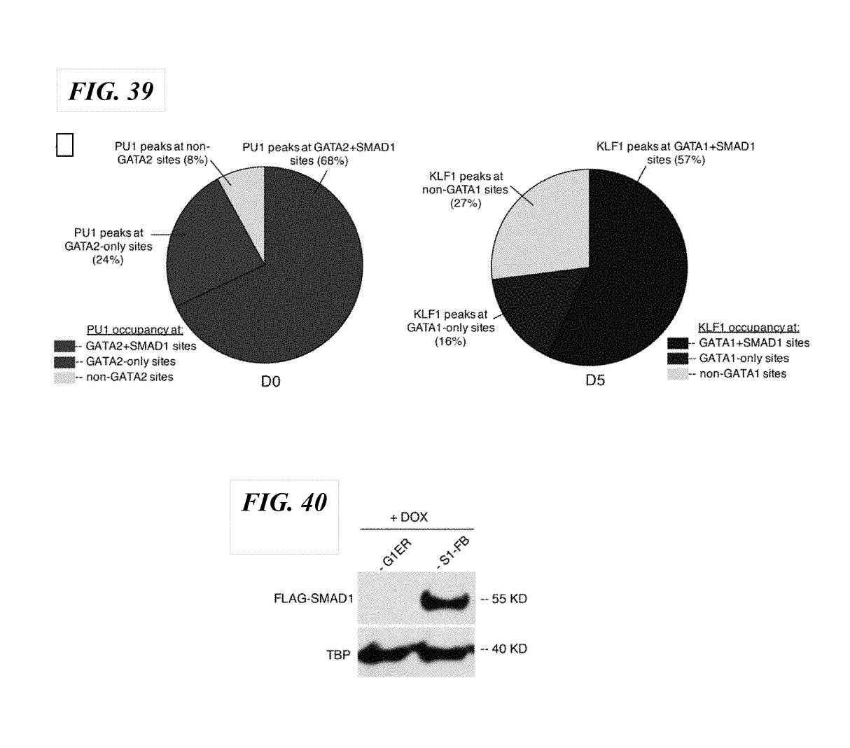

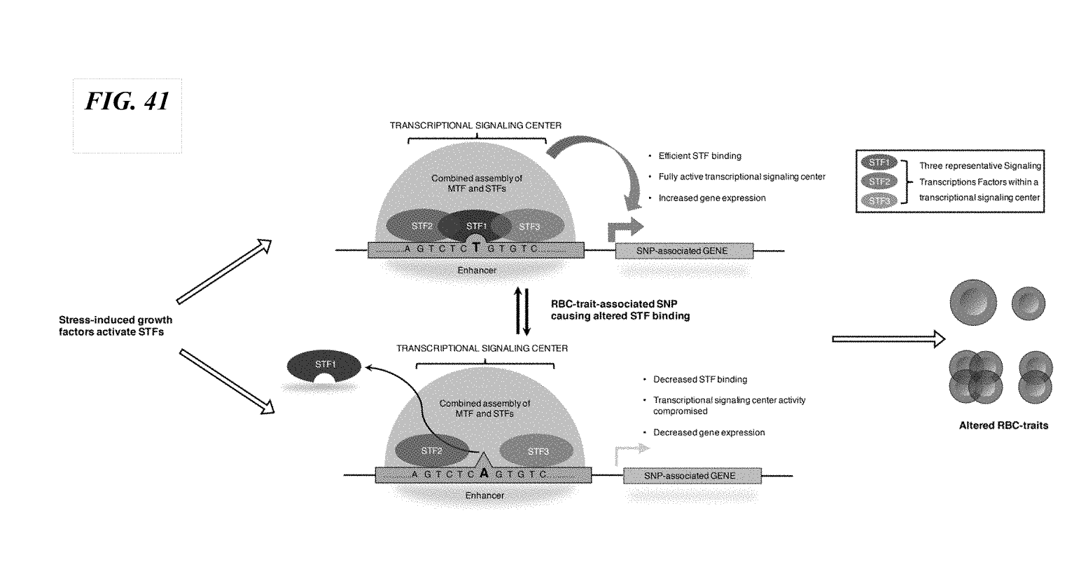

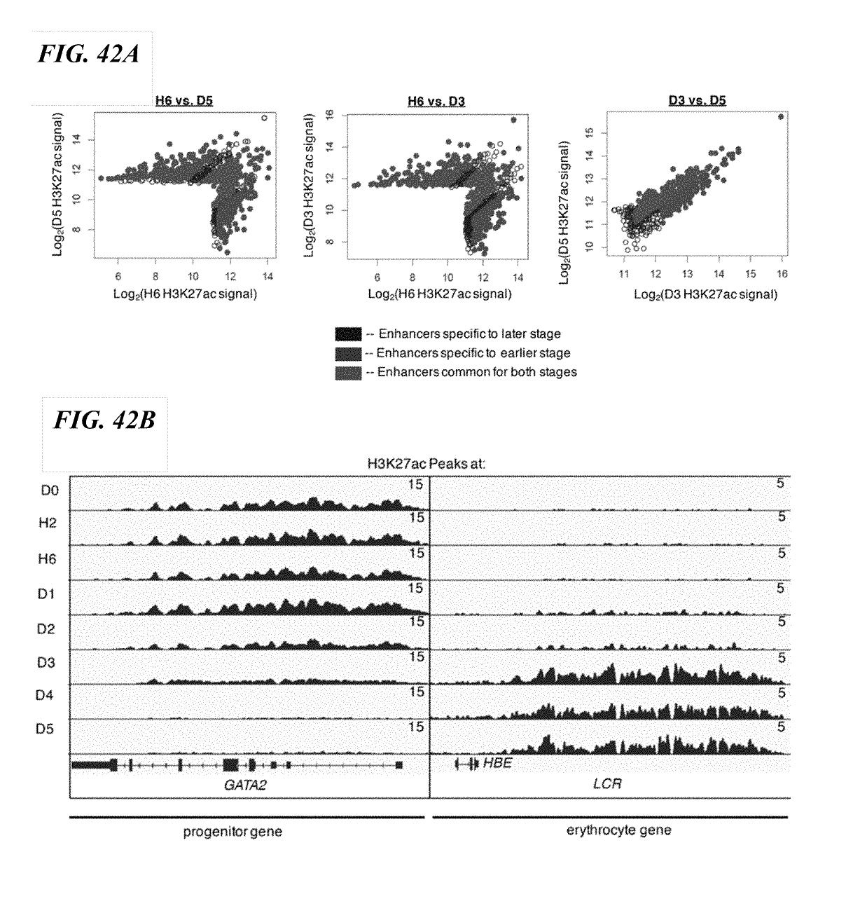

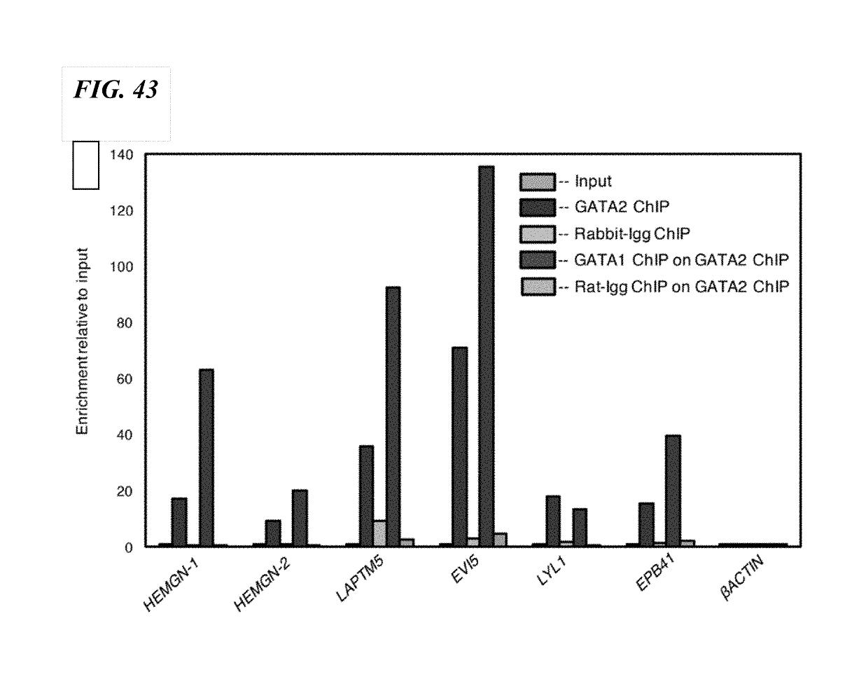

Next it was determined how chromatin accessibility changes throughout the time course. Analysis of Assay for Transposase-Accessible Chromatin followed by high-throughput sequencing (ATAC-seq) data collected from successive differentiation stages confirms an erythroid fate-switch transition around D3 and D4 of differentiation. Since ATAC-seq marks regulatory elements, this indicates a concomitant change in the enhancer landscape, expression program, and transcription factor binding. For example, two progenitor specific genes like FLT3 and CD38 lose ATAC-seq peaks at D3; in contrast, erythrocyte-specific loci, locus control region (LCR) and GYPA acquire prominent peaks from D4 of differentiation (FIG. 6A). Additionally, GREAT analysis of H6-ATACseq-peaks shows enrichment of hotspots of sensitivity near genes in categories that span all hematopoietic lineages in contrast to D5-analysis that shows enrichment mostly for erythoid-related categories (FIG. 11). This is consistent with the known chromatin reorganization that occurs during GATA1 induction (Cheng et al.,2009; Jain et al., 2015; Wu et al., 2011; Zaret and Carroll, 2011). The ATACseq

time-course supports this evidence since regions that show gradual gain in accessibility sites after GATA1 binding on D3 could be identified (FIG. 6B). Comparison of high-confidence GATA-bound versus GATA/SMAD1 co-bound regions shows enrichment for ATAC-seq signal on co-bound regions, indicating that chromatin is more accessible (FIG. 6C). ATAC-seq peaks associated with specific cell-stages strikingly overlap with GATA2/SMAD1 and GATA1/SMAD1 co-bound regions before and after erythroid commitment, respectively (FIG. 6D). However, when GATA factors bind alone, for example at the ALAS2 gene at H6, the regions often lack ATAC-seq signal. Thus, regions where SMAD1 and GATA factors co-localize represent“open chromatin hotspots” associated with tissue-specific genes.

Tissue Specific Co-Factors Associate with GATA/SMAD1 Regions

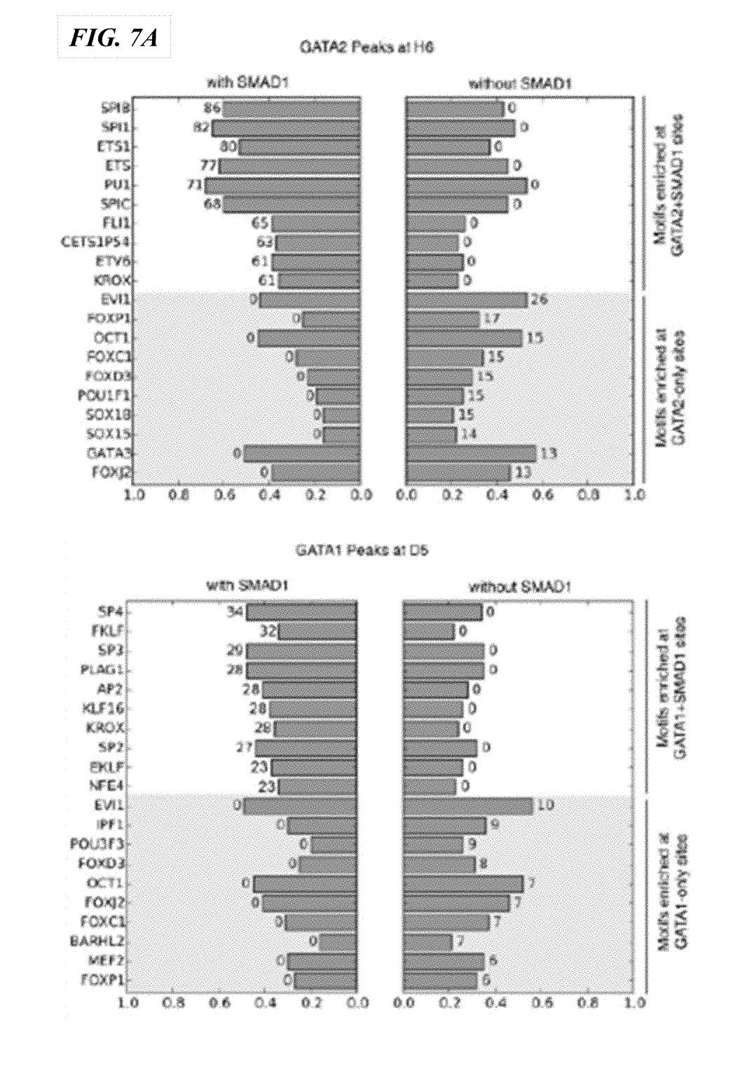

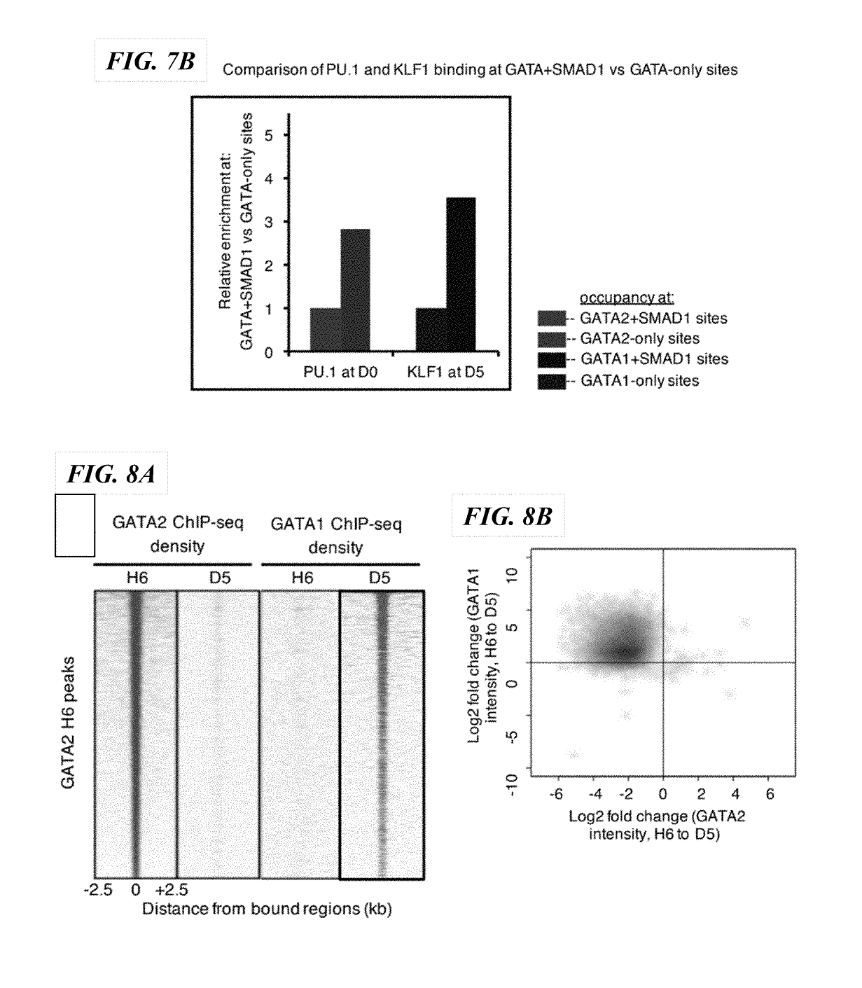

It was then determined which cofactors might be neighboring GATA and SMAD1 binding during erythropoiesis. To identify candidate transcription factors, GATA binding sites identified via ChIP-seq were scanned for known transcription factor binding motifs. Analysis presented herein revealed a marked enrichment of stage-specific transcription factor motifs in the GATA/SMAD1 co-bound regions (e.g. PU1 and Fill motifs in progenitor stage and EKLF/KLF1 and NFE4 motifs in erythrocyte stage) (FIG. 7A, Left and Right Panels). Surprisingly, a similar analysis for the GATA binding sites without SMAD1 co-binding, in both progenitor and differentiated stages, indicates enrichment of common developmental transcription factors, such as, EVI1, OCT1, FOXC1 and POUF (FIG. 7A).

To test this further, ChIP-seq binding data for GATA2 and SMAD1 at progenitor stages (at D0 and H6) presented herein were compared with previously published PU.1 ChIP-seq in CD133-positive umbilical cord blood cells (Novershtern et al., 2011). Overall overlap of binding between individual factors was minimal, which is presumably due to differences in the exact cell type. However, it was observed that 12.6% of GATA2/SMAD1 co-bound regions correlate with PU.1 binding at D0 compared to only 4.8% of the sites where GATA2 binds alone. Also at H6, PU.1 occupancy overlaps with 16.5% of GATA2/SMAD1 co-bund sites compared to

3.2% of GATA2 only sites. Similar comparison with KLF1 ChIP-seq that was performed in CD34+ cells differentiated towards erythrocytes albeit with another protocol (Su et al., 2013) showed that 51.9% of GATA1/SMAD1 co-bound regions at D5 co-localized with KLF1 compared to only 19.5% for the GATA1-alone bound sites. Taken together, the data show at least two-fold enrichment of stage-specific transcription factors (either PU.1 or KLF1) at the GATA/SMAD1 regions compared to GATA-only regions (FIG. 7B). This analysis indicates that lineage regulators (e.g., GATA) and signal-responsive transcription factors (e.g., SMAD1) tend to harbor other cell-type specific co-operating transcription factors creating active transcriptional hubs that play a major role in determining cell-type specificity.

This study provides a detailed genome-wide analysis of the mechanisms that orchestrate human erythropoiesis and reveals how binding of signaling transcription factors with lineage regulators guides cells through specific stages of differentiation. Previous studies indicated that, in blood cells, GATA factors co-localize with signaling transcription factors at specific genomic locations (Trompouki et al., 2011). Such a mechanism could also guide erythroid differentiation by specifying all the subsequent stages from progenitors to committed erythroid cells. Herein, it is shown that manipulating BMP signaling can boost or abrogate human erythroid differentiation. SMAD1, a BMP-responsive factor, co-localizes with the lineage regulators GATA1/2 on stage-specific genes in every step of differentiation. Although GATA2 mainly loses binding sites and GATA1 mainly gains binding sites during differentiation, SMAD1 binding is versatile by constantly gaining and losing sites. It is important to note that, for the purposes of study, high-confidence binding sites that pass very stringent statistical cutoffs were examined, so regions with high but not-significant binding are treated as lacking binding. For all the stages of human erythroid differentiation tested in this study, regions co-bound by SMAD1 and GATA1/2 show higher correlation with increased gene expression. This observation is supported by ATAC-seq and H3K27ac ChIP-seq of respective stages, which show that regions co-bound by SMAD1 and stage-specific GATA factors span open chromatin and active enhancers, in contrast to GATA-only bound regions. A large proportion of stage-specific SEs, that are bound by GATA factors are in fact co-occupied by SMAD1. Thus, GATA/SMAD1-bound regions represent determinants of cell identity that drive erythroid commitment.

Our study establishes SMAD1 as one of the dynamic factors during erythropoiesis in a paradigmatic model that shows how a signaling factor can co-regulate basic cell identity processes. Presumably, all signal responsive factors similar to SMAD1 can converge in the same genomic regions creating regulatory “hubs” that safeguard cell identity. Absence of one signal-responsive factor can be compensated by the presence of others, so that the “hub” remains preserved. This notion is supported by the lack of hematopoietic phenotype in SMAD1/5/8 or β-catenin-knockout mice (Jeannet et al., 2008; Koch et al., 2008; Singbrant et al., 2010) and further validated by a recent study that showed cancer or stem cell-specific super enhancers harbor binding sites for multiple signaling factors (Hnisz et al., 2015). Importantly, this study reveals such differentiation-stage-specific transcriptional hot-spots which are marked by the co-binding of lineage-specific GATA factors and SMAD1.

Genes and regulatory elements co-bound by SMAD1 and GATA factors can be used as a guide for studying stage-specific erythroid differentiation. Many of the SE-associated genes identified here e.g. BCL11A HBS1L-MYB, β-GLOBIN, MTHFR, UROS are known to play central roles in hematopoietic disease (Acharya et al., 2008; Basak et al., 2015; Guo et al., 2014; Lettre et al., 2008). This validates the usefulness of the results for further in-depth study of the regulation of these genes during normal and pathogenic hematopoiesis. Furthermore, DNA polymorphisms have been associated with genes responsive for sickle cell anemia (Lettre et al., 2008). Alignment of these SNPs with the regulatory elements identified in this study can reveal mechanistic insights. Finally, manipulation of such regulatory elements could be a means to edit targeted gene expression in a therapeutic context (Canver et al., 2015) and provide clues for personalized medicine.

To further dissect stage-specific regulatory elements, it is important to discover other co-factors that are part of the regulatory hot-spots in association with SMAD1 and GATA factors. Tissue-specific factor motifs (e.g. PU.1, FLI1 at H6 and KLF1, NFE4 at D5) were identified in the GATA/SMAD1 regions but common factors across the GATA-alone-bound regions (e.g. EVI1, OCT4). These factors likely exert distinct roles in shaping the activity of regulatory elements. It was also shown that chromatin is more open at GATA/SMAD1 co-occupied sites compared to GATA-only sites. This indicates that specific chromatin factors, presumably as part of GATA protein complexes, might regulate co-recruitment of SMAD1 and GATA factors to critical genomic regions. For instance, GATA2 can participate in two different complexes in progenitor cells. A repressor in the progenitor GATA2 complex may prevent SMAD1 binding on erythroid-specific genes whereas another protein can be responsible for the recruitment of SMAD1 in specific GATA2 bound regions. Purification of different GATA-complexes during the same stage across differentiation can reveal specific factors that establish and maintain active regulatory elements where lineage and signal--responsive elements co-localize and exert their functions.

Few studies have defined the intermediate signaling programs associated with stage-specific gene expression. Instead, there has been a focus on gene expression driven by lineage-specific regulators. SMAD1 binding in close proximity to GATA factors marks the genes that are stage-specific and provides an opportunity to identify and examine the function of these genes. In summary, studies presented herein use a human erythroid differentiation system as an example to show that signaling factors co-ordinate with internal cell regulators to control cell fate. Compilation of regions where signal responsive and lineage regulators co-localize, in any system, can reveal the regulatory elements and genes required for cell-type determination.

Experimental Procedures

Cell Culture.

Human CD34+ cells, isolated from peripheral blood of granulocyte colony-stimulating factor-mobilized healthy volunteers, were obtained from the Fred Hutchinson Cancer Research Center. The cells were maintained and differentiated as previously described (Sankaran et al., 2008b; Trompouki et al.,2011).

Immunofluorescence.

CD34+ cells at multiple differentiation stages were fixed with PFA and stained with GATA2 (sc9008), GATA1 (Ab28839) and beta-hemoglobin (sc-21757) antibodies 0/N. Photos were taken using an inverted Nikon Eclipse Ti microscope.

QPCR Analysis.

RNA was extracted from CD34⁺ cells using Trizol. QPCR was performed using QuantStudio 12K Flex. For more information and primer sequences see also supplemental experimental procedures.

Flow Cytometry Analysis.

Control and treated stage-matched CD34 cells were washed in PBS and stained with propidium iodide (PI), 1:60 APC-conjugated CD235a (eBioscience, clone HIR2, 17-9987-42), 1:60 FITC-conjugated CD71 (eBioscience, OKT9, 11-0719-42), 1:60 PE-conjugated CD41a (eBioscience, HIP8, 12-0419-42) and 1:60 PE-conjugated CD11b (eBioscience, ICRF44, 12-0118-42). BD Bioscience LSR II flow cytometer was used to record raw FACS data, which were analyzed subsequently using FlowJo 8.6.9 10.0.7 (TreeStar).

Chromatin Immunoprecipitation, RNA-Seq, ATAC-Seq Experiments and Bioinformatic Analysis.

All procedures were performed in CD34⁺ cells at various time-points during erythroid differentiation.

Motif Analysis.

A set of 881 TF binding site motifs was obtained from (Ziller et al., 2015). FIMO (Grant et al., 2011) was used to scan GATA peaks for occurrences of these motifs. Peaks were deemed to contain a motif if FIMO reported a p-value below 1e-4 at one or more locations within the peak.

Ingenuity Pathway Analysis, GREAT Analysis.

The enriched genes from each category used (RNA-seq, SEs etc) were imported into Ingenuity Pathways Analysis (IPA) (Ingenuity Systems) to analyze functional interactions between the genes.

To analyze the functional nature of the peaks called, from each ATACseq experiments, individual BED files were imported and visualized in GREAT, Stanford University.

Expansion and Differentiation of CD34+ Cells.

Human CD34⁺ cells, isolated from peripheral blood of granulocyte colony-stimulating factor mobilized healthy volunteers, were purchased from the Fred Hutchinson Cancer Research Center. The cells were maintained and differentiated as previously described (Sankaran et al., 2008; Trompouki et al., 2011). Briefly the cells were expanded in StemSpan medium (Stem Cell Technologies Inc.) supplemented with StemSpan CC100 cytokine mix (Stem Cell Technologies Inc.) and 2% P/S for a total of 6 days. After six days of expansion the cells were stimulated for 2 hr with rhBMP4 (R&D) at a final concentration of 25 ng/ml and harvested for performing all the experiments corresponding to D0 time point. For studying differentiated cells after day 6 of expansion, cells were reseeded in differentiation medium (StemSpan SFEM Medium with 2% P/S, 20 ng/ml SCF, 1 U/ml Epo, 5 ng/ml, IL-3, 2 mM dexamethasone, and 1 mM β-estradiol), at a density of 0.5-1 3 10⁶ cells/ml. Prior to harvesting at H2, H6, D1-D8 the cells were treated with 25 ng/ml hrBMP4 for 2 hrs.

For testing the effect of BMP4 and dorsomorphin cells at the beginning of third day of differentiation were treated with either 25 ng/ml hrBMP4 or 20 μM Dorsomorphin till the beginning of fifth day of differentiation. At D5, cells were isolated for flow cytometry and qPCR analysis. Cells treated with DMSO were used for control experiments (FIG. 1C).

Immunofluorescence.

5×10⁴ CD34⁺ cells at specific differentiation stages (H6, D3, D4 and D5) were first washed with PBS and then plated uniformly with 0.8% low melting agarose in each well of a 96 well plate. Upon drying, cells were fixed with 4% PFA for 5 min at RT. After six quick washes with PBS-Triton (0.1%), cells were blocked for 30 min in 4% BSA in PBS-Triton (1%) solution. All the primary antibodies for immuno-staining (rabbit polyclonal GATA2, sc9008; rabbit polyclonal GATA1, Ab28839; mouse monoclonal beta-hemoglobin (37-8), sc-21757) were used at 1:200 dilution in PBS-Triton (1%) and cells were incubated with them at 4° C. overnight. Primary antibody treated cells were washed 3 times with PBS-Triton (1%) for 10 min at RT. The anti-mouse and anti-rabbit Alexafluor 488 conjugated secondary antibodies (Invitrogen: A11029 and Invitrogen: A11034, respectively) were diluted 1:500 in PBS-Triton (1%) for 30 min, RT. After 3×10 min washes using PBS-Triton (1%), cells were stained with DAPI (Invitrogen, D3571) (at 1:1000 dilution). After 3×10 min PBS-Triton (1%) washes at RT, cells were kept in PBS-Triton (1%) and subsequently imaged using an inverted Nikon Eclipse Ti microscope (Andor Technologies). Raw images were processed and analyzed with NIS-Elements D4.00.03 software (FIG. 1B).

qPCR Analysis.

RNA was extracted from CD34⁺ cells without any treatment or treated with hrBMP4 or Dorsomorphin at the specified developmental stages using TRIZOL extraction (Invitrogen), followed by RNeasy column purification (QIAGEN). First strand cDNA synthesis was performed using the Superscript VILO (Invitrogen) and equivalent amounts of starting RNA from all samples. The cDNA was analyzed with the Light Cycler 480 II SYBR green master mix (Applied Biosystems), and the QuantStudio 12K Flex (Applied Biosystems) (FIG. 3D). All samples were prepared in triplicate. The PCR cycle conditions used are: (a) 95° C. for 5 min, (b) [95° C. for 10 sec, 54° C. for 10 sec, 72° C. for 15 sec]×40 cycles. The analysis of Ct values were performed using 2̂-ΔΔT method (Livak and Schmittgen, 2001). The PCR primer-pairs used are:

Primers Used Herein.

| | | SEQ |
|---|---|---|
| Gene | Forward Primer | ID NO: |
| HBB | 5′CACTGGTGGGGTGAATTCTT3′ | 2 |
| ALAS2 | 5′AGCACTGGGCAGCACTGTA3′ | 3 |
| SLC4A1 | 5′TGGTCCTGAGTGTCCAGTTG3′ | 4 |
| UROS | 5′AGAAGAGGCAGTGCTGAGGA3′ | 5 |
| DYRK3 | 5′TTGCCAAATTCTTGAAACAGC3′ | 6 |
| SH2D2 | 5′GTCCCCTAGAAGCCACCTTT3′ | 7 |
| NFATC3 | 5′GCACAATCATCTGGCTCAAG3′ | 8 |
| LMNA | 5′GAGTTCAGCAGAGCCTCCAG3′ | 9 |
| KCNK5 | 5′AGGTCTGGTTCCCTGTGATG3′ | 10 |
| ZFP36L1 | 5′CTTTCTGTCCAGCAGGCAAC3′ | 11 |
| | | SEQ |
| Gene | Reverse Primer | ID NO: |
| HBB | 5′AGCTGCACTGTGACAAGCTG 3′ | 64 |
| ALAS2 | 5′CTGTCATTCGTTCGTCCTCA3′ | 65 |
| SLC4A1 | 5′CTGCAGGACTTCACCAAGG3′ | 66 |
| UROS | 5′TTGGCCTGGATACAGAAGGA3′ | 67 |
| DYRK3 | 5′TCCTTCTGAACCACCTCCAC3′ | 68 |
| SH2D2 | 5′GGGGCTACAGAGGGAAGAGA3′ | 69 |
| NFATC3 | 5′ACGACGAGCTCGACTTCAA3′ | 70 |
| LMNA | 5′GCAAAGTGCGTGAGGAGTTT3′ | 71 |
| KCNK5 | 5′CTGCTCAAGGAGTTCCCGT3′ | 72 |
| ZFP36L1 | 5′GTCTGCCACCATCTTCGACT3′ | 73 |

Chromatin Immunoprecipitation (ChIP).

For ChIP-seq experiments the following antibodies were used: Smad1 (Santa Cruz sc7965X), Gata1 (Santa Cruz sc265X), Gata2 (Santa Cruz sc9008X) and H3K27ac (Abcam ab4729). ChIP experiments were performed as previously described with slight modifications (Lee et al., 2006; Trompouki et al., 2011). Briefly, 20-30 million cells for each ChIP were crosslinked by the addition of 1/10 volume 11% fresh formaldehyde for 10 min at room temperature. The crosslinking was quenched by the addition of 1/20 volume 2.5M Glycine. Cells were washed twice with ice-cold PBS and the pellet was flash-frozen inliquid nitrogen. Cells were kept at 80° C. until the experiments were performed. Cells were lysed in 10 ml of Lysis buffer 1 (50 mM HEPES-KOH, pH 7.5, 140 mM NaCl, 1 mM EDTA, 10% glycerol, 0.5% NP-40, 0.25% Triton X-100, and protease inhibitors) for 10 min at 4 C. After centrifugation, cells were resuspended in 10 ml of Lysis buffer 2 (10 mM Tris-HCl, pH 8.0, 200 mM NaCl, 1 mM EDTA, 0.5 mM EGTA, and protease inhibitors) for 10 min at room temperature. Cells were pelleted and resuspended in 3 ml of Sonication buffer for K562 and U937 and 1 ml for other cells used (10 mM Tris-HCl, pH 8.0, 100 mM NaCl, 1 mM EDTA, 0.5 mM EGTA, 0.1% Na-Deoxycholate, 0.05% N-lauroylsarcosine, and protease Inhibitors) and sonicated in a Bioruptor sonicator for 24-40 cycles of 30s followed by 1 min resting intervals. Samples were centrifuged for 10 min at 18,000 g and 1% of TritonX was added to the supernatant. Prior to the immunoprecipitation, 50 ml of protein G beads (Invitrogen 100-04D) for each reaction were washed twice with PBS, 0.5% BSA twice. Finally the beads were resuspended in 250 ml of PBS, 0.5% BSA and 5 mg of each antibody. Beads were rotated for at least 6 hr at 4 C and then washed twice with PBS, 0.5% BSA. Cell lysates were added to the beads and incubated at 4C overnight. Beads were washed 1x with (20 mM Tris-HCl (pH 8), 150 mM NaCl, 2 mM EDTA, 0.1% SDS, 1% Triton X-100), 1× with (20 mM Tris-HCl (pH 8), 500 mM NaCl, 2 mM EDTA, 0.1% SDS, 1% Triton X-100), 1× with (10 mM Tris-HCl (pH 8), 250 nM LiCl, 2 mM EDTA, 1% NP40) and 1× with TE and finally resuspended in 200 ml elution buffer (50 mM Tris-Hcl, pH 8.0, 10 mM EDTA and 0.5%-1% SDS) Fifty microliters of cell lysates prior to addition to the beads was kept as input. Crosslinking was reversed by incubating samples at 65C for at least 6 hr. Afterwards the cells were treated with RNase and proteinase K and the DNA was extracted by Phenol/Chloroform extraction (FIGS. 2-7).

RNA Sequencing (RNAseq).

RNAseq was performed on CD34⁺ cells for the following time points post-hrBMP4 stimulation: D0, H2, H6 and D1-8. The cells were kept in media described above and treated with hrBMP4 for 2 hrs before collection. RNA from one million cells was isolated using Trizol according to the manufacturer's instructions. The RNA was DNAse treated using the RNase free DNase set from Qiagen (79254) according to the instructions. The whole amount of RNA was treated with the Ribo-Zero Gold kit (Human/Mouse/Rat, Epicentre) according to the manufacturer's instructions. Briefly 225ul of magnetic beads per sample were washed in RNAse-free water five times. After the last wash 65ul of Magnetic Bead resuspension solution was added and the beads were kept at RT till used. For each sample the recommended amount was used according to the manufacturer and the recommended reaction was set-up and incubated at RT for 5 min. The mixture was then transferred to the magnetic beads and incubated at RT for 5 min and 50° C. for 5 min. The ribo-zero treated RNA was then purified with the recommended modified protocol for RNeasy MinElute Cleanup Kit. Finally the ribo-zero treated RNA was used to create multiplexed RNA-seq libraries using the ScriptSeg™ v2 RNA-Seq Library Preparation Kit (Epicentre) according to the manufacturer's instructions. Briefly 500pg of ribo-zero treated RNA was fragmented and used to produce cDNA according to the manufacturer's protocol. The cDNA was cleaned with Agencourt AMPure purification and this was used as a template to produce multiplexed libraries (see library preparation) (FIGS. 3-4).

ChIP-Seq and RNA-Seq Library Preparation.

Briefly, ChIPseq libraries were prepared using the following protocol. End repair of immunoprecipitated DNA was performed using the End-It End-Repair kit (Epicentre, ER81050) and incubating the samples at 25° C. for 45 min. End repaired DNA was purified using AMPure XP Beads (1.8× of the reaction volume) (Agencourt AMPure XP—PCR purification Beads, BeckmanCoulter, A63881) and separating beads using DynaMag-96 Side Skirted Magnet (Life Technologies, 12027). A-tail was added to the end-repaired DNA using NEB Klenow Fragment Enzyme (3′-5′ exo, M0212L), 1×NEB buffer 2 and 0.2 mM dATP (Invitrogen, 18252-015) and incubating the reaction mix at 37° C. for 30 min. A-tailed DNA was cleaned up using AMPure beads (1.8× of reaction volume). Subsequently, cleaned up dA-tailed DNA went through Adaptor ligation reaction using Quick Ligation Kit (NEB, M2200L) following manufacturer's protocol. Adaptor-ligated DNA was first cleaned up using AMPure beads (1.8× of reaction volume), eluted in 100 μl and then size-selected using AMPure beads (0.9× of the final supernatant volume, 90 μl). Adaptor ligated DNA fragments of proper size were enriched with PCR reaction using Fusion High-Fidelity PCR Master Mix kit (NEB, M0531S) and specific index primers supplied in NEBNext Multiplex Oligo Kit for Illumina (Index Primer Set 1, NEB, E7335L). Conditions for PCR used are as follows: 98° C., 30 sec; [98° C., 10 sec; 65° C., 30 sec; 72° C., 30 sec]×15 to 18 cycles; 72° C., 5 min; hold at 4° C. PCR enriched fragments were further size-selected by running the PCR reaction mix in 2% low-molecular weight agarose gel (Bio-Rad, 161-3107) and subsequently purifying them using QIAquick Gel Extraction Kit (28704). Libraries were eluted in 24 μl elution buffer. After measuring concentration in Qubit, all the libraries went through quality control analysis using an Agilent Bioanalyzer. Samples with proper size (250-300 bp) were selected for next generation sequencing using Illumina Hiseq 2000 or 2500 platform.

For the RNA-seq libraries, purified double-stranded cDNA underwent end-repair and dA-tailing reactions following manufacturer's reagents and reaction conditions. The obtained DNAs were used for Adaptor Ligation using adaptors and enzymes provided in NEBNext Multiplex Oligos for Illumina (NEB#E7335) and following kit's reaction conditions. Size selection was performed using AMPure XP Beads (starting with 0.6× of the reaction volume). DNA was eluted in 23 μl of nuclease free water. Eluted DNA was enriched with PCR reaction using Fusion High-Fidelity PCR Master Mix kit (NEB, M0531S) and specific index primers supplied in NEBNext Multiplex Oligo Kit for Illumina (Index Primer Set 1, NEB, E7335L). Conditions for PCR used are as follows: 98° C., 30 sec; [98° C., 10 sec; 65° C., 30 sec; 72° C., 30 sec]×15 cycles; 72° C., 5 min; hold at 4° C. PCR reaction mix was purified using Agencourt AMPure XP Beads and eluted in a final volume of 20 pl. After measuring concentration in Qubit, all the libraries went through quality control analysis using an Agilent Bioanalyzer. Samples with proper size (250-300 bp) were selected for high-throughput sequencing using the Illumina Hiseq 2500 platform.

ChIP-Seq data analysis, Alignment and Visualization.

ChIP-Seq reads were aligned to the human reference genome (hg19) using bowtie (Langmead et al.,2009) with parameters -k 2 -m 2 -S. WIG files for display were created using MACS (Zhang et al., 2008) with parameters -w -S --space=50 --nomodel --shiftsize=200 and were displayed in IGV (Robinson et al., 2011; Thorvaldsdottir et al., 2013).

Peak and Bound Gene Identification.

High-confidence peaks of ChIP-Seq signal were identified using MACS with parameters keep-dup=auto -p le-9 and corresponding input control. Bound genes are RefSeq genes that contact a MACS-defined peak between −10000 bp from the TSS and +5000 bp from the TES.

Super-Enhancer Identification.

Super-enhancers were identified as previously described (Kwiatkowski et al., 2014; Whyte et al.,2013). Briefly, peaks of H3K27ac were determined as described above and were used as input for ROSE (https://github.com/BradnerLab/pipeline) with parameters -t 2000 -s 12500 to stitch proximal enhancers together if they were within 12500 bp and outside promoters. Super-enhancers were assigned to the single most proximal expressed transcript where expressed transcripts are in the top ⅔ of H3K27ac ChIP-Seq read density determined by bamToGFF) in a region+/−500 bp from the TSS with parameters -m l -e 200 -r -d. Super-enhancers bound by SMAD1 or GATA factors (FIG. 5A) contact MACS peaks.

Timepoint-Specific Super-Enhancers.

Super-enhancers were separated into H6-specific, D5-specific, and shared populations (FIGS. 5C, 5D, 11) by determining the H3K27ac ChIP-Seq read counts in the collapsed union of H6 and D5 super-enhancer sets using bamToGFF with parameters -t TRUE to which one pseudocount was added. Th150 with the highest H6/D5 ratio, the 150 with the highest D5/H6 ratio, and the 150 nearest H6=D5 are highlighted. H6-specific, D5-specific and common super-enhancers were considered bound by GATA factors or SMAD1 (FIGS. 5B, S5B) if they contacted a MACS-defined peak.

ChIP-Seq Read Density Heatmaps/Scatterplots.

ChIP-Seq read density heatmaps (FIGS. 2A, 2D, 9A, 9D, 9F) were constructed using bamToGFF (https://github.com/BradnerLab/pipeline) on 4kb regions centered on the peak center with parameters—m 200 -r -d and filtered bam files with at most one read per position. Pairwise sharing read heatmaps (FIGS. 2D, 9F) used the collapsed union of the paired timepoint's peaks as input. Regions were separated into early-specific, late-specific or shared based on whether there were MACS-defined peaks at either timepoint. FIG. 9B scatterplots were constructed on H6 GATA2 peaks using bamToGFF with parameters -m l -t TRUE -r to get RPM-normalized read counts in each region, to which one pseudocount was added before log 2-transform.

ChIP-Seq Peak Heatmaps.

Binary peak/not-peak “heatmaps” (FIG. 2B) were determined by first taking the collapsed union of peaks defined at all five timepoints and determining whether each of these collapsed regions contacted a peak in any of the timepoints.

Gata Switch.

The chromatin factors that are targets of GATA2 to GATA1 switch were identified by overlapping the GATA switch gene list with the 425 chromatin factors that were previously tested for hematopoietic phenotypes in zebrafish (Huang et al., 2013) (FIG. 8C), data not shown.

RNAseq Data Analysis.

For the RNA-seq analysis on FIG. 3C: RNA-Seq reads were mapped to the hg19 revision of the human reference genome using tophat (Trapnell et al., 2009) with -G set to a GTF containing RefSeq transcript locations. Expression values for RefSeq transcripts were determined using RPKM_count.py from the RSeQC package (Wang et al., 2012).

For the RNA-seq analysis on FIG. 3A and FIG. 4: RNA seq reads were mapped to the human reference genome (hg19) using TopHat v2.0.13(Kim et al., 2013) the flags: “no-coverage-search GTF gencode.v19.annotation.gtf” where gencode.v19.annotation.gtf is the Gencode v19 reference transcriptome available at gencodegenes.org. Cufflinks v2.2.1(Trapnell et al., 2013) was used to quantify gene expression and assess the statistical significance of differential gene expression. Briefly, Cuffquant was used to quantify mapped reads against Gencode v19 transcripts of at least 200 bp with biotypes: protein_coding, lincRNA, antisense, processed_transript, sense_intronic, sense_overlapping. Cuffdiff was run on the resulting Cuffquant.cxb files, giving a table of FPKM expression level, fold change and statistical significance for each gene.

Assay for Transposase Accessible Chromatin (ATACseq).

CD34⁺ cells were expanded and differentiated using the protocol mentioned above. Before collection, cells were treated with 25 ng/ml hrBMP4 for 2 hr. 5×10⁴ cells per differentiation stage were harvested by spinning at 500×g for 5 min, 4° C. Cells were washed once with 50 uL of cold 1×PBS and spinned down at 500×g for 5 min, 4° C. After discarding supernatant, cells were lysed using 50 uL cold lysis buffer (10 mM Tris-HCl pH 7.4, 10 mM NaCl, 3 mM MgCl2, 0.1% IGEPAL CA-360) and spinned down immediately at 500×g for 10 mins, 4C. Then the cells were precipitated and kept on ice and subsequently resuspended in 25 uL 2× TD Buffer (Illumina Nextera kit), 2.5 uL Transposase enzyme (Illumina Nextera kit, 15028252) and 22.5 uL Nuclease-free water in a total of 50 uL reaction for 1 hr at 37° C. DNA was then purified using Qiagen MinElute PCR purification kit (28004) in a final volume of 10 uL. Libraries were constructed according to Illumina protocol using the DNA treated with transposase, NEB PCR master mix, Sybr green, universal and library-specific Nextera index primers. The first round of PCR was performed under the following conditions: 72° C., 5 min; 98° C., 30 sec; [98° C., 10 sec; 63° C., 30 sec; 72° C., 1 min]×5 cycles; hold at 4° C. Reactions were kept on ice and using a 5 uL reaction aliquot, the appropriate number of additional cycles required for further amplification was determined in a side qPCR reaction: 98° C., 30 sec; [98° C., 10 sec; 63° C., 30 sec; 72° C., 1 min]×20 cycles; hold at 4° C. Upon determining the additional number of PCR cycles required further for each sample, library amplification was conducted using the following conditions: 98° C., 30 sec; [98° C., 10 sec; 63° C., 30 sec; 72° C., 1 min]×appropriate number of cycles; hold at 4° C. Libraries prepared went through quality control analysis using an Agilent Bioanalyzer. Samples with appropriate nucleosomal laddering profiles were selected for next generation sequencing using Illumina Hiseq 2500 platform (FIG. 6).

ATACseq Data Analysis.

All human ChIP-Seq datasets were aligned to build version NCBI37/HG19 of the human genome using Bowtie2 (version 2.2.1) (Langmead et al., 2012) with the following parameters: --end-to-end, -N0, -L20. The MACS2 version 2.1.0 (Zhang et al., 2008) peak finding algorithm was used to identify regions of ATAC-Seq peaks, with the following parameter --nomodel --shift -100 --extsize 200. A q-value threshold of enrichment of 0.05 was used for all datasets. Correlation of ATACseq data with ChIPseq binding: Reads were mapped to the human genome (hg19) using Bowtie v2.2.5 (Langmead and Salzberg, 2012) with default options. BedTools (Quinlan and Hall, 2010) was used to count the number of ATAC-seq reads under Gata/Smad peaks (+/−2.5kb from peak center; 50 bp bins). Read counts were normalized by library size to get CPM.

Ingenuity Pathway Analysis.

The enriched genes from each category used (RNA-seq, SEs etc) were imported into Ingenuity Pathways Analysis (IPA) (Ingenuity Systems) to analyze functional interactions between the genes. The functional analysis identified the biological functions and/or diseases that were most significant to the dataset. Molecules from the dataset associated with biological functions, canonical pathways and/or diseases in Ingenuity's Knowledge Base were considered for the analysis. Right-tailed Fisher's exact test was used to calculate a p value determining the probability that each biological function and/or disease assigned to that data set is due to chance alone. The applied threshold was of q value of <0.05. For the upstream regulator analysis Ingenuity examines how many known targets of each transcription regulator are present in the database presented herein. The overlap p-value calls likely upstream regulators based on significant overlap between dataset genes and known targets regulated by a transcription factor. The overlap p-value was calculated using Fisher's Exact Test, and significance was attributed to p-values<0.01. Comparison analysis were done using the default settings (FIGS. 3B, 8E, 3D).

### REFERENCES

Acharya, U., Gau, J. T., Horvath, W., Ventura, P., Hsueh, C. T., and Carlsen, W. (2008). Hemolysis and hyperhomocysteinemia caused by cobalamin deficiency: three case reports and review of the literature. J Hematol Oncol 1, 26.
Alvarez-Dominguez, J. R., Hu, W., Yuan, B., Shi, J., Park, S. S., Gromatzky, A. A., van Oudenaarden, A., and Lodish, H. F. (2014). Global discovery of erythroid long noncoding RNAs reveals novel regulators of red cell maturation. Blood 123, 570-581.
Basak, A., Hancarova, M., Ulirsch, J. C., Balci, T. B., Trkova, M., Pelisek, M., Vlckova, M., Muzikova, K., Cermak, J., Trka, J., et al. (2015). BCL11A deletions result in fetal hemoglobin persistence and neurodevelopmental alterations. J Clin Invest 125, 2363-2368.
Bresnick, E. H., Lee, H. Y., Fujiwara, T., Johnson, K. D., and Keles, S. (2010). GATA switches as developmental drivers. J Biol Chem 285, 31087-31093.
Bulger, M., and Groudine, M. (2011). Functional and mechanistic diversity of distal transcription enhancers. Cell 144, 327-339.
Cantor, A. B., and Orkin, S. H. (2002). Transcriptional regulation of erythropoiesis: an affair involving multiple partners. Oncogene 21, 3368-3376.
Canver, M. C., Smith, E. C., Sher, F., Pinello, L., Sanjana, N. E., Shalem, O., Chen, D. D., Schupp, P. G., Vinjamur, D. S., Garcia, S. P., et al. (2015). BCL11A enhancer dissection by Cas9-mediated in situ saturating mutagenesis. Nature.
Cheng, Y., Wu, W., Kumar, S. A., Yu, D., Deng, W., Tripic, T., King, D. C., Chen, K. B., Zhang, Y., Drautz, D., et al. (2009). Erythroid GATA1 function revealed by genome-wide analysis of transcription factor occupancy, histone modifications, and mRNA expression. Genome Res 19, 2172-2184.
Consortium, E. P. (2012). An integrated encyclopedia of DNA elements in the human genome. Nature 489, 57-74.
Detmer, K., and Walker, A. N. (2002). Bone morphogenetic proteins act synergistically with haematopoietic cytokines in the differentiation of haematopoietic progenitors. Cytokine 17, 36-42.
Dore, L. C., Chlon, T. M., Brown, C. D., White, K. P., and Crispino, J. D. (2012). Chromatin occupancy analysis reveals genome-wide GATA factor switching during hematopoiesis. Blood 119, 3724-3733.
Fuchs, O., Simakova, O., Klener, P., Cmejlova, J., Zivny, J., Zavadil, J., and Stopka, T. (2002). Inhibition of Smad5 in human hematopoietic progenitors blocks erythroid differentiation induced by BMP4. Blood Cells Mol Dis 28, 221-233.
Fujiwara, T., O'Geen, H., Keles, S., Blahnik, K., Linnemann, A. K., Kang, Y. A., Choi, K., Farnham, P. J., and Bresnick, E. H. (2009). Discovering hematopoietic mechanisms through genome-wide analysis of GATA factor chromatin occupancy. Mol Cell 36, 667-681.
Grant, C. E., Bailey, T. L., and Noble, W. S. (2011). FIMO: scanning for occurrences of a given motif. Bioinformatics 27, 1017-1018.
Guo, S., Wang, L., Li, X., Nie, G., Li, M., and Han, B. (2014). Identification of a novel UROS mutation in a Chinese patient affected by congenital erythropoietic porphyria. Blood Cells Mol Dis 52, 57-58.
Heinz, S., Romanoski, C. E., Benner, C., and Glass, C. K. (2015). The selection and function of cell type-specific enhancers. Nat Rev Mol Cell Biol 16, 144-154.
Hnisz, D., Abraham, B. J., Lee, T. I., Lau, A., Saint-Andre, V., Sigova, A. A., Hoke, H. A., and Young, R. A. (2013). Super-enhancers in the control of cell identity and disease. Cell 155, 934-947.
Hnisz, D., Schuijers, J., Lin, C. Y., Weintraub, A. S., Abraham, B. J., Lee, T. I., Bradner, J. E., and Young, R. A. (2015). Convergence of developmental and oncogenic signaling pathways at transcriptional super-enhancers. Mol Cell 58, 362-370. Hung, T., and Chang, H. Y. (2010). Long noncoding RNA in genome regulation: prospects and mechanisms. RNA Biol 7, 582-585.
Huang, H. T., Kathrein, K. L., Barton, A., Gitlin, Z., Huang, Y. H., Ward, T. P., Hofmann, O., Dibiase, A., Song, A., Tyekucheva, S., et al. (2013). A network of epigenetic regulators guides developmental haematopoiesis in vivo. Nat Cell Biol 15, 1516-1525.
Iolascon, A., Heimpel, H., Wahlin, A., and Tamary, H. (2013). Congenital dyserythropoietic anemias: molecular insights and diagnostic approach. Blood 122, 2162-2166.
Isern, J., He, Z., Fraser, S. T., Nowotschin, S., Ferrer-Vaquer, A., Moore, R., Hadjantonakis, A. K., Schulz, V., Tuck, D., Gallagher, P. G., et al. (2011). Single-lineage transcriptome analysis reveals key regulatory pathways in primitive erythroid progenitors in the mouse embryo. Blood 117, 4924-4934.
Jain, D., Mishra, T., Giardine, B. M., Keller, C. A., Morrissey, C. S., Magargee, S., Dorman, C. M., Long, M., Weiss, M. J., and Hardison, R. C. (2015). Dynamics of GATA1 binding and expression response in a GATA1-induced erythroid differentiation system. Genom Data 4, 1-7.
Jeannet, G., Scheller, M., Scarpellino, L., Duboux, S., Gardiol, N., Back, J., Kuttler, F., Malanchi, I., Birchmeier, W., Leutz, A., et al. (2008). Long-term, multilineage hematopoiesis occurs in the combined absence of beta-catenin and gamma-catenin. Blood 111, 142-149.
Kim, D., Pertea, G., Trapnell, C., Pimentel, H., Kelley, R., and Salzberg, S. L. (2013). TopHat2: accurate alignment of transcriptomes in the presence of insertions, deletions and gene fusions. Genome Biol 14, R36.
Kwiatkowski, N., Zhang, T., Rahl, P. B., Abraham, B. J., Reddy, J., Ficarro, S. B., Dastur, A., Amzallag, A., Ramaswamy, S., Tesar, B., et al. (2014). Targeting transcription regulation in cancer with a covalent CDK7 inhibitor. Nature 511, 616-620.
Koch, U., Wilson, A., Cobas, M., Kemler, R., Macdonald, H. R., and Radtke, F. (2008). Simultaneous loss of beta- and gamma-catenin does not perturb hematopoiesis or lymphopoiesis. Blood 111, 160-164.
Langmead, B., and Salzberg, S. L. (2012). Fast gapped-read alignment with Bowtie 2. Nat Methods 9, 357-359.
Langmead, B., Trapnell, C., Pop, M., and Salzberg, S. L. (2009). Ultrafast and memory-efficient alignment of short DNA sequences to the human genome. Genome Biol 10, R25.
Lee, T. I., Johnstone, S. E., and Young, R. A. (2006). Chromatin immunoprecipitation and microarray-based analysis of protein location. Nat Protoc 1, 729-748.
Lenox, L. E., Perry, J. M., and Paulson, R. F. (2005). BMP4 and Madh5 regulate the erythroid response to acute anemia. Blood 105, 2741-748.
Lettre, G., Sankaran, V. G., Bezerra, M. A., Araujo, A. S., Uda, M., Sanna, S., Cao, A., Schlessinger, D., Costa, F. F., Hirschhorn, J. N., et al. (2008). DNA polymorphisms at the BCL11A, HBS1L-MYB, and beta-globin loci associate with fetal hemoglobin levels and pain crises in sickle cell disease. Proc Natl Acad Sci USA 105, 11869-11874.
Livak, K. J., and Schmittgen, T. D. (2001). Analysis of relative gene expression data using real-time quantitative PCR and the 2(-Delta Delta C(T)) Method. Methods 25, 402-408.
Mullen, A. C., Orlando, D. A., Newman, J. J., Loven, J., Kumar, R. M., Bilodeau, S., Quinlan, A. R., and Hall, I. M. (2010). BEDTools: a flexible suite of utilities for comparing genomic features. Bioinformatics 26, 841-842.
Reddy, J., Guenther, M. G., DeKoter, R. P., and Young, R. A. (2011). Master transcription factors determine cell-type--specific responses to TGF-beta signaling. Cell 147, 565-576.
Robinson, J. T., Thorvaldsdottir, H., Winckler, W., Guttman, M., Lander, E. S., Getz, G., and Mesirov, J. P. (2011). Integrative genomics viewer. Nat Biotechnol 29, 24-26.
Novershtern, N., Subramanian, A., Lawton, L. N., Mak, R. H., Haining, W. N., McConkey, M. E., Habib, N., Yosef, N., Chang, C. Y., Shay, T., et al. (2011). Densely interconnected transcriptional circuits control cell states in human hematopoiesis. Cell 144, 296-309.
Paralkar, V. R., Mishra, T., Luan, J., Yao, Y., Kossenkov, A. V., Anderson, S. M., Dunagin, M., Pimkin, M., Gore, M., Sun, D., et al. (2014). Lineage and species-specific long noncoding RNAs during erythro-megakaryocytic development. Blood 123, 1927-1937.
Paralkar, V. R., and Weiss, M. J. (2013). Long noncoding RNAs in biology and hematopoiesis. Blood 121, 4842-4846.
Rinn, J. L. (2014). lncRNAs: linking RNA to chromatin. Cold Spring Harb Perspect Biol 6.
Rinn, J. L., and Chang, H. Y. (2012). Genome regulation by long noncoding RNAs. Annu Rev Biochem 81, 145-166.
Sankaran, V. G., Menne, T. F., Xu, J., Akie, T. E., Lettre, G., Van Handel, B., Mikkola, H. K., Hirschhorn, J. N., Cantor, A. B., and Orkin, S. H. (2008a). Human fetal hemoglobin expression is regulated by the developmental stage-specific repressor BCL11A. Science 322, 1839-1842.
Sankaran, V. G., Orkin, S. H., and Walkley, C. R. (2008b). Rb intrinsically promotes erythropoiesis by coupling cell cycle exit with mitochondrial biogenesis. Genes Dev 22, 463-475.
Satpathy, A. T., and Chang, H. Y. (2015). Long noncoding RNA in hematopoiesis and immunity. Immunity 42, 792-804.
Schmerer, M., and Evans, T. (2003). Primitive erythropoiesis is regulated by Smad-dependent signaling in postgastrulation mesoderm. Blood 102, 3196-3205.
Singbrant, S., Karlsson, G., Ehinger, M., Olsson, K., Jaako, P., Miharada, K., Stadtfeld, M., Graf, T., and Karlsson, S. (2010). Canonical BMP signaling is dispensable for hematopoietic stem cell function in both adult and fetal liver hematopoiesis, but essential to preserve colon architecture. Blood 115, 4689-4698.
Stonestrom, A. J., Hsu, S. C., Jahn, K. S., Huang, P., Keller, C. A., Giardine, B. M., Kadauke, S., Campbell, A. E., Evans, P., Hardison, R. C., et al. (2015). Functions of BET proteins in erythroid gene expression. Blood 125, 2825-2834.
Su, M. Y., Steiner, L. A., Bogardus, H., Mishra, T., Schulz, V. P., Hardison, R. C., and Gallagher, P. G.
(2013). Identification of biologically relevant enhancers in human erythroid cells. J Biol Chem 288, 8433-8444.
Thorvaldsdottir, H., Robinson, J. T., and Mesirov, J. P. (2013). Integrative Genomics Viewer (IGV):high-performance genomics data visualization and exploration. Brief Bioinform 14, 178-192.
Trapnell, C., Hendrickson, D. G., Sauvageau, M., Goff, L., Rinn, J. L., and Pachter, L. (2013). Differential analysis of gene regulation at transcript resolution with RNA-seq. Nat Biotechnol 31, 46-53.
Trapnell, C., Pachter, L., and Salzberg, S. L. (2009). TopHat: discovering splice junctions with RNA-Seq. Bioinformatics 25, 1105-1111.
Trompouki, E., Bowman, T. V., Lawton, L. N., Fan, Z. P., Wu, D. C., DiBiase, A., Martin, C. S., Cech, J. N., Sessa, A. K., Leblanc, J. L., et al. (2011). Lineage regulators direct BMP and Wnt pathways to cell-specific programs during differentiation and regeneration. Cell 147, 577-589.
Wang, X., Slebos, R. J., Wang, D., Halvey, P. J., Tabb, D. L., Liebler, D. C., and Zhang, B. (2012). Protein identification using customized protein sequence databases derived from RNA-Seq data. J Proteome Res 11, 1009-1017.
Whyte, W. A., Orlando, D. A., Hnisz, D., Abraham, B. J., Lin, C. Y., Kagey, M. H., Rahl, P. B., Lee, T. I., and Young, R. A. (2013). Master transcription factors and mediator establish super-enhancers at key cell identity genes. Cell 153, 307-319.
Wu, W., Cheng, Y., Keller, C. A., Ernst, J., Kumar, S. A., Mishra, T., Morrissey, C., Dorman, C. M., Chen, K. B., Drautz, D., et al. (2011). Dynamics of the epigenetic landscape during erythroid differentiation after GATA1 restoration. Genome Res 21, 1659-1671.
Xu, J., Shao, Z., Li, D., Xie, H., Kim, W., Huang, J., Taylor, J. E., Pinello, L., Glass, K.,Jaffe, J. D., et al. (2015). Developmental control of polycomb subunit composition by GATA factors mediates a switch to non-canonical functions. Mol Cell 57, 304-316.
Zaret, K. S., and Carroll, J. S. (2011). Pioneer transcription factors: establishing competence for gene expression. Genes Dev 25, 2227-2241.
Zhang, C., and Evans, T. (1996). BMP-like signals are required after the midblastula transition for blood cell development. Dev Genet 18, 267-278.
Zhang, Y., Liu, T., Meyer, C. A., Eeckhoute, J., Johnson, D. S., Bernstein, B. E., Nusbaum, (MACS). Genome Biol 9, R137.
Ziller, M. J., Edri, R., Yaffe, Y., Donaghey, J., Pop, R., Mallard, W., Issner, R., Gifford, C. A., Goren, A., Xing, J., et al. (2015). Dissecting neural differentiation regulatory networks through epigenetic footprinting. Nature 518, 355-359.

Tables

TABLE 3
H6 SE GENES

| | | H6 Specific SE bound | | H6 Specific SE NOT bound | |
|---|---|---|---|---|---|
| H6 Specific SE | | by GATA2 and SMAD1 | | by ATA2 and SMAD1 | |
| RefSeq mRNA | | RefSeq mRNA | | RefSeq mRNA | |
| [e.g. | Associated | [e.g. | Associated | [e.g. | Associated |
| NM_001195597] | Gene Name | NM_001195597] | Gene Name | NM_001195597] | Gene Name |
| NM_000117 | EMD | NM_001040168 | LFNG | NM_000117 | EMD |
| NM_000918 | P4HB | NM_001098637 | PWWP2B | NM_000918 | P4HB |
| NM_001004354 | NRARP | NM_001098670 | RASGRP2 | NM_001004354 | NRARP |
| NM_001007533 | PPP1R27 | NM_001101 | ACTB | NM_001007533 | PPP1R27 |
| NM_001009998 | SSBP4 | NM_001109 | ADAM8 | NM_001009998 | SSBP4 |
| NM_001010938 | TNK2 | NM_001145661 | GATA2 | NM_001010938 | TNK2 |
| NM_001010972 | ZYX | NM_002434 | MPG | NM_001010972 | ZYX |
| NM_001012241 | MSL1 | NM_002558 | P2RX1 | NM_001012241 | MSL1 |
| NM_001012614 | CTBP1 | NM_003120 | SPI1 | NM_001012614 | CTBP1 |
| NM_001013255 | LSP1 | NM_003290 | TPM4 | NM_001013255 | LSP1 |
| NM_001017371 | SP3 | NM_004364 | CEBPA | NM_001017371 | SP3 |
| NM_001018076 | NR3C1 | NM_004479 | FUT7 | NM_001018076 | NR3C1 |
| NM_001040168 | LFNG | NM_006278 | ST3GAL4 | NM_001042454 | TGFB1I1 |
| NM_001042454 | TGFB1I1 | NM_006598 | SFC12A7 | NM_001076684 | UBTF |
| NM_001076684 | UBTF | NM_014615 | GSE1 | NM_001077489 | GNAS |
| NM_001077489 | GNAS | NM_014737 | RASSF2 | NM_001080453 | INTS1 |
| NM_001080453 | INTS1 | NM_014838 | ZBED4 | NM_001098833 | ATXN7L3 |
| NM_001098637 | PWWP2B | NM_015898 | ZBTB7A | NM_001100878 | MROH6 |
| NM_001098670 | RASGRP2 | NM_017617 | NOTCH1 | NM_001110556 | FLNA |
| NM_001098833 | ATXN7L3 | NM_017745 | BCOR | NM_001113496 | 9-Sep |
| NM_001100878 | MROH6 | NM_020530 | OSM | NM_001113755 | TYMP |
| NM_001101 | ACTB | NM_020896 | OSBPL5 | NM_001122681 | SH3BP2 |
| NM_001109 | ADAM8 | NM_030767 | AKNA | NM_001127198 | TMC6 |
| NM_001110556 | FLNA | NM_031991 | PTBP1 | NM_001127215 | GFI1 |
| NM_001113496 | 9-Sep | NM_032152 | PRAM1 | NM_001137601 | ZBTB42 |
| NM_001113755 | TYMP | NM_032310 | C9orf89 | NM_001142298 | SQSTM1 |
| NM_001122681 | SH3BP2 | NM_130807 | MOB3A | NM_001166170 | NEK6 |
| NM_001127198 | TMC6 | NM_144653 | NACC2 | NM_001171816 | RNF166 |
| NM_001127215 | GFI1 | NM_152739 | HOXA9 | NM_001198623 | TNFSF13 |
| NM_001137601 | ZBTB42 | NM_174957 | ATP2A3 | NM_001319 | CSNK1G2 |
| NM_001142298 | SQSTM1 | NM_198532 | C19orf35 | NM_001614 | ACTG1 |
| NM_001145661 | GATA2 | NM_203370 | FAM212A | NM_001619 | ADRBK1 |
| NM_001166170 | NEK6 | | | NM_001694 | ATP6V0C |
| NM_001171816 | RNF166 | | | NM_001909 | CTSD |
| NM_001198623 | TNFSF13 | | | NM_001913 | CUX1 |
| NM_001319 | CSNK1G2 | | | NM_002383 | MAZ |
| NM_001614 | ACTG1 | | | NM_002695 | POLR2E |
| NM_001619 | ADRBK1 | | | NM_003070 | SMARCA2 |
| NM_001694 | ATP6V0C | | | NM_003107 | SOX4 |
| NM_001909 | CTSD | | | NM_003223 | TFAP4 |
| NM_001913 | CUX1 | | | NM_003345 | UBE2I |
| NM_002383 | MAZ | | | NM_003367 | USF2 |
| NM_002434 | MPG | | | NM_003403 | YY1 |
| NM_002558 | P2RX1 | | | NM_003718 | CDK13 |
| NM_002695 | POLR2E | | | NM_003900 | SQSTM1 |
| NM_003070 | SMARCA2 | | | NM_004104 | FASN |
| NM_003107 | SOX4 | | | NM_004195 | TNFRSF18 |
| NM_003120 | SPI1 | | | NM_004207 | SFC16A3 |
| NM_003223 | TFAP4 | | | NM_004561 | OVOL1 |
| NM_003290 | TPM4 | | | NM_004642 | CDK2AP1 |
| NM_003345 | UBE2I | | | NM_004761 | RGL2 |
| NM_003367 | USF2 | | | NM_004807 | HS6ST1 |
| NM_003403 | YY1 | | | NM_004907 | IER2 |
| NM_003718 | CDK13 | | | NM_005022 | PFN1 |
| NM_003900 | SQSTM1 | | | NM_005194 | CEBPB |
| NM_004104 | FASN | | | NM_005224 | ARID3A |
| NM_004195 | TNFRSF18 | | | NM_005539 | INPP5A |
| NM_004207 | SLC16A3 | | | NM_005597 | NFIC |
| NM_004364 | CEBPA | | | NM_005655 | KLF10 |
| NM_004479 | FUT7 | | | NM_006137 | CD7 |
| NM_004561 | OVOL1 | | | NM_006254 | PRKCD |
| NM_004642 | CDK2AP1 | | | NM_006305 | ANP32A |
| NM_004761 | RGL2 | | | NM_006401 | ANP32B |
| NM_004807 | HS6ST1 | | | NM_006494 | ERF |
| NM_004907 | IER2 | | | NM_012401 | PLXNB2 |
| NM_005022 | PFN1 | | | NM_013345 | GPR132 |
| NM_005194 | CEBPB | | | NM_014901 | RNF44 |
| NM_005224 | ARID3A | | | NM_014921 | ADGRL1 |
| NM_005539 | INPP5A | | | NM_015156 | RCOR1 |
| NM_005597 | NFIC | | | NM_015288 | JADE2 |
| NM_005655 | KLF10 | | | NM_015315 | FARP1 |
| NM_006137 | CD7 | | | NM_015894 | STMN3 |
| NM_006254 | PRKCD | | | NM_017572 | MKNK2 |
| NM_006278 | ST3GAL4 | | | NM_018150 | RNF220 |
| NM_006305 | ANP32A | | | NM_018270 | MRGBP |
| NM_006401 | ANP32B | | | NM_018396 | METTF2B |
| NM_006494 | ERF | | | NM_018453 | EAPP |
| NM_006598 | SLC12A7 | | | NM_018957 | SH3BP1 |
| NM_012401 | PLXNB2 | | | NM_020310 | MNT |
| NM_013345 | GPR132 | | | NM_020338 | ZMIZ1 |
| NM_014615 | GSE1 | | | NM_020732 | ARID1B |
| NM_014737 | RASSF2 | | | NM_021034 | IFITM3 |
| NM_014838 | ZBED4 | | | NM_025204 | TRABD |
| NM_014901 | RNF44 | | | NM_030576 | LIMD2 |
| NM_014921 | ADGRL1 | | | NM_030665 | RAI1 |
| NM_015156 | RCOR1 | | | NM_030912 | TRIM8 |
| NM_015288 | JADE2 | | | NM_032051 | PATZ1 |
| NM_015315 | LARP1 | | | NM_032246 | MEX3B |
| NM_015894 | STMN3 | | | NM_032595 | PPP1R9B |
| NM_015898 | ZBTB7A | | | NM_032682 | FOXP1 |
| NM_017572 | MKNK2 | | | NM_032871 | REFT |
| NM_017617 | NOTCH1 | | | NM_033388 | ATG16L2 |
| NM_017745 | BCOR | | | NM_080622 | ABHD16B |
| NM_018150 | RNF220 | | | NM_145690 | YWHAZ |
| NM_018270 | MRGBP | | | NM_145867 | LTC4S |
| NM_018396 | METTL2B | | | NM_153253 | SIPA1 |
| NM_018453 | EAPP | | | NM_177401 | MIDN |
| NM_018957 | SH3BP1 | | | NM_182485 | CPEB2 |
| NM_020310 | MNT | | | NM_182647 | OPRL1 |
| NM_020338 | ZMIZ1 | | | NM_194255 | SLC19A1 |
| NM_020530 | OSM | | | NM_198155 | C21orf33 |
| NM_020732 | ARID1B | | | NM_201384 | PLEC |
| NM_020896 | OSBPL5 | | | | |
| NM_021034 | IFITM3 | | | | |
| NM_025204 | TRABD | | | | |
| NM_030576 | LIMD2 | | | | |
| NM_030665 | RAI1 | | | | |
| NM_030767 | AKNA | | | | |
| NM_030912 | TRIM8 | | | | |
| NM_031991 | PTBP1 | | | | |
| NM_032051 | PATZ1 | | | | |
| NM_032152 | PRAM1 | | | | |
| NM_032246 | MEX3B | | | | |
| NM_032310 | C9orf89 | | | | |
| NM_032595 | PPP1R9B | | | | |
| NM_032682 | FOXP1 | | | | |
| NM_032871 | RELT | | | | |
| NM_033388 | ATG16L2 | | | | |
| NM_080622 | ABHD16B | | | | |
| NM_130807 | MOB3A | | | | |
| NM_144653 | NACC2 | | | | |
| NM_145690 | YWHAZ | | | | |
| NM_145867 | LTC4S | | | | |
| NM_152739 | HOXA9 | | | | |
| NM_153253 | SIPA1 | | | | |
| NM_174957 | ATP2A3 | | | | |
| NM_177401 | MIDN | | | | |
| NM_182485 | CPEB2 | | | | |
| NM_182647 | OPRL1 | | | | |
| NM_194255 | SLC19A1 | | | | |
| NM_198155 | C21orf33 | | | | |
| NM_198532 | C19orf35 | | | | |
| NM_201384 | PLEC | | | | |
| NM_203370 | FAM212A | | | | |

TABLE 4
D5 SE GENES

| | | D5 Specific SE bound by | | D5 Specific SE NOT bound by | |
|---|---|---|---|---|---|
| D5 Specific SE | | GATA1 and SMAD1 | | GATA1 and SMAD1 | |
| RefSeq mRNA | | RefSeq mRNA | | RefSeq mRNA | |
| [e.g. | Associated | [e.g. | Associated | [e.g. | Associated |
| NM_001195597] | Gene Name | NM_001195597] | Gene Name | NM_001195597] | Gene Name |
| NM_000053 | ATP7B | NM_000053 | ATP7B | NM_000560 | CD53 |
| NM_000097 | CPOX | NM_000097 | CPOX | NM_001009608 | SLX4IP |
| NM_000108 | DLD | NM_000108 | DLD | NM_001013694 | SRRD |
| NM_000222 | KIT | NM_000222 | KIT | NM_001039465 | SRSF5 |
| NM_000324 | RHAG | NM_000324 | RHAG | NM_001039703 | NBPF10 |
| NM_000440 | PDE6A | NM_000440 | PDE6A | NM_001098815 | TESPA1 |
| NM_000518 | HBB | NM_000518 | HBB | NM_001105566 | KIF13A |
| NM_000560 | CD53 | NM_000570 | FCGR3B | NM_001128149 | ATXN7 |
| NM_000570 | FCGR3B | NM_000712 | BLVRA | NM_001146 | ANGPT1 |
| NM_000712 | BLVRA | NM_000885 | ITGA4 | NM_001164629 | GTDC1 |
| NM_000885 | ITGA4 | NM_001001548 | CD36 | NM_001190882 | ORC4 |
| NM_001001548 | CD36 | NM_001004023 | DYRK3 | NM_001193514 | SLC30A6 |
| NM_001004023 | DYRK3 | NM_001033024 | FBXO7 | NM_001195396 | ARL4A |
| NM_001009608 | SFX4IP | NM_001033056 | GLUL | NM_001198979 | SMAP2 |
| NM_001013694 | SRRD | NM_001036 | RYR3 | NM_002886 | RAP2B |
| NM_001033024 | FBXO7 | NM_001039570 | KREMEN1 | NM_002933 | RNASE1 |
| NM_001033056 | GLUL | NM_001040177 | AKR1E2 | NM_003760 | EIF4G3 |
| NM_001036 | RYR3 | NM_001114091 | CDC27 | NM_004674 | ASH2L |
| NM_001039465 | SRSF5 | NM_001127593 | FCGR3A | NM_006472 | TXNIP |
| NM_001039570 | KREMEN1 | NM_001128176 | THRB | NM_013330 | NME7 |
| NM_001039703 | NBPF10 | NM_001131062 | SAP30L | NM_014959 | CARD8 |
| NM_001040177 | AKR1E2 | NM_001143974 | ASAH2 | NM_015909 | NBAS |
| NM_001098815 | TESPA1 | NM_001144964 | NEDD4L | NM_021038 | MBNL1 |
| NM_001105566 | KIF13A | NM_001145353 | ELF1 | NM_024408 | NOTCH2 |
| NM_001114091 | CDC27 | NM_001174071 | SERINC5 | NM_052863 | SCGB3A1 |
| NM_001127593 | FCGR3A | NM_001178117 | MINPP1 | NM_080657 | RSAD2 |
| NM_001128149 | ATXN7 | NM_001184879 | CD84 | NM_080921 | PTPRC |
| NM_001128176 | THRB | NM_001193484 | LIMS1 | NM_203281 | BMX |
| NM_001131062 | SAP30L | NM_001195260 | PDE4DIP | | |
| NM_001143974 | ASAH2 | NM_001199180 | ATP2C1 | | |
| NM_001144964 | NEDD4L | NM_001924 | GADD45A | | |
| NM_001145353 | ELF1 | NM_002664 | PLEK | | |
| NM_001146 | ANGPT1 | NM_002732 | PRKACG | | |
| NM_001164629 | GTDC1 | NM_002736 | PRKAR2B | | |
| NM_001174071 | SERINC5 | NM_002888 | RARRES1 | | |
| NM_001178117 | MINPP1 | NM_003126 | SPTA1 | | |
| NM_001184879 | CD84 | NM_003215 | TEC | | |
| NM_001190882 | ORC4 | NM_003473 | STAM | | |
| NM_001193484 | LIMS1 | NM_003558 | PIP5K1B | | |
| NM_001193514 | SFC30A6 | NM_003939 | BTRC | | |
| NM_001195260 | PDE4DIP | NM_004310 | RHOH | | |
| NM_001195396 | ARF4A | NM_004360 | CDH1 | | |
| NM_001198979 | SMAP2 | NM_004445 | EPHB6 | | |
| NM_001199180 | ATP2C1 | NM_004866 | SCAMP1 | | |
| NM_001924 | GADD45A | NM_004929 | CALB1 | | |
| NM_002664 | PEEK | NM_004973 | JARID2 | | |
| NM_002732 | PRKACG | NM_005028 | PIP4K2A | | |
| NM_002736 | PRKAR2B | NM_005112 | WDR1 | | |
| NM_002886 | RAP2B | NM_005124 | NUP153 | | |
| NM_002888 | RARRES1 | NM_005139 | ANXA3 | | |
| NM_002933 | RNASE1 | NM_005330 | HBE1 | | |
| NM_003126 | SPTA1 | NM_005574 | LMO2 | | |
| NM_003215 | TEC | NM_005595 | NFIA | | |
| NM_003473 | STAM | NM_006313 | USP15 | | |
| NM_003558 | PIP5K1B | NM_006620 | HBS1F | | |
| NM_003760 | EIF4G3 | NM_007066 | PKIG | | |
| NM_003939 | BTRC | NM_012081 | ELL2 | | |
| NM_004310 | RHOH | NM_014283 | SUCO | | |
| NM_004360 | CDH1 | NM_014585 | SFC40A1 | | |
| NM_004445 | EPHB6 | NM_014751 | MTSS1 | | |
| NM_004674 | ASH2L | NM_014787 | DNAJC6 | | |
| NM_004866 | SCAMP1 | NM_014800 | ELMO1 | | |
| NM_004929 | CALB1 | NM_015194 | MYOID | | |
| NM_004973 | JARID2 | NM_015365 | AMMECR1 | | |
| NM_005028 | PIP4K2A | NM_017787 | WBP1L | | |
| NM_005112 | WDR1 | NM_018142 | INTS10 | | |
| NM_005124 | NUP153 | NM_018361 | AGPAT5 | | |
| NM_005139 | ANXA3 | NM_018602 | DNAJA4 | | |
| NM_005330 | HBE1 | NM_020476 | ANK1 | | |
| NM_005574 | LMO2 | NM_020640 | DCUN1D1 | | |
| NM_005595 | NFIA | NM_020700 | PPM1H | | |
| NM_006313 | USP15 | NM_020850 | RANBP10 | | |
| NM_006472 | TXNIP | NM_022307 | ICA1 | | |
| NM_006620 | HBS1L | NM_022464 | SIL1 | | |
| NM_007066 | PKIG | NM_022893 | BCL11A | | |
| NM_012081 | ELL2 | NM_024669 | ANKRD55 | | |
| NM_013330 | NME7 | NM_024948 | FAM188A | | |
| NM_014283 | SUCO | NM_030627 | CPEB4 | | |
| NM_014585 | SLC40A1 | NM_030797 | FAM49A | | |
| NM_014751 | MTSS1 | NM_030877 | CTNNBL1 | | |
| NM_014787 | DNAJC6 | NM_032012 | TMEM245 | | |
| NM_014800 | ELMO1 | NM_033179 | OR51B4 | | |
| NM_014959 | CARD8 | NM_058243 | BRD4 | | |
| NM_015194 | MYO1D | NM_130831 | OPA1 | | |
| NM_015365 | AMMECR1 | NM_138799 | MBOAT2 | | |
| NM_015909 | NBAS | NM_147156 | SGMS1 | | |
| NM_017787 | WBP1L | NM_152528 | WDSUB1 | | |
| NM_018142 | INTS10 | NM_152726 | MICU2 | | |
| NM_018361 | AGPAT5 | NM_152789 | FAM133B | | |
| NM_018602 | DNAJA4 | NM_152835 | PDIK1F | | |
| NM_020476 | ANK1 | NM_152991 | EED | | |
| NM_020640 | DCUN1D1 | NM_153371 | LNX2 | | |
| NM_020700 | PPM1H | NM_173791 | PDZD8 | | |
| NM_020850 | RANBP10 | NM_174902 | LDLRAD3 | | |
| NM_021038 | MBNL1 | NM_174916 | UBR1 | | |
| NM_022307 | ICA1 | NM_197978 | HEMGN | | |
| NM_022464 | SIL1 | NM_198892 | BMP2K | | |
| NM_022893 | BCL11A | | | | |
| NM_024408 | NOTCH2 | | | | |
| NM_024669 | ANKRD55 | | | | |
| NM_024948 | FAM188A | | | | |
| NM_030627 | CPEB4 | | | | |
| NM_030797 | FAM49A | | | | |
| NM_030877 | CTNNBL1 | | | | |
| NM_032012 | TMEM245 | | | | |
| NM_033179 | OR51B4 | | | | |
| NM_052863 | SCGB3A1 | | | | |
| NM_058243 | BRD4 | | | | |
| NM_080657 | RSAD2 | | | | |
| NM_080921 | PTPRC | | | | |
| NM_130831 | OPA1 | | | | |
| NM_138799 | MBOAT2 | | | | |
| NM_147156 | SGMS1 | | | | |
| NM_152528 | WDSUB1 | | | | |
| NM_152726 | MICU2 | | | | |
| NM_152789 | FAM133B | | | | |
| NM_152835 | PDIK1L | | | | |
| NM_152991 | EED | | | | |
| NM_153371 | LNX2 | | | | |
| NM_173791 | PDZD8 | | | | |
| NM_174902 | LDLRAD3 | | | | |
| NM_174916 | UBR1 | | | | |
| NM_197978 | HEMGN | | | | |
| NM_198892 | BMP2K | | | | |
| NM_203281 | BMX | | | | |

TABLE 5
SNPs on the H3K27Ac peaks and the ist of Transcription factor motifs that they create or destroy: In Active Enhancer Regions

| Nearest | | Ref | Obs | | |
|---|---|---|---|---|---|
| Gene | SNP | allele | allele | Ref allele motif | Obs allele motif |
| BCL11A | rs243032 | C | T | ESR1_HUMAN.H10MO.S, | Rarb_DBD_3, RARA_full_3, |
| | | | | RREB1_HUMAN.H10MO.D, | RARG_DBD_2, RARA_DBD_3, |
| | | | | RUNX2_DBD_2, RUNX3_DBD_3 | Rarg_DBD_2, |
| | | | | | RARG_HUMAN.H10MO.C, |
| | | | | | RORA_DBD_1 |
| | rs9967849 | C | T | X | ESRRA_DBD_6, ESRRG_full_1, |
| | rs2540913 | A | G | CREB3_full_2, | ETV7_HUMAN.H10MO.D |
| | | | | HLTF_HUMAN.H10MO.D | |
| | | | | (SMARCA3), | |
| | | | | MAFG_full, Mafb_DBD_2 | |
| | rs925484 | C | G | GATA1_HUMAN.H10MO.A, | X |
| | | | | TAL1_HUMAN.H10MO.A | |
| | rs2137281 | C | T | X | Rarg_DBD_3, Rarb_DBD_2, |
| | | | | | NANOG_HUMAN.H10MO.A, |
| | | | | | RARG_DBD_3 |
| | rs2137283 | C | G | RARB_full, RARG_full_1, | X |
| | | | | RARA_full_1, | |
| | | | | NR1I3_HUMAN.H10MO.S, | |
| | | | | Rarg_DBD_1, Rara_DBD_3, | |
| | | | | NR1I2_HUMAN.H10MO.S, | |
| | | | | RARG_DBD_1 | |
| | rs7606173 | G | C | TFAP2C_full_3 | RFX5_HUMAN.H10MO.A, |
| | | | | | ZBT7B_HUMAN.H10MO.D, |
| | | | | | RFX2_HUMAN.H10MO.C |
| TFRC | rs2300774 | C | T | BCF11A_HUMAN.H10MO.C, | GATA3_HUMAN.H10MO.C, |
| | | | | ATOH1_HUMAN.H10MO.D | GATA4_HUMAN.H10MO.B |
| | | | | BCF11A polyclonal antibody | |
| | | | | (BF1797) or of monoclonal | |
| | | | | antibody (MAb) mAb123 | |
| | rs9858727 | A | T | TBP_HUMAN.H10MO.C, | GATA2_HUMAN.H10MO.A, |
| | | | | ONEC2_HUMAN.H10MO.D | GATA4_HUMAN.H10MO.B, |
| | | | | | GATA3_HUMAN.H10MO.C, |
| | | | | | CPEB1_full, |
| | | | | | GATA6_HUMAN.H10MO.B, |
| | | | | | GATA1_HUMAN.H10MO.S |
| | rs11185506 | G | C | FOSL2_HUMAN.H10MO.A, | ZIC3_HUMAN.H10MO.C, |
| | | | | NFKB2_DBD, | TBX1_HUMAN.H10MO.D, |
| | | | | HEN1_HUMAN.H10MO.C, | NR1H4_HUMAN.H10MO.C, |
| | | | | JUNB_HUMAN.H10MO.C, | GLI2_HUMAN.H10MO.B, |
| | | | | JUN_HUMAN.H10MO.A, | GLI2_DBD_1 |
| | | | | NFAT5_HUMAN.H10MO.D, | |
| | | | | FOSB_HUMAN.H10MO.C, | |
| | | | | FOSF1_HUMAN.H10MO.A, | |
| | | | | NFKB1_DBD, | |
| | | | | JUND_HUMAN.H10MO.A, | |
| | | | | FOS_HUMAN.H10MO.A | |
| | rs3761717 | G | C | SOX8_DBD_2, | X |
| | | | | ZEP1_HUMAN.H10MO.D, | |
| | | | | NR1D1_HUMAN.H10MO.C, | |
| | | | | TEAD3_DBD_1 | |
| | rs13072608 | C | T | EGR1_full, Egr3_DBD, | Hic1_DBD_2, |
| | | | | AHR_HUMAN.H10MO.B | TGIF1_HUMAN.H10MO.D, |
| | | | | | P73_HUMAN.H10MO.S |
| | rs9859260 | C | T | ZNF713_full | NR2E1_full_2 |
| | rs9859401 | C | A | NFKB1_DBD, EBF1_full, | ZIC3_HUMAN.H10MO.C, |
| | | | | COE1_HUMAN.H10MO.A | STAT2_HUMAN.H10MO.B, |
| | | | | | GFI2_HUMAN.H10MO.B |
| | rs3804139 | A | G | POU3F4_DBD_1, MEF2B_full, | ESRRA_DBD_3 |
| | | | | MEF2D_HUMAN.H10MO.C, | |
| | | | | MEF2B_HUMAN.H10MO.D, | |
| | | | | POU5F1P1_DBD_1, MEF2D_DBD, | |
| | | | | MEF2C_HUMAN.H10MO.C, | |
| | | | | MEF2A_HUMAN.H10MO.A, | |
| | | | | MEF2A_DBD, Pou2f2_DBD2, | |
| | | | | POU2F3_DBD_1 | |
| | rs11915082 | G | A | WT1_HUMAN.H10MO.D, | MTF1_HUMAN.H10MO.C, |
| | | | | PLAG1_HUMAN.H10MO.D, | ZN713_HUMAN.H10MO.D |
| | | | | KLF16_HUMAN.H10MO.D, | |
| | | | | PURA_HUMAN.H10MO.D, | |
| | | | | EGR1_HUMAN.H10MO.S, | |
| | | | | NR2C2_HUMAN.H10MO.A, | |
| | | | | SP3_HUMAN.H10MO.B | |
| | rs12631246 | G | T | GLIS3_DBD, | NKX32_HUMAN.H10MO.C |
| | | | | ZN219_HUMAN.H10MO.D, | |
| | | | | ZN148_HUMAN.H10MO.D, | |
| | | | | PLAG1_HUMAN.H10MO.S, | |
| | | | | GLIS1_DBD, | |
| | | | | PLAL1_HUMAN.H10MO.D, | |
| | | | | GLIS2_HUMAN.H10MO.D, | |
| | | | | ZNF740_full, GLIS2_DBD | |
| KIT | rs218259 | A | G | X | ZBTB7A_HUMAN.H10MO.D, |
| | | | | | THA_HUMAN.H10MO.C, |
| | | | | | TFCP2_HUMAN.H10MO.D, |
| | | | | | ZKSC1_HUMAN.H10MO.C, |
| | | | | | Mafb_DBD_3 |
| | rs172629 | C | G | X | RARG_full_1, NR2F6_DBD_1 |
| | rs218262 | C | A | ZBTB7A_HUMAN.H10MO.D, | ZNF238_full, ZNF238_DBD, |
| | | | | PLAL1_HUMAN.H10MO.D, | SP1_HUMAN.H10MO.C |
| | | | | ZFX_HUMAN.H10MO.C, | |
| | | | | NFKB1_HUMAN.H10MO.B | |
| | rs218264 | A | T | MA0150.2 | X |
| | | | | (NFE2L2), MA0591.1 | |
| LRRC16 | rs17318575 | C | G | E2F3_DBD_1, E2F2_DBD_1 | FOXO3_full_3 |
| TRIM38 | rs9467647 | A | G | SOX4_HUMAN.H10MO.C, | E2F6_HUMAN.H10MO.C |
| | | | | GATA6_HUMAN.H10MO.B, | |
| | | | | SMAD1_HUMAN.H10MO.D, | |
| | | | | NR0B1_HUMAN.H10MO.D | |
| | rs10946800 | C | T | BRAC_HUMAN.H10MO.D, | ZN410_HUMAN.H10MO.D, |
| | | | | E2F3_DBD_1, | TYY1_HUMAN.H10MO.A |
| | | | | ETV5_HUMAN.H10MO.D, | |
| | | | | ETV7_HUMAN.H10MO.D, | |
| | | | | TBX19_HUMAN.H10MO.D | |
| | rs12195653 | A | G | FOXL1_full_2, | MEF2B_full, |
| | | | | FOXF1_HUMAN.H10MO.D, | MEF2D_HUMAN.H10MO.C, |
| | | | | FOXJ2_HUMAN.H10MO.C, | MEF2D_DBD, MEF2A_DBD |
| | | | | FOXF2_HUMAN.H10MO.D, | |
| | | | | EVX1_HUMAN.H10MO.D, | |
| | | | | FOXC2_DBD_2, FOXC1_DBD_1, | |
| | | | | FOXD3_HUMAN.H10MO.D, | |
| | | | | Foxj3_DBD_4, | |
| | | | | FOXG1_HUMAN.H10MO.D, | |
| | | | | FOXO1_HUMAN.H10MO.C, | |
| | | | | FOXC2_DBD_3, | |
| | | | | FOXJ3_HUMAN.H10MO.A, | |
| | | | | FOXL1_HUMAN.H10MO.D, | |
| | | | | IRF7_DBD_2, | |
| | | | | SRY_HUMAN.H10MO.B, | |
| | | | | Foxc1_DBD_1, | |
| | | | | FOXA3_HUMAN.H10MO.C | |
| | rs9467652 | A | G | X | NRF1_full, |
| | | | | | ELF3_HUMAN.H10MO.D |
| | rs199739 | T | C | TBX20_HUMAN.H10MO.D, | WT1_HUMAN.H10MO.D, |
| | | | | MTF1_HUMAN.H10MO.C, | ZN740_HUMAN.H10MO.D, |
| | | | | TBX1_DBD_2 | Zfp740_DBD, |
| | | | | | ZSCA4_HUMAN.H10MO.D, |
| | | | | | ZN148_HUMAN.H10MO.D, |
| | | | | | SMAD1_HUMAN.H10MO.D, |
| | | | | | EGR2_HUMAN.H10MO.C |
| | rs9467656 | A | G | IRX2_DBD, IRX5_DBD | X |
| | rs2013063 | G | A | X | ZEP2_HUMAN.H10MO.D, |
| | | | | | MAF_HUMAN.H10MO.B, |
| | | | | | STAT4_HUMAN.H10MO.D |
| | rs12216125 | C | T | X | POU3F1_DBD_1, |
| | | | | | POU3F2_DBD_2 |
| HIST1H3A | rs2157050 | G | A | ZN350_HUMAN.H10MO.C | RARA_DBD_2 |
| HIST1H4A | rs9467664 | A | T | IRF1_HUMAN.H10MO.A, | ELK1_full_2, FOXG1_DBD_2, |
| | | | | CTCFL_HUMAN.H10MO_A, | Foxk1_DBD_1, FOXK1_DBD, |
| | | | | HINFP1_full_1 | SMAD1_HUMAN.H10MO.D |
| | rs9379818 | G | T | RFX4_HUMAN.H10MO.D, | FOXG1_DBD_2 |
| | | | | RFX4_DBD_1, | |
| | | | | TFCP2_HUMAN.H10MO.D | |
| HIST1H4B | rs2032449 | G | C | EGR1_HUMAN.H10MO.A, | CTCFL_HUMAN.H10MO.A, |
| | | | | ZN148_HUMAN.H10MO.D | CTCF_HUMAN.H10MO.A |
| | rs3752419 | G | A | TBX20_DBD_3, | X |
| | | | | PAX2_HUMAN.H10MO.D, | |
| | | | | TBX21_HUMANH10MO.D, | |
| | | | | TBX4_HUMAN.H10MO.D, | |
| | | | | MGA_DBD_1, | |
| | | | | MAZ_HUMAN.H10MO.A, | |
| | | | | TBR1_DBD, NFIA_full_2, | |
| | | | | TBX21_DBD_2, Rarg_DBD_1, | |
| | | | | TBX2_full_2, TBX21_full_1, | |
| | | | | TBR1_HUMAN.H10MO.D, | |
| | | | | TBR1_full, TCF4_full, | |
| | | | | TBX4_DBD_1, | |
| | | | | NFIA_HUMAN.H10MO.S, | |
| | | | | TBX15_DBD_2, EOMES_DBD_1, | |
| | | | | RARA_full_1, | |
| | | | | SP1_HUMAN.H10MO.C, | |
| | | | | HES7_HUMAN.H10MO.D, | |
| | | | | Hic1_DBD_2, MGA_DBD_2, | |
| | | | | TBX1_HUMAN.H10MO.D, | |
| | | | | TBX21_full_2, | |
| | | | | TBX1_DBD_3, NFIX_full_3, | |
| | | | | NFIX_full_2, TBX5_DBD_1, | |
| | | | | Rara_DBD_3, RARG_DBD_1 | |
| | rs1540276 | A | T | FOXJ3_HUMAN.H10MO.S, | TEAD3_DBD_2, |
| | | | | HXB7_HUMAN.H10MO.C, | MNX1_HUMAN.H10MO.D, |
| | | | | POU3F2_DBD_1, | TEAD1_full_1, TEAD4_DBD |
| | | | | HXB6_HUMAN.H10MO.D, | |
| | | | | PBX2_HUMAN.H10MO.C, | |
| | | | | POU3F1_DBD_2, | |
| | | | | HXC6_HUMAN.H10MO.D, | |
| | | | | PKNX1_HUMAN.H10MO.D, | |
| | | | | POU3F3_DBD_2, | |
| | | | | HXA10_HUMAN.H10MO.C | |
| HIST1H2AB | rs4401650 | A | G | ARID3A_HUMAN.H10MO.D | X |
| HIST1H2BB | rs7753826 | T | A | SOX9_DBD, | LHX2_DBD_2, |
| | | | | SOX5_HUMAN.H10MO.C, | ZN410_HUMAN.H10MO.D, |
| | | | | BPTF_HUMAN.H10MO.D, | ZN143_HUMAN.H10MO.A, |
| | | | | POU4F2_full, | STAT1_HUMAN.H10MO.S |
| | | | | SOX9_HUMAN.H10MO.B, | |
| | | | | ARI5B_HUMAN.H10MO.C, | |
| | | | | POU4F3_DBD, | |
| | | | | POU4F2_DBD, | |
| | | | | PO4F3_HUMAN.H10MO.D, | |
| | | | | SOX13_HUMAN.H10MO.D, | |
| | | | | SRY_HUMAN.H10MO.B | |
| | rs2032447 | T | C | RARA_full_2, RARG_full_3, | X |
| | | | | RARA_DBD_1, | |
| | | | | ALX1_HUMAN.H10MO.B | |
| HIST1H3C | rs7756117 | G | A | NKX31_HUMAN.H10MO.C, | NFIL3_HUMAN.H10MO.C, |
| | | | | Foxj3_DBD_1 | FUBP1_HUMAN.H10MO.D, |
| | | | | | FOXH1_HUMAN.H10MO.A |
| HIST1H1C | rs807212 | C | T | MECP2_HUMAN.H10MO.C | X |
| HFE | rs2794719 | A | C | FOXG1_DBD_1 | X |
| HIST1H4C | rs198855 | A | T | HOMEZ_DBD | NGN2_HUMAN.H10MO.D, |
| | | | | | BHLHE22_DBD, OLIG1_DBD, |
| | | | | | OLIG1_HUMAN.H10MO.D, |
| | | | | | OLIG3_DBD, |
| | | | | | BHE22_HUMAN.H10MO.D, |
| | | | | | BHLHE23_DBD, OLIG2_full, |
| | | | | | OLIG2_DBD |
| | rs198853 | C | T | E2F5_HUMAN.H10MO.B | E2F3_DBD_1, |
| | | | | | MAFK_HUMAN.H10MO.S, |
| | | | | | E2F2_DBD_3, E2F3_DBD_2, |
| | | | | | IRF5_HUMAN.H10MO.D, |
| | | | | | IRX3_HUMANH10MO.D |
| | rs198851 | T | G | ETS1_HUMAN.H10MO.C, | MEIS3_DBD_1, |
| | | | | FLI1_HUMAN.H10MO.A, | MEIS3_HUMAN.H10MO.D, |
| | | | | CREM_HUMAN.H10MO.C | TGIF1_HUMAN.H10MO.S, |
| | | | | | MEIS2_DBD_2, |
| | | | | | MEIS2_HUMAN.H10MO.B |
| HIST1H2BC | rs198823 | G | T | P63_HUMAN.H10MO.S | X |
| HIST1H2BO | rs13219787 | A | T | TBX5_HUMAN.H10MO.D, | ETV6_full_1 |
| | | | | EOMES_HUMAN.H10MO.D, | |
| | | | | TBX1_DBD_3, TBX4_DBD_1 | |
| FRS3 | rs11962743 | C | G | X | BATF_HUMAN.H10MO.A |
| | rs3761781 | C | T | X | ARID3A_HUMAN.H10MO.D, |
| | | | | | EMX1_DBD_2, TP63_DBD, |
| | | | | | FOXP3_HUMAN.H10MOD |
| PRICKLE4 | rs9394831 | C | T | MAZ_HUMAN.H10MO.A, | X |
| | | | | TFCP2_HUMAN.H10MO.D | |
| | rs8393 | G | T | TEF_HUMAN.H10MO.D | Rara_DBD_2, Rarg_DBD_2, |
| | | | | | PO2F3_HUMAN.H10MO.D, |
| | | | | | ZNF435_full, |
| | | | | | FOXF2_HUMAN.H10MO.D, |
| | | | | | EVX1_HUMAN.H10MO.D, |
| | | | | | ZSC16_HUMAN.H10MO.D |
| USP49 | rs9357366 | T | G | CPEB1_HUMAN.H10MO.D, | RREB1_HUMAN.H10MO.D, |
| | | | | HXD10_HUMAN.H10MO.D, | ZN713_HUMAN.H10MO.D |
| | | | | SOX9_HUMAN.H10MO.B | |
| | rs2249703 | A | G | FOXJ3_HUMAN.H10MO.S, | ZNF713_full |
| | | | | Foxj3_DBD_2, | |
| | | | | BPTF_HUMAN.H10MO.D, | |
| | | | | MCR_HUMAN.H10MO.D, | |
| | | | | BCL6_HUMAN.H10MO.C, | |
| | | | | FOXJ2_DBD_1 | |
| MED20 | rs9357371 | A | C | MA0463.1 | X |
| | rs2274578 | C | G | X | X |
| BYSL | rs2479724 | C | T | PROX1_HUMAN.H10MO.D, | X |
| | | | | SP2_HUMAN.H10MO.C | |
| CCND3 | rs3218108 | G | A | ZSC16_HUMAN.H10MO.D | MEF2A_HUMAN.H10MO.A, |
| | | | | | MEF2C_HUMAN.H10MO.C |
| | rs1051130 | A | C | SMAD1_HUMAN.H10MO.D | REF_HUMAN.H10MO.C, |
| | | | | | NFYC_HUMAN.H10MO.B, |
| | | | | | PBX3_HUMAN.H10MO.B, |
| | | | | | NFKB2_DBD, |
| | | | | | HEN1_HUMAN.H10MO.C, |
| | | | | | YBOX1_HUMAN.H10MO.D, |
| | | | | | AP2B_HUMAN.H10MO.B, |
| | | | | | RELB_HUMAN.H10MO.C, |
| | | | | | TF65_HUMAN.H10MO.A, |
| | | | | | NFKB1_DBD, |
| | | | | | SRBP2_HUMAN.H10MO.B |
| | rs3218097 | C | T | PRDM4_HUMAN.H10MO.D | HIC2_HUMAN.H10MO.D, |
| | | | | | NFIX_full_2 |
| | rs3218086 | A | G | X | GLI1_HUMAN.H10MO.C, |
| | | | | | GLI3_HUMAN.H10MO.B |
| | rs9349204 | A | G | X | SOX10_full_2 |
| | rs7766960 | T | G | MNX1_HUMAN.H10MO.D | PRRX1_HUMAN.H10MO.D, |
| | | | | | THRB_DBD_2 |
| | rs10947997 | G | T | MEOX2_DBD_1, EVX1_DBD, | LHX3_HUMAN.H10MO.C, |
| | | | | DLX2_HUMAN.H10MO.D, | HNF1A_HUMAN.H10MO.A, |
| | | | | DLX1_DBD, Hoxa2_DBD, | HOXB5_DBD, HNF1B_full_2, |
| | | | | RARA_DBD_2, | PAX4_HUMAN.H10MO.D, |
| | | | | NKX62_HUMAN.H10MO.D | ZFHX3_HUMAN.H10MO.D, |
| | | | | | Zfp652_DBD, |
| | | | | | ZN652_HUMAN.H10MO.D, |
| | | | | | HNF1A_full, HOXD8_DBD |
| | rs16895128 | A | G | OTX2_DBD_1, | MTF1_DBD, |
| | | | | ALX4_HUMAN.H10MO.D, | INSM1_HUMAN.H10MO.C, |
| | | | | Alx4_DBD, | YBOX1_HUMAN.H10MO.D |
| | | | | PHX2B_HUMAN.H10MO.D, | |
| | | | | CART1_DBD, PHOX2A_DBD, | |
| | | | | PRRX1_full_2, OTX1_DBD_1, | |
| | | | | PHOX2B_full, Alx1_DBD_2, | |
| | | | | Arx_DBD, DRGX_HUMAN.H10MO.D, | |
| | | | | DRGX_DBD, ARX_HUMAN.H10MO.D, | |
| | | | | PHOX2B_DBD, | |
| | | | | LBX2_DBD_1, ALX4_DBD, | |
| | | | | ALX3_HUMAN.H10MO.D, | |
| | | | | UNC4_HUMAN.H10MO.D, | |
| | | | | Otx1_DBD_1, | |
| | | | | PHX2A_HUMAN.H10MO.D, | |
| | | | | ARX_DBD, ALX3_full_2, | |
| | | | | ALX1_HUMAN.H10MO.B | |
| | rs9349205 | G | A | TFAP4_HUMAN.H10MO.C, | TBX1_HUMAN.H10MO.D |
| | | | | ZN784_HUMAN.H10MO.D | |
| | rs11970772 | T | A | THA_HUMAN.H10MO.C, | X |
| | | | | Rara_DBD_1, | |
| | | | | CUX1_HUMAN.H10MO.C, | |
| | | | | AP2A_HUMAN.H10MO.C | |
| | rs11968166 | A | G | X | HIF1A_HUMAN.H10MO.A, |
| | | | | | HEY2_HUMAN.H10MO.D, |
| | | | | | EPAS1_HUMAN.H10MO.D |
| | rs4478405 | C | T | AHR_HUMAN.H10MO.B | Tp73DBD |
| TAF8 | rs4445045 | C | T | ZN148_HUMAN.H10MO.D, | SRBP2_HUMAN.H10MO.B |
| | | | | ZIC3_full, Zic3_DBD, ZIC1_full, | |
| | | | | ZIC4_HUMAN.H10MO.D, | |
| | | | | ZIC4_DBD | |
| | rs9381120 | T | A | NR6A1_HUMAN.H10MO.B | X |
| | rs9367126 | C | T | CLOCK_HUMAN.H10MO.D, | X |
| | | | | MA0073.1, MA0528.1, | |
| | | | | PRDM4_HUMAN.H10MO.D, | |
| | | | | ZN143_HUMAN.H10MO.A | |
| | rs9357384 | G | A | TEAD1_HUMAN.H10MO.D, | X |
| | | | | RREB1_HUMAN.H10MO.D, | |
| | | | | TBX2_HUMAN.H10MO.D | |
| | rs4554318 | C | T | HESX1_HUMAN.H10MO.D | X |
| CD164 | rs6568571 | A | T | ATF2_HUMAN.H10MO.B, | NFIX_full_4, NFIB_full, |
| | | | | JDP2_DBD_2, | NFIA_full_1, NFIA_HUMAN.H10MO.C, |
| | | | | CREM_HUMAN.H10MO.C, | TLX1_HUMAN.H10MO.D |
| | | | | JDP2_HUMAN.H10MO.D, | |
| | | | | JDP2_full_2, | |
| | | | | CREB5_HUMAN.H10MO.D, | |
| | | | | PAX2_HUMAN.H10MO.S, | |
| | | | | Jdp2_DBD_2 | |
| | rs9374080 | T | C | ONECUT3_DBD, ONECUT2_DBD, | RARA_DBD_3, |
| | | | | ONECUT1_DBD, | RARA_HUMAN.H10MO.C, |
| | | | | PBX2_HUMAN.H10MO.C, | STA5A_HUMAN.H10MO.B, |
| | | | | PAX7_DBD, | RARA_full_3, |
| | | | | PAX3_HUMAN.H10MO.D, | GFI1B_HUMAN.H10MO.C |
| | | | | ONECUT1_full, | |
| | | | | PAX7_HUMAN.H10MO.D, | |
| | | | | BCL6B_DBD | |
| | rs1341271 | T | A | ZN232_HUMAN.H10MO.D, | MSX2_HUMAN.H10MO.D |
| | | | | MEF2D_HUMAN.H10MO.C, | |
| | | | | MAFF_HUMAN.H10MO.A, | |
| | | | | MAFK_DBD_2, MAFG_full | |
| | rs9400273 | A | G | X | SMAD4_HUMAN.H10MO.C, |
| | | | | | ISL2_DBD, NKX2-3_DBD, |
| | | | | | NKX2-3_full, NKX2-8_full, |
| | | | | | NKX2-8_DBD, |
| | | | | | HMX2_DBD, NKX3-2_DBD, |
| | | | | | ISL2_HUMAN.H10MO.D |
| HBS1L | rs1547247 | G | A | P63_HUMAN.H10MO.S | POU3F4_DBD_1, |
| | | | | | NGN2_HUMAN.H10MO.D, |
| | | | | | PO3F4_HUMAN.H10MO.D, |
| | | | | | POU1F1_DBD_2, |
| | | | | | PO2F2_HUMAN.H10MO.D, |
| | | | | | POU3F1_DBD_1, |
| | | | | | POU5F1P1_DBD_1, |
| | | | | | POU3F3_DBD_1, POU2F1_DBD_1, |
| | | | | | P5F1B_HUMAN.H10MO.D, |
| | | | | | POU3F2_DBD_2, |
| | | | | | POU2F2_DBD_1, |
| | | | | | GATA5_HUMAN.H10MO.D, |
| | | | | | Pou2f2_DBD_2, |
| | | | | | POU2F3_DBD_1 |
| | rs13220662 | G | A | X | SOX10_HUMAN.H10MO.D, |
| | | | | | FUBP1_HUMAN.H10MO.D, |
| | | | | | TCF7L2_HUMAN.H10MO.A |
| | rs9483783 | T | C | ZN384_HUMAN.H10MO.C, | STA5B_HUMAN.H10MO.C |
| | | | | ETV2_HUMAN.H10MO.D | |
| | rs9376090 | T | C | TFAP2A_DBD_4, | MECP2_HUMAN.H10MO.C |
| | | | | TFAP2C_full_3, | |
| | | | | TFAP2C_DBD_2, TFAP2A_DBD_2, | |
| | | | | KLF15_HUMAN.H10MO.D, | |
| | | | | TFAP2B_DBD_2 | |
| | rs9389266 | G | T | X | GATA3_HUMAN.H10MO.C, |
| | | | | | PO6F1_HUMAN.H10MO.D, |
| | | | | | GATA1_HUMAN.H10MO.S, |
| | | | | | GATA4_HUMAN.H10MO.B, |
| | | | | | Irx3_DBD |
| | rs2210366 | A | G | NKX28_HUMAN.H10MO.C, | ZN143_HUMAN.H10MO.A |
| | | | | NKX21_HUMAN.H10MO.D, | |
| | | | | PRDM4_HUMAN.H10MO.D | |
| | rs7775698 | C | T | CTCFL_HUMAN.H10MO.A | CDC5L_HUMAN.H10MO.D |
| | rs7776054 | A | G | SOX21_DBD_2, | X |
| | | | | SOX17_HUMAN.H10MO.D, | |
| | | | | SOX9_full_1, SOX7_full_1 | |
| | rs9399137 | T | C | Foxg1_DBD_1, | MLXPL_HUMAN.H10MO.D |
| | | | | FOXA1_HUMAN.H10MO.A, | |
| | | | | FOXC2_DBD_2, | |
| | | | | FOXJ3_DBD_3, | |
| | | | | FOXL1_HUMAN.H10MO.D, | |
| | | | | Foxc1_DBD_1, | |
| | | | | FOXF1_full_2, FOXB1_full, | |
| | | | | FOXO3_HUMAN.H10MO.B, | |
| | | | | FOXC1_DBD_1, | |
| | | | | FOXG1_HUMAN.H10MO.D, | |
| | | | | FOXJ3_HUMAN.H10MO.A, | |
| | | | | FOXJ2_DBD_3 | |
| | rs9389268 | A | G | ZBTB7A_DBD, | RUNX2_DBD_3 |
| | | | | SRBP1_HUMAN.H10MO.B, | |
| | | | | RUNX3_HUMAN.H10MO.C, | |
| | | | | KLF12_HUMAN.H10MO.D, | |
| | | | | KLF8_HUMAN.H10MO.C, | |
| | | | | TBX4_DBD_2 | |
| | rs11759553 | A | T | Tp53_DBD_1 | NR4A2_HUMAN.H10MO.C, |
| | | | | | NR2C1_HUMAN.H10MO.C,, |
| | | | | | RARG_HUMAN.H10MO.C, |
| | | | | | NR4A3_HUMAN.H10MO.D, |
| | | | | | PPARG_HUMAN.H10MO.S, |
| | | | | | RXRG_DBD_1, RARA_full_3, |
| | | | | | RARG_DBD_2, RXRB_DBD, |
| | | | | | Nr2f6_DBD_2, RXRA_DBD_1, |
| | | | | | NR2F6_full, |
| | | | | | RXRA_HUMAN.H10MO.C, |
| | | | | | Rxrb_DBD, |
| | rs9373124 | T | C | X | NR6A1_HUMAN.H10MO.B |
| | rs1074849 | A | G | Foxj3_DBD_2, POU1F1_DBD_2, | PO5F1_HUMAN.H10MO.A, |
| | | | | FOXJ3_DBD_1, FOXJ2_DBD_2, | PO3F1_HUMAN.H10MO.C, |
| | | | | FOXQ1_HUMAN.H10MO.C, | P5F1B_HUMAN.H10MO.D |
| | | | | Foxj3_DBD_3, | |
| | | | | PO6F1_HUMAN.H10MO.D, | |
| | | | | PO3F3_HUMAN.H10MO.D, | |
| | | | | SOX10_full_2, SOX8_DBD_1, | |
| | | | | POU4F3_DBD, FOXJ3_DBD_2, | |
| | | | | SOX13_HUMAN.H10MO.D, | |
| | | | | SRY_HUMAN.H10 | |
| | rs4895440 | A | T | X | NFAC1_HUMAN.H10MO.S, |
| | | | | | NFKB1_HUMAN.H10MO.B, |
| | | | | | RELB_HUMAN.H10MO.C |
| | rs4895441 | A | G | X | NKX2-3_DBD, |
| | | | | | HMX2_DBD, |
| | | | | | NKX23_HUMAN.H10MO.D, |
| | | | | | Nkx3-1_DBD, NKX3-1_full |
| | rs9376092 | A | C | X | SOX10_HUMAN.H10MO.D, |
| | | | | | Foxg1_DBD_1, |
| | | | | | SRY_HUMAN.H10MO.B |
| | rs9389269 | C | T | SOX4_HUMAN.H10MO.C, | SOX15_full_1 |
| | | | | SOX9_DBD, | |
| | | | | SOX9_HUMAN.H10MO.B | |
| | rs10484494 | C | T | RFX4_DBD_2 | X |
| | rs9494145 | T | C | HXD13_HUMAN.H10MO.D | ZIC2_HUMAN.H10MO.C, |
| | | | | (HOXD13) | ZIC1_HUMAN.H10MO.B, |
| | | | | | ZNF410_DBD, |
| | | | | | EGR4_HUMAN.H10MO.D |
| MYB | rs6924609 | A | G | MAFF_HUMAN.H10MO.A, | TEAD3_DBD_2, SOX10_full_2, |
| | | | | MAFK_HUMAN.H10MO.A, | TEAD4_HUMAN.H10MO.A, |
| | | | | SPDEF_DBD_3, | TEAD1_full_1, TEAD4_DBD, |
| | | | | Pou2f2_DBD_1, | FOXG1_DBD_1, |
| | | | | SRYDBD_2, SOX7_full_1 | TEAD3_DBD_1 |
| | rs1320962 | G | A | ETV5_HUMAN.H10MO.D, | IRF9_HUMAN.H10MO.C |
| | | | | BCL11A_HUMAN.H10MO.C | |
| | rs1320963 | A | G | PRDM1_HUMAN.H10MO.C, | PPARG_HUMAN.H10MO.A, |
| | | | | BCF11A_HUMAN.H10MO.C, | IRF5_HUMAN.H10MO.D, |
| | | | | PRDM1_full, | RXRA_HUMAN.H10MO.C |
| | | | | IRF1_HUMAN.H10MO.A | |
| | rs9494149 | C | T | STAT4_HUMAN.H10MO.D | X |
| | rs9376095 | T | C | TBX2_HUMAN.H10MO.D, | Tcfap2a_DBD_1, |
| | | | | EBF1_full, | TCF7L1_HUMAN.H10MO.D, |
| | | | | YBOX1_HUMAN.H10MO.D, | TFAP2C_DBD_1, TFAP2A_DBD_5, |
| | | | | COE1_HUMAN.H10MO.A | TFAP2A_DBD_1 |
| | rs6929404 | C | A | NR4A3_HUMAN.H10MO.D, | PO3F3_HUMAN.H10MO.D, |
| | | | | RUNX2_DBD_1 | DLX5_HUMAN.H10MO.D |
| | rs2078213 | C | T | IRF4_full, | RARG_full_2, |
| | | | | PLAG1_HUMAN.H10MO.D, | ZN524_HUMAN.H10MO.D |
| | | | | IRF8_full, | |
| | | | | NR1I2_HUMAN.H10MO.C, | |
| | | | | IRF5_full_1 | |
| | rs17064262 | T | C | TF2LX_HUMAN.H10MO.D, | X |
| | | | | SOX18_HUMAN.H10MO.D | |
| | rs11965277 | G | A | X | Nr2f6_DBD_1, |
| | | | | | MNT_HUMAN.H10MO.D, |
| | | | | | RARG_HUMAN.H10MO.C, |
| | | | | | RARA_DBD_2, |
| | | | | | Rarb_DBD_1, RARB_full, |
| | | | | | RARG_full_1, |
| | | | | | NR2E1_HUMAN.H10MO.D, |
| | | | | | NR2F6_DBD_1, |
| | | | | | Rara_DBD_3, RARG_DBD_1 |
| | rs210962 | C | T | SP4_HUMAN.H10MO.D, | X |
| | | | | SMAD3_HUMAN.H10MO.C | |
| | rs1022506 | C | T | LHX6_full_2, HNF4A_DBD_1 | HMX3_DBD, |
| | | | | | HMX3_HUMAN.H10MO.D, |
| | | | | | SOX10_full_2, |
| | | | | | HMX1_DBD, SOX8_DBD_1, |
| | | | | | NKX2-3_DBD, NKX3-2_DBD, |
| | | | | | HXC10_HUMAN.H10MO.D, |
| | | | | | NKX22_HUMAN.H10MO.D, |
| | | | | | NKX2-3_full, SOX9_full_2, |
| | | | | | Nkx3-1_DBD, SOX9_full_1, |
| | | | | | Atf4_DBD, |
| | | | | | NKX23_HUMAN.H10MO.D, |
| | | | | | SOX21_DBD_2, |
| | rs11154794 | C | T | HLTF_HUMAN.H10MO.D | NFYC_HUMAN.H10MO.B |
| | | | | (SMARCA3), | |
| | | | | NFYB_HUMAN.H10MO.A, | |
| | | | | FOXI1_HUMAN.H10MO.B | |
| | rs12663543 | A | G | X | EOMES_HUMAN.H10MO.D, |
| | | | | | EVX1_DBD |
| | rs12660713 | A | G | RREB1_HUMAN.H10MO.D | MYB_HUMAN.H10MO.C, |
| | | | | | ZKSC1_HUMAN.H10MO.C |
| CITED2 | rs628751 | C | A | RUNX2_DBD_1, | MEF2A_HUMAN.H10MO.A, |
| | | | | FOXG1_HUMAN.H10MO.D, | FOXJ3_DBD_3, |
| | | | | EVX1_HUMAN.H10MO.D, | ETV2_HUMAN.H10MO.D |
| | | | | Foxc1_DBD_1 | |
| | rs643381 | C | A | AP2A_HUMAN.H10MO.C, | ZN143_HUMAN.H10MO.A, |
| | | | | PLAG1_HUMAN.H10MO.S, | ZIC1_full, |
| | | | | ZIC1_HUMAN.H10MO.B, | Egr1_mouse_DBD_mutant_DBD, |
| | | | | TBX15_HUMAN.H10MO.D, | GLIS3_DBD, |
| | | | | ESRRA_DBD_2, | GLIS1_HUMAN.H10MO.D, |
| | | | | RARA_HUMAN.H10MO.C, | ZIC4_HUMAN.H10MO.D |
| | | | | TBX1_HUMAN.H10MO.D, | |
| | | | | TFAP2C_full_1, | |
| | | | | SP3_HUMAN.H10MO.B, | |
| | | | | EGR4_HUMAN.H10MO.D, | |
| | | | | TFAP2B_DBD_1, | |
| | | | | TFAP2C_DBD_1, | |
| | | | | TFAP2A_DBD_5, | |
| | | | | EGR2_HUMAN.H10MO.C, | |
| | | | | ESRRA_DBD_6, | |
| | | | | TFAP2A_DBD_1, | |
| | | | | SP2_HUMAN.H10MO.C, | |
| | | | | AP2C_HUMAN.H10MO.A, | |
| | | | | COE1_HUMAN.H10MO.A, | |
| | | | | Tcfap2a_DBD_1, | |
| | | | | SRBP2_HUMAN.H10MO.B | |
| | rs592423 | A | C | X | DDIT3_HUMAN.H10MO.C |
| | | | | | (CEBP/Z), |
| | | | | | HFTF_HUMAN.H10MO.D |
| | | | | | (SMARCA3), ZNF410_DBD |
| IKZF1 | rs12534526 | G | A | PAX2_DBD, PAX5_DBD | TBX1_DBD_2, BMAL1_HUMAN.H10MO.C, |
| | | | | | Mlx_DBD, TFE3_DBD, Bhlhb2_DBD_2, |
| | | | | | BHLHE41_full, MLXIPF_full, |
| | | | | | BHLHB3_full, CEBPZ_HUMAN.H10MO.D, |
| | | | | | ARNTL_DBD, BHE41_HUMAN.H10MO.D, |
| | | | | | BHLHB2_DBD, Bhlhb2_DBD_1, MLX_full, |
| | | | | | MLX_HUMAN.H10MO.D, BHE40_HUMAN.H10MO.A |
| | rs12718597 | C | A | X | MTF1_HUMAN.H10MO.C |
| | rs12718598 | T | C | X | XBP1_HUMAN.H10MO.C, CREB3L1_full_2, |
| | | | | | ATF6A_HUMAN.H10MO.B, HINFP1_full_2, |
| | | | | | MAZ_HUMAN.H10MO.A, CR3L2_HUMAN.H10MO.D, |
| | | | | | HIF1A_HUMAN.H10MO.A, XBP1_DBD_2, |
| | | | | | CREB3L1_DBD_4, SP3_HUMAN.H10MO.B |
| | rs7385935 | A | G | X | KLF1_HUMAN.H10MO.C, |
| | | | | | ZSCA4_HUMAN.H10MO.D, |
| | | | | | PURA_HUMAN.H10MO.D |
| RCF1 | rs13284787 | A | G | POU1F1_DBD_2, POU3F2_DBD_2, | PRGR_HUMAN.H10MO.S, |
| | | | | POU3F1_DBD_1, | GCR_HUMAN.H10MO.S, |
| | | | | POU3F3_DBD_1, | Ar_DBD |
| | | | | HSF2_HUMAN.H10MO.A | |
| | rs10758656 | A | G | GATA4_DBD, | X |
| | | | | GATA1_HUMAN.H10MO.A, | |
| | | | | TAL1_HUMAN.H10MO.A, | |
| | | | | GATA5_DBD, GATA3_DBD, | |
| | | | | GATA6_HUMAN.H10MO.B, | |
| | | | | GATA1_HUMAN.H10MO.S, | |
| | | | | GATA3_full | |
| | rs10758658 | G | A | Rara_DBD_2, RARG_full_2, | X |
| | | | | RARG_DBD_2, Rarg_DBD_2, | |
| | | | | RARA_HUMAN.H10MO.C | |
| MARCH8 | rs10900218 | A | G | IRF7_DBD_1 | ZN713_HUMAN.H10MO.D, |
| | | | | | SMAD2_HUMAN.H10MO.C |
| HK1 | rs4745982 | T | G | COT2_HUMAN.H10MO.A, | CTCF_HUMAN.H10MO.A, |
| | | | | Rara_DBD_1, Rarg_DBD_3, | SP4_HUMAN_H10MO.D |
| | | | | Rarb_DBD_2, | |
| | | | | NR2C2_HUMAN.H10MO.A, | |
| | | | | RARA_full_2 | |
| FNTB | rs11628273 | C | T | HFTF_HUMAN.H10MO.D | X |
| | | | | (SMARCA3) | |
| MAST1 | rs2290689 | A | G | Klf12_DBD | ZNF740_full, |
| | | | | | KFF13_HUMAN.H10MO.D, |
| | | | | | EGR1_HUMAN.H10MO.S, |
| | | | | | ZN219_HUMAN.H10MO.D, |
| | | | | | EGR1_HUMAN.H10MO.A, |
| | | | | | ZN148_HUMAN.H10MO.D, |
| | | | | | KLF16_HUMAN.H10MO.D |
| DNASE2 | rs7249143 | G | T | X | NR4A2_HUMAN.H10MO.C, |
| | | | | | NR2C1_HUMAN.H10MO.C, |
| | | | | | ESRRA_DBD_3, |
| | | | | | RARG_full_3, |
| | | | | | PPARD_HUMAN.H10MO.D, |
| | | | | | NR4A1_HUMAN.H10MO.C, |
| | | | | | RORA_DBD1, |
| | | | | | PPARG_HUMAN.H10MO.S, |
| | | | | | Rarb_DBD_3, Rarg_DBD_3, |
| | | | | | RARA_full_3, RXRB_DBD, |
| | | | | | Nr2f6_DBD_2, NR2F6_full, |
| | | | | | NR2F1_DBD_3, ESRRG_full_1, |
| | | | | | PPARG_HUMAN.H10MO.A, |
| | | | | | Rara_DBD_1, RXRA_full_1, |
| | | | | | NR4A2_full_3, |
| | | | | | SOX18_HUMAN.H10MO.D, |
| | | | | | RARB_HUMAN.H10MO.D, |
| | | | | | NR2F1_DBD_1, |
| | | | | | RARG_full_2, |
| | | | | | NR2C2_HUMAN.H10MO.A, |
| | | | | | NR2E3_HUMAN.H10MO.C, |
| | | | | | ESRRG_full_3, |
| GCDH | rs11085824 | A | G | X | Rarb_DBD_2, Esrra_DBD_1, |
| | | | | | ESRRA_DBD_2, Vdr_DBD, |
| | | | | | ESRRG_full_2, VDR_full, |
| | | | | | RARA_DBD_1, |
| | | | | | ESRRA_DBD_5 |
| | rs2242517 | T | G | GLIS3_DBD, ZBTB7B_full, | Egr1_mouse_DBD_mutant_DBD, |
| | | | | GFIS1_HUMAN.H10MO.D | WT1_HUMAN.H10MO.D, |
| | | | | | KLF1_HUMAN.H10MO.C, |
| | | | | | KLF12_HUMAN.H10MO.D, |
| | | | | | KLF16_DBD, |
| | | | | | MAZ_HUMAN.H10MO.A, |
| | | | | | ASCL2_HUMAN.H10MO.D, |
| | | | | | ZIC2_HUMAN.H10MO.C, |
| | | | | | ZIC1_HUMAN.H10MO.B, |
| | | | | | FOXK1_HUMAN.H10MO.D, |
| | | | | | SPIC_HUMAN.H10MO.D, |
| | | | | | SP4_HUMAN.H10MO.D, |
| | | | | | ZN219_HUMAN.H10MO.D, |
| | | | | | EGR1_HUMAN.H10MO.A, |
| | | | | | ARNT2_HUMAN.H10MO.D, |
| | | | | | SP1_HUMAN.H10MO.C, |
| | | | | | SP2_HUMAN.H10MO.C, |
| | | | | | GLIS2_HUMAN.H10MO.D, |
| | | | | | KLF3_HUMAN.H10MO.D, |
| | | | | | EGR3_DBD, FOXO1_DBD_3, |
| | | | | | SP3_DBD, |
| | | | | | CLOCK_HUMAN.H10MO.D |
| FARSA | rs2965214 | G | A | GSC2_HUMAN.H10MO.D, | RARG_full_3 |
| | | | | E2F6_HUMAN.H10MO.C, | |
| | | | | ZN639_HUMAN.H10MO.D, | |
| | | | | PLAG1_HUMAN.H10MO.D, | |
| | | | | SPIC_HUMAN.H10MO.D, | |
| | | | | TBX15_HUMAN.H10MO.D, | |
| | | | | MAZ_HUMAN.H10MO.A | |
| CALR | rs1010222 | A | G | NR2E1_HUMAN.H10MO.D | TBX15_DBD_1, TBX20_DBD_2, |
| | | | | | TBX1_DBD_2, |
| | | | | | HEY2_HUMAN.H10MO.D |
| LMF2 | rs762669 | G | A | EGR1_DBD, HES5_DBD_1, | NGN2_HUMAN.H10MO.D, |
| | | | | CREB3L1_DBD_3, EGR2_full, | MITF_HUMAN.H10MO.C, |
| | | | | USF1_HUMAN.H10MO.A, | PKNX2_HUMAN.H10MO.D |
| | | | | BMAL1_HUMAN.H10MO.C, | |
| | | | | MNT_DBD, | |
| | | | | ZKSC1_HUMAN.H10MO.C, | |
| | | | | MAX_HUMAN.H10MO.A, | |
| | | | | CR3L1_HUMAN.H10MO.D, | |
| | | | | MYCN_HUMAN.H10MO.B, | |
| | | | | EGR1_HUMAN.H10MO.S, | |
| | | | | MAX_DBD_2, HEY1_DBD, | |
| | | | | CREB3L1_DBD_2, HEY2_DBD, | |
| | | | | MYC_HUMAN.H10MO.A, | |
| | | | | HES5_HUMAN.H10MO.D, | |
| | | | | GLIS1_HUMAN.H10MO.D, | |
| | | | | EGR2_DBD, | |
| | | | | USF2_HUMAN.H10MO.A, | |
| | | | | HEY2_full, CREB3Ll_full_1, | |
| | | | | HEY1_HUMAN.H10MO.D, | |
| | | | | CLOCK_DBD, | |
| | | | | EPAS1_HUMAN.H10MO.D | |
| ODF3B | rs140521 | C | A | XBP1_DBD_1, | LHX2_DBD_2, |
| | | | | THB_HUMAN.H10MO.C, | VSX1_HUMAN.H10MO.D, |
| | | | | GMEB2_HUMAN.H10MO.D | DLX4_HUMAN.H10MO.D, |
| | | | | | MEOX2_DBD_2, |
| | | | | | SRF_HUMAN.H10MO.A, |
| | | | | | DLX6_HUMAN.H10MO.D, |
| | | | | | DLX1_HUMAN.H10MO.D |
| NRG4 | rs4886755 | A | G | POU1F1_DBD_2, PRDM1_full, | IRF5_HUMAN.H10MO.D, |
| | | | | PRDM1_HUMAN.H10MO.C, | FOXJ2_DBD_3 |
| | | | | STAT2_HUMAN.H10MO.B | |
| MPG | rs2562181 | A | G | Meis3_DBD_2, PKNOX1_DBD, | Rarb_DBD_2, |
| | | | | Meis2_DBD_2, TGIF_1_DBD, | NR2C1_HUMAN.H10MO.C, |
| | | | | TGIF2_DBD, | GRHL1_DBD_1, Esrra_DBD_1, |
| | | | | TGIF2_HUMAN.H10MO.D | Rara_DBD_1 |
| SLC12A7 | rs4535497 | C | A | PO5F1_HUMAN.H10MO.A, | NR4A2_full_2, |
| | | | | SOX2_HUMAN.H10MO.B, | GFI1B_HUMAN.H10MO.C, |
| | | | | NANOG_HUMAN.H10MO.A | BCL6B_DBD, |
| | | | | | HNF4A_HUMAN.H10MO.A, |
| | | | | | GFI1_HUMAN.H10MO.C |
| ANK1 | rs4737009 | G | A | X | SRF_HUMAN.H10MO.A |
| NPRL3 | rs7197554 | A | C | EGR1_DBD, EGR4_DBD_2, | PLAG1_HUMAN.H10MO.S, |
| | | | | EGR1_full, EGR2_full, | ZIC2_HUMAN.H10MO.C, |
| | | | | EGR1_HUMAN.H10MO.S, | ZIC1_HUMAN.H10MO.B, |
| | | | | KLF14_DBD, | ZN219_HUMAN.H10MO.D, |
| | | | | FOXG1_HUMAN.H10MO.D | GLIS2_HUMAN.H10MO.D, |
| | | | | | KLF3_HUMAN.H10MO.D, |
| | | | | | TBX1_HUMAN.H10MO.D, |
| | | | | | PROX1_HUMAN.H10MO.D, |
| | | | | | IRX2_HUMAN.H10MO.D |
| CDT1 | rs2608604 | A | G | X | HXD13_HUMAN.H10MO.D, |
| | | | | | SRF_full |
| THRB | rs1505307 | T | C | SNAI2_HUMAN.H10MO.C, | X |
| | | | | TFE3_HUMAN.H10MO.C, | |
| | | | | ARNTL_DBD | |
| RBPMS | rs2979489 | G | A | ESRRG_full_1, | NR4A2_HUMAN.H10MO.C, |
| | | | | NR1D1_HUMAN.H10MO.C | RXRG_HUMAN.H10MO.B, |
| | | | | | MEF2B_HUMAN.H10MO.D, |
| | | | | | NR4A2_full_3, |
| | | | | | PPARG_HUMAN.H10MO.S, |
| | | | | | Rarb_DBD_1, Foxj3_DBD_4, |
| | | | | | FOXJ2_DBD_3, MEF2A_DBD, |
| | | | | | CDX2_HUMAN.H10MO.C, |
| | | | | | NR2F1_DBD_3 |
| NCOA4 | rs17720193 | T | C | Esrra_DBD_2, | GRHF1_DBD_1 |
| | | | | ERR3_HUMAN.H10MO.B, | |
| | | | | ESRRG_full_1, | |
| | | | | NR6A1_HUMAN.H10MO.B | |
| RBM38 | rs737092 | T | C | X | NKX23_HUMAN.H10MO.D, |
| | | | | | NKX3-1_full, Nkx3-1_DBD, |
| | | | | | SMAD3_HUMAN.H10MO.C, |
| | | | | | MAFG_HUMAN.H10MO.C, |
| | | | | | NKX3-2_DBD |
| ACTL6B | rs2075672 | A | G | ETV7_HUMAN.H10MO.D | SRF_HUMAN.H10MO.A, |
| | | | | | FLI1_HUMAN.H10MO.A, |
| | | | | | ELF2_HUMAN.H10MO.C, |
| | | | | | ELF1_HUMAN.H10MO.A |
| SH2B3 | rs3184504 | C | T | MBD2_HUMAN.H10MO.B | FLI1_DBD_2 |
| ABO | rs495828 | G | T | FOXD1_HUMAN.H10MO.D, | FOXD3_DBD_1, FOXC2_DBD_1, |
| | | | | TBX3_HUMAN.H10MO.D | FOXJ3_HUMAN.H10MO.S, |
| | | | | | FOXC1_DBD_2, |
| | | | | | PO3F2_HUMAN.H10MO.D, |
| | | | | | POU6F2_DBD_2, |
| | | | | | FOXD2_HUMAN.H10MO.D, |
| | | | | | FOXB1_DBD_2, |
| | | | | | FOXD2_DBD_1 |
| ATXN2 | rs653178 | A | G | Tp53_DBD_1 | FOXG1_DBD_2, ZSCAN4_full |
| BRAP | rs11065987 | A | G | X | VDR_full |
| KLF1 | rs12609744 | C | T | X | X |
| MOSPD3 | rs12532878 | A | G | X | HIC2_DBD, |
| | | | | | HIC2_HUMAN.H10MO.D, |
| TFR2 | rs7385804 | A | C | PITX3_HUMAN.H10MO.D | TEAD1_full_2, |
| | | | | | TBX2_HUMAN.H10MO.D, |
| | | | | | TEAD3_DBD_1 |
| PFCF2 | rs2060597 | C | T | SPIB_DBD, SPI1_full, | ELF3_full |
| | | | | IRF9_HUMAN.H10MO.C | |
| OR51V1 | rs140522 | A | G | X | X |

TABLE 6
Open Chromatin Regions: SNPs on the H3K27Ac peaks and the ist of Transcription
factor motifs that they create or destroy: ATACseq-SNPs

| Nearest | | ref | obs | | |
|---|---|---|---|---|---|
| Gene | SNP | allele | allele | ref allele motif | obs allele motif |
| BCF11A | rs243032 | C | T | ESR1_HUMAN.H10MO.S, | Rarb_DBD_3, RARA_full_3, |
| | | | | RREB1_HUMAN.H10MO.D, | RARG_DBD_2, RARA_DBD_3, |
| | | | | RUNX2_DBD_2, | Rarg_DBD_2, |
| | | | | RUNX3_DBD_3 | RARG_HUMAN.H10MO.C, |
| | | | | | RORA_DBD_1 |
| | rs2540913 | A | G | CREB3_full_2, | ETV7.HUMAN.H10MO.D |
| | | | | HLTF_HUMAN.H10MO.D | |
| | | | | (SMARCA3), MAFG_full, Mafb_DBD_2 | |
| | rs7606173 | G | C | TFAP2C_full_3 | RFX5_HUMAN.H10MO.A, |
| | | | | | ZBT7B_HUMAN.H10MO.D, |
| | | | | | RFX2_HUMAN.H10MO.C |
| TFRC | rs13072608 | C | T | EGR1_full, Egr3_DBD, | Hic1_DBD_2, |
| | | | | AHR_HUMAN.H10MO.B | TGIF1_HUMAN.H10MO.D, |
| | | | | | P73_HUMAN.H10MO.S |
| | rs9859260 | C | T | ZNF713_full | NR2E1_full_2 |
| | rs9859401 | C | A | NFKB1_DBD, EBF1_full, | ZIC3_HUMAN.H10MO.C, |
| | | | | COE1_HUMAN.H10MO_A | STAT2_HUMAN.H10MO.B, |
| | | | | | GLI2_HUMAN.H10MO.B |
| | rs11915082 | G | A | WT1_HUMAN.H10MO.D, | MTF1_HUMAN.H10MO.C, |
| | | | | PLAG1_HUMAN.H10MO.D, | ZN713_HUMAN.H10MO.D |
| | | | | KFF16_HUMAN.H10MO.D, | |
| | | | | PURA_HUMAN.H10MO.D, | |
| | | | | EGR1_HUMAN.H10MO.S, | |
| | | | | NR2C2_HUMAN.H10MO.A, | |
| | | | | SP3_HUMAN.H10MO.B | |
| KIT | rs218259 | A | G | X | ZBTB7A_HUMAN.H10MO.D, |
| | | | | | THA_HUMAN.H10MO.C, |
| | | | | | TFCP2_HUMAN.H10MO.D, |
| | | | | | ZKSC1_HUMAN.H10MO.C, |
| | | | | | Mafb_DBD_3 |
| | rs172629 | C | G | X | RARG_full_1, NR2F6_DBD_1 |
| | rs218264 | A | T | MA0150.2 | X |
| | | | | (NFE2L2), MA0591.1 | |
| TRIM38 | rs9467652 | A | G | X | NRF1_full, ELF3_HUMAN.H10MO.D |
| | rs9467656 | A | G | IRX2_DBD, IRX5_DBD | X |
| HIST1H3A | rs2157050 | G | A | ZN350_HUMAN.H10MO.C | RARA_DBD_2 |
| HIST1H4A | rs9467664 | A | T | IRF1_HUMAN.H10MOA, | ELK1_full_2, FOXG1_DBD_2, |
| | | | | CTCFL_HUMAN.H10MO.A, | Foxk1_DBD_1, |
| | | | | HINFP1_full_1 | FOXK1_DBD, |
| | | | | | SMAD1_HUMAN.H10MO.D |
| HIST1H4B | rs2032449 | G | C | EGR1_HUMAN.H10MO.A, | CTCFL_HUMAN.H10MO.A, |
| | | | | ZN148_HUMAN.H10MO.D | CTCF_HUMAN.H10MO.A |
| | rs3752419 | G | A | TBX20_DBD_3, | X |
| | | | | PAX2_HUMAN.H10MO.D, | |
| | | | | TBX21_HUMAN.H10MO.D, | |
| | | | | TBX4_HUMAN.H10MO.D, | |
| | | | | MGA_DBD_1, | |
| | | | | MAZ_HUMAN.H10MO.A, | |
| | | | | TBR1_DBD, NFIA_full_2, | |
| | | | | TBX21_DBD_2, | |
| | | | | Rarg_DBD_1, TBX2_full_2, | |
| | | | | TBX21_full_1, | |
| | | | | TBR1_HUMAN.H10MO.D, | |
| | | | | TBR1_full, TCF4_full, | |
| | | | | TBX4_DBD_1, | |
| | | | | NFIA_HUMAN.H10MO.S, | |
| | | | | TBX15_DBD_2, EOMES_DBD_1, | |
| | | | | RARA_full_1, SP1_HUMAN.H10MO.C, | |
| | | | | HES7_HUMAN.H10MO.D, | |
| | | | | Hic1_DBD_2, MGA_DBD_2, | |
| | | | | TBX1_HUMAN.H10MO.D, | |
| | | | | TBX21_full_2, TBX1_DBD_3, | |
| | | | | NFIX_full_3, NFIX_full_2, | |
| | | | | TBX5_DBD_1, Rara_DBD_3, | |
| | | | | RARG_DBD_1 | |
| HIST1H2BB | rs2032447 | T | C | RARA_full_2, RARG_full_3, | X |
| | | | | RARA_DBD_1, | |
| | | | | ALX1_HUMAN.H10MO.B | |
| HIST1H3C | rs7756117 | G | A | NKX31_HUMAN.H10MO.C, | NFIL3_HUMAN.H10MO.C, |
| | | | | Foxj3_DBD_1 | FUBP1_HUMAN.H10MO.D, |
| | | | | | FOXH1_HUMAN.H10MO.A |
| HIST1H1C | rs807212 | C | T | MECP2_HUMAN.H10MO.C | X |
| HIST1H4C | rs198853 | C | T | E2F5_HUMAN.H10MO.B | E2F3_DBD_1, |
| | | | | | MAFK_HUMAN.H10MO.S, |
| | | | | | E2F2_DBD_3, E2F3_DBD_2, |
| | | | | | IRF5_HUMAN.H10MO.D, |
| | | | | | IRX3_HUMAN.H10MO.D |
| | rs198851 | T | G | ETS1_HUMAN.H10MO.C, | MEIS3_DBD_1, |
| | | | | FLI1_HUMAN.H10MO.A, | MEIS3_HUMAN.H10MO.D, |
| | | | | CREM_HUMAN.H10MO.C | TGIF1_HUMAN.H10MO.S, |
| | | | | | MEIS2_DBD_2, |
| | | | | | MEIS2_HUMAN.H10MO.B |
| HIST1H2BC | rs198823 | G | T | P63_HUMAN.H10MO.S | X |
| HIST1H2BO | rs13219787 | A | T | TBX5_HUMAN.H10MO.D, | ETV6_full_1 |
| | | | | EOMES_HUMAN.H10MO.D, | |
| | | | | TBX1_DBD_3, TBX4_DBD_1 | |
| USP49 | rs2249703 | A | G | FOXJ3_HUMAN.H10MO.S, | ZNF713_full |
| | | | | Foxj3_DBD_2, | |
| | | | | BPTF_HUMAN.H10MO.D, | |
| | | | | MCR_HUMAN.H10MO.D, | |
| | | | | BCL6_HUMAN.H10MO.C, | |
| | | | | FOXJ2_DBD_1 | |
| MED20 | rs2274578 | C | G | X | X |
| CCND3 | rs1051130 | A | C | SMAD1_HUMAN.H10MO.D | REL_HUMAN.H10MO.C, |
| | | | | | NFYC_HUMAN.H10MO.B, |
| | | | | | PBX3_HUMAN.H10MO.B, |
| | | | | | NFKB2_DBD, |
| | | | | | HEN1_HUMAN.H10MO.C, |
| | | | | | YBOX1_HUMAN.H10MO.D, |
| | | | | | AP2B_HUMAN.H10MO.B, |
| | | | | | RELB_HUMAN.H10MO.C, |
| | | | | | TF65_HUMAN.H10MO.A, |
| | | | | | NFKB1_DBD, |
| | | | | | SRBP2_HUMAN.H10MO.B |
| | rs3218086 | A | G | X | GLI1_HUMAN.H10MO.C, |
| | | | | | GLI3_HUMAN.H10MO.B |
| | rs9349205 | G | A | TFAP4_HUMAN.H10MO.C, | TBX1_HUMAN.H10MO.D |
| | | | | ZN784_HUMAN.H10MO.D | |
| | rs11968166 | A | G | X | HIF1A_HUMAN.H10MO.A, |
| | | | | | HEY2_HUMAN.H10MO.D, |
| | | | | | EPAS1_HUMAN.H10MO.D |
| TAF8 | rs9381120 | T | A | NR6A1_HUMAN.H10MO.B | X |
| | rs9357384 | G | A | TEAD1_HUMAN.H10MO.D, | X |
| | | | | RREB1_HUMAN.H10MO.D, | |
| | | | | TBX2_HUMAN.H10MO.D | |
| | rs4554318 | C | T | HESX1_HUMAN.H10MO.D | X |
| HBS1L | rs13220662 | G | A | X | SOX10_HUMAN.H10MO.D, |
| | | | | | FUBP1_HUMAN.H10MO.D, |
| | | | | | TCF7L2_HUMAN.H10MO.A |
| | rs9376090 | T | C | TFAP2A_DBD_4, | MECP2_HUMAN.H10MO.C |
| | | | | TFAP2C_full_3, | |
| | | | | TFAP2C_DBD_2, | |
| | | | | TFAP2A_DBD_2, | |
| | | | | KLF15_HUMAN.H10MO.D, | |
| | | | | TFAP2B_DBD_2 | |
| | rs2210366 | A | G | NKX28_HUMAN.H10MO.C, | ZN143_HUMAN.H10MO.A |
| | | | | NKX21_HUMAN.H10MO.D, | |
| | | | | PRDM4_HUMAN.H10MO.D | |
| | rs7775698 | C | T | CTCFL_HUMAN.H10MO_A | CDC5L_HUMAN.H10MO.D |
| | rs7776054 | A | G | SOX21_DBD_2, | X |
| | | | | SOX17_HUMAN.H10MO.D, | |
| | | | | SOX9_full_1, SOX7_full_1 | |
| | rs9399137 | T | C | Foxg1_DBD_1, | MLXPL_HUMAN.H10MO.D |
| | | | | FOXA1_HUMAN.H10MO.A, | |
| | | | | FOXC2_DBD_2, | |
| | | | | FOXJ3_DBD_3, | |
| | | | | FOXL1_HUMAN.H10MO.D, | |
| | | | | Foxc1_DBD_1, | |
| | | | | FOXL1_full_2, FOXB1_full, | |
| | | | | FOXO3_HUMAN.H10MO.B, | |
| | | | | FOXC1_DBD_1, | |
| | | | | FOXG1_HUMAN.H10MO.D, | |
| | | | | FOXJ3_HUMAN.H10MO.A, | |
| | | | | FOXJ2_DBD_3 | |
| | rs9389268 | A | G | ZBTB7A_DBD, | RUNX2_DBD_3 |
| | | | | SRBP1_HUMAN.H10MO.B, | |
| | | | | RUNX3_HUMAN.H10MO.C, | |
| | | | | KLF12_HUMAN.H10MO.D, | |
| | | | | KLF8_HUMAN.H10MO.C, | |
| | | | | TBX4_DBD_2 | |
| MYB | rs9376095 | T | C | TBX2_HUMAN.H10MO.D, | Tcfap2a_DBD_1, |
| | | | | EBF1_full, | TCF7F1_HUMAN.H10MO.D, |
| | | | | YBOX1_HUMAN.H10MO.D, | TFAP2C_DBD_1, |
| | | | | COE1_HUMAN.H10MO.A | TFAP2A_DBD_5, |
| | | | | | TFAP2A_DBD_1 |
| | rs210962 | C | T | SP4_HUMAN.H10MO.D, | X |
| | | | | SMAD3_HUMAN.H10MO.C | |
| CITED2 | rs643381 | C | A | AP2A_HUMAN.H10MO.C, | ZN143_HUMAN.H10MO.A, |
| | | | | PLAG1_HUMAN.H10MO.S, | ZIC1_full, |
| | | | | ZIC1_HUMAN.H10MO.B, | Egr1_mouse_DBD_mutant_DBD, |
| | | | | TBX15_HUMAN.H10MO.D, | GLIS3_DBD, |
| | | | | ESRRA_DBD_2, | GLIS1_HUMAN.H10MO.D, |
| | | | | RARA_HUMAN.H10MO.C, | ZIC4_HUMAN.H10MO.D |
| | | | | TBX1_HUMAN.H10MO.D, | |
| | | | | TFAP2C_full_1, | |
| | | | | SP3_HUMAN.H10MO.B, | |
| | | | | EGR4_HUMAN.H10MO.D, | |
| | | | | TFAP2B_DBD_1, | |
| | | | | TFAP2C_DBD_1, | |
| | | | | TFAP2A_DBD_5, | |
| | | | | EGR2_HUMAN.H10MO.C, | |
| | | | | ESRRA_DBD_6, | |
| | | | | TFAP2A_DBD_1, | |
| | | | | SP2_HUMAN.H10MO.C, | |
| | | | | AP2C_HUMAN.H10MO.A, | |
| | | | | COE1_HUMAN.H10MO.A, | |
| | | | | Tcfap2a_DBD_1, | |
| | | | | SRBP2_HUMAN.H10MO.B | |
| | rs592423 | A | C | X | DDIT3_HUMAN.H10MO.C |
| | | | | | (CEBP/Z), |
| | | | | | HLTF_HUMAN.H10MO.D |
| | | | | | (SMARCA3), ZNF410_DBD |
| IKZF1 | rs12718597 | C | A | X | MTF1_HUMAN.H10MO.C |
| | rs12718598 | T | C | X | XBP1_HUMAN.H10MO.C, |
| | | | | | CREB3L1_full_2, |
| | | | | | ATF6A_HUMAN.H10MO.B, |
| | | | | | HINFP1_full_2, |
| | | | | | MAZ_HUMAN.H10MO.A, |
| | | | | | CR3L2_HUMAN.H10MO.D, |
| | | | | | HIF1A_HUMAN.H10MO.A, |
| | | | | | XBP1_DBD_2, CREB3L1_DBD_4, |
| | | | | | SP3_HUMAN.H10MO.B |
| | rs7385935 | A | G | X | KLF1_HUMAN.H10MO.C, |
| | | | | | ZSCA4_HUMAN.H10MO.D, |
| | | | | | PURA_HUMAN.H10MO.D |
| RCL1 | rs13284787 | A | G | POU1F1_DBD_2, | PRGR_HUMAN.H10MO.S, |
| | | | | POU3F2_DBD_2, | GCR_HUMAN.H10MO.S, |
| | | | | POU3F1_DBD_1, | Ar_DBD |
| | | | | POU3F3_DBD_1, | |
| | | | | HSF2_HUMAN.H10MO.A | |
| | rs10758656 | A | G | GATA4_DBD, | X |
| | | | | GATA1_HUMAN.H10MO.A, | |
| | | | | TAL1_HUMAN.H10MO.A, | |
| | | | | GATA5_DBD, | |
| | | | | GATA3_DBD, | |
| | | | | GATA6_HUMAN.H10MO.B, | |
| | | | | GATA1_HUMAN.H10MO.S, | |
| | | | | GATA3_full | |
| | rs10758658 | G | A | Rara_DBD_2, RARG_full_2, | X |
| | | | | RARG_DBD_2, | |
| | | | | Rarg_DBD_2, | |
| | | | | RARA_HUMAN.H10MO.C | |
| MARCH8 | rs10900218 | A | G | IRF7_DBD_1 | ZN713_HUMAN.H10MO.D, |
| | | | | | SMAD2_HUMAN.H10MO.C |
| FNTB | rs11628273 | C | T | HLTF_HUMAN.H10MO.D | X |
| | | | | (SMARCA3) | |
| MAST1 | rs2290689 | A | G | Klf12_DBD | ZNF740_full, |
| | | | | | KLF13_HUMAN.H10MO.D, |
| | | | | | EGR1_HUMAN.H10MO.S, |
| | | | | | ZN219_HUMAN.H10MO.D, |
| | | | | | EGR1_HUMAN.H10MO.A, |
| | | | | | ZN148_HUMAN.H10MO.D, |
| | | | | | KFF16_HUMAN.H10MO.D |
| DNASE2 | rs7249143 | G | T | X | NR4A2_HUMAN.H10MO.C, |
| | | | | | NR2C1_HUMAN.H10MO.C, |
| | | | | | ESRRA_DBD_3, RARG_full_3, |
| | | | | | PPARD_HUMAN.H10MO.D, |
| | | | | | NR4A1_HUMAN.H10MO.C, |
| | | | | | RORA_DBD_1, |
| | | | | | PPARG_HUMAN.H10MO.S, |
| | | | | | Rarb_DBD_3, Rarg_DBD_3, |
| | | | | | RARA_full_3, RXRB_DBD, |
| | | | | | Nr2f6_DBD_2, NR2F6_full, |
| | | | | | NR2F1_DBD_3, ESRRG_full_1, |
| | | | | | PPARG_HUMAN.H10MO.A, |
| | | | | | Rara_DBD_1, RXRA_full_1, |
| | | | | | NR4A2_full_3, |
| | | | | | SOX18_HUMAN.H10MO.D, |
| | | | | | RARB_HUMAN.H10MO.D, |
| | | | | | NR2F1_DBD_1, RARG_full_2, |
| | | | | | NR2C2_HUMAN.H10MO.A, |
| | | | | | NR2E3_HUMAN.H10MO.C, |
| | | | | | ESRRG_full_3, |
| GCDH | rs11085824 | A | G | X | Rarb_DBD_2, Esrra_DBD_1, |
| | | | | | ESRRA_DBD_2, Vdr_DBD, |
| | | | | | ESRRG_full_2, VDR_full, |
| | | | | | RARA_DBD_1, ESRRA_DBD_5 |
| | rs2242517 | T | G | GLIS3_DBD, ZBTB7B_full, | Egr1_mouse_DBD_mutant_DBD, |
| | | | | GLIS_1_HUMAN.H10MO.D | WT1_HUMAN.H10MO.D, |
| | | | | | KLF1_HUMAN.H10MO.C, |
| | | | | | KLF12_HUMAN.H10MO.D, |
| | | | | | KLF16_DBD, |
| | | | | | MAZ_HUMAN.H10MO.A, |
| | | | | | ASCL2_HUMAN.H10MO.D, |
| | | | | | ZIC2_HUMAN.H10MO.C, |
| | | | | | ZIC1_HUMAN.H10MO.B, |
| | | | | | FOXK1_HUMAN.H10MO.D, |
| | | | | | SPIC_HUMAN.H10MO.D, |
| | | | | | SP4_HUMAN.H10MO.D, |
| | | | | | ZN219_HUMAN.H10MO.D, |
| | | | | | EGR1_HUMAN.H10MO.A, |
| | | | | | ARNT2_HUMAN.H10MO.D, |
| | | | | | SP1_HUMAN.H10MO.C, |
| | | | | | SP2_HUMAN.H10MO.C, |
| | | | | | GLIS2_HUMAN.H10MO.D, |
| | | | | | KLF3_HUMAN.H10MO.D, |
| | | | | | EGR3_DBD, FOXO1_DBD_3, |
| | | | | | SP3_DBD, |
| | | | | | CLOCK_HUMAN.H10MO.D |
| FARSA | rs2965214 | G | A | GSC2_HUMAN.H10MO.D, | RARG_full_3 |
| | | | | E2F6_HUMAN.H10MO.C, | |
| | | | | ZN639_HUMAN.H10MO.D, | |
| | | | | PLAG1_HUMAN.H10MO.D, | |
| | | | | SPIC_HUMAN.H10MO.D, | |
| | | | | TBX15_HUMAN.H10MO.D, | |
| | | | | MAZ_HUMAN.H10MO.A | |
| CALR | rs1010222 | A | G | NR2E1_HUMAN.H10MO.D | TBX15_DBD_1, |
| | | | | | TBX20_DBD_2, |
| | | | | | TBX1_DBD_2, |
| | | | | | HEY2_HUMAN.H10MO.D |
| ODF3B | rs140521 | C | A | XBP1_DBD_1, | LHX2_DBD_2, |
| | | | | THB_HUMAN.H10MO.C, | VSX1_HUMAN.H10MO.D, |
| | | | | GMEB2_HUMAN.H10MO.D | DLX4_HUMAN.H10MO.D, |
| | | | | | MEOX2_DBD_2, |
| | | | | | SRF_HUMAN.H10MO.A, |
| | | | | | DLX6_HUMAN.H10MO.D, |
| | | | | | DLX1_HUMAN.H10MO.D |
| NRG4 | rs4886755 | A | G | POU1F1_DBD_2, | IRF5_HUMAN.H10MO.D, |
| | | | | PRDM1_full, | FOXJ2_DBD_3 |
| | | | | PRDM1_HUMAN.H10MO.C, | |
| | | | | STAT2_HUMAN.H10MO.B | |
| SLC12A7 | rs4535497 | C | A | PO5F1_HUMAN.H10MO.A, | NR4A2_full_2, |
| | | | | SOX2_HUMAN.H10MO.B, | GFI1B_HUMAN.H10MO.C, |
| | | | | NANOG_HUMAN.H10MO.A | BCL6B_DBD, |
| | | | | | HNF4A_HUMAN.H10MO.A, |
| | | | | | GFI1_HUMAN.H10MO.C |
| ANK1 | rs4737009 | G | A | X | SRF_HUMAN.H10MO.A |
| THRB | rs1505307 | T | C | SNAI2_HUMAN.H10MO.C, | X |
| | | | | TFE3_HUMAN.H10MO.C, | |
| | | | | ARNTL_DBD | |
| RBPMS | rs2979489 | G | A | ESRRG_full_1, | NR4A2_HUMAN.H10MO.C, |
| | | | | NR1D1_HUMAN.H10MO.C | RXRG_HUMAN.H10MO.B, |
| | | | | | MEF2B_HUMAN.H10MO.D, |
| | | | | | NR4A2_full_3, |
| | | | | | PPARG_HUMAN.H10MO.S, |
| | | | | | Rarb_DBD_1, Foxj3_DBD_4, |
| | | | | | FOXJ2_DBD_3, MEF2A_DBD, |
| | | | | | CDX2_HUMAN.H10MO.C, |
| | | | | | NR2F1_DBD_3 |
| NCOA4 | rs17720193 | T | C | Esrra_DBD_2, | GRHL1_DBD_1 |
| | | | | ERR3_HUMAN.H10MO.B, | |
| | | | | ESRRG_full_1, | |
| | | | | NR6A1_HUMAN.H10MO.B | |
| RBM38 | rs737092 | T | C | X | NKX23_HUMAN.H10MO.D, |
| | | | | | NKX3-1_full, Nkx3-1_DBD, |
| | | | | | SMAD3_HUMAN.H10MO.C, |
| | | | | | MAFG_HUMAN.H10MO.C, |
| | | | | | NKX3-2_DBD |
| ACTL6B | rs2075672 | A | G | ETV7_HUMAN.H10MO.D | SRF_HUMAN.H10MO.A, |
| | | | | | FLI1_HUMANH.10MO.A, |
| | | | | | ELF2_HUMAN.H10MO.C, |
| | | | | | ELF1_HUMAN.H10MO.A |
| SH2B3 | rs3184504 | C | T | MBD2_HUMAN.H10MO.B | FLI1_DBD_2 |
| MPST | rs135167 | A | G | X | TCF4_DBD, MSC_full, |
| | | | | | NHLH1_full, |
| | | | | | HTF4_HUMAN.H10MO.B |
| BRAP | rs11065987 | A | G | X | VDR_full |
| MOSPD3 | rs12532878 | A | G | X | HIC2_DBD, |
| | | | | | HIC2_HUMAN.H10MO.D, |
| OR51V1 | rs140522 | A | G | X | X |

Sequences

LOCUS AAA36737; 513 aa linear PRI 13 Apr. 2001. DEFINITION-transforming growth factor-beta BMP protein [Homo sapiens], e.g., ACCESSION AAA36737, VERSION AAA36737.1.

(SEQ ID NO: 12)
MPGLGRRAQW LCWWWGLLCS CCGPPPLRPP LPAAAAAAAG
GQLLGDGGSP GRTEQPPPSP QSSSGFLYRR LKTQEKREMQ
KEILSVLGLP HRPRPLHGLQ QPQPPALRQQ EEQQQQQQLP
RGEPPPGRLK SAPLFMLDLY NALSADNDED GASEGERQQS
WPHEAASSSQ RRQPPPGAAH PLNRKSLLAP GSGSGGASPL
TSAQDSAFLN DADMVMSFVN LVEYDKEFSP RQRHHKEFKF
NLSQIPEGEV VTAAEFRIYK DCVMGSFKNQ TFLISIYQVL
QEHQHRDSDL FLLDTRVVWA SEEGWLEFDI TATSNLWVVT
PQHNMGLQLS VVTRDGVHVH PRAAGLVGRD GPYDKQPFMV
AFFKVSEVHV RTTRSASSRR RQQSRNRSTQ SQDVARVSSA
SDYNSSELKT ACRKHELYVS FQDLGWQDWI IAPKGYAANY
CDGECSFPLN AHMNATNHAI VQTLVHLMNP EYVPKPCCAP
TKLNAISVLY FDDNSNVILK KYRNMVVRAC GCH

LOCUS NP_001334843; 408 aa linear PRI 9 Apr. 2017. DEFINITION bone morphogenetic protein 4 isoform a preproprotein [Homo sapiens], e.g., ACCESSION NP_001334843, VERSION NP_001334843.1. DBSOURCE REFSEQ: accession NM_001347914.

(SEQ ID NO: 13)
MIPGNRMLMV VLLCQVLLGG ASHASLIPET GKKKVAEIQG
HAGGRRSGQS HELLRDFEAT LLQMFGLRRR PQPSKSAVIP
DYMRDLYRLQ SGEEEEEQIH STGLEYPERP ASRANTVRSF
HHEEHLENIP GTSENSAFRF LFNLSSIPEN EVISSAELRL
FREQVDQGPD WERGFHRINI YEVMKPPAEV VPGHLITRLL
DTRLVHHNVT RWETFDVSPA VLRWTREKQP NYGLAIEVTH
LHQTRTHQGQ HVRISRSLPQ GSGNWAQLRP LLVTFGHDGR
GHALTRRRRA KRSPKHHSQR ARKKNKNCRR HSLYVDFSDV
GWNDWIVAPP GYQAFYCHGD CPFPLADHLN STNHAIVQTL
VNSVNSSIPK ACCVPTELSA ISMLYLDEYD KVVLKNYQEM
VVEGCGCR.

SEQ ID NO: 13 is, e.g., Isoform Accessions of BMP4: NP_{—} 001334841.1; Accession: NP 001334844.1; Accession: NP 001334842.1; Accession: NP 001334843.1; Accession: NP 001334845.1; Accession: AMM63596; Accession: AMM45324.1 (partial); Accession: AMM45323.

1. A method for modulating erythropoiesis comprising contacting a CD34⁺ cell with an agent that alters occupancy at a signaling center in the genome of the cell, wherein the signaling center comprises
a DNA binding site for a lineage-specific regulator; and
a DNA binding site for a signal-responsive transcription factor, wherein increasing gene expression at the signaling center promotes erythropoiesis.

2. The method of claim 1, wherein the signaling center further comprises a tissue-specific transcription factor DNA binding motif.

3. The method of claim 1, wherein the agent that alters occupancy at the signaling center is an agent that induces or inhibits binding of the signal-responsive transcription factor to the signaling center.

4. (canceled)

5. The method of claim 1, wherein the signal-responsive transcription factor is selected from the group consisting of SMAD1, SMAD5, SMAD8, β-catenin, LEF/TCF, STAT5, RARA, BCL11A, TCF7L2, CREB3L, CREB, CREM, CTCF, IRF7, RELB, AP2B, NFKB2, PAX, PPARG, RXRA, RARG, RARB, E2F6m TBX20, TBX1, NFIA, NFIB, ZN350, TCF4, EGR1, and THRB.

6. The method of claim 1, wherein the agent that alters occupancy at the signaling center in the genome is an agonist of a signaling pathway selected from the group consisting of: nuclear hormone receptor, cAMP pathway, MAPK pathway, JAK-STAT pathway, NFKB pathway, Wnt pathway, TGFβ/BMP pathway, LIF pathway, BDNF pathway, PGE2 pathway, and NOTCH pathway.

7. (canceled)

8. The method of claim 1, wherein the lineage-specific regulator is the transcription factor GATA1 or GATA2.

9. The method of claim 1, wherein the signaling center comprises:
a. a signal-responsive binding site for transcription factor SMAD1 and a lineage-specific regulator binding site for the transcription factor GATA1, and wherein the agent that alters occupancy at the signaling center increases expression of one or more genes selected from Table 4, or from Lengthy Table S6; or
b. a signal-responsive transcription factor binding site for SMAD1 and a lineage-specific regulator binding site for the transcription factor GATA2, and wherein the agent that alters occupancy at the signaling center increases expression of one or more genes selected from Table 3, or from Lengthy Table S6

10. (canceled)

11. The method of claim 9, wherein the agent that alters occupancy at the signaling center signaling center is an agent that activates the transcription factor SMAD1.

12. (canceled)

13. (canceled)

14. The method of claim 11, wherein the agent that activates SMAD1 is selected from the group consisting of: PD407824, MK-8776, LY-2606368 and LY-2603618, BMP4, BMP2, BMP7, isoliquirtigenin, apigenin, 4′-hydroxychalcone, a checkpoint kinase 1 (CHK1) inhibitor, an agonist of a BMP receptor kinase, and diosmetin.

15. The method of claim 1, wherein the signaling center comprises the signal-responsive binding site for transcription factor SMAD1 and the lineage-specific regulator binding site for the transcription factor GATA1 or GATA2, and wherein co-binding of either SMAD1/GATA1 or SMAD/GATA2 at the signaling center alters expression of long non-coding RNAs (IncRNAS) from Lengthy Table S5.

16. The method of claim 1, wherein the CD34⁺ cell is derived from a source selected from the group consisting of: bone marrow, peripheral blood, cord blood and derived from induced pluripotent stem cells.

17. (canceled)

18. A method for treating a disease associated with aberrant erythropoiesis comprising correcting the DNA of a CD34⁺ cell that is present at the site of a signaling center, wherein the signaling center associated with normal erythropoiesis comprises
a DNA binding site for a lineage-specific regulator; and
a DNA binding site for a signal-responsive transcription factor.

19. The method of claim 18, wherein the correction of the DNA restores the binding of the signal-responsive transcription factor to the signaling center, or restores binding of the native signal-responsive transcription factor to the signaling center restoring wild-type expression of one or more genes selected from Table 5 or Table 6.

20. The method of claim 18, wherein the lineage-specific regulator is transcription factor GATA1 or GATA2.

21. The method of claim 18, wherein the signal-responsive transcription factor is selected from the group consisting of SMAD1, SMAD5, SMAD8, β-catenin, LEF/TCF, STAT5, RARA, BCL11A, TCF7L2, CREB3L, CREB, CREM, CTCF, IRF7, RELB, AP2B, NFKB2, PAX, PPARG, RXRA, RARG, RARB, E2F6m TBX20, TBX1, NFIA, NFIB, ZN350, TCF4, EGR1, and THRB.

22. The method of claim 18, wherein the signaling center further comprises a tissue-specific transcription factor DNA binding motif.

23. The method of claim 18, wherein the DNA is corrected using a gene editing tool.

24. (canceled)

25. The method of claim 18, wherein the disease associated with aberrant erythropoiesis is selected from the group consisting of: leukemia, lymphoma, inherited anemia, inborn errors of metabolism, aplastic anemia, beta-thalassemia, Blackfan-Diamond syndrome, globoid cell leukodystrophy, sickle cell anemia, severe combined immunodeficiency, X-linked lymphoproliferative syndrome, Wiskott-Aldrich syndrome, Hunter's syndrome, Hurler's syndrome Lesch Nyhan syndrome, osteopetrosis, chemotherapy rescue of the immune system, and an autoimmune disease.

26. The method of claim 18, wherein the signal-responsive binding site is the binding site for the transcription factor SMAD1, and wherein restoring binding of SMAD1 to the signaling center increases expression of one or more genes selected from Table 4, from Table 3, or from Lengthy Table S6.

27.-30. (canceled)

31. The method of claim 18, wherein the CD34⁺ cell is transplanted into the subject after correction of the DNA at the site of the signaling center.
